(19) United States
(12) Patent Application Publication
(10) Pub. No.: US 2023/0146354 A1
(43) Pub. Date: May 11, 2023

(54) LIGHT-EMITTING DEVICE, LIGHT-EMITTING APPARATUS, ORGANIC COMPOUND, ELECTRONIC APPLIANCE, AND LIGHTING DEVICE

(71) Applicant: SEMICONDUCTOR ENERGY LABORATORY CO., LTD., Atsugi-shi (JP)

(72) Inventors: Sachiko KAWAKAMI, Atsugi (JP); Kaori OGITA, Isehara (JP); Nozomi KOMATSU, Atsugi (JP); Tsunenori SUZUKI, Yokohama (JP); Naoaki HASHIMOTO, Sagamihara (JP); Satoshi SEO, Sagamihara (JP); Kyoko TAKEDA, Atsugi (JP)

(21) Appl. No.: 17/819,429

(22) Filed: Aug. 12, 2022

(30) Foreign Application Priority Data
Aug. 20, 2021 (JP) 2021-134705
Jan. 28, 2022 (JP) 2022-011987

(51) Int. Cl. H01L 51/00

(52) U.S. Cl. CPC H01L 51/0074; H01L 51/0073; H01L 51/5016

(57) ABSTRACT

A light-emitting device including at least a light-emitting layer between an anode and a cathode; the light-emitting layer contains at least a light-emitting substance; the light-emitting substance is a substance emitting fluorescent light; a first organic compound represented by General Formula (G1) is included between the anode and the cathode;

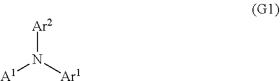

in General Formula (G1), Ar¹ represents a substituted or unsubstituted fluorenyl group, Ar² represents a substituted or unsubstituted aryl group having 6 to 13 carbon atoms, and A¹ represents a substituted or unsubstituted dibenzofuranyl group or a substituted or unsubstituted dibenzothiophenyl group; when at least one of Ar¹, Ar², and A¹ has one or more substituents, the substituents each independently represent an alkyl group having 1 to 4 carbon atoms or an aryl group having 6 to 13 carbon atoms. The aryl group exclude a heteroaryl group. The substituents may be bonded to each other to form a ring.

### BACKGROUND OF THE INVENTION

### 1. Field of the Invention

One embodiment of the present invention relates to an organic compound. One embodiment of the present invention further relates to a light-emitting device, a light-emitting apparatus, an electronic appliance, and a lighting device each of which uses the organic compound. Note that one embodiment of the present invention is not limited to the above technical field. The technical field of one embodiment of the present invention disclosed in this specification and the like relates to an object, a method, or a manufacturing method. One embodiment of the present invention relates to a process, a machine, manufacture, or a composition of matter. Specifically, examples of the technical field of one embodiment of the present invention disclosed in this specification include a semiconductor device, a display device, a liquid-crystal device, a light-emitting apparatus, a lighting device, a power storage device, a memory device, an imaging device, a driving method thereof, and a manufacturing method thereof.

### 2. Description of the Related Art

In recent years, research and development have been extensively conducted on light-emitting devices (also referred to as light-emitting elements) using electroluminescence (EL). In the basic structure of such a light-emitting device, a layer containing a light-emitting substance is interposed between a pair of electrodes. By voltage application to this device, light emission from the light-emitting substance can be obtained.

The light-emitting device is a self-luminous device and thus has advantages over a liquid crystal display, such as high visibility of the pixels and no need of backlight, and is considered to be suitable as a flat panel display element. Another major advantage of the light-emitting device is that it can be fabricated to be thin and lightweight. Moreover, such light-emitting devices also have a feature that response speed is extremely fast.

Furthermore, since the light-emitting device can be formed in a film form, it is possible to provide planar light emission; thus, a large-area element utilizing planar light emission can be easily formed. Thus, a large-area device having a planar emission surface can be easily formed. This feature is difficult to achieve with point light sources typified by incandescent lamps and LEDs or linear light sources typified by fluorescent lamps; thus, the light-emitting devices also have great potential as planar light sources, which can be used for lighting devices and the like.

Such light-emitting devices utilizing electroluminescence can be broadly classified according to whether a light-emitting substance is an organic compound or an inorganic compound. In the case of an organic EL device in which a layer containing an organic compound used as a light-emitting substance is provided between a pair of electrodes, application of voltage to the device causes injection of electrons from a cathode and holes from an anode into the layer containing the organic compound having a light-emitting property and thus current flows. The injected electrons and holes then lead the organic compound to its excited state, whereby light emission is obtained from the excited organic compound.

The excited state formed by an organic compound can be a singlet excited state (S*) or a triplet excited state (T*). Light emission from the singlet excited state is called fluorescence, and light emission from the triplet excited state is called phosphorescence.

In improving device characteristics of the light-emitting device, there are many problems which depend on a substance, and in order to solve the problems, improvement of a device structure, development of a substance, and the like have been carried out. For example, Patent Document 1 discloses a carbazole derivative having a high hole-transport property as an organic compound that can be used for forming a light-emitting device with high emission efficiency.

### REFERENCE

[Patent Document 1] Japanese Published Patent Application No. 2009-298767

### SUMMARY OF THE INVENTION

As described above, for the improvement of the characteristics of a light-emitting device, it is necessary to develop an organic compound with characteristics suitable for a light-emitting device. An object of one embodiment of the present invention is to provide a novel organic compound having a hole-transport property and a low highest occupied molecular orbital (HOMO) level. Another object is to provide a light-emitting device with high emission efficiency by using a novel organic compound of one embodiment of the present invention. Another object is to provide a fluorescent light-emitting device with high emission efficiency by using a novel organic compound of one embodiment of the present invention. In addition, another object is to provide a light-emitting device with low power consumption.

One embodiment of the present invention is a light-emitting device including at least a light-emitting layer between an anode and a cathode. In the light-emitting device, the light-emitting layer contains at least a light-emitting substance, the light-emitting substance is a substance emitting fluorescent light, and a first organic compound represented by General Formula (G1) is included between the anode and the cathode.

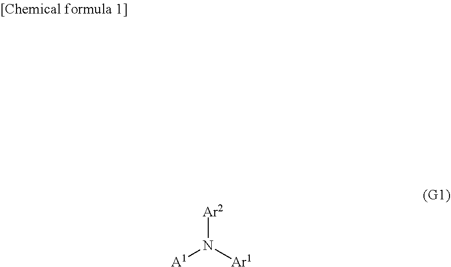

In General Formula (G1) above, Ar¹ represents a substituted or unsubstituted fluorenyl group, Ar² represents a substituted or unsubstituted aryl group having 6 to 13 carbon atoms, and A¹ represents a substituted or unsubstituted dibenzofuranyl group or a substituted or unsubstituted dibenzothiophenyl group. Note that when at least one of Ar¹, Ar², and A¹ has one or more substituents, the substituents each independently represent an alkyl group having 1 to 4 carbon atoms or an aryl group having 6 to 13 carbon atoms. The aryl group exclude a heteroaryl group. The substituents may be bonded to each other to form a ring.

Another embodiment of the present invention is a light-emitting device including at least a light-emitting layer between an anode and a cathode. In the light-emitting device, the light-emitting layer contains at least a light-emitting substance, the light-emitting substance is a substance emitting fluorescent light, and a first organic compound represented by General Formula (G2) is included between the anode and the cathode.

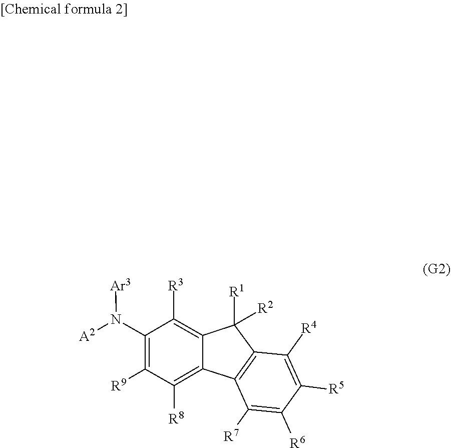

In General Formula (G2), Ar³ represents a substituted or unsubstituted aryl group having 6 to 13 carbon atoms, A² represents a substituted or unsubstituted dibenzofuranyl group or a substituted or unsubstituted dibenzothiophenyl group, and R¹ to R⁹ each independently represent hydrogen (including deuterium), an alkyl group having 1 to 4 carbon atoms, or an aryl group having 6 to 13 carbon atoms. When one or both of Ar³ and A² have one or more substituents, the substituents each independently represent an alkyl group having 1 to 4 carbon atoms or an aryl group having 6 to 13 carbon atoms. The aryl group exclude a heteroaryl group. The substituents may be bonded to each other to form a ring.

Another embodiment of the present invention is a light-emitting device including at least a light-emitting layer between an anode and a cathode. In the light-emitting device, the light-emitting layer contains at least a light-emitting substance, the light-emitting substance is a substance emitting fluorescent light, and a first organic compound represented by General Formula (G3) is included between the anode and the cathode.

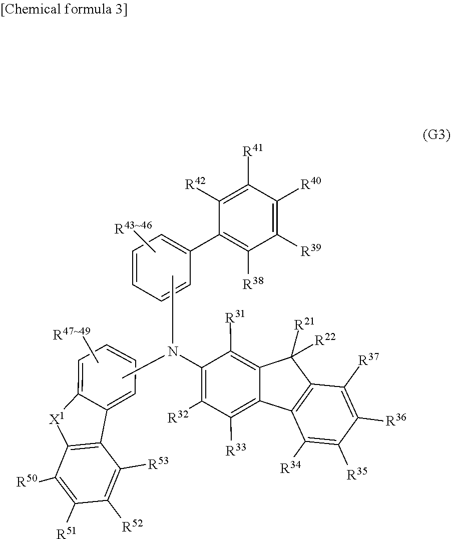

In General Formula (G3), X¹ represents oxygen or sulfur, R²¹, R²², and R³¹ to R³⁷ each independently represent hydrogen (including deuterium), an alkyl group having 1 to 4 carbon atoms, or an aryl group having 6 to 13 carbon atoms, R³⁸ to R⁴⁶ each independently represent hydrogen (including deuterium), an alkyl group having 1 to 4 carbon atoms, or an aryl group having 6 to 13 carbon atoms, R⁴⁷ to R⁵³ each independently represent hydrogen (including deuterium), an alkyl group having 1 to 4 carbon atoms, or an aryl group having 6 to 13 carbon atoms. Note that the aryl group exclude a heteroaryl group. At least two of the substituents represented by R²¹, R²², and R³¹ to R³⁷ may be bonded to each other to form a ring, at least two of the substituents represented by R³⁸ to R⁴⁶ may be bonded to each other to form a ring, and at least two of the substituents represented by R⁴⁷ to R⁵³ may be bonded to each other to form a ring.

Another embodiment of the present invention is a light-emitting device including at least a light-emitting layer between an anode and a cathode. In the light-emitting device, the light-emitting layer contains a light-emitting substance and a first organic compound, the light-emitting substance is a substance emitting fluorescent light, and the first organic compound is represented by General Formula (G1).

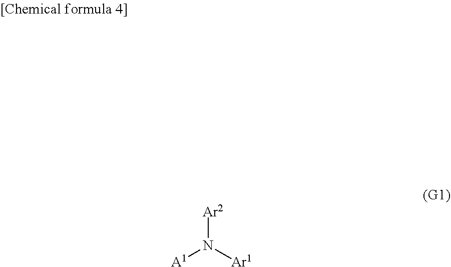

In General Formula (G1) above, Ar¹ represents a substituted or unsubstituted fluorenyl group, Ar² represents a substituted or unsubstituted aryl group having 6 to 13 carbon atoms, and A¹ represents a substituted or unsubstituted dibenzofuranyl group or a substituted or unsubstituted dibenzothiophenyl group. Note that when at least one of Ar¹, Ar², and A¹ has one or more substituents, the substituents each independently represent an alkyl group having 1 to 4 carbon atoms or an aryl group having 6 to 13 carbon atoms. The aryl group exclude a heteroaryl group. The substituents may be bonded to each other to form a ring.

Another embodiment of the present invention is a light-emitting device including at least a light-emitting layer between an anode and a cathode. In the light-emitting device, the light-emitting layer contains a light-emitting substance and a first organic compound, the light-emitting substance is a substance emitting fluorescent light, and the first organic compound is represented by General Formula (G2).

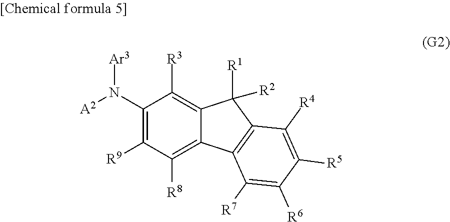

In General Formula (G2), Ar³ represents a substituted or unsubstituted aryl group having 6 to 13 carbon atoms, A² represents a substituted or unsubstituted dibenzofuranyl group or a substituted or unsubstituted dibenzothiophenyl group, and R¹ to R⁹ each independently represent hydrogen (including deuterium), an alkyl group having 1 to 4 carbon atoms, or an aryl group having 6 to 13 carbon atoms. When one or both of Ar³ and A² have one or more substituents, the substituents each independently represent an alkyl group having 1 to 4 carbon atoms or an aryl group having 6 to 13 carbon atoms. The aryl group exclude a heteroaryl group. The substituents may be bonded to each other to form a ring.

Another embodiment of the present invention is a light-emitting device including at least a light-emitting layer between an anode and a cathode. In the light-emitting device, the light-emitting layer contains a light-emitting substance and a first organic compound, the light-emitting substance is a substance emitting fluorescent light, and the first organic compound is represented by General Formula (G3).

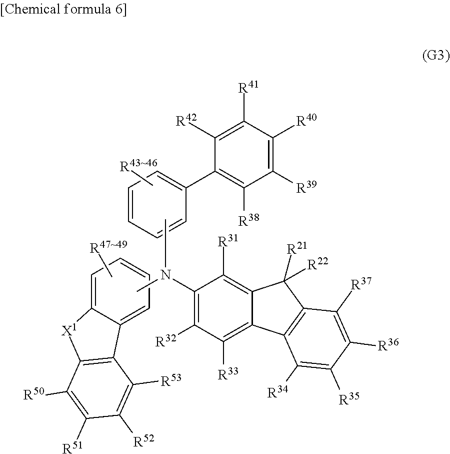

In General Formula (G3), X¹ represents oxygen or sulfur, R²¹, R²², and R³¹ to R³⁷ each independently represent hydrogen (including deuterium), an alkyl group having 1 to 4 carbon atoms, or an aryl group having 6 to 13 carbon atoms, R³⁸ to R⁴⁶ each independently represent hydrogen (including deuterium), an alkyl group having 1 to 4 carbon atoms, or an aryl group having 6 to 13 carbon atoms, R⁴⁷ to R⁵³ each independently represent hydrogen (including deuterium), an alkyl group having 1 to 4 carbon atoms, or an aryl group having 6 to 13 carbon atoms. Note that the aryl group exclude a heteroaryl group. At least two of the substituents represented by R²¹, R²², and R³¹ to R³⁷ may be bonded to each other to form a ring, at least two of the substituents represented by R³⁸ to R⁴⁶ may be bonded to each other to form a ring, and at least two of the substituents represented by R⁴⁷ to R⁵³ may be bonded to each other to form a ring.

Another embodiment of the present invention is a light-emitting device including at least a light-emitting layer between an anode and a cathode, and a first layer between the light-emitting layer and the anode. In the light-emitting device, the light-emitting layer contains a light-emitting substance, the light-emitting substance is a substance emitting fluorescent light, and the first layer contains a first organic compound represented by General Formula (G1).

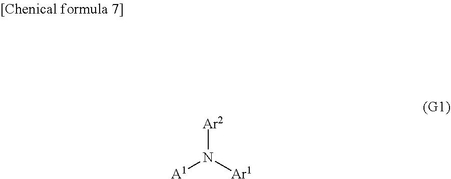

In General Formula (G1) above, Ar¹ represents a substituted or unsubstituted fluorenyl group, Ar² represents a substituted or unsubstituted aryl group having 6 to 13 carbon atoms, and A¹ represents a substituted or unsubstituted dibenzofuranyl group or a substituted or unsubstituted dibenzothiophenyl group. Note that when at least one of Ar¹, Ar², and A¹ has one or more substituents, the substituents each independently represent an alkyl group having 1 to 4 carbon atoms or an aryl group having 6 to 13 carbon atoms. The aryl group exclude a heteroaryl group. The substituents may be bonded to each other to form a ring.

Another embodiment of the present invention is a light-emitting device including at least a light-emitting layer between an anode and a cathode, and a first layer between the light-emitting layer and the anode. In the light-emitting device, the light-emitting layer contains a light-emitting substance, the light-emitting substance is a substance emitting fluorescent light, and the first layer contains a first organic compound represented by General Formula (G2).

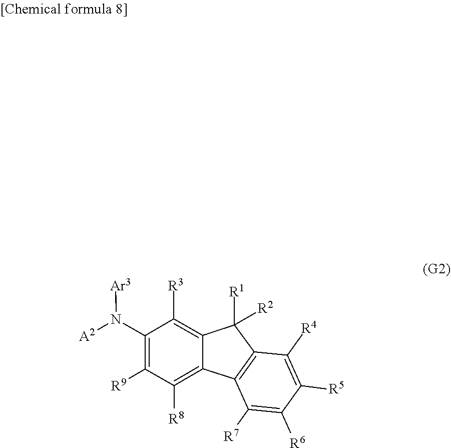

In General Formula (G2), Ar³ represents a substituted or unsubstituted aryl group having 6 to 13 carbon atoms, A² represents a substituted or unsubstituted dibenzofuranyl group or a substituted or unsubstituted dibenzothiophenyl group, and R¹ to R⁹ each independently represent hydrogen (including deuterium), an alkyl group having 1 to 4 carbon atoms, or an aryl group having 6 to 13 carbon atoms. In addition, when one or both of Ar³ and A² have one or more substituents, the substituents each independently represent an alkyl group having 1 to 4 carbon atoms or an aryl group having 6 to 13 carbon atoms. The aryl group exclude a heteroaryl group. The substituents may be bonded to each other to form a ring.

Another embodiment of the present invention is a light-emitting device including at least a light-emitting layer between an anode and a cathode, and a first layer between the light-emitting layer and the anode. In the light-emitting device, the light-emitting layer contains a light-emitting substance, the light-emitting substance is a substance emitting fluorescent light, and the first layer contains a first organic compound represented by General Formula (G3).

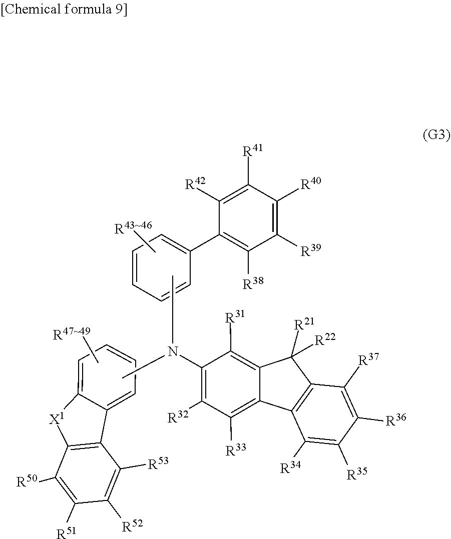

In General Formula (G3), X¹ represents oxygen or sulfur, R²¹, R²², and R³¹ to R³⁷ each independently represent hydrogen (including deuterium), an alkyl group having 1 to 4 carbon atoms, or an aryl group having 6 to 13 carbon atoms, R³⁸ to R⁴⁶ represent hydrogen (including deuterium), an alkyl group having 1 to 4 carbon atoms, or an aryl group having 6 to 13 carbon atoms, R⁴⁷ to R⁵³ each independently represent hydrogen (including deuterium), an alkyl group having 1 to 4 carbon atoms, or an aryl group having 6 to 13 carbon atoms. Note that the aryl group exclude a heteroaryl group. At least two of the substituents represented by R²¹, R²², and R³¹ to R³⁷ may be bonded to each other to form a ring, at least two of the substituents represented by R³⁸ to R⁴⁶ may be bonded to each other to form a ring, and at least two of the substituents represented by R⁴⁷ to R⁵³ may be bonded to each other to form a ring.

Another embodiment of the present invention is any one of the above-described light-emitting devices in which the first layer is in contact with the light-emitting layer.

Another embodiment of the present invention is any one of the above-described light-emitting devices in which the first layer is in contact with the anode.

Another embodiment of the present invention is any one of the above-described light-emitting devices in which the first organic compound is represented by any one of Structural Formulae (100), (101), (105), (136), (200), and (400).

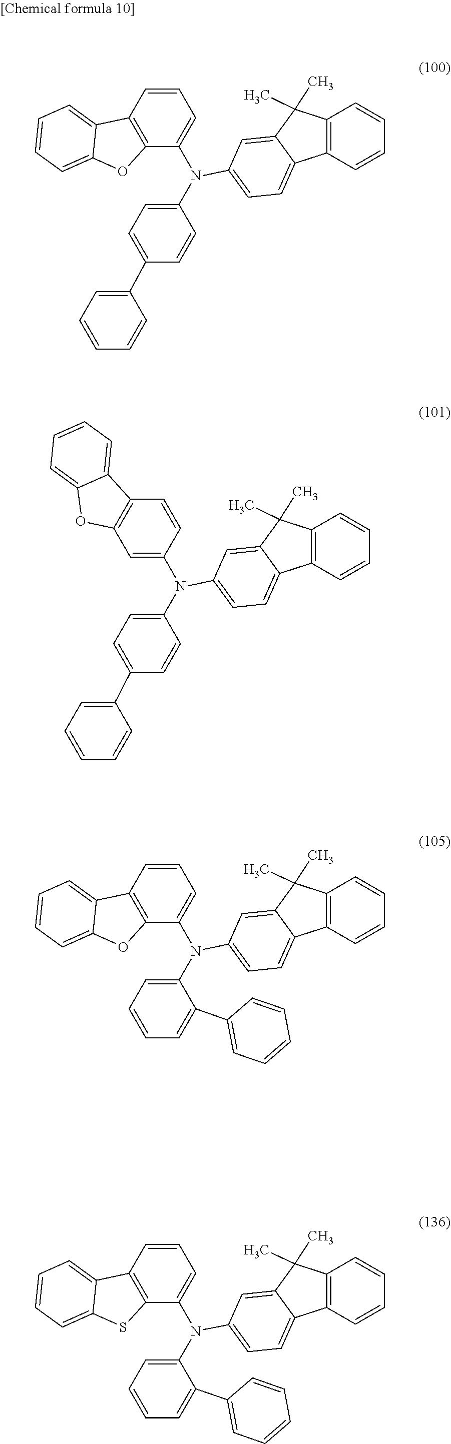 [SMILES: CC1(C)c2ccccc2-c2ccc(N(c3ccc(-c4ccccc4)cc3)c3cccc4c3oc3ccccc34)cc21 ; CC1(C)c2ccccc2-c2ccc(N(c3ccc(-c4ccccc4)cc3)c3ccc4c(c3)oc3ccccc34)cc21 ; CC1(C)c2ccccc2-c2ccc(N(c3ccccc3-c3ccccc3)c3cccc4c3oc3ccccc34)cc21 ; CC1(C)c2ccccc2-c2ccc(N(c3ccccc3-c3ccccc3)c3cccc4c3sc3ccccc34)cc21]

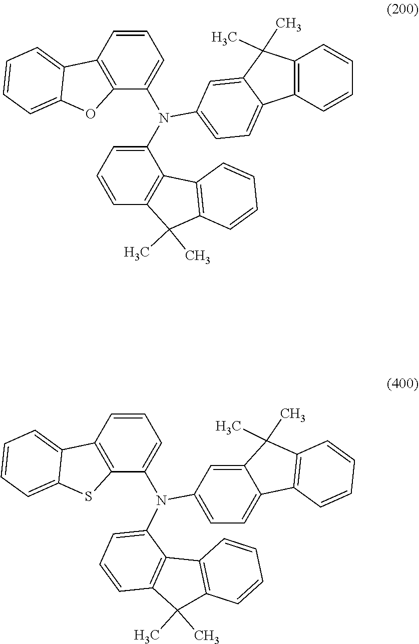 [SMILES: CC1(C)c2ccccc2-c2ccc(N(c3cccc4c3-c3ccccc3C4(C)C)c3cccc4c3oc3ccccc34)cc21 ; CC1(C)c2ccccc2-c2ccc(N(c3cccc4c3-c3ccccc3C4(C)C)c3cccc4c3sc3ccccc34)cc21]

Another embodiment of the present invention is any one of the above-described light-emitting devices in which the difference between the lowest singlet excited level and the lowest triplet excited level of the light-emitting substance is 0.3 eV or more.

Another embodiment of the present invention is any one of the above-described light-emitting devices in which the light-emitting substance is a substance emitting blue light.

Another embodiment of the present invention is a light-emitting apparatus including the light-emitting device according to any one of the above structures and a transistor or a substrate.

Another embodiment of the present invention is a light-emitting apparatus including the light-emitting device according to any one of the above structures and a light-receiving device, in which the light-receiving device includes a light-receiving layer between a first electrode and a second electrode, and the light-receiving layer contains the first organic compound.

Another embodiment of the present invention is a light-emitting apparatus including the light-emitting device according to any one of the above structures and a light-receiving device, in which the light-receiving device includes a light-receiving layer between a first electrode and a second electrode, the light-receiving layer includes at least an active layer, and the active layer contains the first organic compound.

Another embodiment of the present invention is a light-emitting apparatus including the light-emitting device according to any one of the above structures and a light-receiving device, in which the light-receiving device includes a light-receiving layer between a first electrode and a second electrode, the light-receiving layer includes at least an active layer, and a second layer between the active layer and the first electrode, and the second layer contains the first organic compound.

Another embodiment of the present invention is a light-emitting apparatus including the light-emitting device according to any one of the above structures and a light-receiving device, in which the light-receiving device includes a light-receiving layer between a first electrode and a second electrode, and the light-receiving layer includes at least an active layer, and the first layer between the active layer and the first electrode.

Another embodiment of the present invention is an organic compound represented by General Formula (G11).

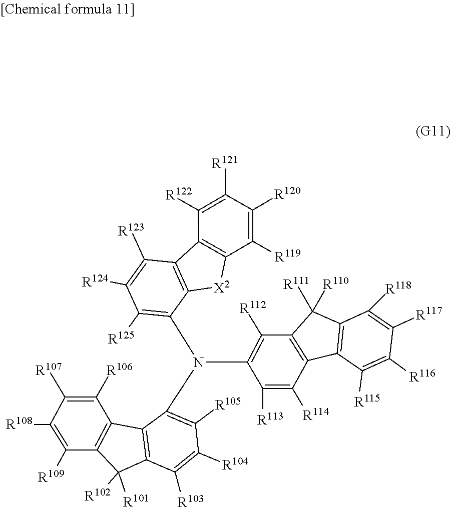

In General Formula (G11), X² represents oxygen or sulfur, and R¹⁰³ to R¹⁰⁹, R¹¹² to R¹¹⁸, and R¹¹⁹ to R¹²⁵ each independently represent hydrogen (including deuterium), a substituted or unsubstituted alkyl group having 1 to 6 carbon atoms, or a substituted or unsubstituted aryl group having 6 to 13 carbon atoms, and R¹⁰¹, R¹⁰², R¹¹⁰, and R¹¹¹ each independently represent hydrogen (including deuterium), a substituted or unsubstituted alkyl group having 1 to 6 carbon atoms, or a substituted or unsubstituted aryl group having 6 to 13 carbon atoms. In addition, R¹⁰⁶, R¹¹⁴, and R¹¹⁵ each represent a substituent other than an unsubstituted phenyl group. When R¹⁰¹ and R¹⁰² are each an unsubstituted phenyl group, R¹¹⁰ and R¹¹¹ each represent a substituent other than a methyl group. When R¹¹⁰ and R¹¹¹ are each an unsubstituted phenyl group, R¹⁰¹ and R¹⁰² each represent a substituent other than a methyl group.

Another embodiment of the present invention is an organic compound represented by General Formula (G11).

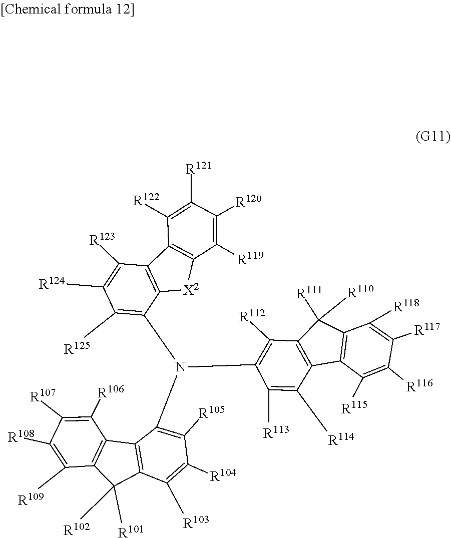

In General Formula (G11), X² represents oxygen or sulfur, and R¹⁰³ to R¹⁰⁹, R¹¹² to R¹¹⁸, and R¹¹⁹ to R¹²⁵ each independently represent hydrogen (including deuterium), a substituted or unsubstituted alkyl group having 1 to 6 carbon atoms, or a substituted or unsubstituted aryl group having 6 to 13 carbon atoms, and R¹⁰¹, R¹⁰², R¹¹⁰, and R¹¹¹ each independently represent hydrogen (including deuterium), a methyl group, or an unsubstituted phenyl group. Note that when R¹⁰¹ and R¹⁰² are each an unsubstituted phenyl group, R¹¹⁰ and R¹¹¹ each represent a substituent other than a methyl group, and when R¹¹⁰ and R¹¹¹ are each an unsubstituted phenyl group, R¹⁰¹ and R¹⁰² each represent a substituent other than a methyl group.

Another embodiment of the present invention is an organic compound represented by General Formula (G12).

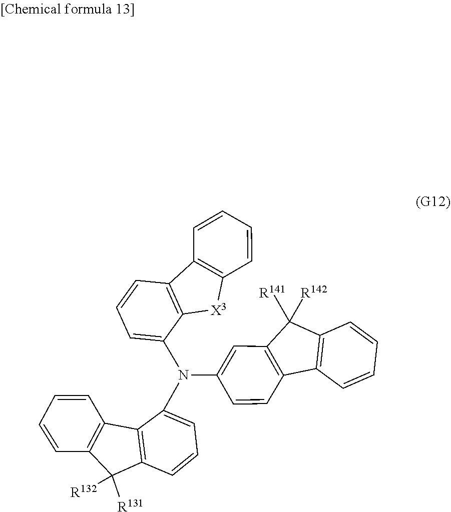

In General Formula (G12), X³ represents oxygen or sulfur, and three or all of R¹³¹, R¹³², R¹⁴¹, and R¹⁴² represent the same substituent, and the substituent represents hydrogen (including deuterium), a methyl group, or an unsubstituted phenyl group, and the other thereof represents hydrogen (including deuterium), a methyl group, or an unsubstituted phenyl group, and some or all of hydrogen atoms may be deuterium.

Another embodiment of the present invention is an organic compound represented by either Structural Formula (200) or Structural Formula (400),

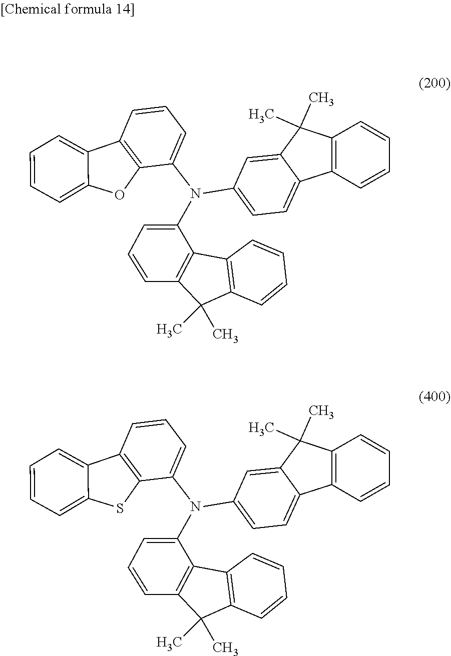 [SMILES: CC1(C)c2ccccc2-c2ccc(N(c3cccc4c3-c3ccccc3C4(C)C)c3cccc4c3oc3ccccc34)cc21 ; CC1(C)c2ccccc2-c2ccc(N(c3cccc4c3-c3ccccc3C4(C)C)c3cccc4c3sc3ccccc34)cc21]

Another embodiment of the present invention is an organic compound represented by General Formula (G21).

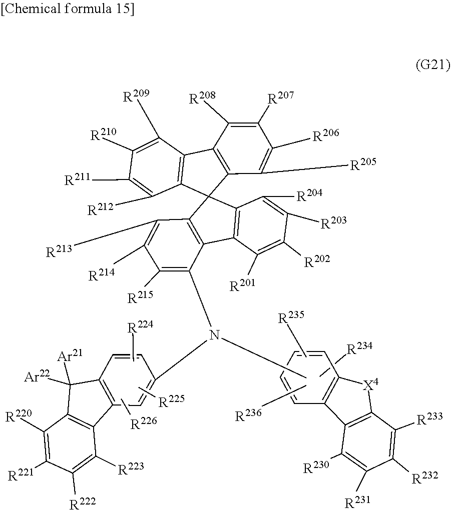

In General Formula (G21), Ar²¹ and Ar²² each independently represent a substituted or unsubstituted aryl group having 6 to 30 carbon atoms, R²⁰¹ to R²¹¹, R²²⁰ to R²²⁶, and R²³⁰ to R²³⁶ each independently represent hydrogen (including deuterium), a substituted or unsubstituted alkyl group having 1 to 6 carbon atoms, a substituted or unsubstituted cycloalkyl group having 3 to 10 carbon atoms, a substituted or unsubstituted aryl group having 6 to 30 carbon atoms, or a substituted or unsubstituted heteroaryl group having 3 to 30 carbon atoms, and X⁴ represents sulfur or oxygen. Note that the substituents represented by Ar²¹ and Ar²² may be bonded to each other to form a ring.

Another embodiment of the present invention is an organic compound represented by General Formula (G22).

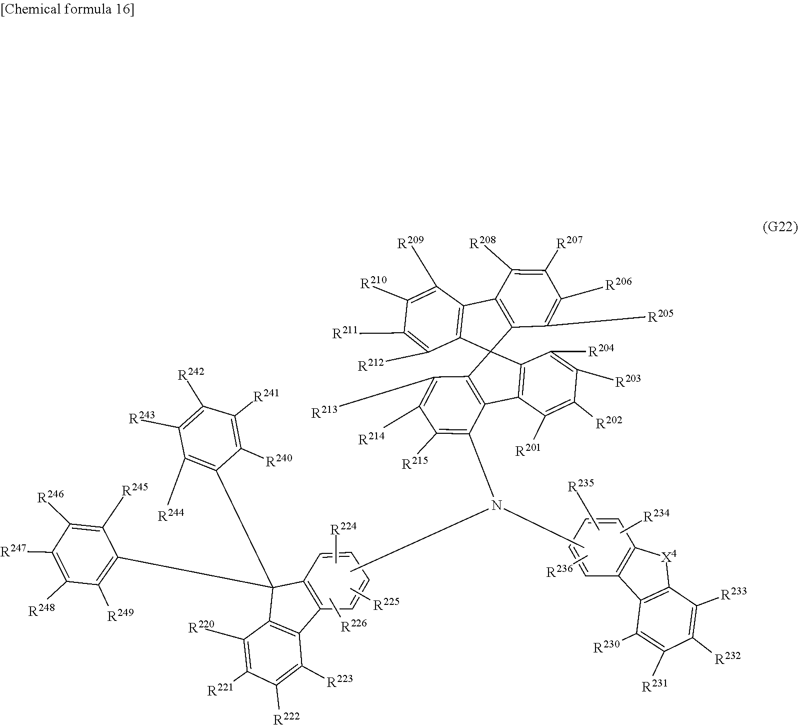

In General Formula (G22), R²⁰¹ to R²¹⁵, R²²⁰ to R²²⁶, R²³⁰ to R²³⁶, and R²⁴⁰ to R²⁴⁹ each independently represent hydrogen (including deuterium), a site to be bonded, a substituted or unsubstituted alkyl group having 1 to 6 carbon atoms, a substituted or unsubstituted cycloalkyl group having 3 to 10 carbon atoms, a substituted or unsubstituted aryl group having 6 to 30 carbon atoms, or a substituted or unsubstituted heteroaryl group having 3 to 30 carbon atoms, and X⁴ represents sulfur or oxygen. Note that the substituents represented by R²⁴⁴ and R²⁴⁵ may be directly bonded to each other to form a spirobifluorene ring.

Another embodiment of the present invention is an organic compound represented by General Formula (G23).

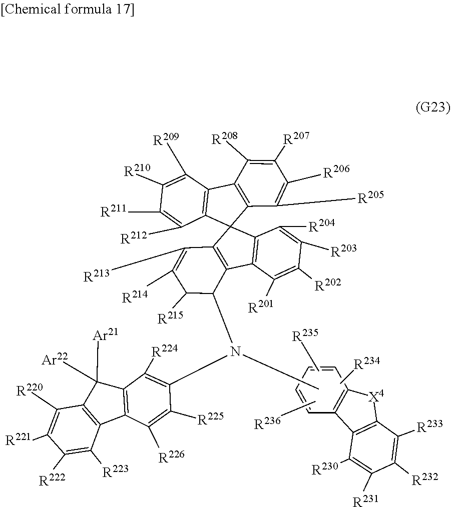

In General Formula (G23), Ar²¹ and Ar²² each independently represent a substituted or unsubstituted aryl group having 6 to 30 carbon atoms, R²⁰¹ to R²¹⁵, R²²⁰ to R²²⁶, and R²³⁰ to R²³⁶ each independently represent hydrogen (including deuterium), a substituted or unsubstituted alkyl group having 1 to 6 carbon atoms, a substituted or unsubstituted cycloalkyl group having 3 to 10 carbon atoms, a substituted or unsubstituted aryl group having 6 to 30 carbon atoms, or a substituted or unsubstituted heteroaryl group having 3 to 30 carbon atoms, and X⁴ represents sulfur or oxygen. Note that the substituents represented by Ar²¹ and Ar²² may be bonded to each other to form a ring.

Another embodiment of the present invention is an organic compound represented by General Formula (G24).

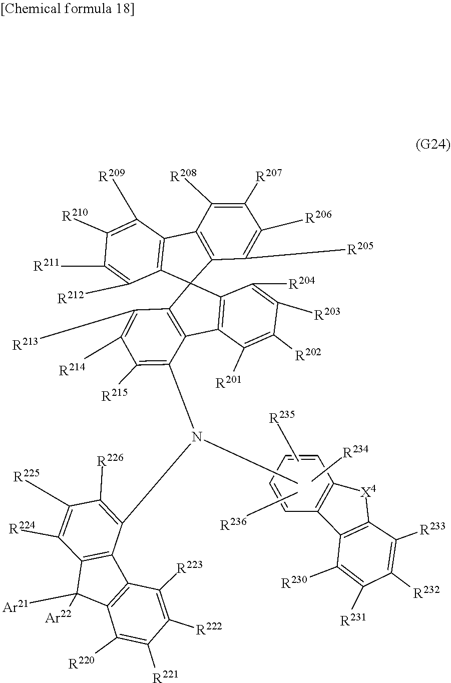

In General Formula (G24), Ar²¹ and Ar²² each independently represent a substituted or unsubstituted aryl group having 6 to 30 carbon atoms, R²⁰¹ to R²¹¹, R²²⁰ to R²²⁶, and R²³⁰ to R²³⁶ each independently represent hydrogen (including deuterium), a substituted or unsubstituted alkyl group having 1 to 6 carbon atoms, a substituted or unsubstituted cycloalkyl group having 3 to 10 carbon atoms, a substituted or unsubstituted aryl group having 6 to 30 carbon atoms, or a substituted or unsubstituted heteroaryl group having 3 to 30 carbon atoms, and X⁴ represents sulfur or oxygen. Note that the substituents represented by Ar²¹ and Ar²² may be bonded to each other to form a ring.

Another embodiment of the present invention is an organic compound represented by General Formula (G25).

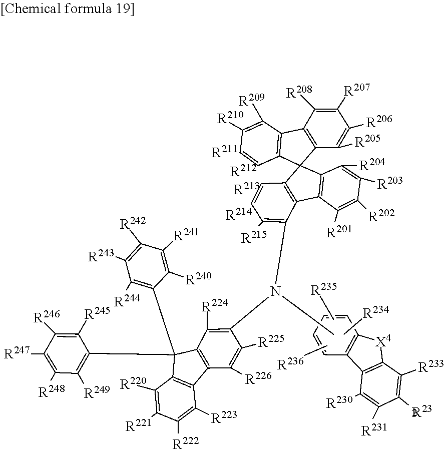

In General Formula (G25), R²⁰¹ to R²¹⁵, R²²⁰ to R²²⁶, R²³⁰ to R²³⁶, and R²⁴⁰ to R²⁴⁹ each independently represent hydrogen (including deuterium), a bonding site, a substituted or unsubstituted alkyl group having 1 to 6 carbon atoms, a substituted or unsubstituted cycloalkyl group having 3 to 10 carbon atoms, a substituted or unsubstituted aryl group having 6 to 30 carbon atoms, or a substituted or unsubstituted heteroaryl group having 3 to 30 carbon atoms, and X⁴ represents sulfur or oxygen. Note that the substituents represented by R²⁴⁴ and R²⁴⁵ may be bonded to each other to form a spirobifluorene ring.

Another embodiment of the present invention is an organic compound represented by General Formula (G26).

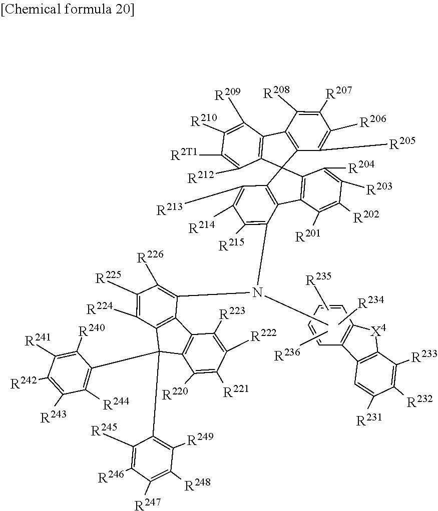

In General Formula (G26), R²⁰¹ to R²¹⁵, R²²⁰ to R²²⁶, R²³⁰ to R²³⁶, and R²⁴⁰ to R²⁴⁹ each independently represent hydrogen (including deuterium), a site to be bonded, a substituted or unsubstituted alkyl group having 1 to 6 carbon atoms, a substituted or unsubstituted cycloalkyl group having 3 to 10 carbon atoms, a substituted or unsubstituted aryl group having 6 to 30 carbon atoms, or a substituted or unsubstituted heteroaryl group having 3 to 30 carbon atoms, and X⁴ represents sulfur or oxygen. Note that the substituents represented R²⁴⁴ and R²⁴⁵ may be directly bonded to each other to form a spirobifluorene ring.

Another embodiment of the present invention is an organic compound represented by General Formula (G27).

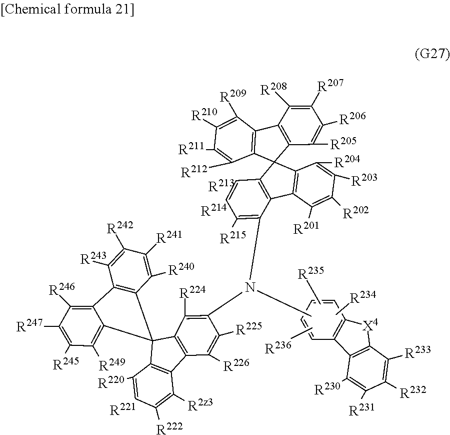

In General Formula (G27), R²⁰¹ to R²¹⁵, R²²⁰ to R²²⁶, R²³⁰ to R²³⁶, R²⁴⁰ to R²⁴³, and R²⁴⁶ to R²⁴⁹ each independently represent hydrogen (including deuterium), a site to be bonded, a substituted or unsubstituted alkyl group having 1 to 6 carbon atoms, a substituted or unsubstituted cycloalkyl group having 3 to 10 carbon atoms, a substituted or unsubstituted aryl group having 6 to 30 carbon atoms, or a substituted or unsubstituted heteroaryl group having 3 to 30 carbon atoms, and X⁴ represents sulfur or oxygen.

Another embodiment of the present invention is an organic compound represented by General Formula (G28).

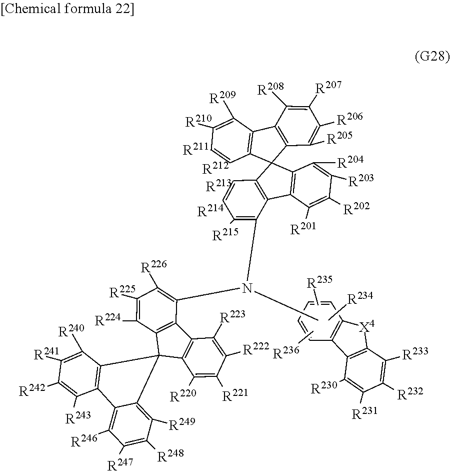

In General Formula (G28), R²⁰¹ to R²¹⁵, R²²⁰ to R²²⁶, R²³⁰ to R²³⁶, R²⁴⁰ to R²⁴³, and R²⁴⁶ to R²⁴⁹ each independently represent hydrogen (including deuterium), a site to be bonded, a substituted or unsubstituted alkyl group having 1 to 6 carbon atoms, a substituted or unsubstituted cycloalkyl group having 3 to 10 carbon atoms, a substituted or unsubstituted aryl group having 6 to 30 carbon atoms, or a substituted or unsubstituted heteroaryl group having 3 to 30 carbon atoms, and X⁴ represents sulfur or oxygen.

Another embodiment of the present invention is any one of the above-described organic compounds in which carbon at a 2-position of the dibenzofuranyl group or the dibenzothiophenyl group is bonded to nitrogen.

Another embodiment of the present invention is any one of the above-described organic compounds in which carbon at a 4-position of the dibenzofuranyl group or the dibenzothiophenyl group is bonded to nitrogen.

Another embodiment of the present invention is any one of the above-described organic compounds in which X⁴ represents oxygen.

Another embodiment of the present invention is any of the above-described organic compounds in which any one of R²⁴⁰ to R²⁴⁴ and any one of R²⁴⁵ to R²⁴⁹ are each a substituted or unsubstituted phenyl group.

Another embodiment of the present invention is any of the above-described organic compounds in which R²⁴⁰ and R²⁴⁵ are each a substituted or unsubstituted phenyl.

Another embodiment of the present invention is any of the above-described organic compounds in which R²⁴² and R²⁴⁷ are each a substituted or unsubstituted phenyl group.

Another embodiment of the present invention is a light-emitting device using any of the above organic compounds.

Another embodiment of the present invention is a light-emitting apparatus including the above light-emitting device, and a transistor or a substrate.

Another embodiment of the present invention is an electronic appliance including the above-described light-emitting apparatus; and a sensor unit, an input unit, or a communication unit.

Another embodiment of the present invention is a lighting device including the above-described light-emitting apparatus and a housing.

According to one embodiment of the present invention, a novel organic compound which has a low HOMO level and a hole-transport property can be provided. A light-emitting device with high emission efficiency can be provided by using a novel organic compound of one embodiment of the present invention. In addition, a light-emitting device that can emit fluorescent light with high emission efficiency can be provided by using a novel organic compound of one embodiment of the present invention. Furthermore, a light-emitting element, a light-emitting device, an electronic appliance, or a lighting device with low power consumption can be provided.

### BRIEF DESCRIPTION OF THE DRAWINGS

In the accompanying drawings:

FIGS. 1A to 1C illustrate structures of light-emitting devices of according to an embodiment;

FIGS. 2A to 2E illustrate structures of light-emitting devices according to an embodiment;

FIGS. 3A to 3D illustrate a light-emitting apparatus according to an embodiment;

FIGS. 4A to 4C illustrate a fabrication method of the light-emitting apparatus according to the embodiment;

FIGS. 5A to 5C illustrate the fabrication method of the light-emitting apparatus according to an embodiment;

FIGS. 6A to 6C illustrate the fabrication method of the light-emitting apparatus according to an embodiment;

FIGS. 7A to 7D illustrate the fabrication method of the light-emitting apparatus according to the embodiment;

FIGS. 8A to 8E illustrate the fabrication method of the light-emitting apparatus according to the embodiment;

FIGS. 9A to 9F illustrate an apparatus and pixel arrangements according to an embodiment;

FIGS. 10A to 10C illustrate pixel circuits according to the embodiment;

FIG. 11 illustrates a light-emitting apparatus according to the embodiment;

FIGS. 12A to 12E illustrate electronic appliances according to an embodiment;

FIGS. 13A to 13E illustrate electronic appliances according to the embodiment;

FIGS. 14A and 14B illustrate electronic appliances according to the embodiment;

FIGS. 15A and 15B illustrate a lighting device according to an embodiment;

FIG. 16 illustrates lighting devices according to an embodiment;

FIGS. 17A to 17C illustrate a light-emitting device and a light-receiving device according to an embodiment;

FIGS. 18A and 18B illustrate a light-emitting device and a light-receiving device according to the embodiment;

FIG. 19 illustrates a structure of a light-emitting device in examples;

FIGS. 20A and 20B show a ¹H-NMR spectrum of ThFAF(4);

FIGS. 21A and 21B show a ¹H-NMR spectrum of FrFAF(4);

FIG. 22 shows an absorption spectrum and an emission spectrum of a toluene solution of FrFAF(4);

FIG. 23 shows luminance-current density characteristics of light-emitting devices 1 to 3;

FIG. 24 shows current efficiency-luminance characteristics of the light-emitting devices 1 to 3;

FIG. 25 shows luminance-voltage characteristics of the light-emitting devices 1 to 3;

FIG. 26 shows current-voltage characteristics of the light-emitting devices 1 to 3;

FIG. 27 shows the external quantum efficiency-luminance characteristics of the light-emitting devices 1 to 3;

FIG. 28 shows emission spectra of the light-emitting devices 1 to 3;

FIG. 29 shows the luminance-current density characteristics of light-emitting devices 4 to 7;

FIG. 30 shows the current efficiency-luminance characteristics of the light-emitting devices 4 to 7;

FIG. 31 shows the luminance-voltage characteristics of the light-emitting devices 4 to 7;

FIG. 32 shows the current-voltage characteristics of the light-emitting devices 4 to 7;

FIG. 33 shows external quantum efficiency-luminance characteristics of the light-emitting devices 4 to 7;

FIG. 34 shows emission spectra of the light-emitting devices 4 to 7;

FIGS. 35A and 35B show a ¹H-NMR spectrum of Fr(2)FASF(4)-02;

FIG. 36 shows the luminance-current density characteristics of light-emitting devices 8 and 9;

FIG. 37 shows the current efficiency-luminance characteristics of the light-emitting devices 8 and 9;

FIG. 38 shows the luminance-voltage characteristics of the light-emitting devices 8 and 9;

FIG. 39 shows the current-voltage characteristics of the light-emitting devices 8 and 9;

FIG. 40 shows external quantum efficiency-luminance characteristics of the light-emitting devices 8 and 9;

FIG. 41 shows emission spectra of the light-emitting devices 8 and 9; and

FIG. 42 is a graph showing a change in luminance over driving time of the light-emitting devices 8 and 9.

### DETAILED DESCRIPTION OF THE INVENTION

### Embodiment 1

This embodiment will describe a light-emitting device of one embodiment of the present invention.

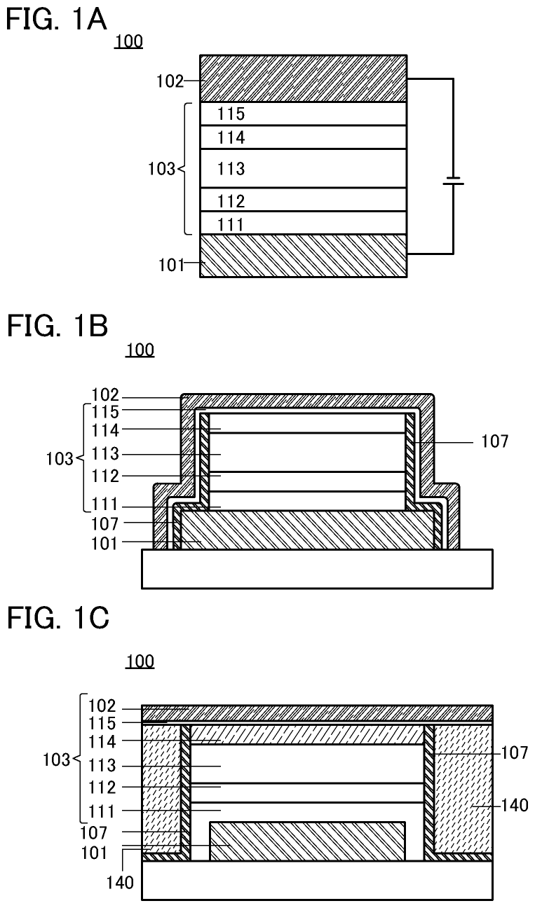

FIG. 1A illustrates a structure of a light-emitting device 100 of one embodiment of the present invention. As illustrated in FIG. 1A, the light-emitting device 100 includes a first electrode 101, a second electrode 102, and an EL layer 103 between the first electrode 101 and the second electrode 102. In the EL layer 103, a hole-injection layer 111, a hole-transport layer 112, a light-emitting layer 113, an electron-transport layer 114, and an electron-injection layer 115 are sequentially stacked.

The light-emitting layer 113 contains at least a light-emitting substance.

As the light-emitting substance, a substance which emits fluorescent light (fluorescent substance) can be used. In other words, as the light-emitting substance, a light-emitting substance that converts singlet excitation energy into light emission can be used. To put it differently, a light-emitting substance that can convert its singlet excitation energy into light emission and has the difference (ΔE_{ST}) between the lowest singlet excited level and the lowest triplet excited level of 0.3 eV or more, can be used. Thus, the EL layer 103 can emit fluorescent light. A light-emitting device of one embodiment of the present invention contains a substance emitting fluorescent light and thus can also be a fluorescent light-emitting device.

As the light-emitting substance, a substance that emits blue light can also be used. Thus, the EL layer 103 is capable of emitting blue light. Note that in this specification and the like, the substance that emits blue light refers to a light-emitting substance that has a maximum peak of an emission spectrum in a wavelength range from 400 nm to 490 nm.

Specific examples of the fluorescent substance will be described in Embodiment 2.

In the light-emitting device 100 of one embodiment of the present invention, the EL layer 103 contains a first organic compound.

The first organic compound is an aromatic amine having a structure in which a nitrogen atom is bonded to a fluorenyl group, an aryl group, a dibenzofuranyl group, or a dibenzothiophenyl group. The first organic compound having such a structure easily accepts holes (that is, the first organic compound has a hole-transport property). Accordingly, when the first organic compound is used for the EL layer 103 containing a substance emitting fluorescent light, the hole-injection property, the hole-transport property, and the like to the EL layer 103 can be improved, and thus, the carrier balance of the EL layer 103 is expected to be improved. As a result, the recombination probability in the EL layer 103 can be increased and thus the substance exhibiting fluorescence can efficiently emit light. Furthermore, the first organic compound is used for a layer which needs to contain a material with a high hole-transport property or a high hole-injection property in the EL layer 103, whereby the driving voltage of the light-emitting device 100 can be reduced, resulting in lowering the power consumption of the light-emitting device 100.

For example, the hole-injection layer 111 can be formed using the first organic compound. The hole-injection layer 111 is a layer that is in contact with the first electrode 101 and needs to contain a material with a high hole-injection property. For example, a material having a high hole-injection property in which the first organic compound and an organic acceptor material (electron accepting material) are combined can be used for the hole-injection layer 111. The organic acceptor material extracts electrons from the first organic compound, so that holes are generated in the hole-injection layer 111 and the holes are injected into the light-emitting layer 113 through the hole-transport layer 112. In that case, the first organic compound easily receives holes because electrons are easily extracted from the first organic compound. Accordingly, the driving voltage of the light-emitting device 100 can be reduced.

Note that the hole-injection layer 111 may be formed to have a single-layer structure of a mixed material containing the first organic compound and the organic acceptor material or may be a stacked-layer structure in which a layer containing the first organic compound and another layer containing the organic acceptor material may be stacked.

For example, the hole-transport layer 112 can be formed using the first organic compound. The hole-transport layer 112 is a layer that is in contact with the light-emitting layer 113 and should contain a material with a hole-transport property. When the first organic compound is used for the hole-transport layer 112, holes injected from the first electrode 101 can be transported to the light-emitting layer 113. Accordingly, the driving voltage of the light-emitting device 100 can be reduced.

For example, the light-emitting layer 113 can be formed using the first organic compound. As described above, the light-emitting layer 113 contains at least a light-emitting substance. That is, the light-emitting layer 113 can be formed with a combination of a light-emitting substance and the first organic compound. The first organic compound has an excellent hole-transport property, and especially when the first organic compound is used in combination with an electron-transport material, the carrier balance in the light-emitting layer can be easily adjusted, so that the emission efficiency of the light-emitting device 100 can be improved. Moreover, an advantageous effect of improving a hole-injection property to the light-emitting layer can be expected, so that the driving voltage of the EL light-emitting device can be reduced, resulting in lowering the power consumption of the EL light-emitting device.

Note that the layer that can contain the first organic compound is not limited to the EL layer 103, the hole-injection layer 111, the hole-transport layer 112, and the light-emitting layer 113, and any layer provided between the first electrode 101 and the second electrode 102 can contain the first organic compound.

Depending on circumstances, the layer containing the first organic compound may be formed over the second electrode 102.

The layer that can contain the first organic compound is not limited to an EL layer in a light-emitting device, but can be used for a light-receiving layer of a light-receiving device, for example. With the use of the first organic compound for a light-receiving layer of a light-receiving device, a hole-injection property, a hole-transport property, and the like to the light-receiving layer can be improved. For example, the first organic compound can be used for an active layer, a hole-injection layer, or a hole-transport layer of a light-receiving device.

A structure of the light-receiving device that can contain the first organic compound will be described in detail in Embodiments 2 and 8.

A specific example of the first organic compound is an organic compound represented by General Formula (G1) below.

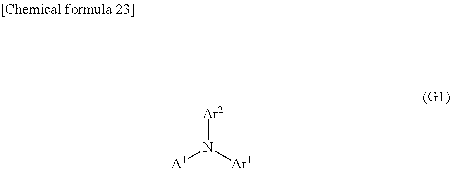

In General Formula (G1), Ar¹ represents a substituted or unsubstituted fluorenyl group, Ar² represents a substituted or unsubstituted aryl group having 6 to 13 carbon atoms, and A¹ represents either a substituted or unsubstituted dibenzofuranyl group or a substituted or unsubstituted dibenzothiophenyl group. Note that when at least one of Ar¹, Ar², and A¹ has one or more substituents, the substituents each independently represent an alkyl group having 1 to 4 carbon atoms or an aryl group having 6 to 13 carbon atoms. Note that the aryl group exclude a heteroaryl group. The substituents may be bonded to each other to form a ring.

Another specific example of the first organic compound is an organic compound represented by General Formula (G2) below.

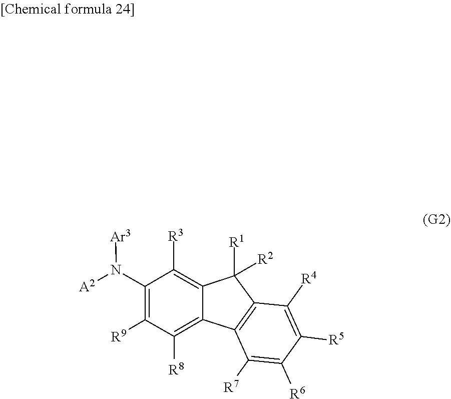

In General Formula (G2), Ar³ represents a substituted or unsubstituted aryl group having 6 to 13 carbon atoms, and A² represents either a substituted or unsubstituted dibenzofuranyl group or a substituted or unsubstituted dibenzothiophenyl group. R¹ to R⁹ each independently represent hydrogen (including deuterium), an alkyl group having 1 to 4 carbon atoms, or an aryl group having 6 to 13 carbon atoms. When one or both of Ar³ and A² have one or more substituents, the substituents each independently represent an alkyl group having 1 to 4 carbon atoms or an aryl group having 6 to 13 carbon atoms. Note that the aryl group exclude a heteroaryl group. The substituents may be bonded to each other to form a ring.

Specific examples of the alkyl group having 1 to 4 carbon atoms in R¹ to R⁹ in General Formula (G2) include a methyl group, an ethyl group, a propyl group, an isopropyl group, a butyl group, a sec-butyl group, an isobutyl group, and a tert-butyl group. Specific examples of the aryl group having 6 to 13 carbon atoms in R¹ to R⁹ in General Formula (G2) include a phenyl group, a tolyl group, a xylyl group, a mesityl group, a biphenyl group, a naphthyl group, and a fluorenyl group. As described above, at least two of the substituents may be bonded to each other to form a ring; a spirobifluorenyl group is regarded as a group having substituents which are bonded to each other to form a ring (that is, a spirobifluorenyl group is a compound in which two phenyl groups in a 9,9-diphenylfluorenyl group are bonded to form a ring).

Specific examples of Ar² in General Formula (G1) and Ar³ in General Formula (G2) include substituents represented by Structural Formulae (1-1) to (1-11). Structural Formulae (1-7) to (1-11) show specific examples of Ar² and Ar³ having a substituent. The structure represented by Structural Formula (1-10) is a case where the substituents are bonded to each other to form a ring.

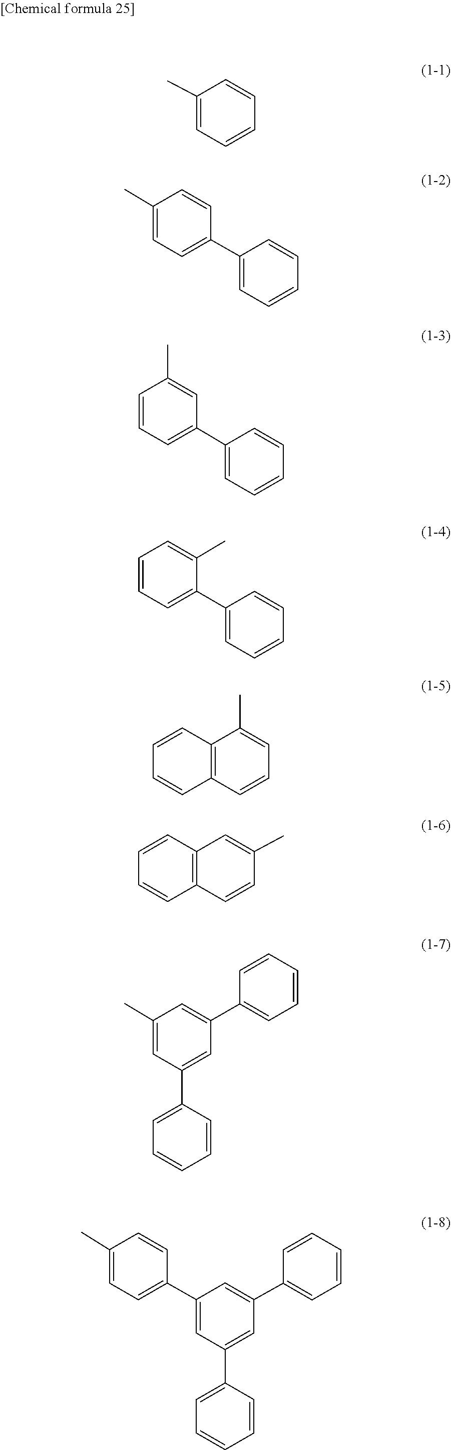

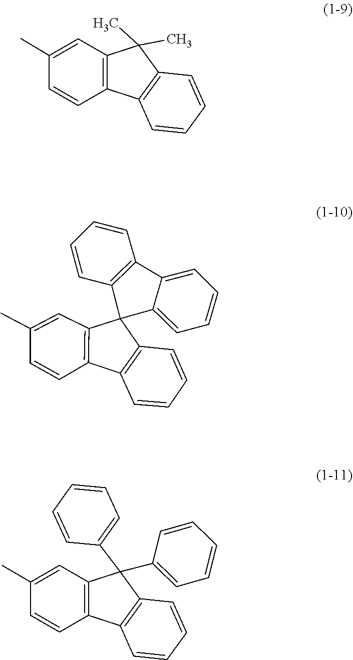

Specific examples of an alkyl group having 1 to 4 carbon atoms that are substituents available on Ar¹ and Ar² in General Formula (G1) and Ar³ and Ar⁴ in General Formulae (G2)include a methyl group, an ethyl group, a propyl group, an isopropyl group, a butyl group, a sec-butyl group, an isobutyl group, and a tert-butyl group. Specific examples of the aryl group having 6 to 13 carbon atoms that are substituents available on Ar¹ and Ar² in General Formula (G1) and Ar³ and Ar⁴ in General Formulae (G2) include a phenyl group, a tolyl group, a xylyl group, a mesityl group, a biphenyl group, a naphthyl group, and a fluorenyl group. As described above, the substituents may be bonded to each other to form a ring; a spirobifluorenyl group is regarded as a group having substituents which are bonded to each other to form a ring (that is, a spirobifluorenyl group is a compound in which two phenyl groups in a 9,9-diphenylfluorenyl group are bonded to form a ring).

Note that the substituent represented by Structural Formula (1-4) shown above is preferably used as Ar² in General Formula (G1) and Ar³ in General Formula (G2). Accordingly, the planarity of Ar² or Ar³ and an unshared electron pair of nitrogen is decreased and the conjugation is hard to be extended, which can increase the electron density on nitrogen probably. Accordingly, since the hole-transport property of the first organic compound can be improved, the driving voltage of the light-emitting device 100 can be reduced. In addition, since an increase in the evaporation temperature of the first organic compound is inhibited, a stable film can be formed by an evaporation method. The light-emitting device 100 can be expected to have higher heat resistance. Furthermore, the light-emitting device 100 can be expected to have higher reliability.

Another specific example of the first organic compound is an organic compound represented by General Formula (G3) below.

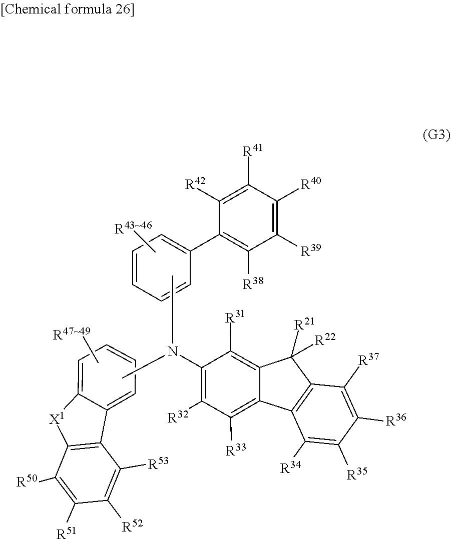

In General Formula (G3), X¹ represents oxygen or sulfur, R²¹, R²², and R³¹ to R³⁷ each independently represent hydrogen (including deuterium), an alkyl group having 1 to 4 carbon atoms, or an aryl group having 6 to 13 carbon atoms, R³⁸ to R⁴⁶ each independently represent hydrogen (including deuterium), an alkyl group having 1 to 4 carbon atoms, or an aryl group having 6 to 13 carbon atoms, and R⁴⁷ to R⁵³ each independently represent hydrogen (including deuterium), an alkyl group having 1 to 4 carbon atoms, or an aryl group having 6 to 13 carbon atoms. Note that the aryl group exclude a heteroaryl group. In addition, at least two of the substituents represented by R²¹, R²², and R³¹ to R³⁷ may be bonded to each other to form a ring. At least two of the substituents represented by R³⁸ to R⁴⁶ may be bonded to each other to form a ring. In addition, at least two of the substituents represented by R⁴⁷ to R⁵³ may be bonded to each other to form a ring.

Another specific example of the first organic compound is an organic compound represented by General Formula (G4) below.

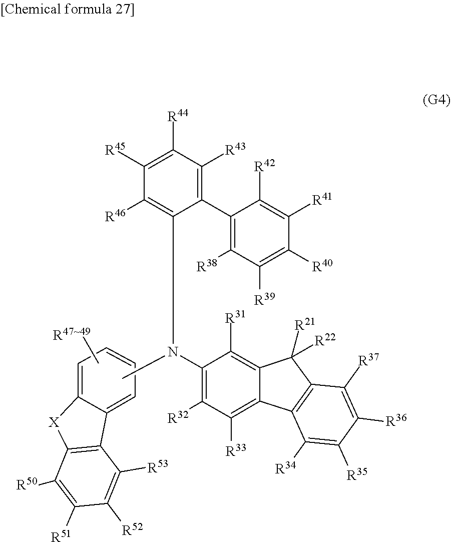

In General Formula (G4) above, X represents oxygen or sulfur, R²¹, R²², and R³¹ to R³⁷ each independently represent hydrogen (including deuterium), an alkyl group having 1 to 4 carbon atoms, or an aryl group having 6 to 13 carbon atoms, R³⁸ to R⁴⁶ each independently represent hydrogen (including deuterium), an alkyl group having 1 to 4 carbon atoms, or an aryl group having 6 to 13 carbon atoms, and R⁴⁷ to R⁵³ each independently represent hydrogen (including deuterium), an alkyl group having 1 to 4 carbon atoms, or an aryl group having 6 to 13 carbon atoms. Note that the aryl group excludes a heteroaryl group. In addition, at least two of the substituents represented by R²¹, R²², and R³¹ to R³⁷ may be bonded to each other to form a ring. At least two of the substituents represented by R³¹ to R⁴⁶ may be bonded to each other to form a ring. In addition, at least two of the substituents represented by R⁴⁷ to R⁵³ may be bonded to each other to form a ring.

General Formula (G4) is different from General Formula (G3) in that a biphenyl group is bonded to nitrogen at the ortho position. Accordingly, the planarity of the biphenyl group and an unshared electron pair of nitrogen is decreased and the conjugation is hard to be extended, which can increase the electron density on nitrogen probably. Thus, since the hole-transport property of the first organic compound can be improved, the driving voltage of the light-emitting device 100 can be reduced. In addition, since an increase in the evaporation temperature of the first organic compound is inhibited, a stable film can be formed by an evaporation method. The light-emitting device 100 can be expected to have higher heat resistance. Furthermore, the light-emitting device 100 can be expected to have higher reliability.

Specific examples of an alkyl group having 1 to 4 carbon atoms in R²¹, R²², R³¹ to R³⁷, R³⁸ to R⁴⁶, and R⁴⁷ to R⁵³ in General Formulae (G3) and (G4) include a methyl group, an ethyl group, a propyl group, an isopropyl group, a butyl group, a sec-butyl group, an isobutyl group, and a tert-butyl group. Specific examples of the aryl group having 6 to 13 carbon atoms in R²¹, R²², R³¹ to R³⁷, R³⁸ to R⁴⁶, and R⁴⁷ to R⁵³ in General Formulae (G3) and (G4) include a phenyl group, a tolyl group, a xylyl group, a mesityl group, a biphenyl group, a naphthyl group, and a fluorenyl group. Furthermore, at least two of the substituents represented by R²¹, R²², and R³¹ to R³⁷ may be bonded to each other to form a ring; for example, a spirobifluorenyl group is regarded as a group having substituents which are bonded to each other to form a ring (that is, a spirobifluorenyl group is a compound in which two phenyl groups in a 9,9-diphenylfluorenyl group are bonded to form a ring). In addition, at least two of the substituents represented by R³⁸ to R⁴⁶ may be bonded to each other to form a ring. At least two of the substituents represented by R⁴⁷ to R⁵³ may be bonded to each other to form a ring. For example, each of a 9,9-dimethylfluorenyl group, a 9,9-diphenylfluorenyl group, and a spirobifluorenyl group can be regarded as to be a compound in which R³⁸ is bonded to any one of R⁴³ to R⁴⁶ to form a fluorene ring.

The following are specific examples of the organic compounds of one embodiment of the present invention each having any one of the above structures represented by General Formulae (G1) to (G4) above.

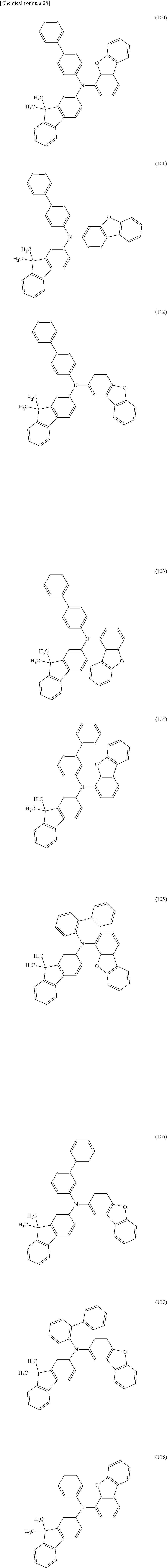

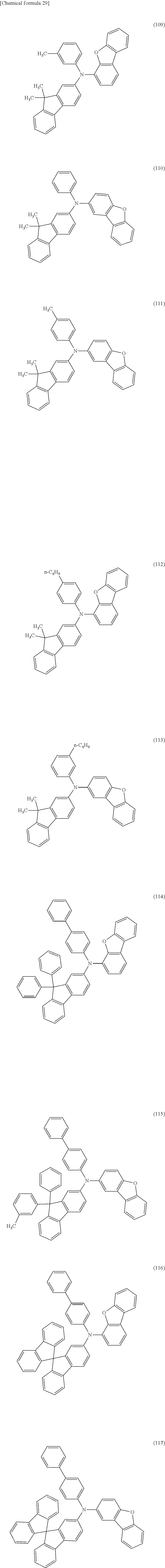

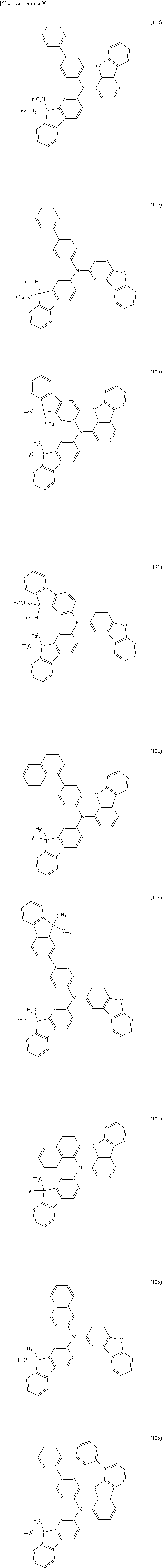

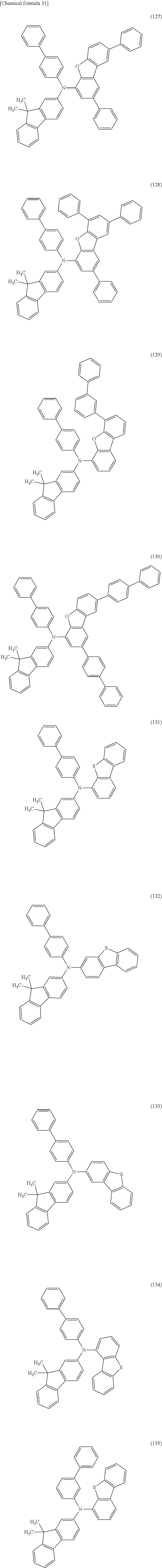

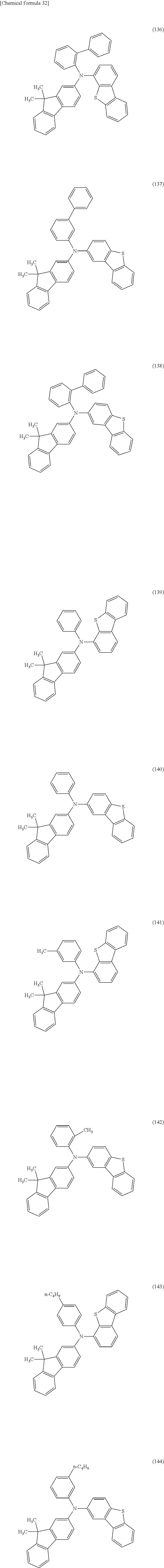

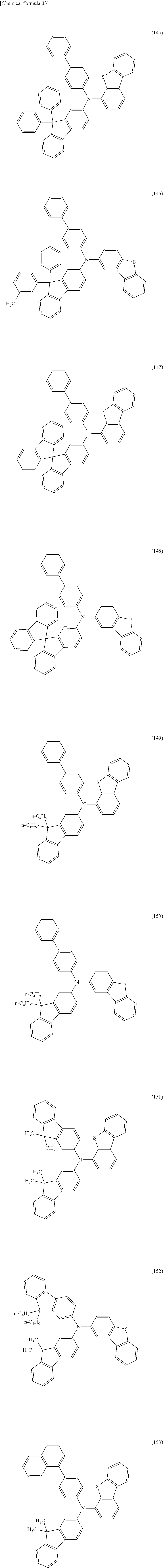

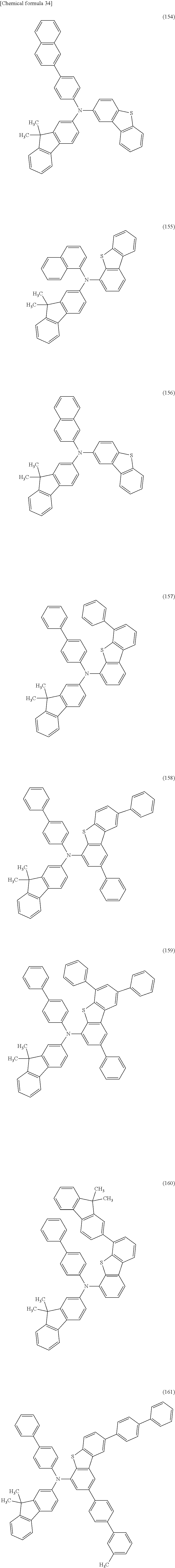

[chemical structure]

[chemical structure]

[chemical structure]

[chemical structure]

[chemical structure]

[chemical structure]

[chemical structure]

[chemical structure]

[chemical structure]

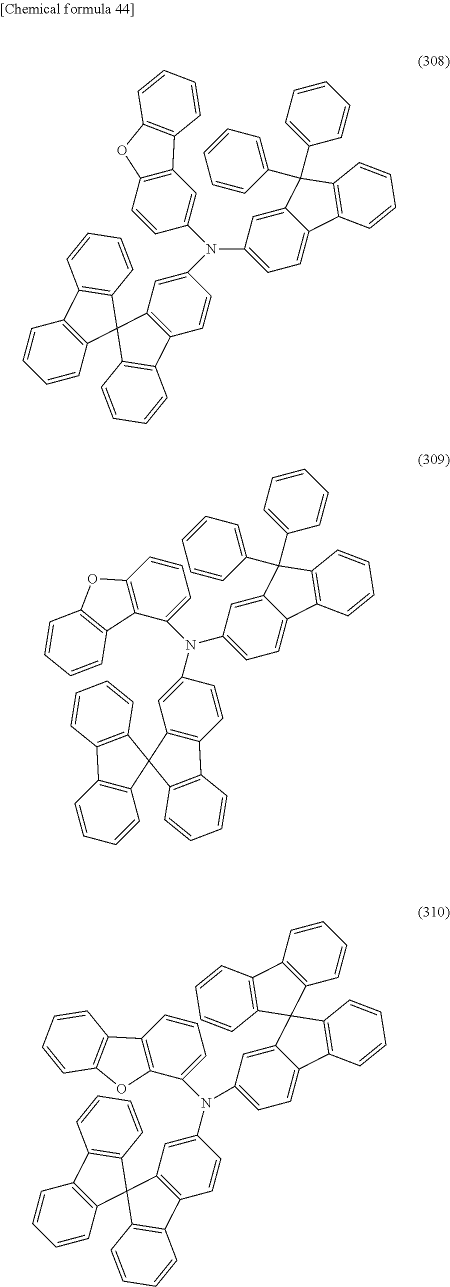

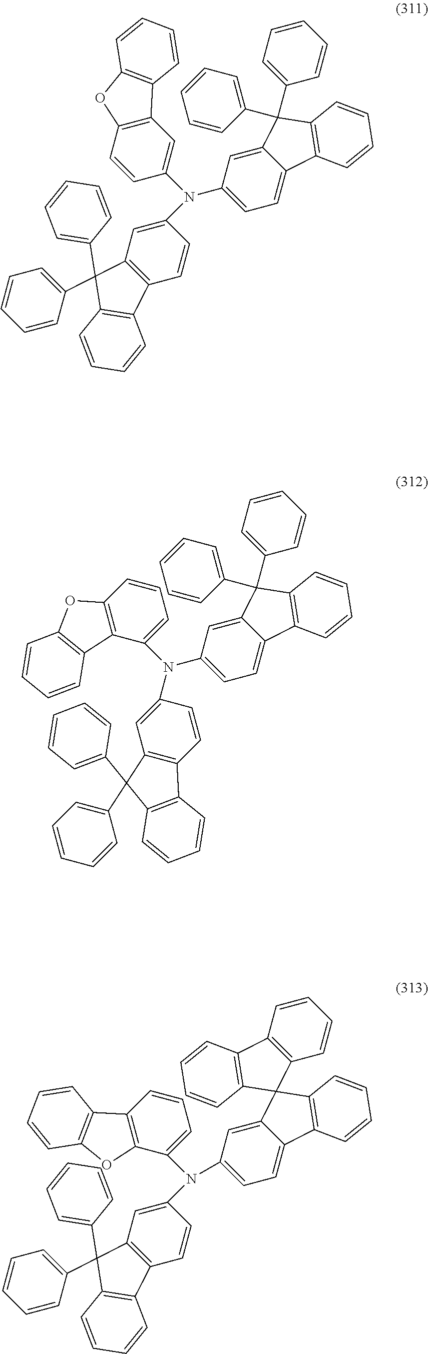

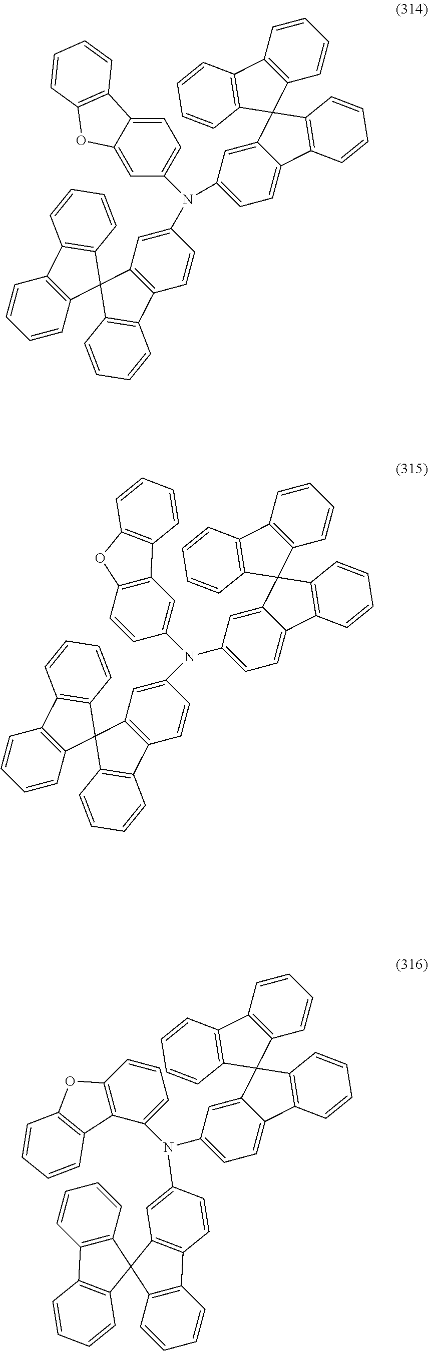

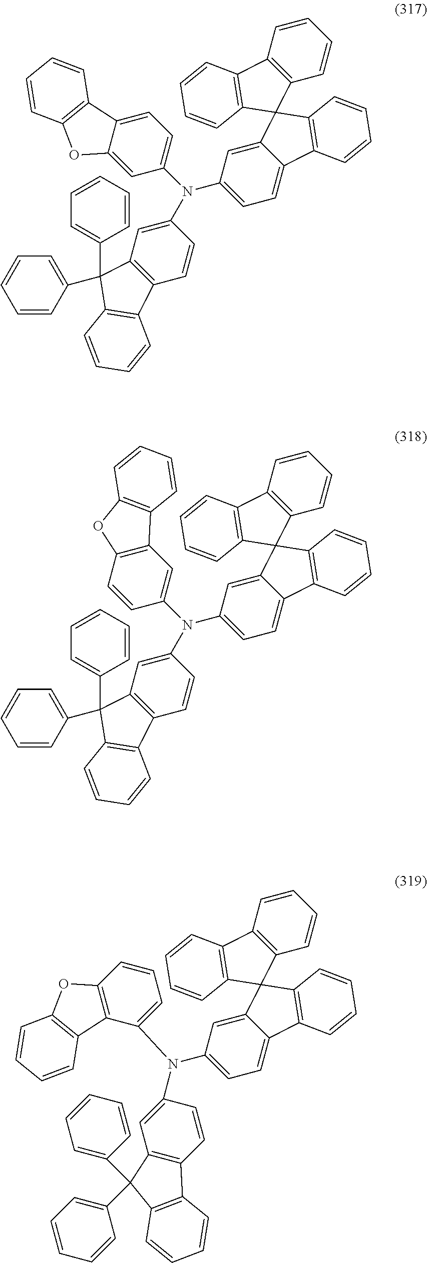

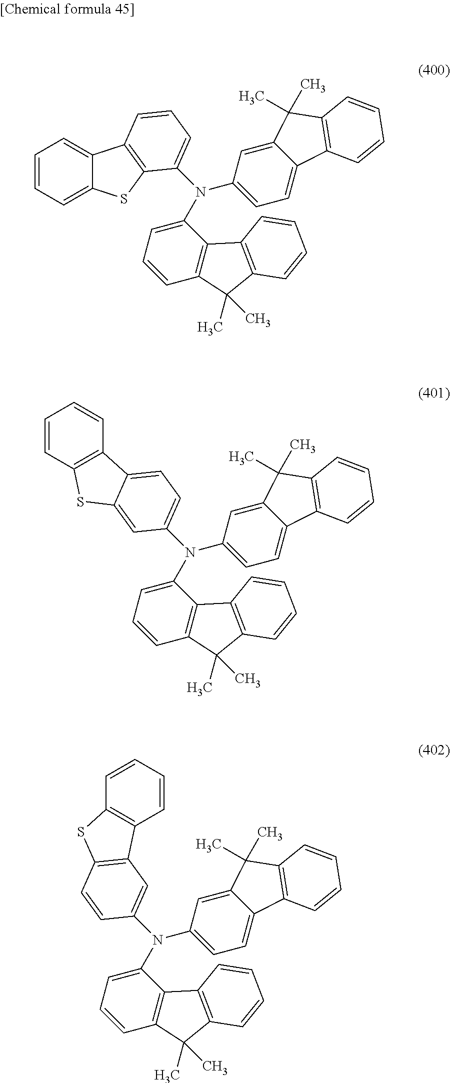

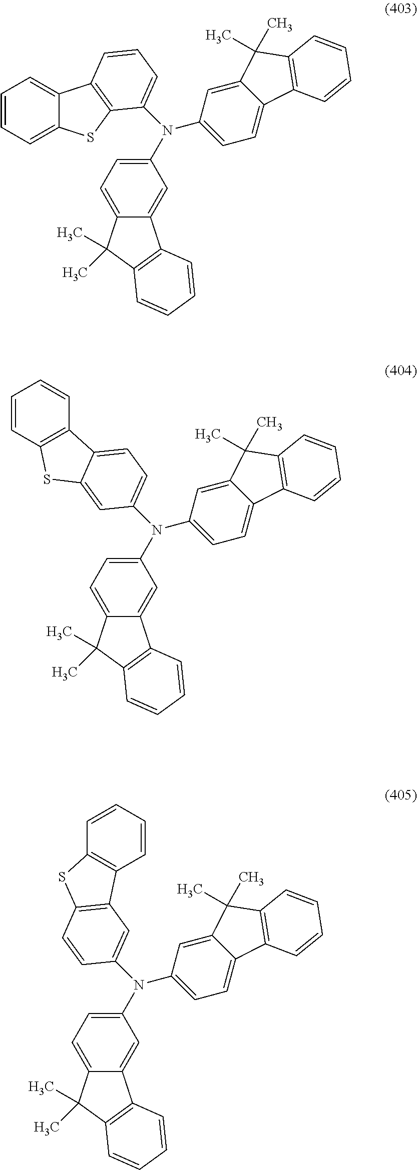

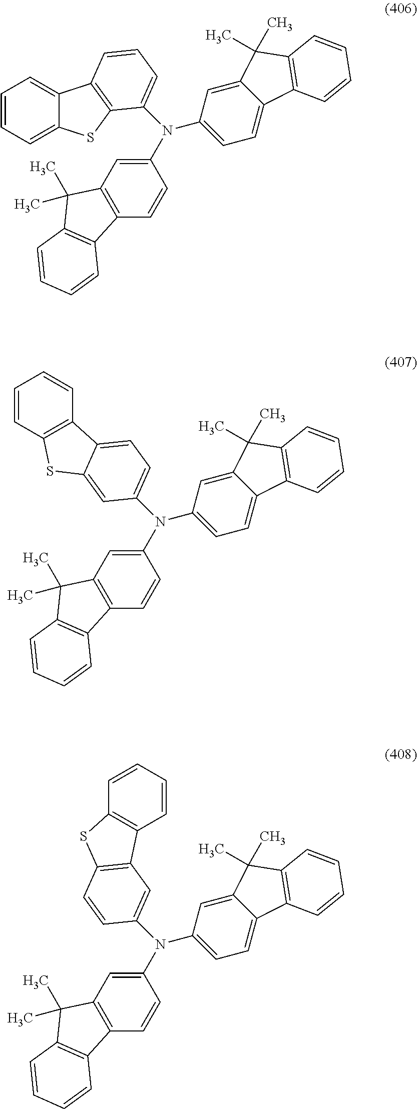

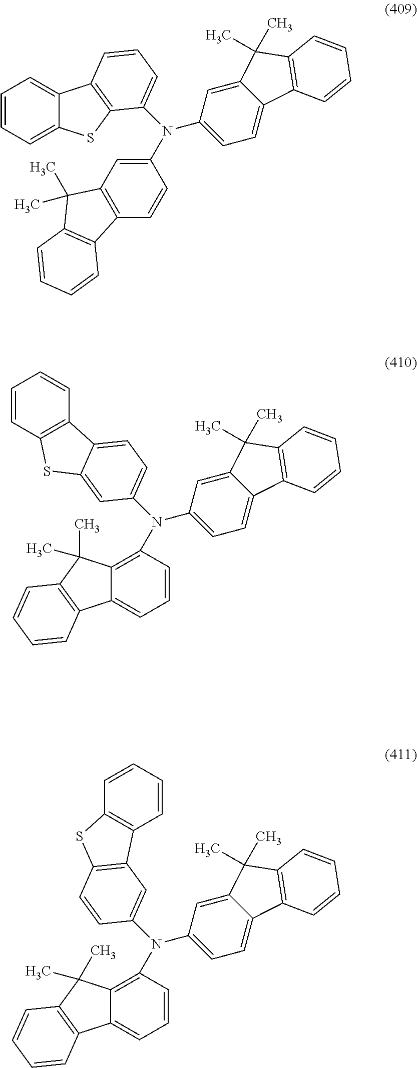

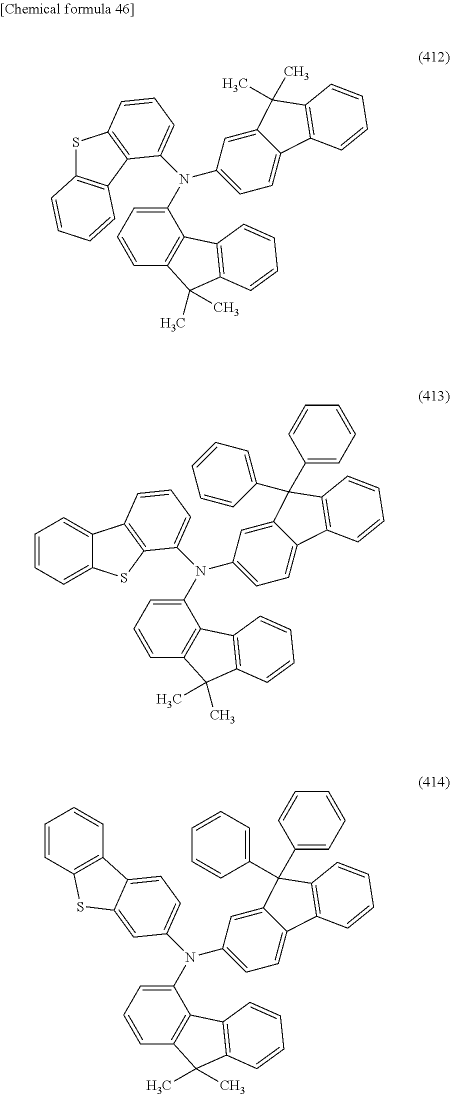

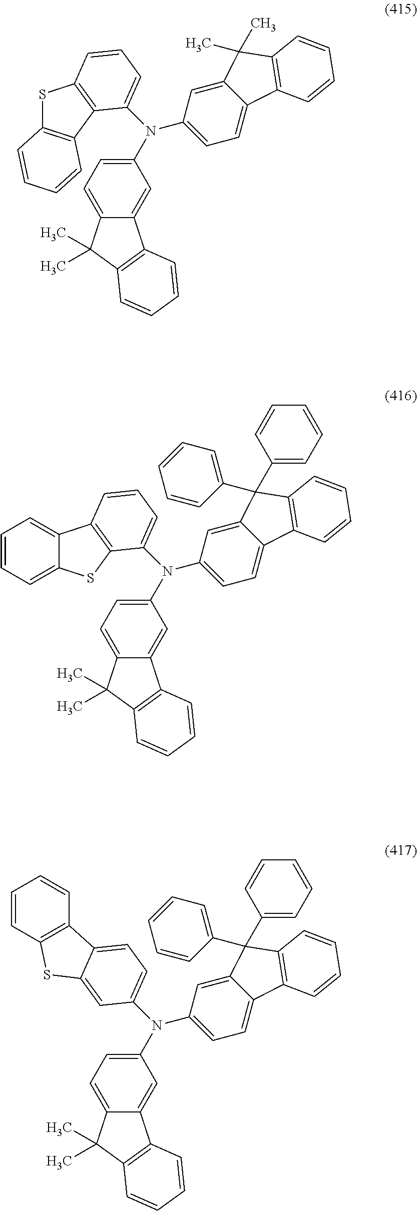

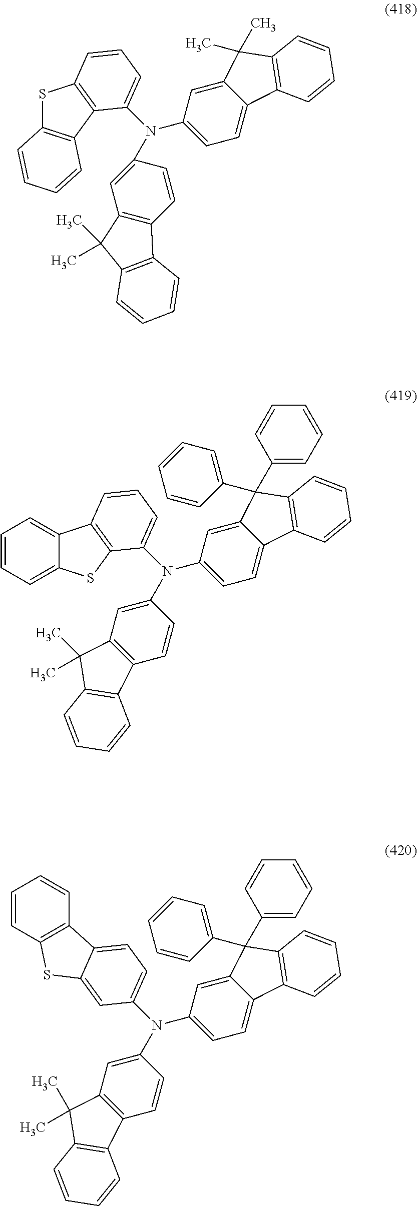

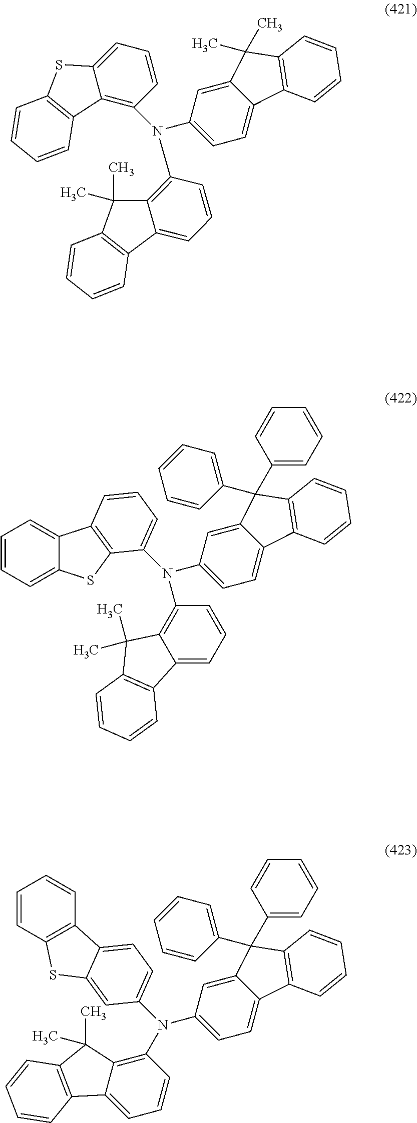

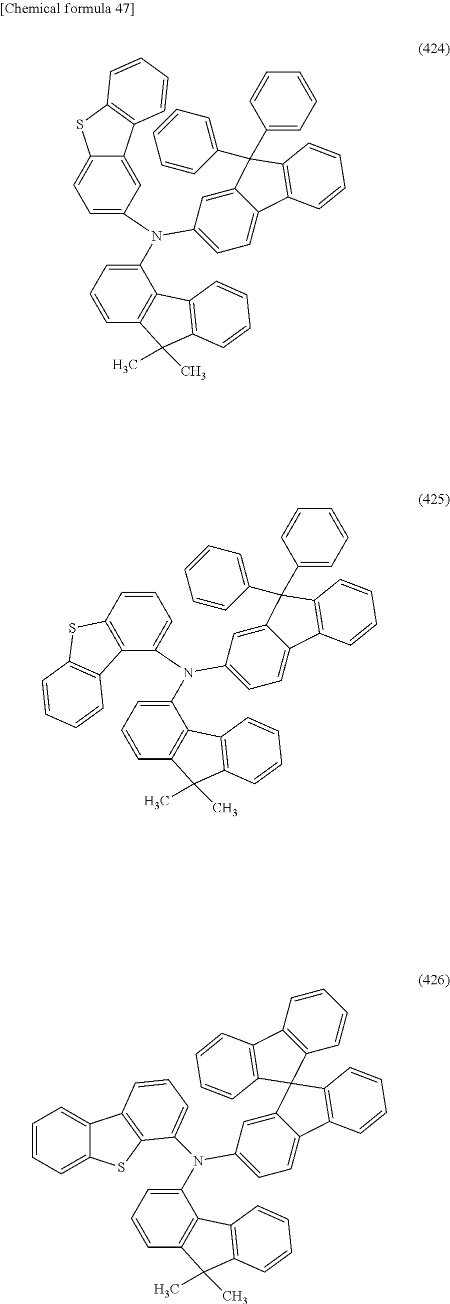

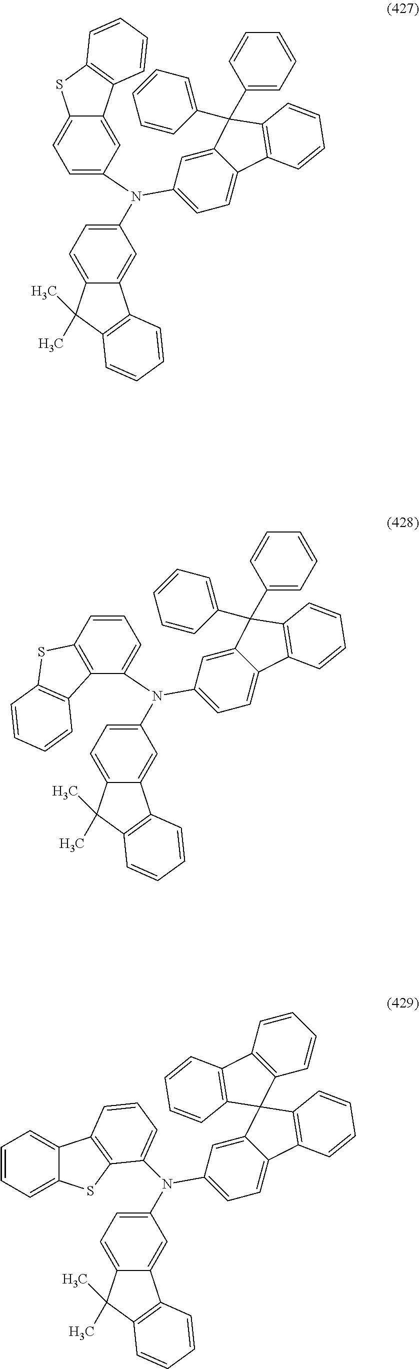

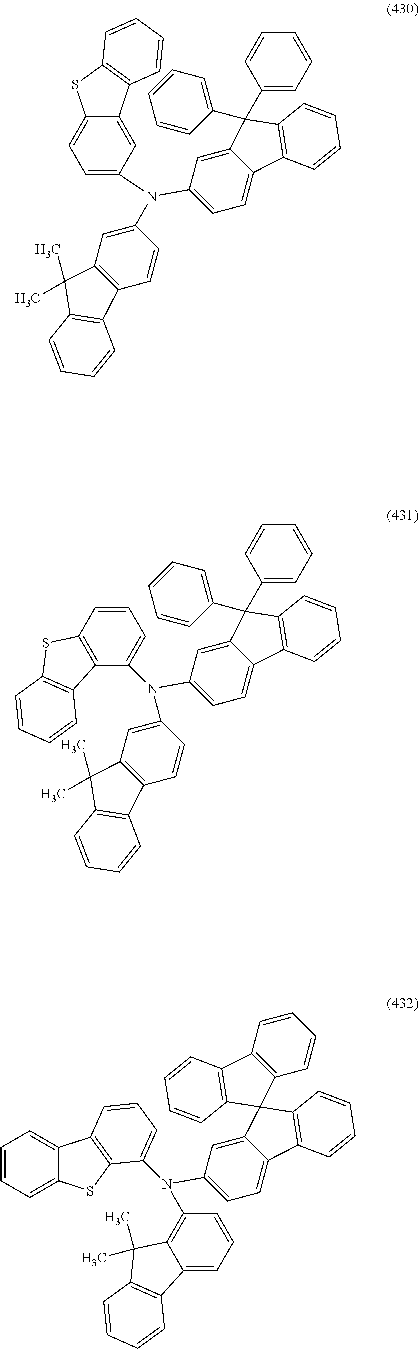

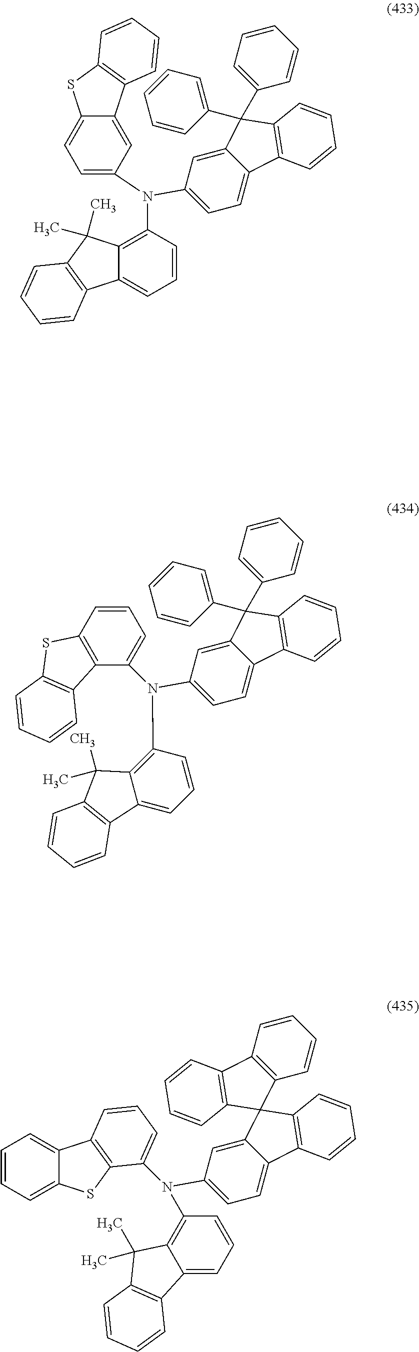

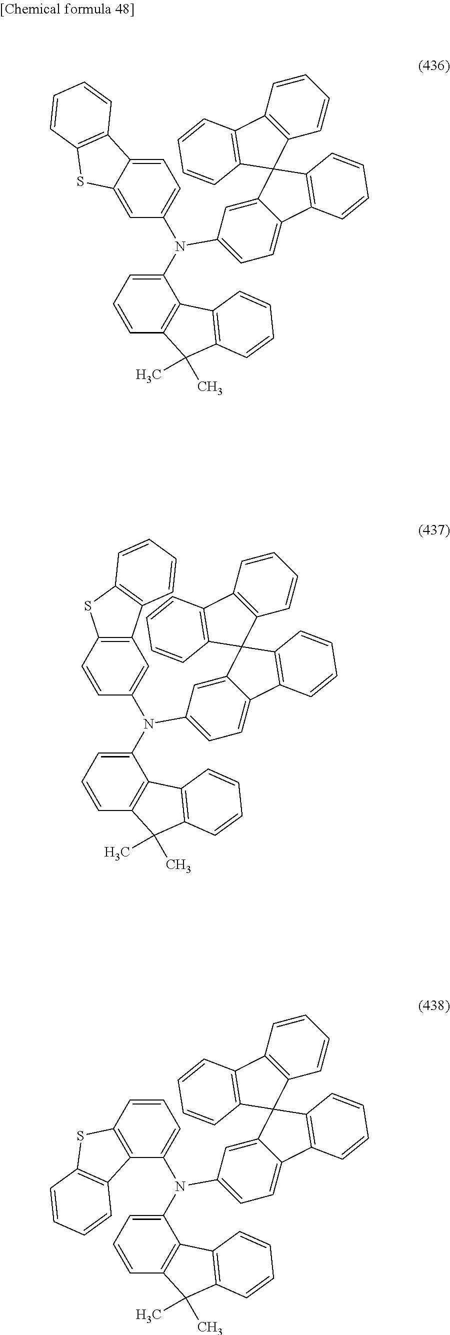

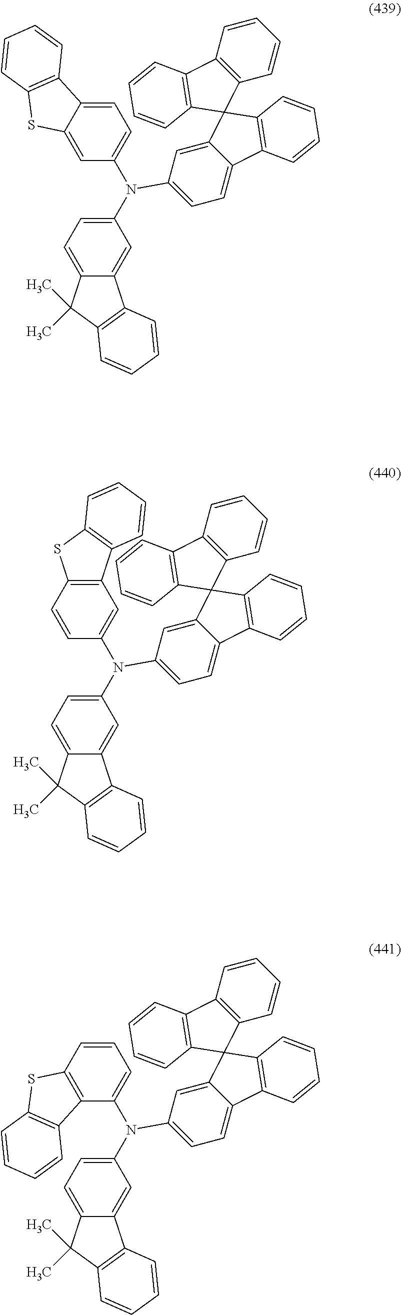

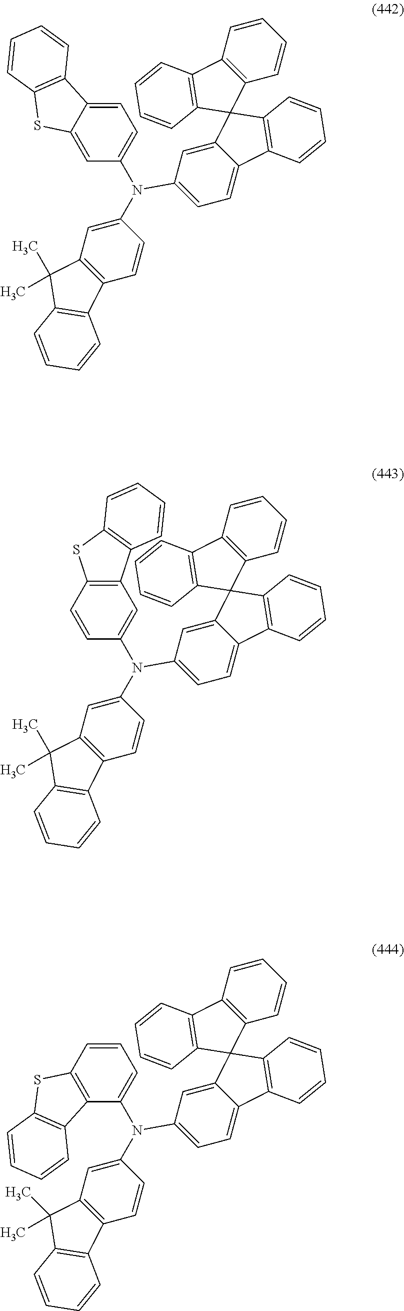

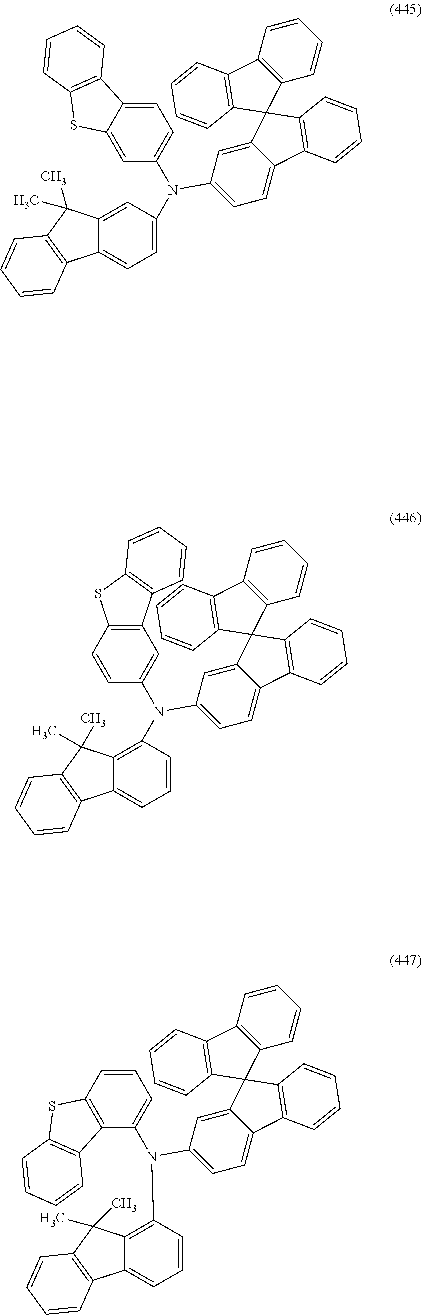

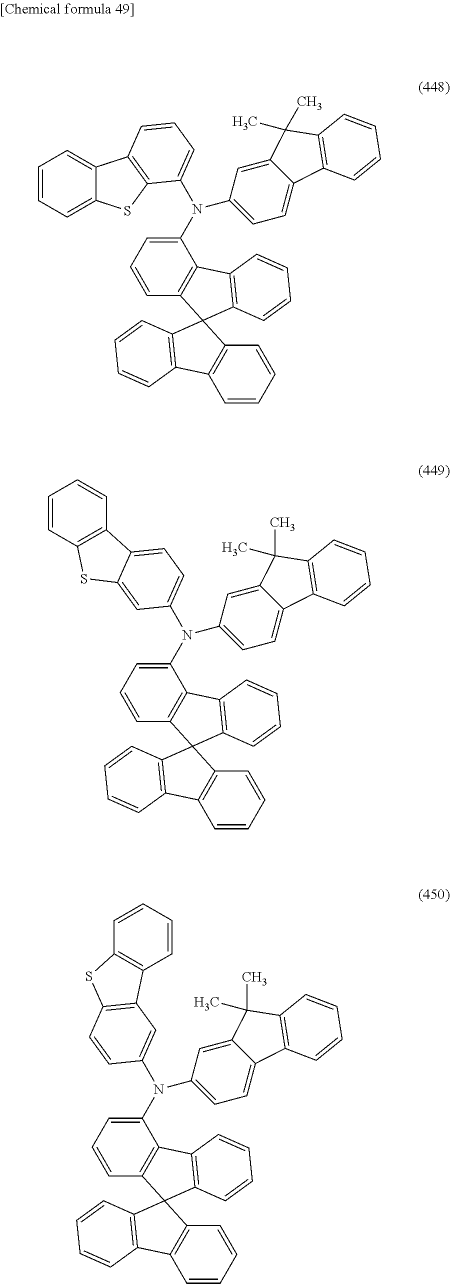

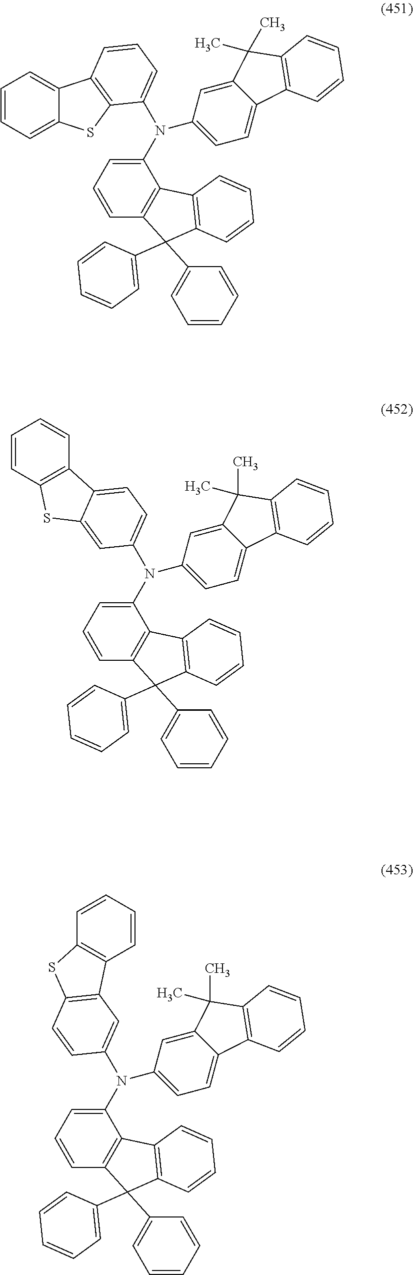

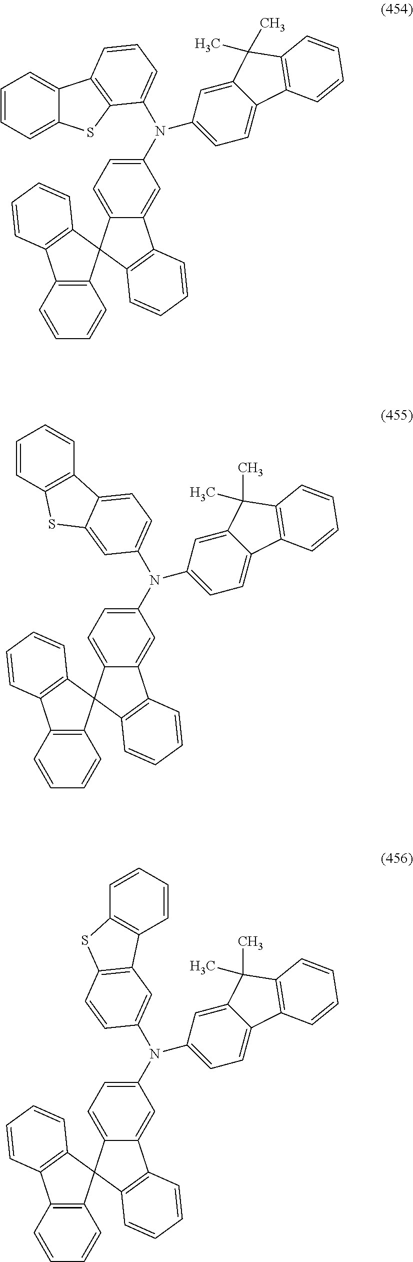

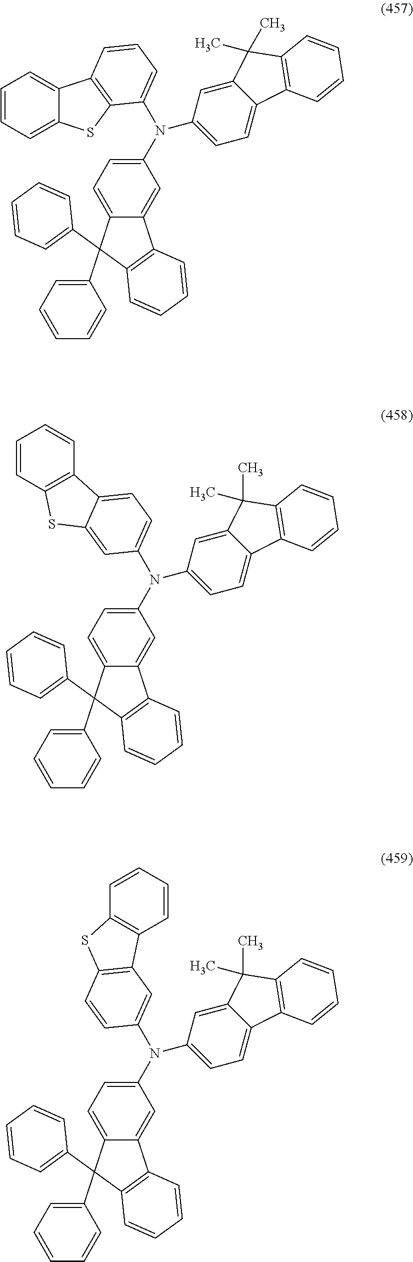

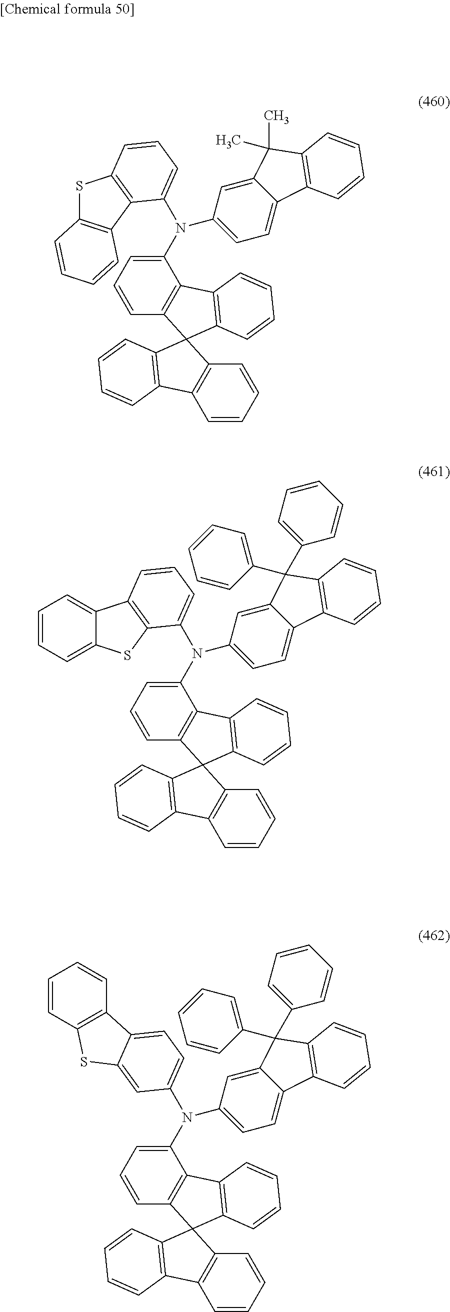

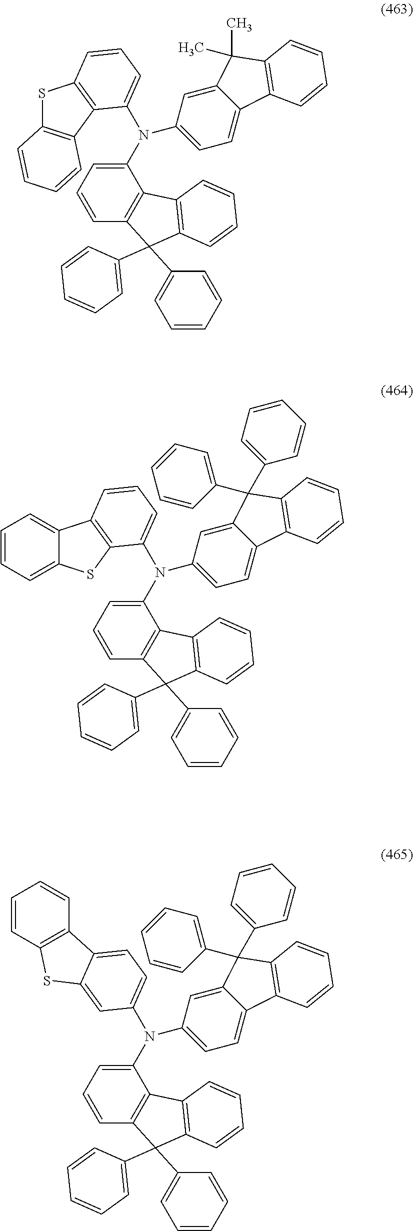

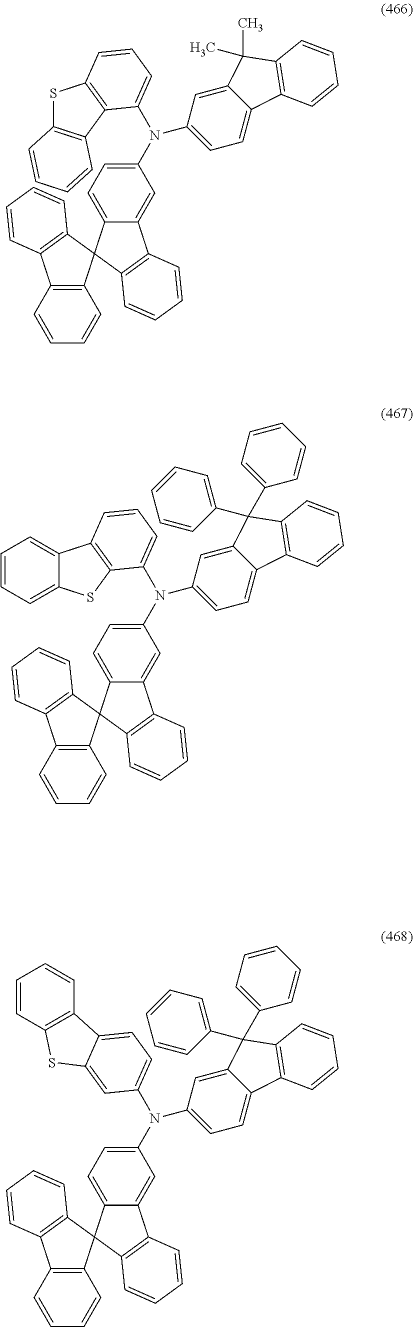

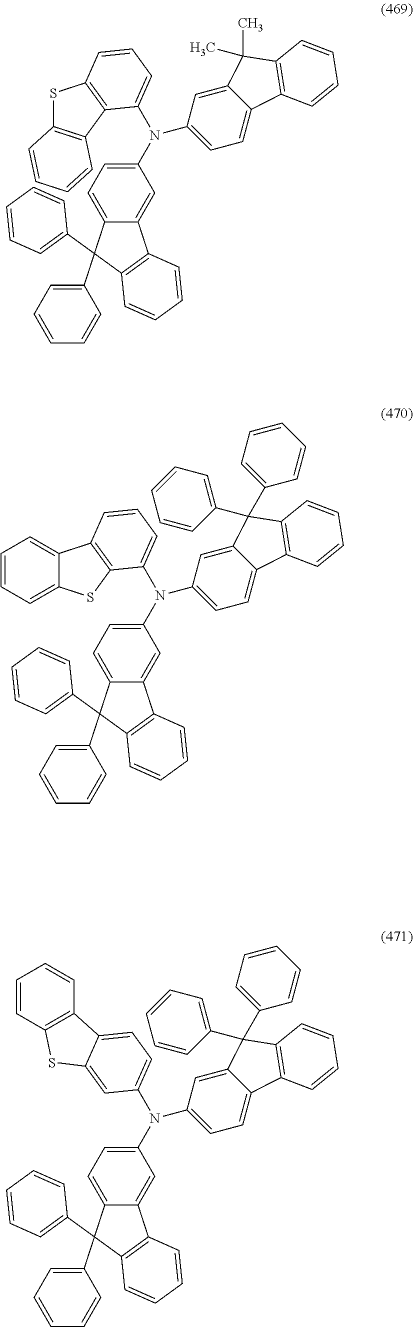

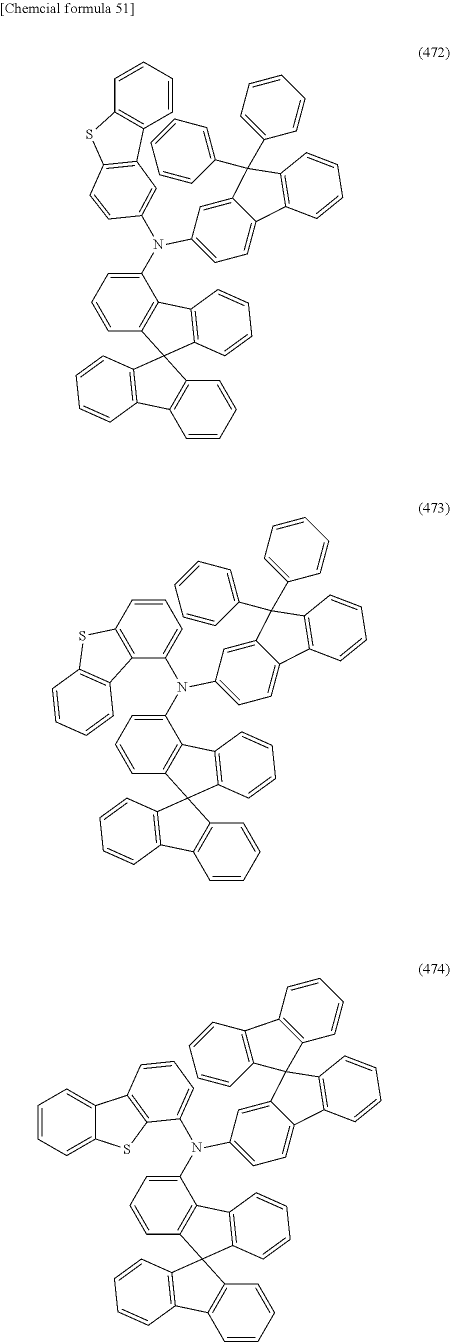

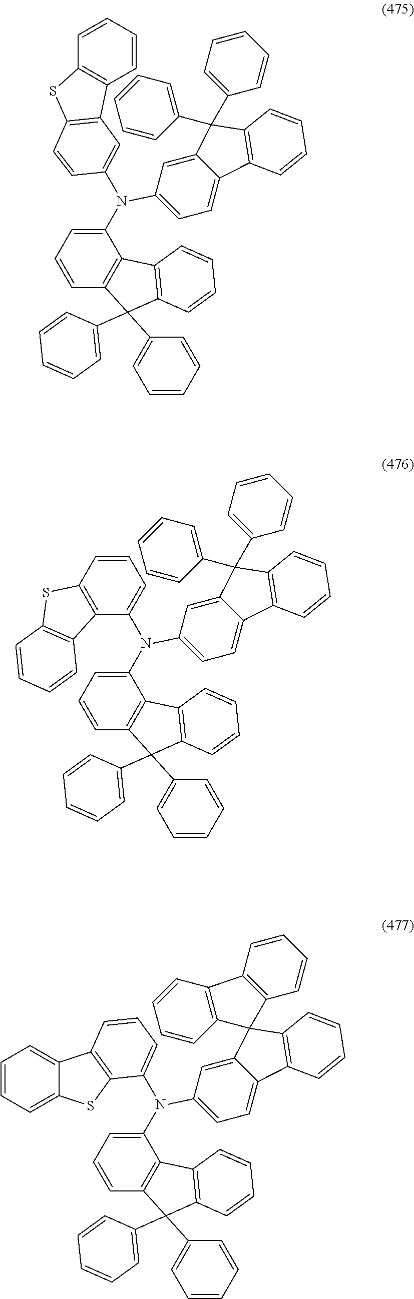

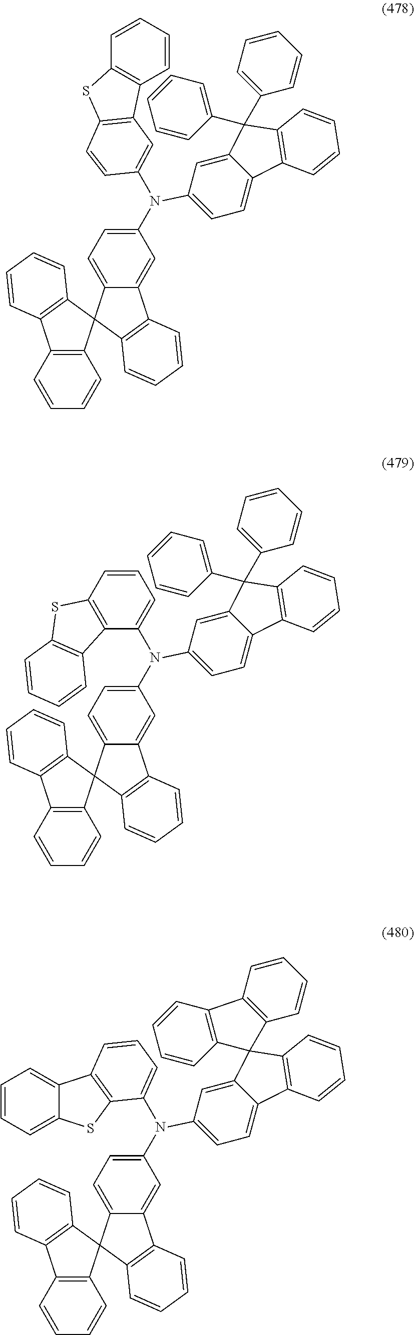

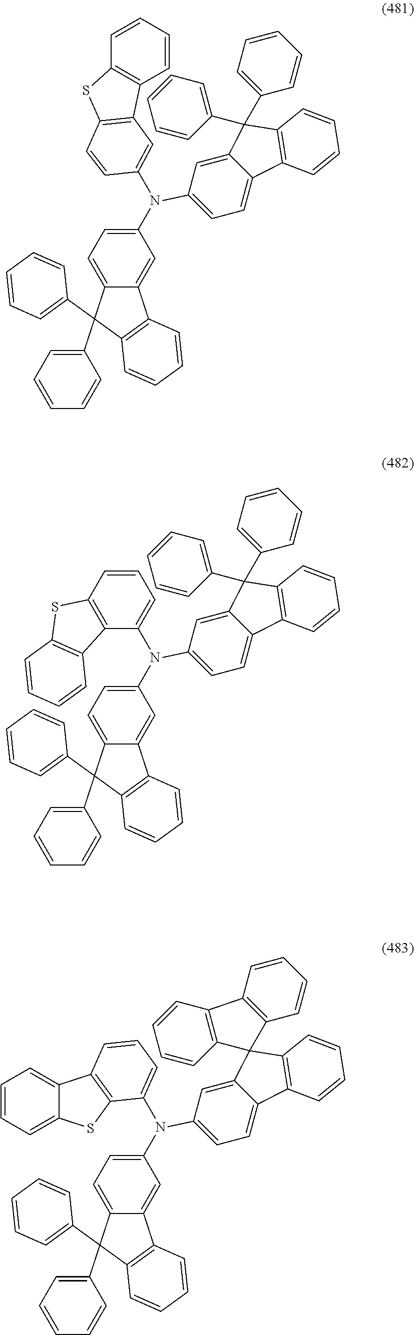

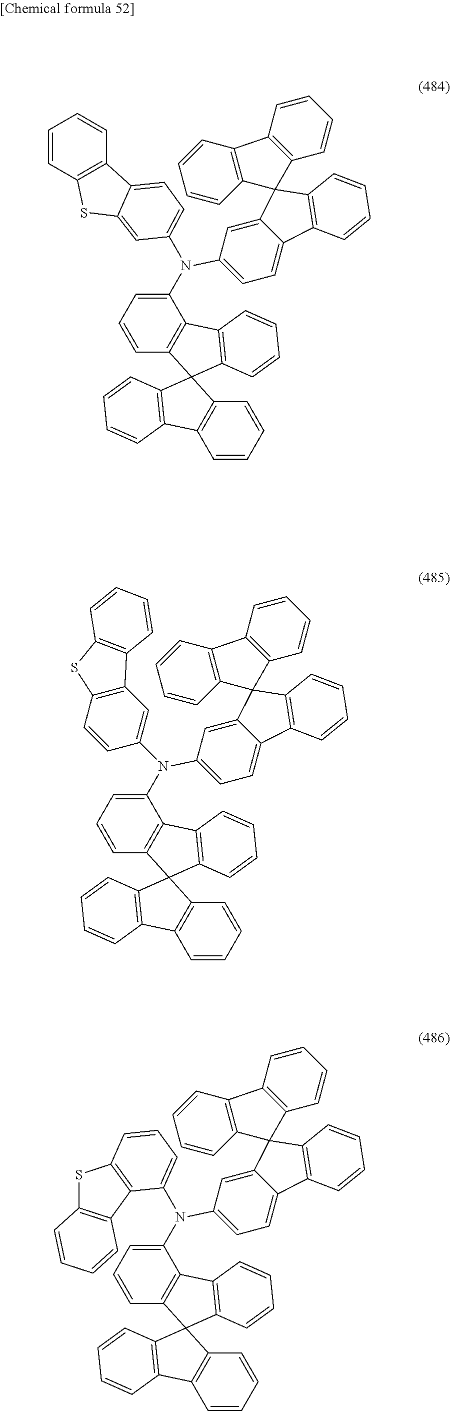

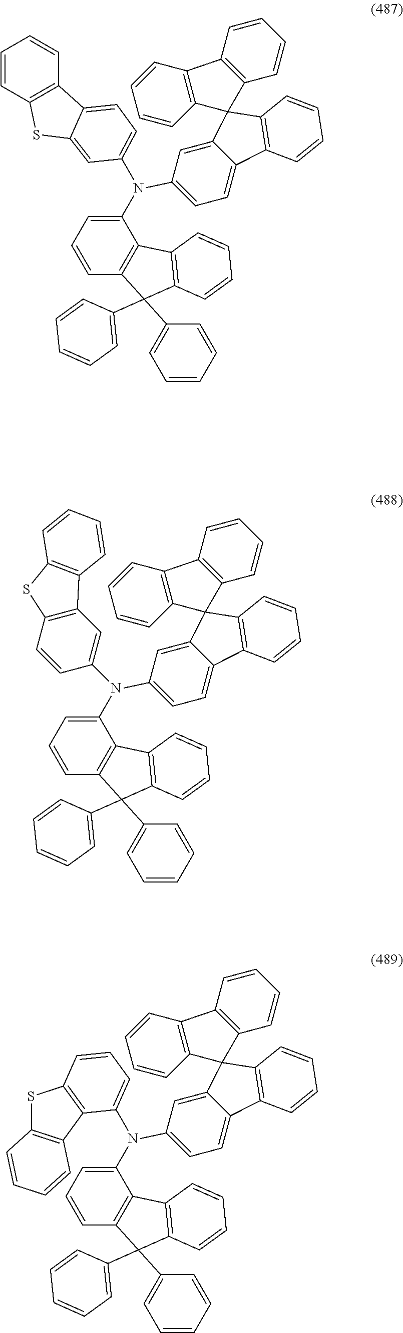

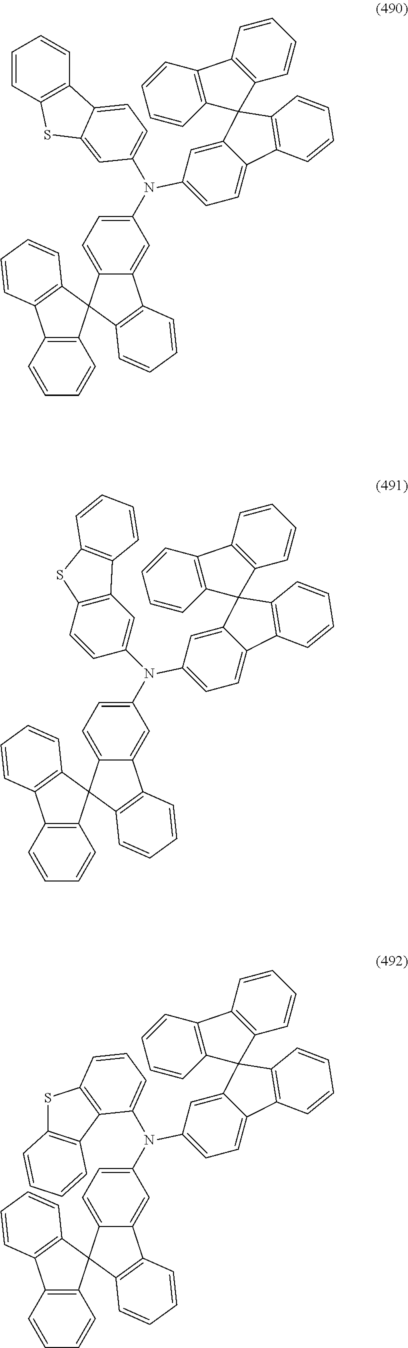

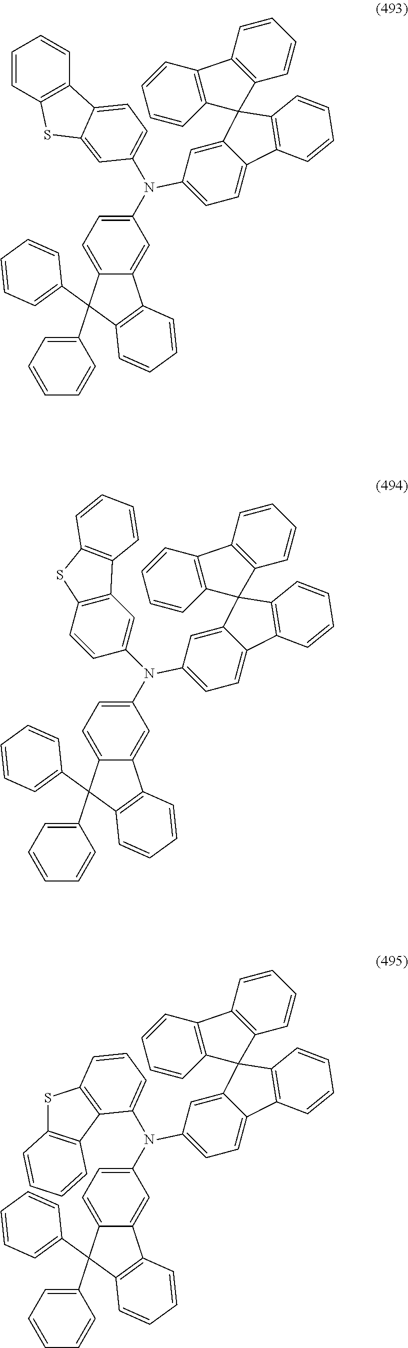

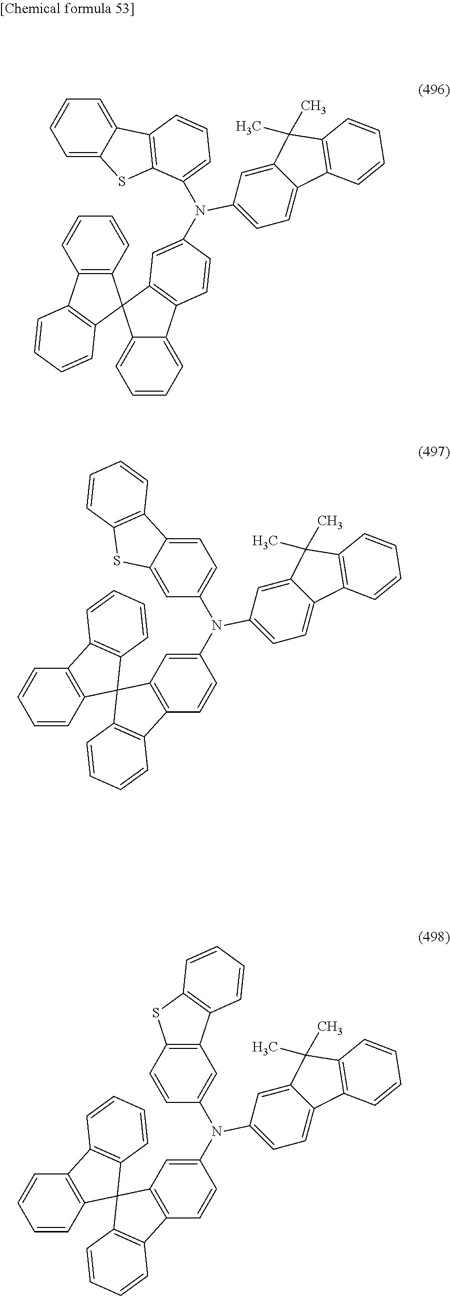

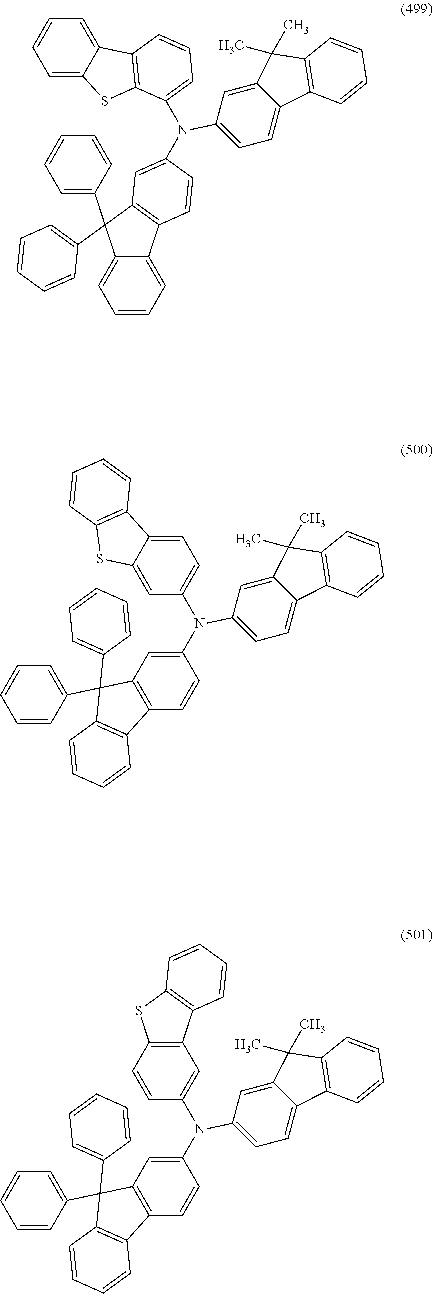

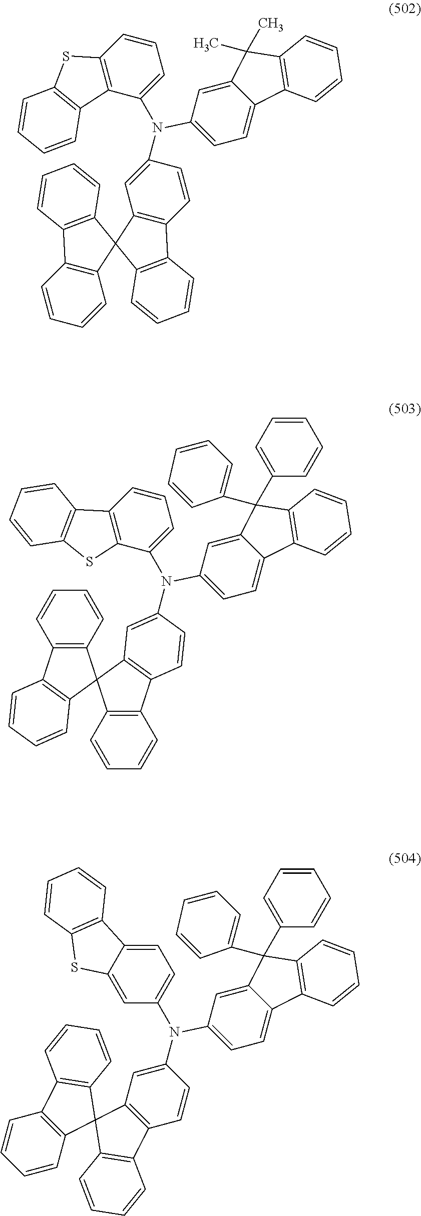

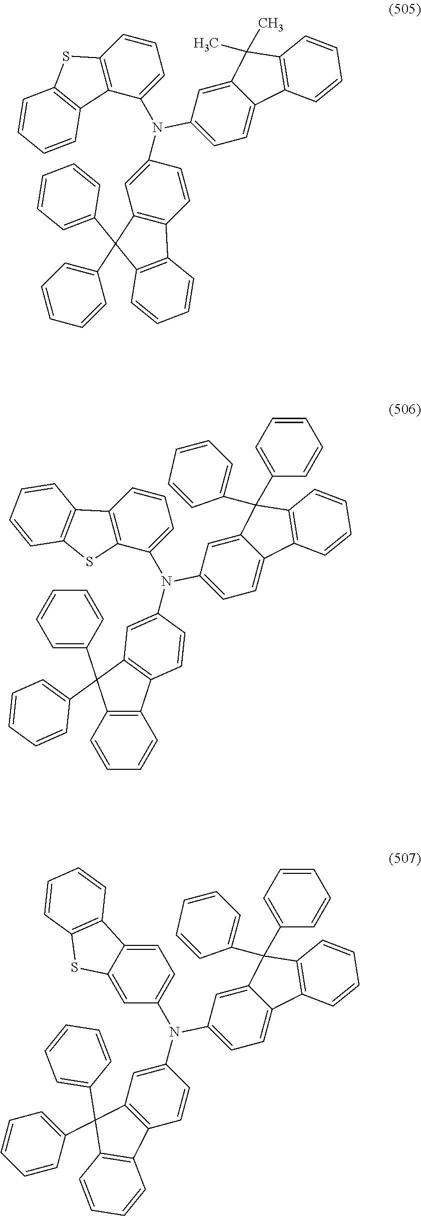

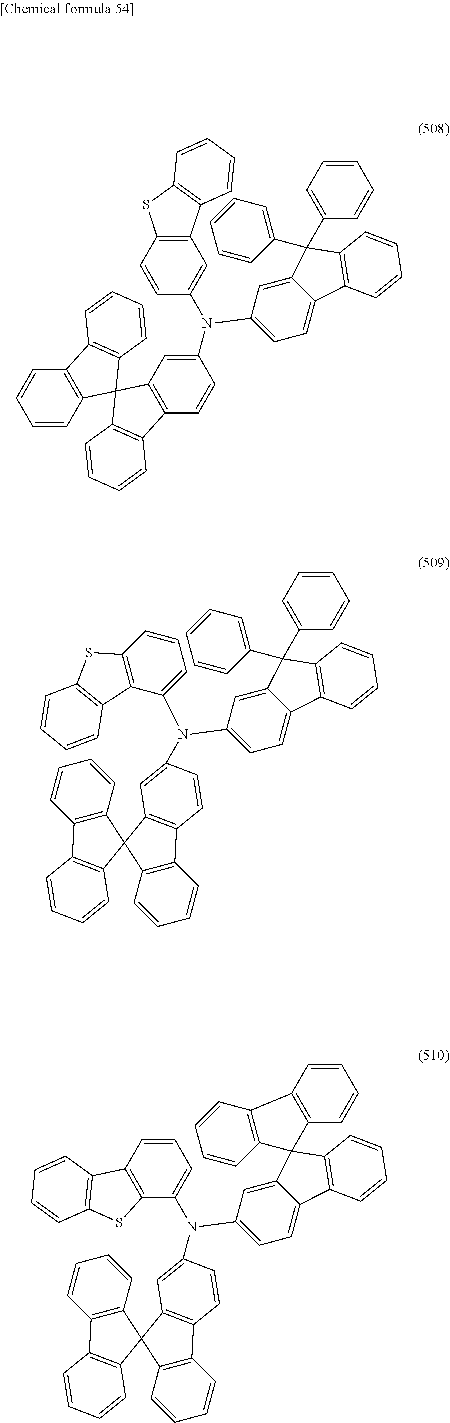

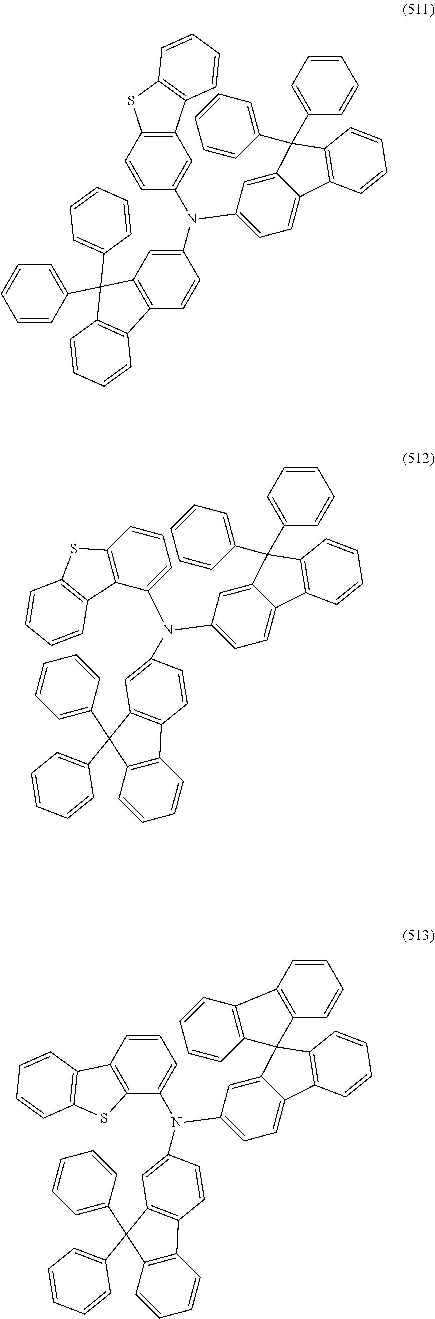

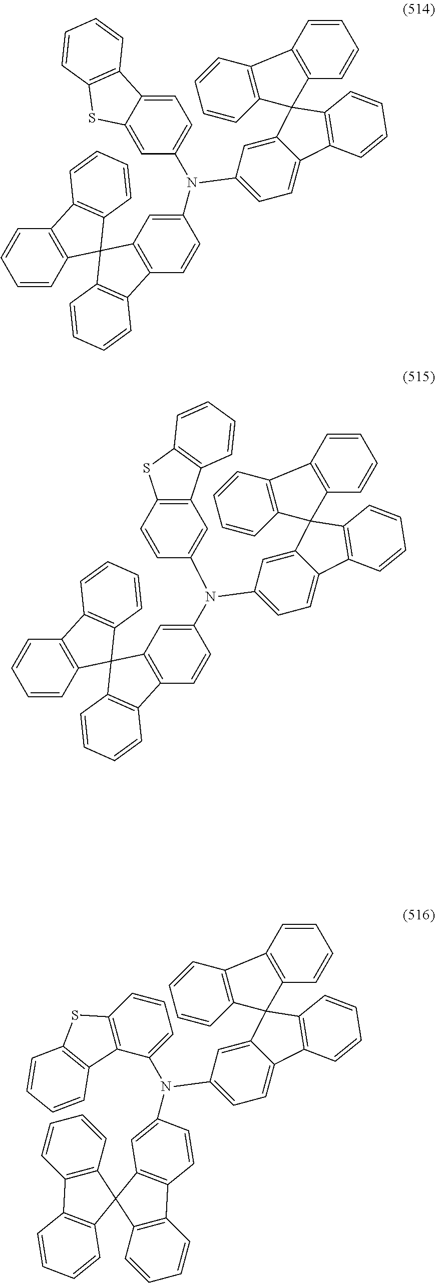

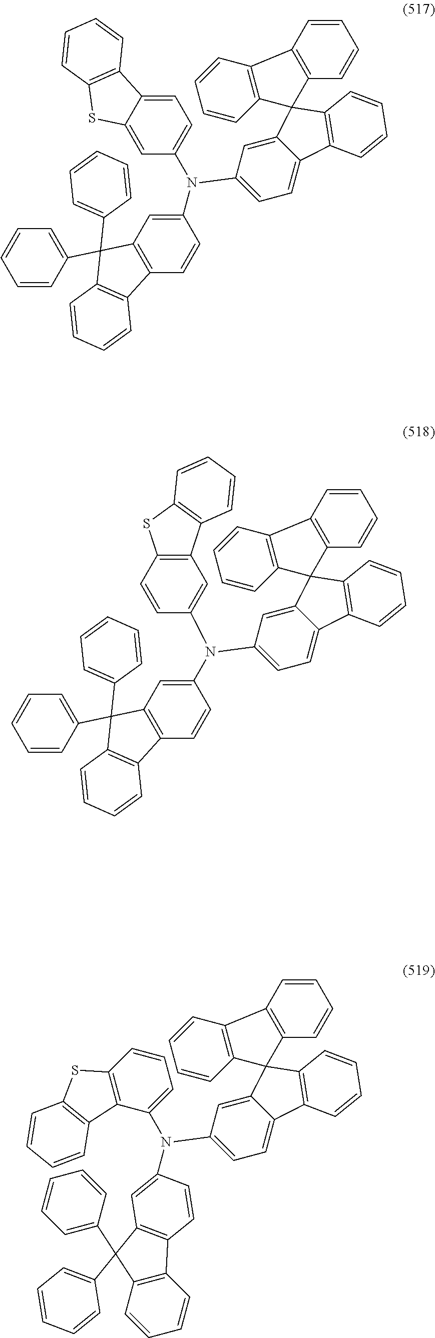

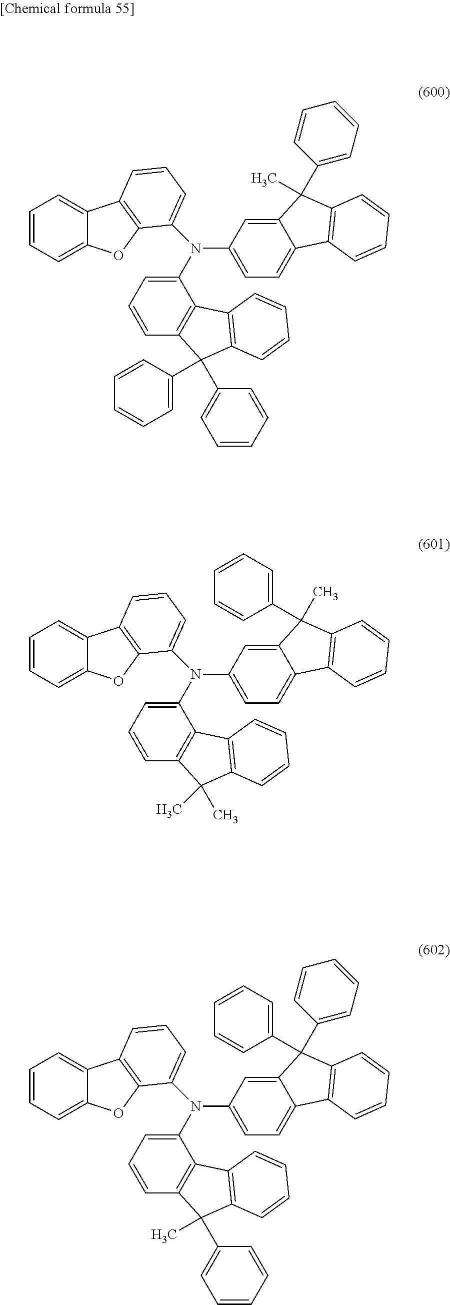

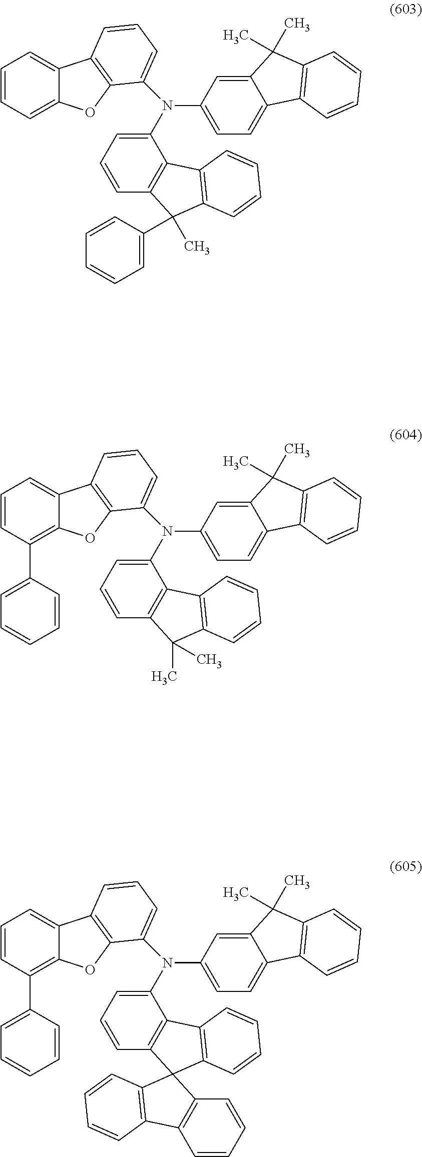

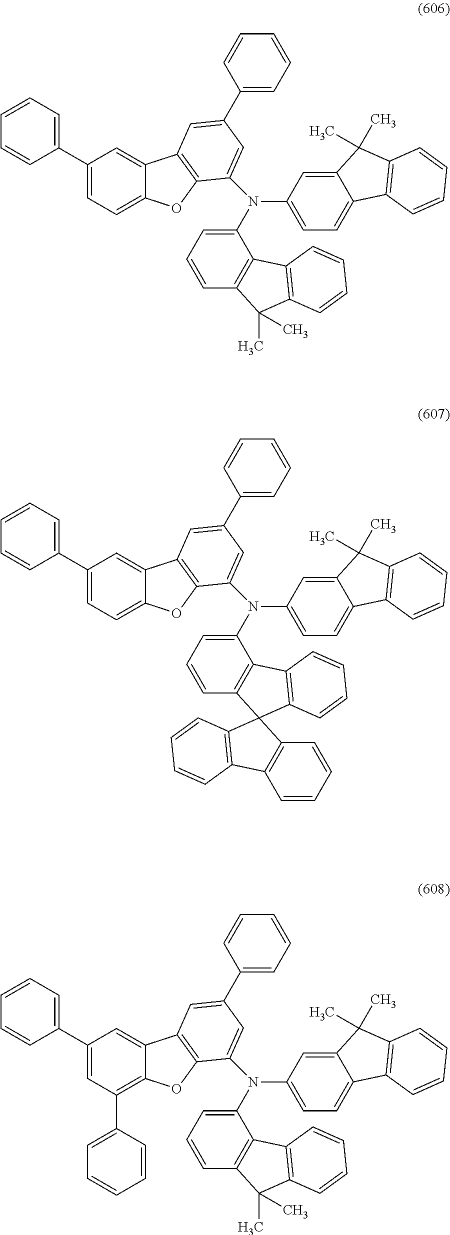

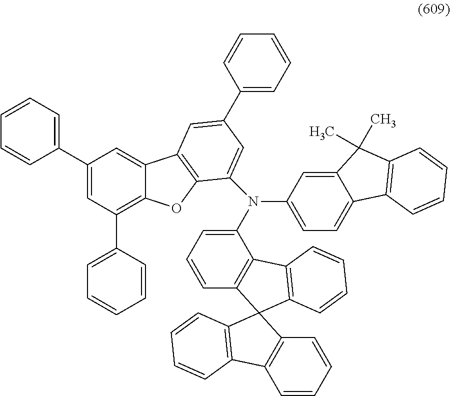

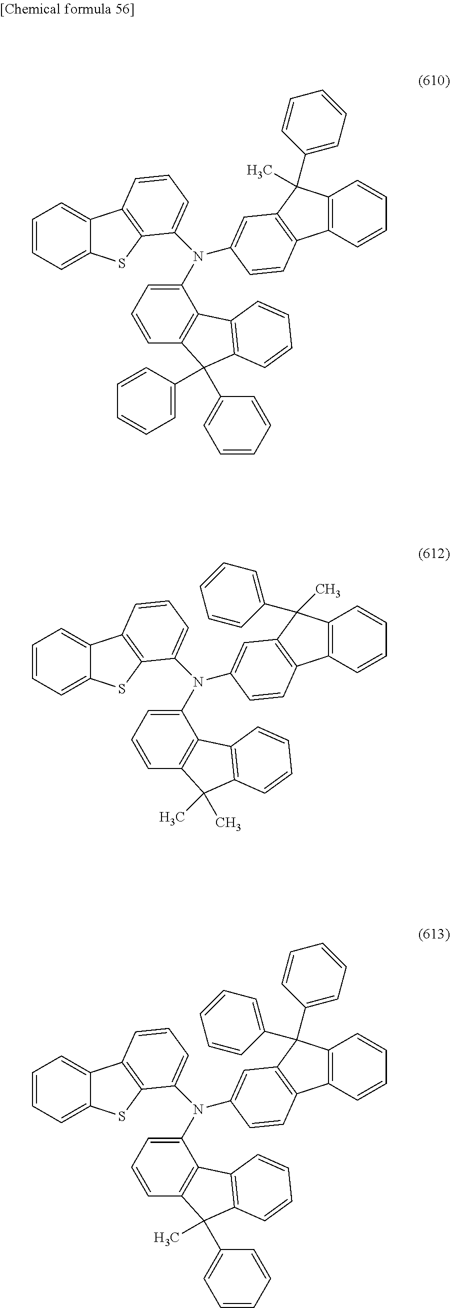

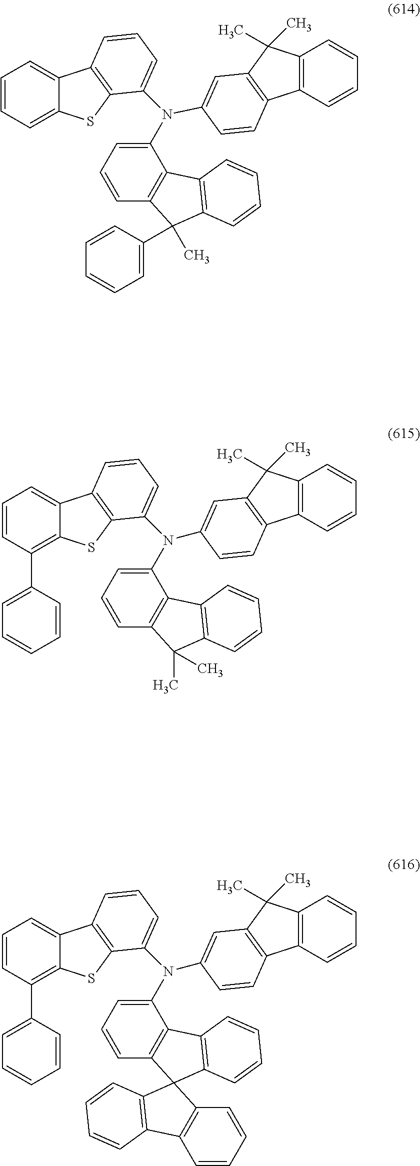

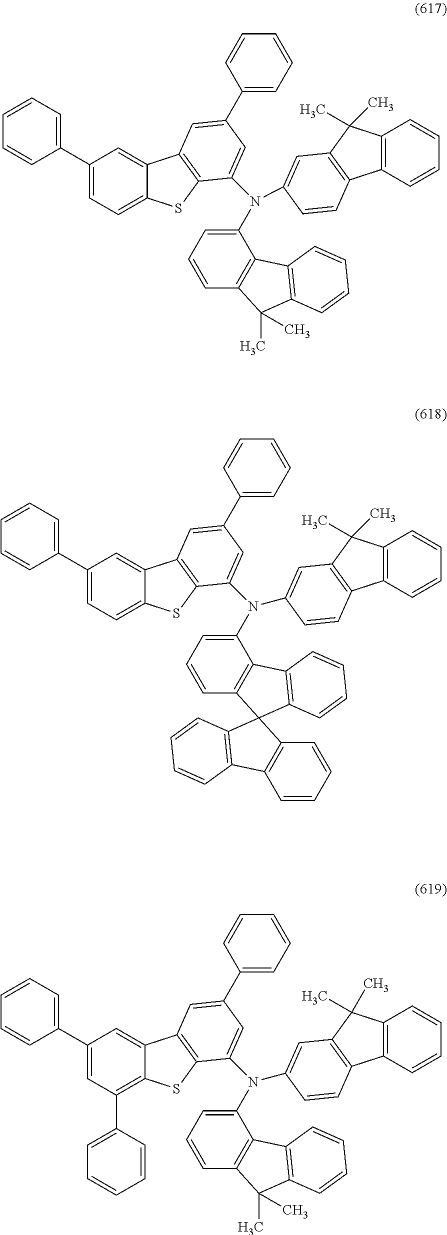

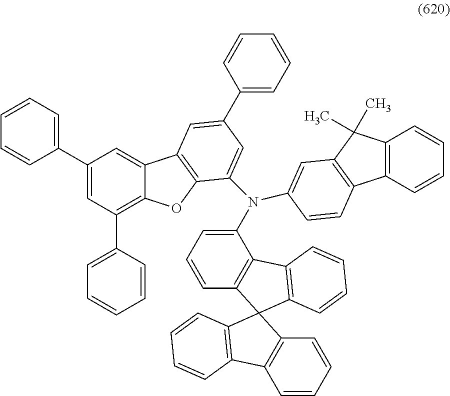

The organic compounds represented by the above Structural Formulae (100) to (161), (200) to (319), (400) to (519), and (600) to (620) are examples of the organic compounds represented by General Formulae (G1) to (G4); however, the organic compound that can be used for the light-emitting device of one embodiment of the present invention is not limited to the examples.

Specific examples of the first organic compound include an organic compound represented by General Formula (G11) below. Note that the use of the organic compound represented by General Formula (G11) below is not limited to the light-emitting device of one embodiment of the present invention. The organic compound represented by General Formula (G11) is an organic compound that has a hole-transport property and a low HOMO level and that can be suitably used for a variety of kinds of light-emitting devices.

As described above, one embodiment of the present invention is the organic compound represented by General Formula (G11).

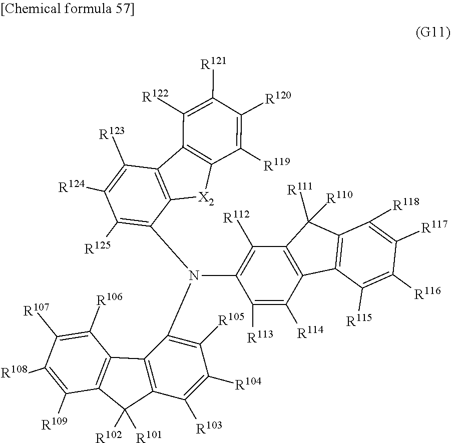

In General Formula (G11), X² represents oxygen or sulfur, and R¹⁰³ to R¹⁰⁹, R¹¹² to R¹¹⁸, and R¹¹⁹ to R¹²⁵ each independently represent hydrogen (including deuterium), a substituted or unsubstituted alkyl group having 1 to 6 carbon atoms, or a substituted or unsubstituted aryl group having 6 to 13 carbon atoms, and R¹⁰¹, R¹⁰², R¹¹⁰, and R¹¹¹ each independently represent hydrogen (including deuterium), a substituted or unsubstituted alkyl group having 1 to 6 carbon atoms, or a substituted or unsubstituted aryl group having 6 to 13 carbon atoms. Note that R¹⁰⁶, R¹¹⁴, and R¹¹⁵ each represent a substituent other than an unsubstituted phenyl group. In the case where R¹⁰¹ and R¹⁰² are each an unsubstituted phenyl group, R¹¹⁰ and R¹¹¹ each represent a substituent other than a methyl group. In the case where R¹¹⁰ and R¹¹¹ are each an unsubstituted phenyl group, R¹⁰¹ and R¹⁰² each represent a substituent other than a methyl group.

Alternatively, in General Formula (G11), X² represents oxygen or sulfur, R¹⁰³ to R¹⁰⁹, R¹¹² to R¹¹⁸, and R¹¹⁹ to R¹²⁵ each represent hydrogen (including deuterium), a substituted or unsubstituted alkyl group having 1 to 6 carbon atoms, or a substituted or unsubstituted aryl group having 6 to 13 carbon atoms, and R¹⁰¹, R¹⁰², R¹¹⁰, and R¹¹¹ each independently represent hydrogen (including deuterium), a methyl group, or an unsubstituted phenyl group. In the case where R¹⁰¹ and R¹⁰² are each an unsubstituted phenyl group, R¹¹⁰ and R¹¹¹ each represent a substituent other than a methyl group. In the case where R¹¹⁰ and R¹¹¹ are each an unsubstituted phenyl group, R¹⁰¹ and R¹⁰² each represent a substituent other than a methyl group.

In General Formula (G11), in the case where each of R¹⁰⁶, R¹¹⁴, and R¹¹⁵ has a phenyl group, specifically, a phenyl group at the 4-position or the 5-position of the fluorene skeleton, the compound has a difficulty in accepting holes; therefore, no substituent or a substituent other than a phenyl group is preferred.

In addition, in the case in which General Formula (G11) has both a diphenylfluorene skeleton and a dimethylfluorene skeleton, the synthesis method of the compound becomes complicated, leading to a high cost. For example, a compound having two diphenylfluorene skeletons or a compound having two dimethylfluorene skeletons might be produced as a by-product and thus a high purity is likely to be difficult to achieve. In General Formula (G11), in the case in which R¹⁰¹ and R¹⁰² are each an unsubstituted phenyl group, R¹¹⁰ and R¹¹¹ are each preferably a substitute other than a methyl group, and in the case in which R¹¹⁰ and R¹¹¹ are each an unsubstituted phenyl group, R¹⁰¹ and R¹⁰² are each preferably a substitute other than a methyl group.

Specific examples of an alkyl group having 1 to 6 carbon atoms in R¹⁰³ to R¹⁰⁹, R¹¹² to R¹¹⁸, and R¹¹⁹ to R¹²¹ in General Formula (G11) include a methyl group, an ethyl group, a propyl group, an isopropyl group, a butyl group, a sec-butyl group, an isobutyl group, and a tert-butyl group. Specific examples of the aryl group having 6 to 13 carbon atoms in R¹⁰³ to R¹⁰⁹, R¹¹² to R¹¹⁸, and R¹¹⁹ to R¹²⁵ in General Formula (G11) include a phenyl group, a tolyl group, a xylyl group, a mesityl group, a biphenyl group, a naphthyl group, and a fluorenyl group. Furthermore, at least two of the substituents may be bonded to each other to form a ring; a spirobifluorenyl group is regarded as a group having substituents which are bonded to each other to form a ring (that is, a spirobifluorenyl group is a compound in which two phenyl groups in a 9,9-diphenylfluorenyl group are bonded to form a ring).

In addition, another specific example of the first organic compound is an organic compound represented by General Formula (G12) below. Note that the use of the organic compound represented by General Formula (G12) below is not limited to the light-emitting device of one embodiment of the present invention. The organic compound represented by General Formula (G12) is an organic compound that has a hole-transport property and a low HOMO level and that can be suitably used for a variety of kinds of light-emitting devices.

As described above, one embodiment of the present invention is the organic compound represented by General Formula (G12).

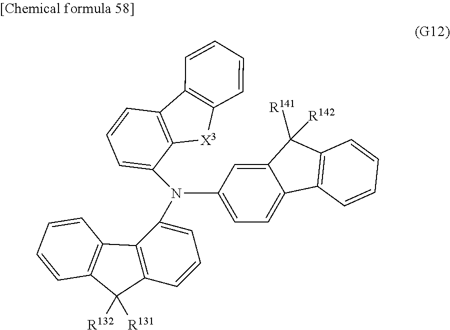

In General Formula (G12), X³ represents oxygen or sulfur, three or all of R¹³¹, R¹³², R¹⁴¹, and R¹⁴² represent the same substituent, the substituent represents hydrogen (including deuterium), a methyl group, or an unsubstituted phenyl group, and the other thereof represents hydrogen (including deuterium), a methyl group, or an unsubstituted phenyl group. Note that some or all of hydrogen atoms in General Formula (G12) may be deuterium.

Note that in General Formula (G12), all of R¹³¹, R¹³², R¹⁴¹, and R¹⁴² preferably have the same substituent, in which case the compound can be easily produced and thus is easily highly purified. In addition, when three of R¹³¹, R¹³², R¹⁴¹, and R¹⁴² represent the same substituent and the other has a different substituent, the organic compound contains two fluorene rings whose combinations of the substituents bonded to carbon of the 9-positions are different from each other, and thus the organic compound can be expected to have high heat resistance. Consequently, a highly heat-resistant organic device can be provided.

Specific examples of structures represented by the above General Formulae (G11) and (G12) are included in the specific examples of the structures represented by General Formulae (G1) to (G4). Specific examples of the structures represented by the above General Formulae (G11) and (G12) extracted from the specific examples of the structures represented by General Formulae (G1) to (G4) are given below.

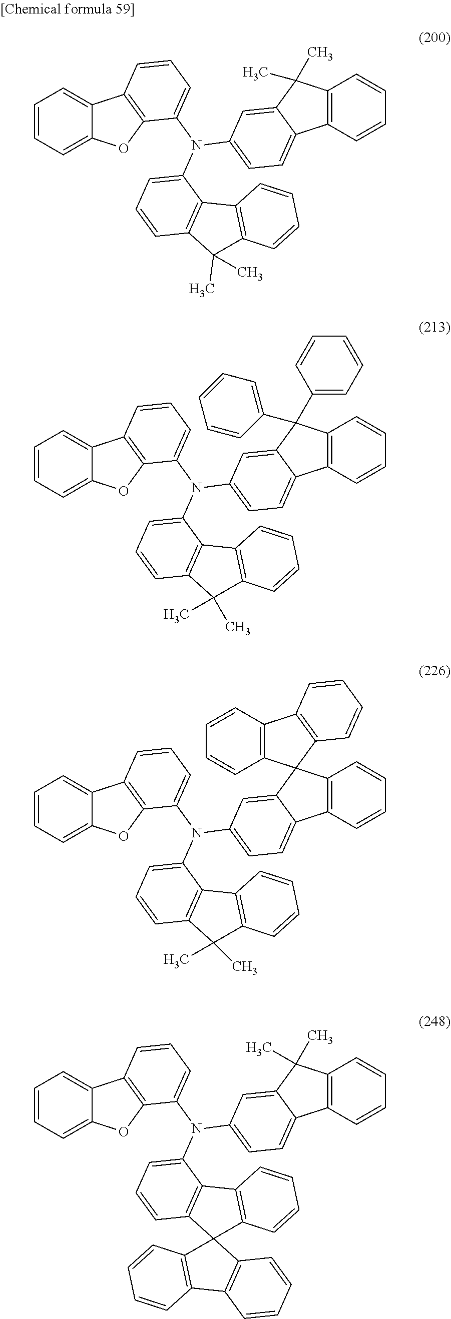

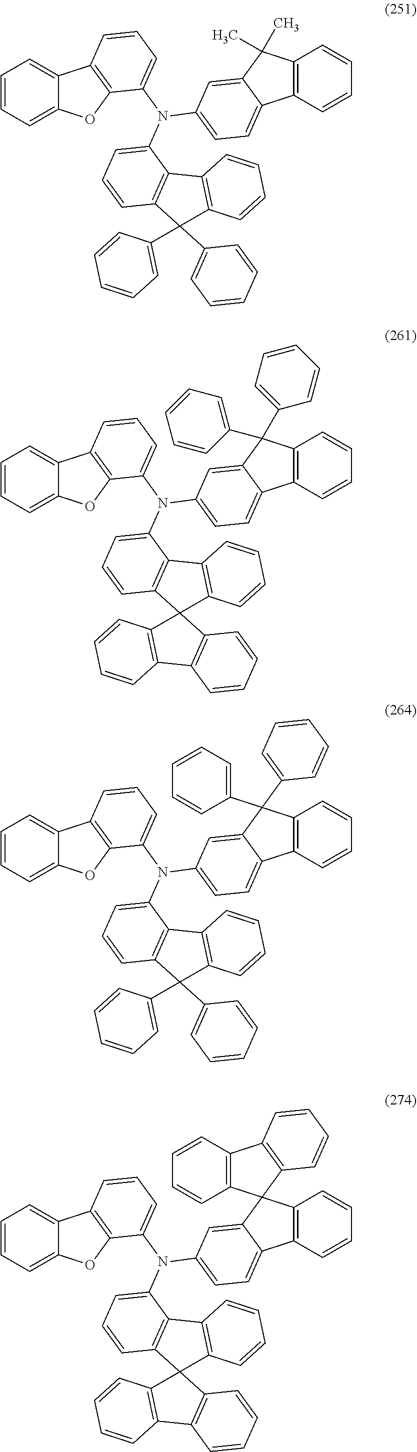

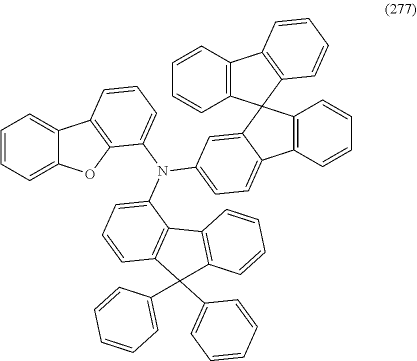

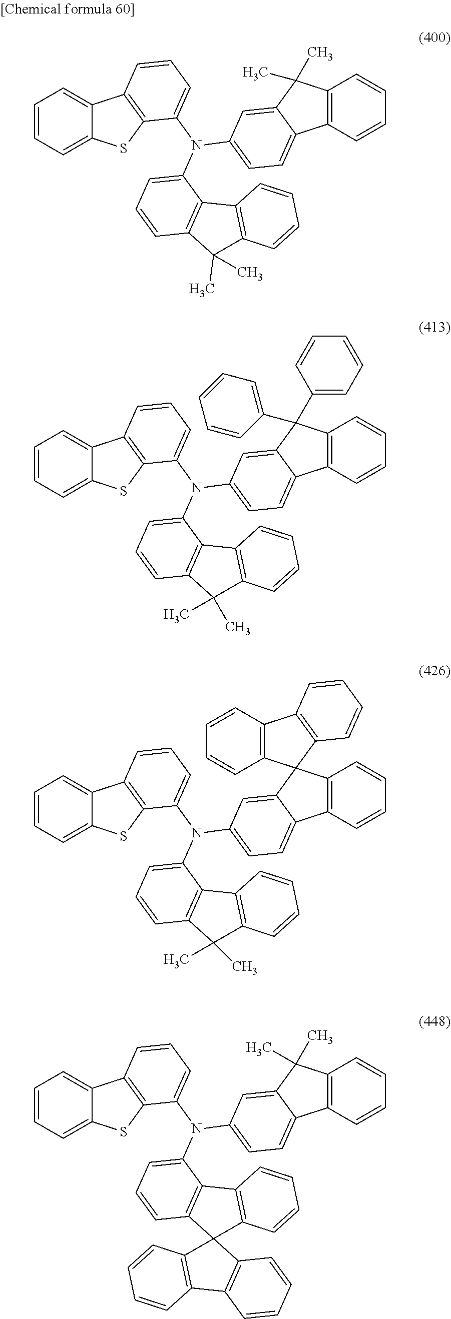

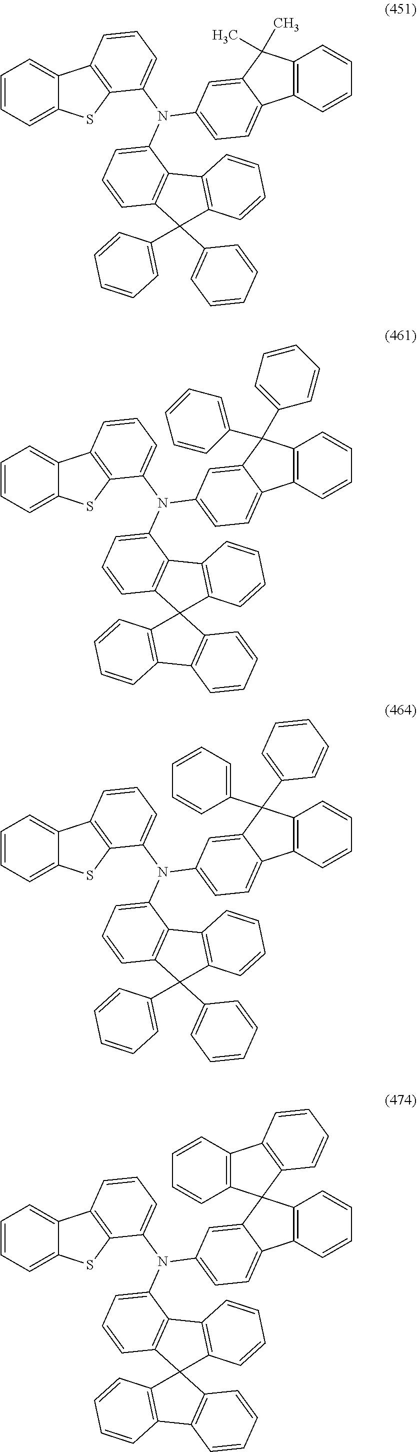

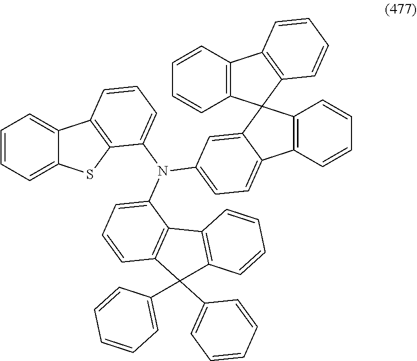

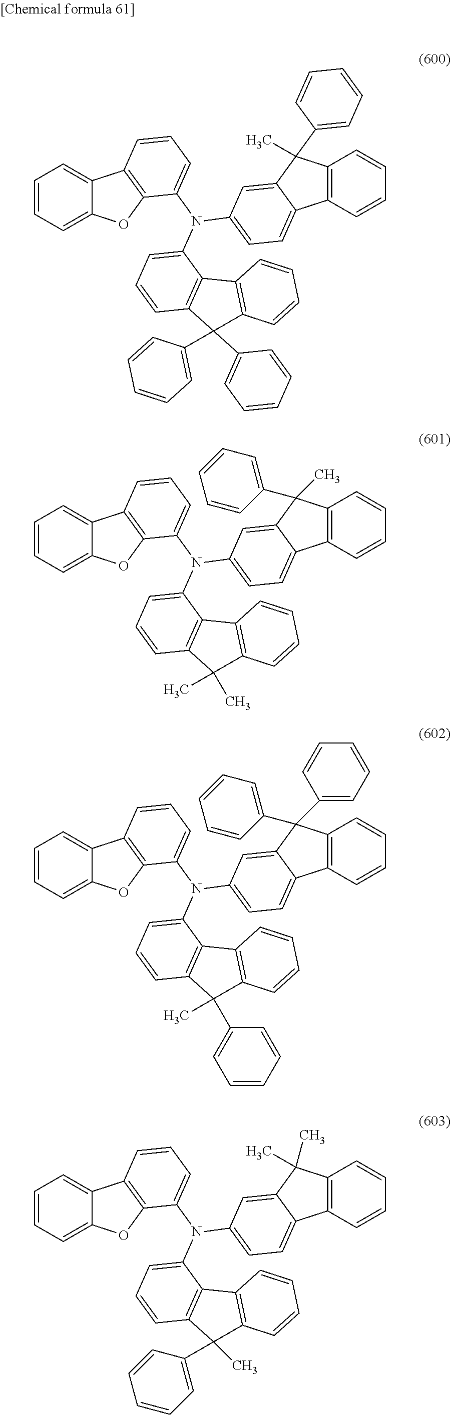

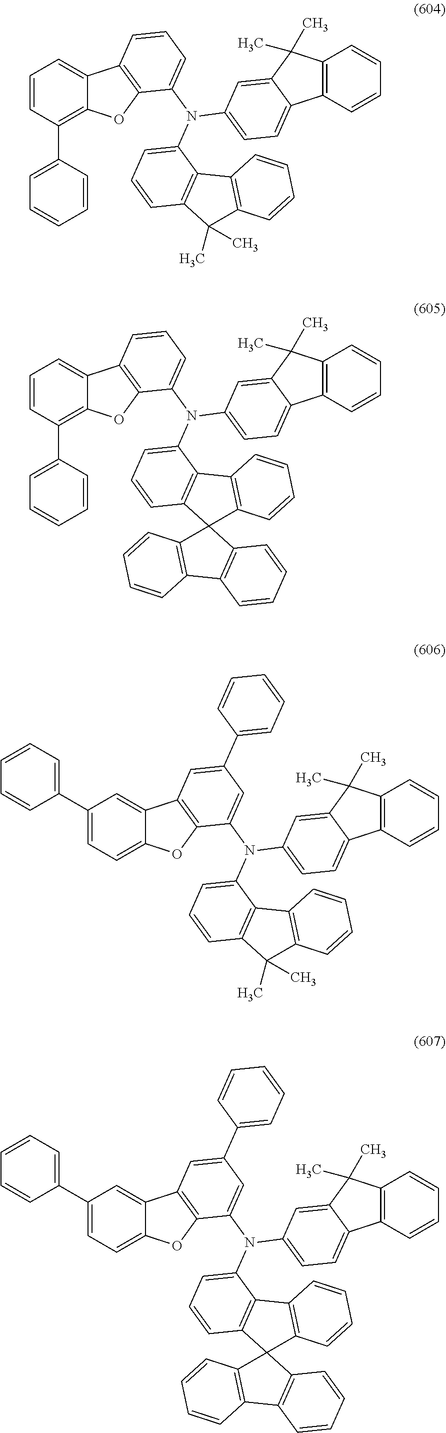

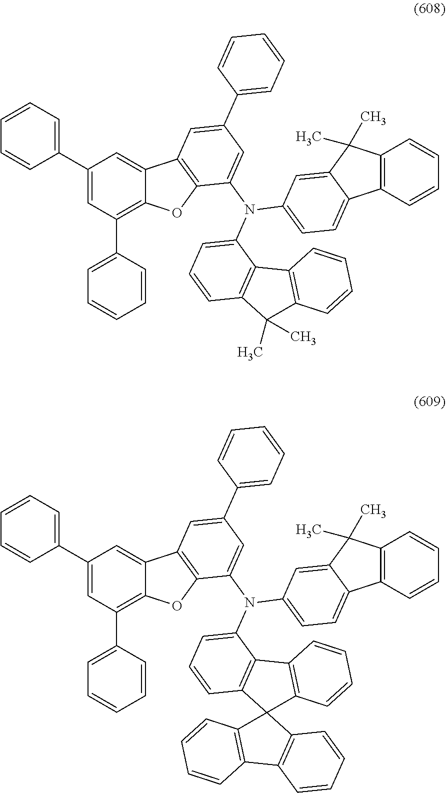

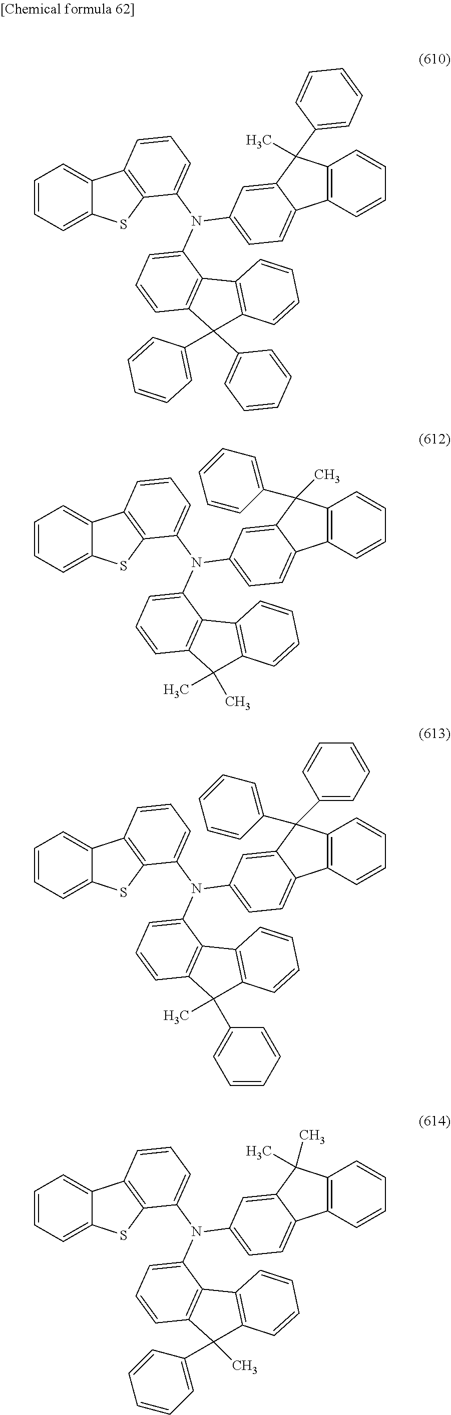

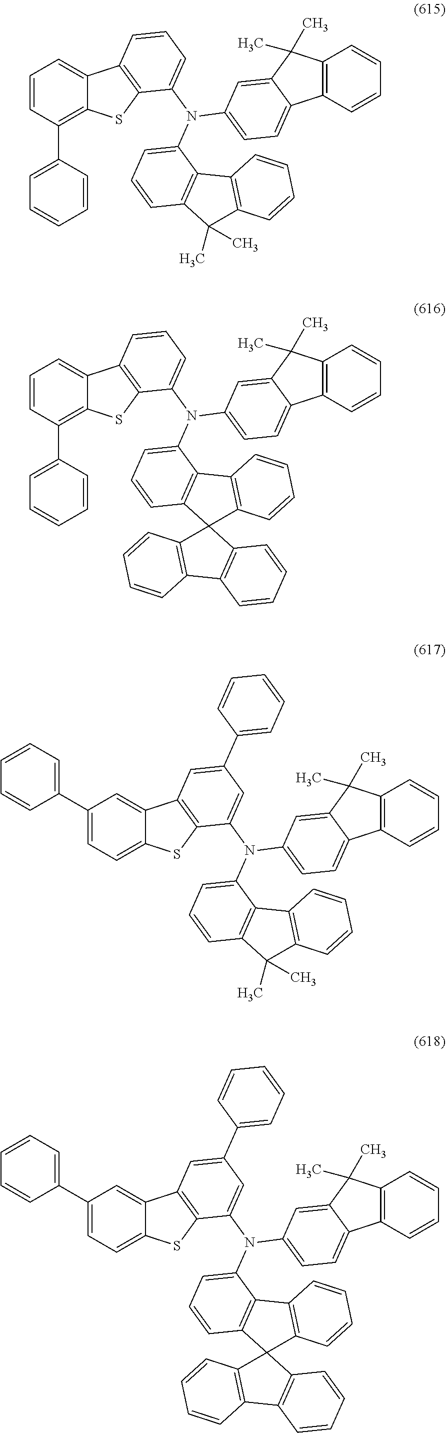

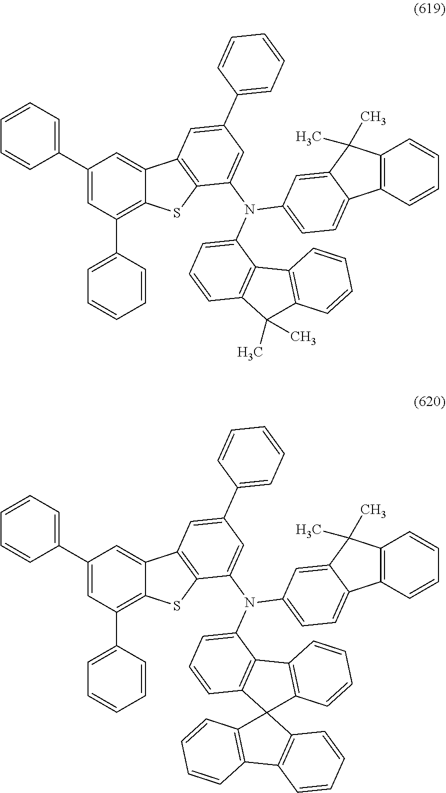

The organic compounds represented by the above structural formulae are specific examples of structures represented by General Formulae (G11) and (G12) extracted from the specific examples of the structures represented by General formulae (G1) to (G4), and the organic compound of one embodiment of the present invention is not limited to the examples.

Next, a method for synthesizing the organic compound of one embodiment of the present invention represented by General Formula (G11) will be described.

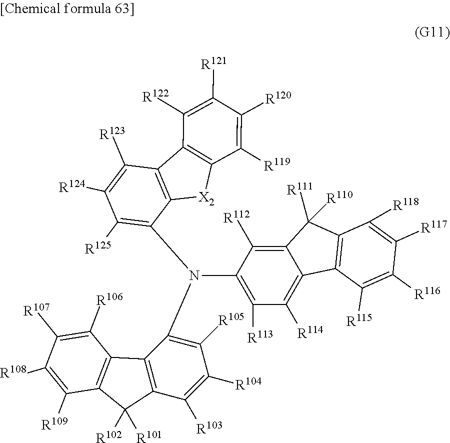

In General Formula (G11), X² represents oxygen or sulfur, R¹⁰³ to R¹⁰⁹, R¹¹² to R¹¹⁸, and R¹¹⁹ to R¹²⁵ each independently represent hydrogen (including deuterium), a substituted or unsubstituted alkyl group having 1 to 6 carbon atoms, or a substituted or unsubstituted aryl group having 6 to 13 carbon atoms, and R¹⁰¹, R¹⁰², R¹¹⁰, and R¹¹¹ each independently represent hydrogen (including deuterium), a substituted or unsubstituted alkyl group having 1 to 6 carbon atoms, or a substituted or unsubstituted aryl group having 6 to 13 carbon atoms. Note that R¹⁰⁶, R¹¹⁴, and R¹¹⁵ each represent a substituent other than an unsubstituted phenyl group. In the case where R¹⁰¹ and R¹⁰² are each an unsubstituted phenyl group, R¹¹⁰ and R¹¹¹ each represent a substituent other than a methyl group. In the case where R¹¹⁰ and R¹¹¹ are each an unsubstituted phenyl group, R¹⁰¹ and R¹⁰² each represent a substituent other than a methyl group.

Synthesis Schemes (A-1) and (A-2), Synthesis Schemes (A-3) and (A-4), Synthesis Schemes (A-5) and (A-6), and Synthesis Schemes (A-7) and (A-8) of the organic compound of one embodiment of the present invention represented by General Formula (G11) are shown below. The organic compound of one embodiment of the present invention represented by General Formula (G11) can be synthesized by any of Synthesis Schemes (A-1) and (A-2), Synthesis Schemes (A-3) and (A-4), Synthesis Schemes (A-5) and (A-6), and Synthesis Schemes (A-7) and (A-8).

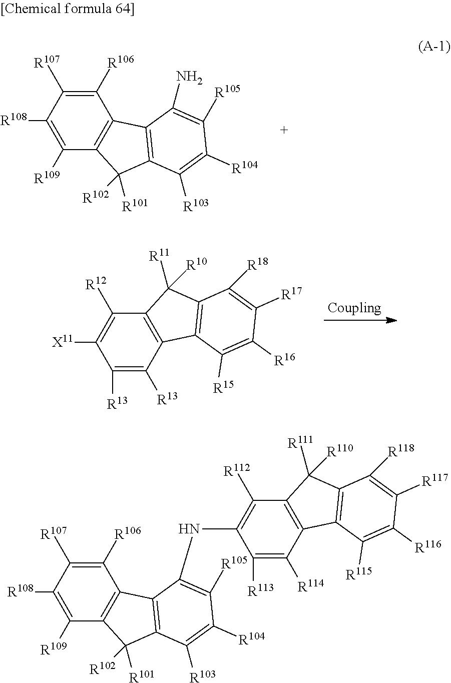

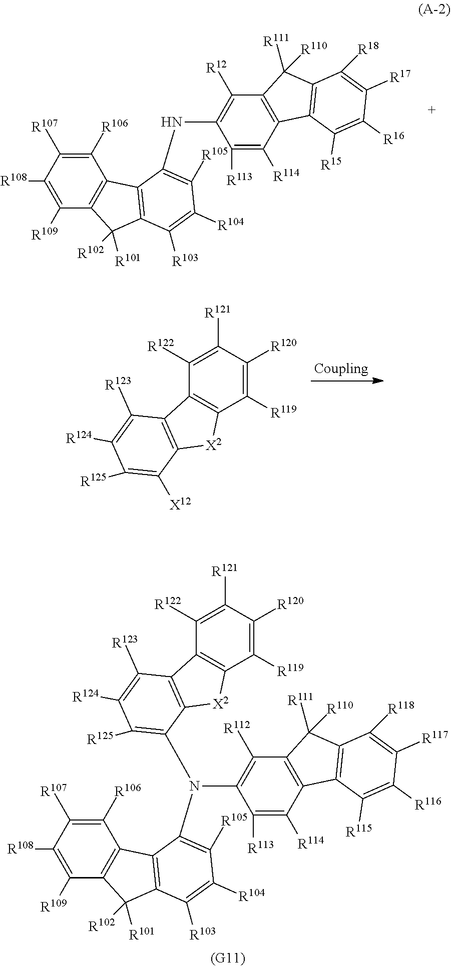

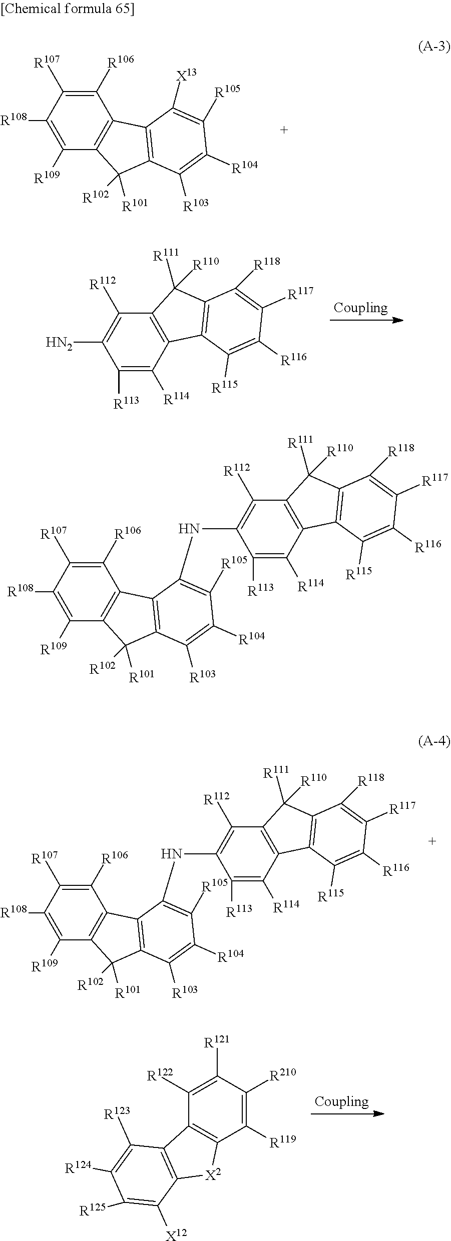

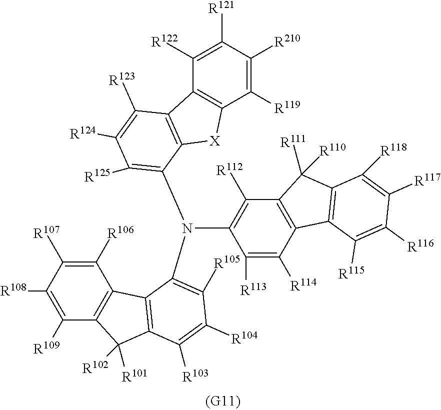

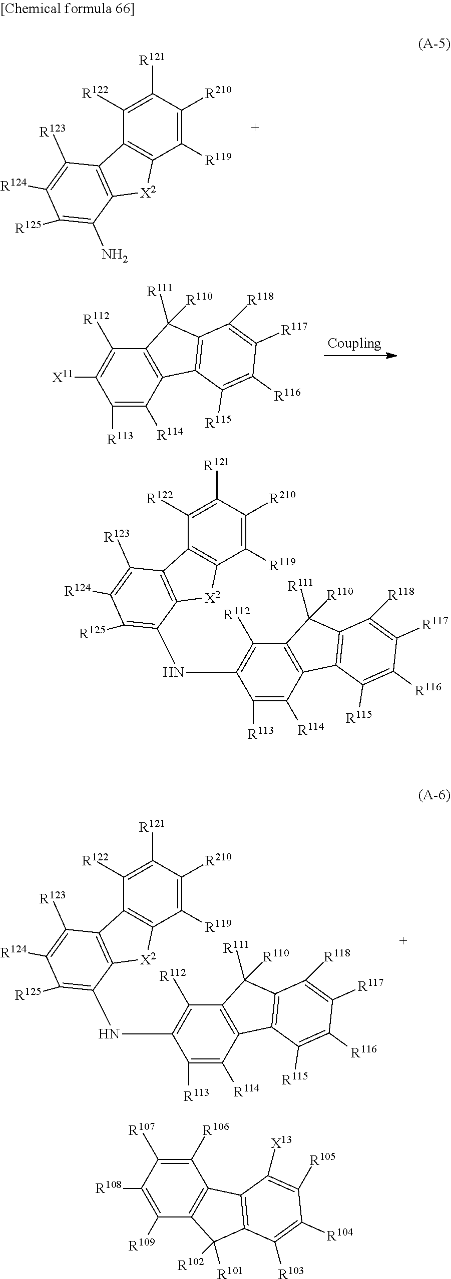

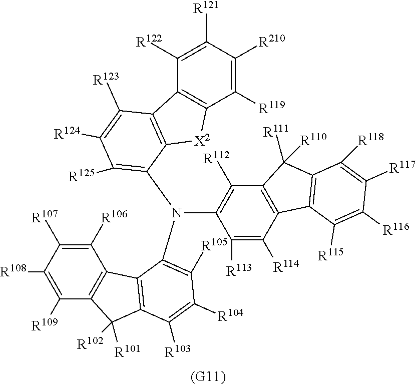

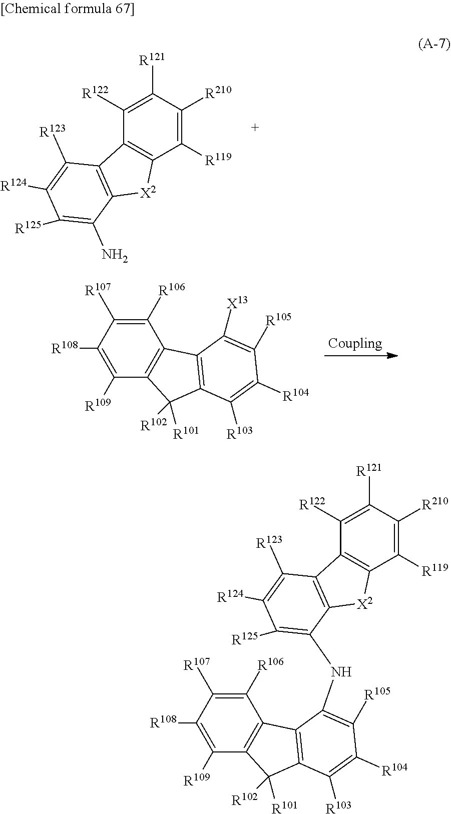

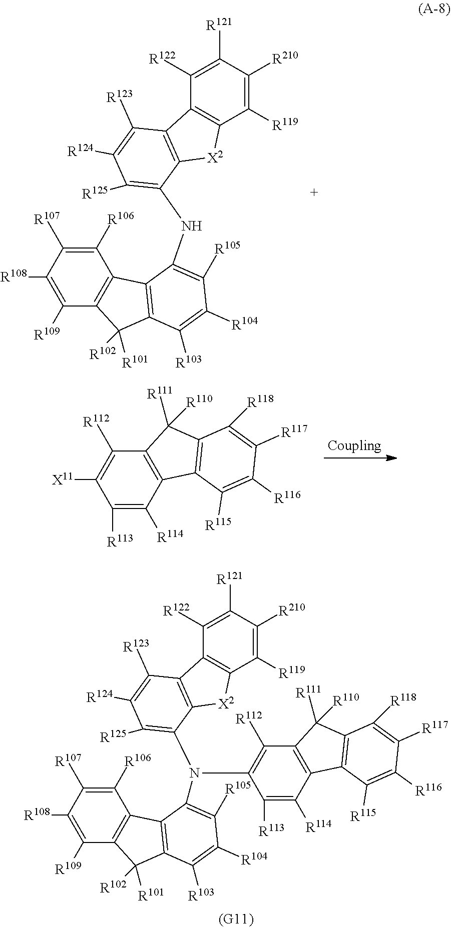

In Synthesis Schemes (A-1) and (A-2), Synthesis Schemes (A-3) and (A-4), Synthesis Schemes (A-5) and (A-6), and Synthesis Schemes (A-7) and (A-8), X² represents oxygen or sulfur, and R¹⁰³ to R¹⁰⁹, R¹¹² to R¹¹⁶, and R¹¹⁹ to R¹²⁵ each represent hydrogen (including deuterium), a substituted or unsubstituted alkyl group having 1 to 6 carbon atoms, or a substituted or unsubstituted aryl group having 6 to 13 carbon atoms. R¹⁰¹, R¹⁰², R¹¹⁰, and R¹¹¹ each independently represent hydrogen (including deuterium), a substituted or unsubstituted alkyl group having 1 to 6 carbon atoms, or a substituted or unsubstituted aryl group having 6 to 13 carbon atoms. Note that R¹⁰⁶, R¹¹⁴, and R¹¹⁵ each represent a substituent other than an unsubstituted phenyl group. In the case where R¹⁰¹ and R¹⁰² are each an unsubstituted phenyl group, R¹¹⁰ and R¹¹¹ each represent a substituent other than a methyl group. In the case where R¹¹⁰ and R¹¹¹ are each an unsubstituted phenyl group, R¹⁰¹ and R¹⁰² each represent a substituent other than a methyl group. Furthermore, X¹¹ to X¹³ each independently represent a halogen or a trifluoromethanesulfonic acid group, and preferably represent chlorine, bromine, or iodine.

As shown in Synthesis Schemes (A-1) and (A-2), a secondary amine is formed by a coupling reaction between a fluorene compound having an amino group and a fluorene compound having a halogen or a trifluoromethanesulfonic acid group. Then, by a coupling reaction between the secondary amine compound and a dibenzofluorene compound having a halogen or a trifluoromethanesulfonic acid group or a dibenzothiophene compound having a halogen or a trifluoromethanesulfonic acid group, the target substance represented by General Formula (G11) can be obtained.

As shown in Synthesis Schemes (A-3) and (A-4) described above, a secondary amine is formed by a coupling reaction between a fluorene compound having an amino group and a fluorene compound having a halogen or a trifluoromethanesulfonic acid group. Then, by a coupling reaction between the secondary amine compound and a dibenzothiophene compound having a halogen or a trifluoromethanesulfonic acid group, the target substance represented by General Formula (G11) can be obtained.

As shown in Synthesis Schemes (A-5) and (A-6) described above, a secondary amine can be formed by a coupling reaction between a dibenzofuran compound having an amino group or a dibenzothiophene compound having an amino group and a fluorene compound having a halogen or a trifluoromethanesulfonic acid group. Then, by a coupling reaction between the secondary amine compound and a fluorene compound having a halogen or a trifluoromethanesulfonic acid group, the target substance represented by General Formula (G11) can be obtained.

As shown in Synthesis Schemes (A-7) and (A-8) described above, a secondary amine can be formed by a coupling reaction between a dibenzofuran compound having an amino group or a dibenzothiophene compound having an amino group and a fluorene compound having a halogen or a trifluoromethanesulfonic acid group. Then, by a coupling reaction between the secondary amine compound and a fluorene compound having a halogen or a trifluoromethanesulfonic acid group, the target substance represented by General Formula (G11) can be obtained.

As shown in Synthesis Schemes (A-1) and (A-2), Synthesis Schemes (A-3) and (A-4), Synthesis Schemes (A-5) and (A-6), and Synthesis Schemes (A-7) and (A-8), the target substance represented by General Formula (G11) can be obtained through coupling reactions in any order, and thus any material can be selected.

In Synthesis Schemes (A-1) and (A-2), Synthesis Schemes (A-3) and (A-4), Synthesis Schemes (A-5) and (A-6), and Synthesis Schemes (A-7) and (A-8), in the case where the Buchwald-Hartwig reaction using a palladium catalyst is employed, a palladium compound such as bis(dibenzylideneacetone)palladium(0), palladium(II) acetate, [1,1-bis(diphenylphosphino)ferrocene]palladium(II) dichloride, tetrakis(triphenylphosphine)palladium(0), or allylpalladium(II) chloride dimer and a ligand such as tri(tert-butyl)phosphine, tri(n-hexyl)phosphine, tricyclohexylphosphine, di(1-adamantyl)-n-butylphosphine, 2-dicyclohexylphosphino-2′,6′-dimethoxybiphenyl, tri(ortho-tolyl)phosphine, (S)-(6,6′-dimethoxybiphenyl-2,2′-diyl)bis(diisopropylphosphine) (abbreviation: cBRIDP), or 4,5-bis(diphenylphosphino)-9,9-dimethylxanthene, can be used. Through the reaction, an organic base such as sodium tert-butoxide, an inorganic base such as potassium carbonate, cesium carbonate, or sodium carbonate, or the like can be used. As a solvent in the reaction, toluene, xylene, benzene, tetrahydrofuran, dioxane, or the like can be used. Reagents that can be used for the reaction are not limited to the above reagents. Alternatively, a compound in which an organotin group is bonded to an amino group can be used instead of the compound having an amino group.

In Synthesis Schemes (A-1) and (A-2), Synthesis Schemes (A-3) and (A-4), Synthesis Schemes (A-5) and (A-6), and Synthesis Schemes (A-7) and (A-8), an Ullmann reaction using copper or a copper compound can be employed. Copper or a copper compound can be used for the reaction. Examples of the base to be used include an inorganic base such as potassium carbonate. Examples of the solvent that can be used in the reaction include 1,3-dimethyl-3,4,5,6-tetrahydro-2(1H)pyrimidinone (DMPU), toluene, xylene, and benzene. In the Ullmann reaction, the target substance can be obtained in a shorter time and in a higher yield when the reaction temperature is 100° C. or higher; therefore, it is preferable to use DMPU or xylene, which have high boiling temperatures. Furthermore, a reaction temperature of 150° C. or higher is further preferred, and accordingly, DMPU is preferred. The reagents that can be used in the reaction are not limited to the above reagents.

As described above, the organic compound of one embodiment of the present invention represented by General Formula (G11) can be synthesized, and an organic compound having an amino group, which is a material for the synthesis, can be synthesized through Synthesis Schemes (A-9) and (A-10).

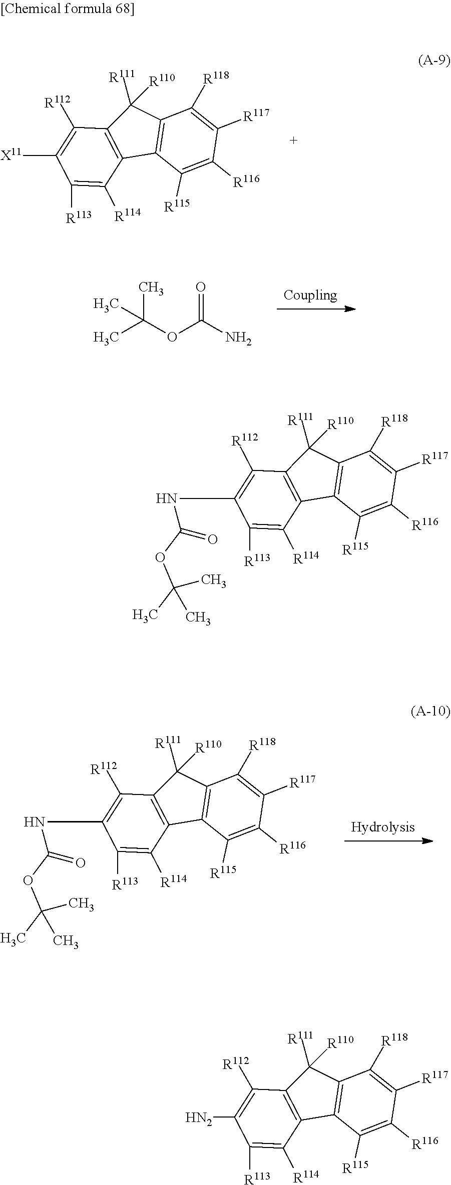

In Synthesis Schemes (A-9) and (A-10), R¹⁰³ to R¹⁰⁹ are the same as those in General Formula (G11) and X¹¹ is the same as those in Synthesis Schemes (A-1), (A-5), and (A-8).

In Synthesis Scheme (A-9), in the case where a coupling reaction using a palladium catalyst is employed, a palladium compound such as bis(dibenzylideneacetone)palladium(0), palladium(II) acetate, [1,1-bis(diphenylphosphino)ferrocene]palladium(II) dichloride, tetrakis(triphenylphosphine)palladium(0), or allylpalladium(II) chloride dimer and a ligand such as tri(tert-butyl)phosphine, tri(n-hexyl)phosphine, tricyclohexylphosphine, di(1-adamantyl)-n-butylphosphine, 2-dicyclohexylphosphino-2′,6′-dimethoxybiphenyl, tri(ortho-tolyl)phosphine, cBRIDP or 4,5-bis(diphenylphosphino)-9,9-dimethylxanthene can be used. In the reaction, an organic base such as sodium tert-butoxide, an inorganic base such as potassium carbonate, cesium carbonate, or sodium carbonate, or the like can be used. As a solvent in the reaction, toluene, xylene, benzene, tetrahydrofuran, dioxane, or the like can be used. Reagents that can be used for the reaction are not limited to the above reagents. Alternatively, a compound in which an organotin group is bonded to an amino group can be used instead of the compound having an amino group.

In addition, the Ullmann reaction using copper or a copper compound can be performed in Synthesis Scheme (A-9). Examples of the base to be used include an inorganic base such as potassium carbonate. Examples of the solvent that can be used in the reaction include 1,3-dimethyl-3,4,5,6-tetrahydro-2(1H)pyrimidinone (DMPU), toluene, xylene, and benzene. In the Ullmann reaction, the target substance can be obtained in a shorter time and in a higher yield when the reaction temperature is 100° C. or higher; therefore, it is preferable to use DMPU or xylene, which have high boiling temperatures. Furthermore, a reaction temperature of 150° C. or higher is further preferred, and accordingly, DMPU is preferred. The reagents that can be used in the reaction are not limited to the above reagents.

In the case where the hydrolysis reaction shown in Synthesis Scheme (A-10) is performed, a preferred acid is an acid not causing a dehydration reaction, such as trifluoroacetic acid, trifluoromethanesulfonic acid, acetic acid, hydrochloric acid, or hydrobromic acid, or a preferred base is a sodium hydroxide solution, a potassium hydroxide solution, or the like.

The organic compounds having an amino group in Synthesis Schemes (A-1), (A-3), (A-5), and (A-7) can be synthesized through a reaction similar to Synthesis Schemes (A-9) and (A-10) above, and can undergo amination as shown in Synthesis Schemes (A-11) and (A-12). The amination shown in Synthesis Schemes (A-11) and (A-12) can be performed by a synthesis method similar to the reaction shown in Synthesis Schemes (A-9) and (A-10).

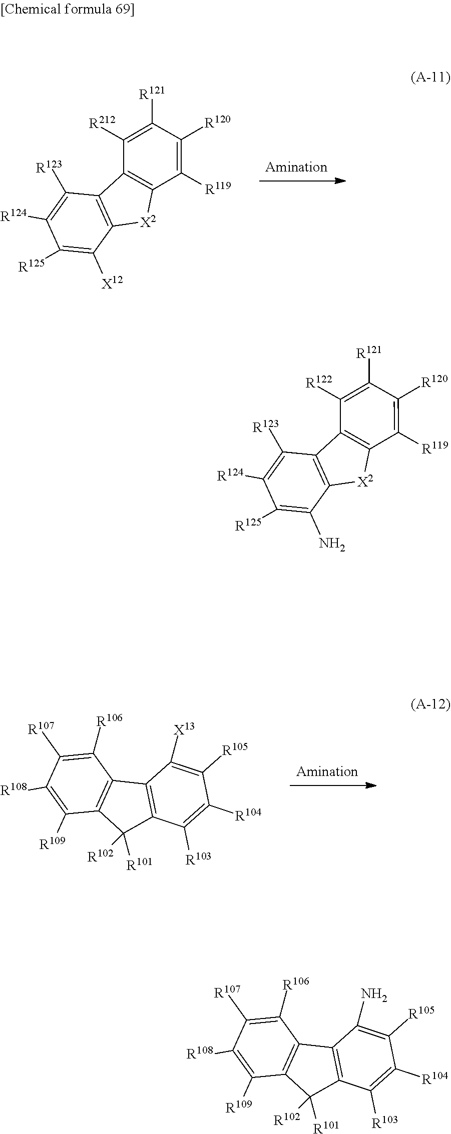

Although an example of a method for synthesizing the organic compound of one embodiment of the present invention is described above, the present invention is not limited thereto and any other synthesis method may be employed.

As specific examples of the first organic compound, organic compounds represented by General Formulae (G21) to (G28) can be given. Note that the use of the organic compounds represented by General Formulae (G21) to (G28) are not limited to the light-emitting device of one embodiment of the present invention. The organic compounds represented by General Formulae (G21) to (G28) are an organic compound that has a hole-transport property and a low HOMO level and that can be suitably used for a variety of kinds of light-emitting devices.

The organic compounds represented by General Formulae (G21) to (G28) each include a spirobifluorenyl group. By containing the spirobifluorenyl group, the glass transition point (Tg) of the organic compound can be increased and heat resistance can be increased. Furthermore, carbon at the 4-position of the spirobifluorenyl group is bonded to amine (nitrogen), whereby an organic compound with high heat resistance and stable film quality can be obtained. The heat resistance of the light-emitting device using such an organic compound can also be improved.

One or both of HOMO levels and carrier-transport properties of the organic compounds represented by General Formulae (G21) to (G28) can be controlled by changing the position of each substituent or the like. Accordingly, an organic compound with a hole-transport property and an optimal HOMO level can be provided for the light-emitting device.

Another embodiment of the present invention is the organic compound represented by General Formula (G21) as described above.

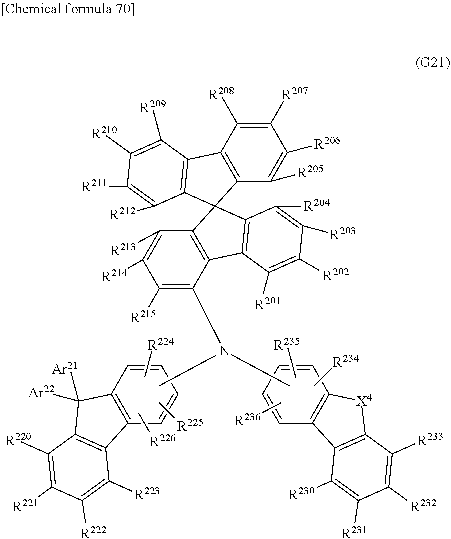

In General Formula (G21), Ar²¹ and Ar²² each independently represent a substituted or unsubstituted aryl group having 6 to 30 carbon atoms, R²⁰¹ to R²¹⁵, R²²⁰ to R²²⁶, and R²³⁰ to R²³⁶ each independently represent hydrogen (including deuterium), a substituted or unsubstituted alkyl group having 1 to 6 carbon atoms, a substituted or unsubstituted cycloalkyl group having 3 to 10 carbon atoms, a substituted or unsubstituted aryl group having 6 to 30 carbon atoms, or a substituted or unsubstituted heteroaryl group having 3 to 30 carbon atoms, and X⁴ represents sulfur or oxygen. Note that the substituents represented by Ar²¹ and Ar²² may be bonded to each other to form a ring.

The organic compound having such a molecular structure has a low HOMO level and a high hole-transport property; therefore, when the organic compound is applied to a light-emitting device, the light-emitting device can be driven at low voltage and with low power consumption. Furthermore, a highly reliable device can be provided. By using a novel organic compound of one embodiment of the present invention, a light-emitting device with high emission efficiency can be provided.

As described above, one embodiment of the present invention is the organic compound represented by General Formula (G22).

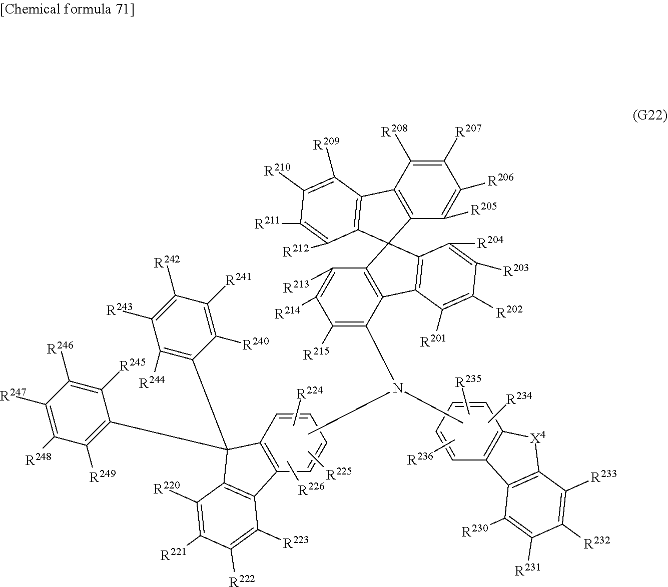

In General Formula (G22), R²⁰¹ to R²¹⁵, R²²⁰ to R²²⁶, R²³⁰ to R²³⁶, and R²⁴⁰ to R²⁴⁹ each independently represent hydrogen (including deuterium), a site to be bonded, a substituted or unsubstituted alkyl group having 1 to 6 carbon atoms, a substituted or unsubstituted cycloalkyl group having 3 to 10 carbon atoms, a substituted or unsubstituted aryl group having 6 to 30 carbon atoms, or a substituted or unsubstituted heteroaryl group having 3 to 30 carbon atoms, and X⁴ represents sulfur or oxygen. Note that the substituents represented by R²⁴⁴ and R²⁴⁵ may be directly bonded to each other to form a spirobifluorene ring.

The organic compound having such a molecular structure has a low HOMO level and a high hole-transport property; therefore, when the organic compound is applied to a light-emitting device, the light-emitting device can be driven at low voltage and with low power consumption. Furthermore, a highly reliable device can be provided. By using a novel organic compound of one embodiment of the present invention, a light-emitting device with high emission efficiency can be provided.

As described above, one embodiment of the present invention is an organic compound represented by General Formula (G23).

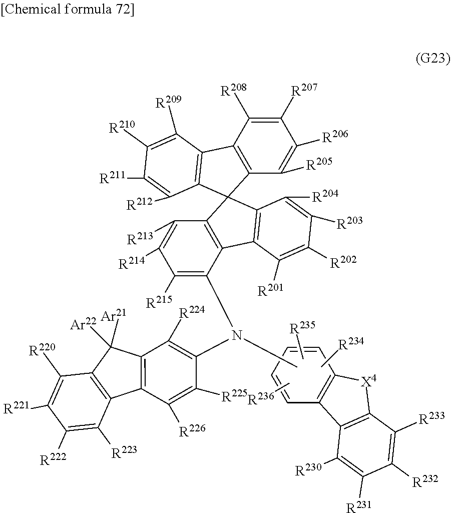

In General Formula (G23), Ar²¹ and Ar²² each independently represent a substituted or unsubstituted aryl group having 6 to 30 carbon atoms; R²⁰¹ to R²¹⁵, R²²⁰ to R²²⁶, and R²³⁰ to R²³⁶ each independently represent hydrogen (including deuterium), a substituted or unsubstituted alkyl group having 1 to 6 carbon atoms, a substituted or unsubstituted cycloalkyl group having 3 to 10 carbon atoms, a substituted or unsubstituted aryl group having 6 to 30 carbon atoms, or a substituted or unsubstituted heteroaryl group having 3 to 30 carbon atoms, and X⁴ represents sulfur or oxygen. Note that the substituents represented by Ar²¹ and Ar²² may be bonded to each other to form a ring.

With an organic compound having such a molecular structure, the planarity of the fluorenyl group and an unshared electron pair of nitrogen is increased and the conjugation is easily extended, which makes it possible to allow the HOMO level of the organic compound to be higher, as compared with a molecular structure in which nitrogen is bonded to the other positions than the 2-position of the fluorenyl group.

As described above, one embodiment of the present invention is the organic compound represented by General Formula (G24).

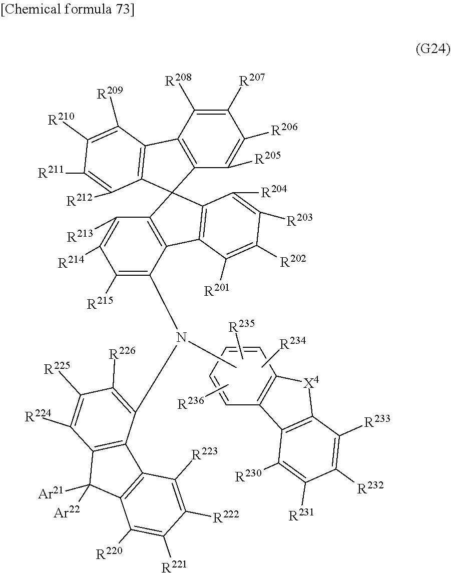

In General Formula (G24), Ar²¹ and Ar²² each independently represent a substituted or unsubstituted aryl group having 6 to 30 carbon atoms; R²⁰¹ to R²¹⁵, R²²⁰ to R²²⁶, and R²³⁰ to R²³⁶ each independently represent hydrogen (including deuterium), a substituted or unsubstituted alkyl group having 1 to 6 carbon atoms, a substituted or unsubstituted cycloalkyl group having 3 to 10 carbon atoms, a substituted or unsubstituted aryl group having 6 to 30 carbon atoms, or a substituted or unsubstituted heteroaryl group having 3 to 30 carbon atoms; and X⁴ represents sulfur or oxygen. Note that the substituents represented by Ar²¹ and Ar²² may be bonded to each other to form a ring.

With such an organic compound having such a molecular structure, the planarity of the fluorenyl group and an unshared electron pair of nitrogen is decreased and the conjugation is hard to be extended, which makes it possible to allow the HOMO level of the organic compound to be lower, as compared with a molecular structure in which nitrogen is bonded to the other positions than the 4-position of the fluorenyl group.

As described above, one embodiment of the present invention is an organic compound represented by General Formula (G25).

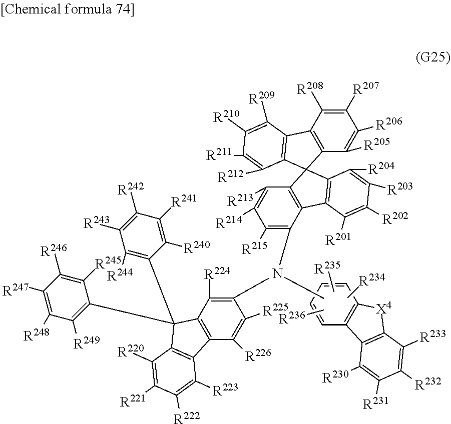

In General Formula (G25), R²⁰¹ to R²¹⁵, R²²⁰ to R²²⁶, R²³⁰ to R²³⁶, and R²⁴⁰ to R²⁴⁹ each independently represent hydrogen (including deuterium), a site to be bonded, a substituted or unsubstituted alkyl group having 1 to 6 carbon atoms, a substituted or unsubstituted cycloalkyl group having 3 to 10 carbon atoms, a substituted or unsubstituted aryl group having 6 to 30 carbon atoms, or a substituted or unsubstituted heteroaryl group having 3 to 30 carbon atoms, and X⁴ represents sulfur or oxygen. Note that the substituents represented by R²⁴⁴ and R²⁴⁵ may be directly bonded to each other to form a spirobifluorene ring.

With an organic compound having such a molecular structure, the planarity of the fluorenyl group and an unshared electron pair of nitrogen is increased and the conjugation is easily extended, which makes it possible to increase the HOMO level of the organic compound, as compared with a molecular structure in which nitrogen is bonded to any of the other positions than the 2-position of the fluorenyl group. Furthermore, with a molecular structure in which two phenyl groups are bonded to the 9-position of the fluorene, the hole mobility can be improved. Thus, when the compound with the molecular structure is applied to a light-emitting device, the light-emitting device can be driven at a lower voltage and lower power consumption.

As described above, one embodiment of the present invention is the organic compound represented by General Formula (G26).

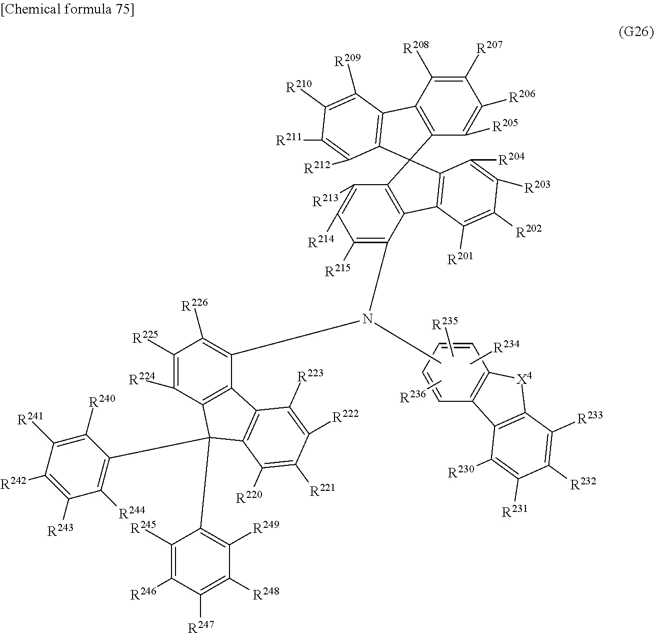

In General Formula (G26), R²⁰¹ to R²¹⁵, R²²⁰ to R²²⁶, R²³⁰ to R²³⁶, and R²⁴⁰ to R²⁴⁹ each independently represent hydrogen (including deuterium), a site to be bonded, a substituted or unsubstituted alkyl group having 1 to 6 carbon atoms, a substituted or unsubstituted cycloalkyl group having 3 to 10 carbon atoms, a substituted or unsubstituted aryl group having 6 to 30 carbon atoms, a substituted or unsubstituted heteroaryl group having 3 to 30 carbon atoms, and X⁴ represents sulfur or oxygen. Note that the substituents represented by R²⁴⁴ and R²⁴⁵ may be directly bonded to each other to form a spirobifluorene ring.

With an organic compound having such a molecular structure, the planarity of the fluorenyl group and an unshared electron pair of nitrogen is decreased and the conjugation is hard to be extended, which makes it possible to allow the HOMO level of the organic compound to be lower, as compared with a molecular structure in which nitrogen is bonded to any of the other positions than the 4-position of the fluorenyl group. Furthermore, with a molecular structure in which two phenyl groups are bonded to the 9-position of the fluorene, the hole mobility can be improved. Thus, when the compound with the molecular structure is applied to a light-emitting device, the light-emitting device can be driven at a lower voltage and lower power consumption.

As described above, one embodiment of the present invention is the organic compound represented by General Formula (G27).

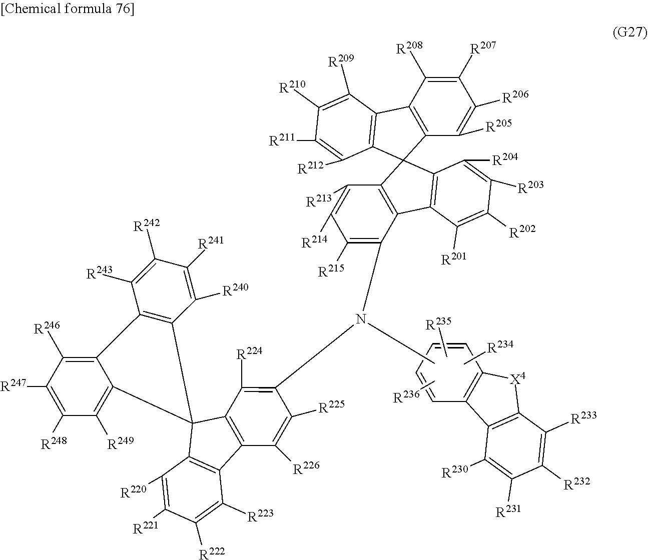

In General Formula (G27), R²⁰¹ to R²¹⁵, R²²⁰ to R²²⁶, R²³⁰ to R²³⁶, R²⁴⁰ to R²⁴³, and R²⁴⁶ to R²⁴⁹ each independently represent hydrogen (including deuterium), a bond, a substituted or unsubstituted alkyl group having 1 to 6 carbon atoms, a substituted or unsubstituted cycloalkyl group having 3 to 10 carbon atoms, a substituted or unsubstituted aryl group having 6 to 30 carbon atoms, a substituted or unsubstituted heteroaryl group having 3 to 30 carbon atoms, and X⁴ represents sulfur or oxygen.

With an organic compound having such a molecular structure, the planarity in the fluorenyl group is improved and the conjugation is extended, which makes it possible to allow the HOMO level of the organic compound to be higher.

Furthermore, such a structure containing two spirobifluorenyl groups is preferable, in which case heat resistance of the organic compound is further improved.

As described above, one embodiment of the present invention is an organic compound represented by General Formula (G28).

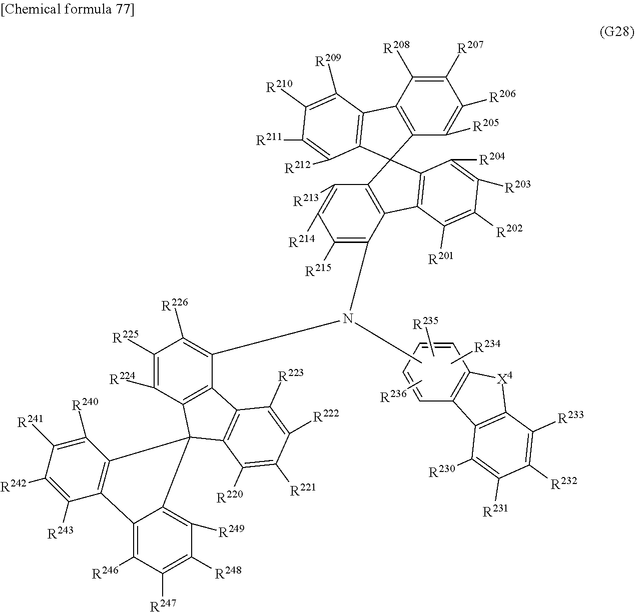

In General Formula (G28), R²⁰¹ to R²¹⁵, R²²⁰ to R²²⁶, R²³⁰ to R²³⁶, R²⁴⁰ to R²⁴³, and R²⁴⁶ to R²⁴⁹ each independently represent hydrogen (including deuterium), a bond, a substituted or unsubstituted alkyl group having 1 to 6 carbon atoms, a substituted or unsubstituted cycloalkyl group having 3 to 10 carbon atoms, a substituted or unsubstituted aryl group having 6 to 30 carbon atoms, a substituted or unsubstituted heteroaryl group having 3 to 30 carbon atoms, and X⁴ represents sulfur or oxygen.

With such an organic compound having such a molecular structure, the planarity of the fluorenyl group and an unshared electron pair of nitrogen is decreased and the conjugation is hard to be extended, which makes it possible to allow the HOMO level of the organic compound to be lower, as compared with a molecular structure in which nitrogen is bonded to any of the other positions than the 4-position of the fluorenyl group.

Furthermore, such a structure containing two spirobifluorenyl groups is preferable, in which case heat resistance of the organic compound is further improved.

Note that in General Formulae (G21) to (G28), carbon at the 2-position of the dibenzofuranyl group or the dibenzothiophenyl group is preferably bonded to nitrogen.

By adopting such a molecular structure for an organic compound, the steric hindrance in the molecule can be made small, and the conjugation between nitrogen and the dibenzofuranyl group or the dibenzothiophenyl group is easily expanded, allowing the HOMO level to be higher. Furthermore, the hole-transport property (mobility) can be improved, and a device capable of being driven at a low voltage can be provided. Thus, an electronic appliance with low power consumption can be provided.

In General Formulae (G21) to (G28), carbon at the 4-position of the dibenzofuranyl group or the dibenzothiophenyl group is preferably bonded to nitrogen.

The organic compound with such a molecular structure enables a device to have low power consumption and high reliability.

In the organic compounds represented by General Formulae (G21) to (G28), X⁴ preferably represents oxygen. The organic compound with such a molecular structure can have a low refractive index, enabling a light-emitting device with high emission efficiency.

In the organic compounds represented by General Formulae (G22), (G25), and (G26), any one of R²⁴⁰ to R²⁴⁴ and any one of R²⁴⁵ to R²⁴⁹ each preferably represent a substituted or unsubstituted phenyl group. With such a molecular structure, the organic compounds can have an extremely high hole mobility. Moreover, with the use of the organic compounds, a device that can be driven at a low voltage and a device with low power consumption can be provided.

In the organic compounds represented by General Formulae (G22), (G25), and (G26), preferably, R²⁴⁰ and R²⁴⁵ each represent a substituted or unsubstituted phenyl group. With such a molecular structure, the organic compounds can have an extremely high hole mobility and high heat resistance. Moreover, with the use of the organic compounds, a device that can be driven at a low voltage and a device with low power consumption can be provided. Moreover, a highly reliable device can be provided.

In the organic compounds represented by General Formulae (G22), (G25), and (G26), preferably, R²⁴² and R²⁴⁷ each represent a substituted or unsubstituted phenyl group.

In the organic compounds represented by General Formulae (G22), (G25), and (G26), any one of R²⁴⁰ to R²⁴⁴ and any one of R²⁴⁵ to R²⁴⁹ preferably represent a substituted or unsubstituted phenyl group, whereby the planarity in surroundings of R²⁴⁰ and R²⁴⁵ is improved. Thus, the hole-transport property can be improved. With such molecular structures, an organic compound with excellent heat resistance and an organic compound with a stable film quality can be provided. Furthermore, with the use of the organic compounds, a device that can be driven at a low voltage and a device with low power consumption can be provided. Moreover, a highly reliable device can be provided.

In General Formulae (G21) to (G28), specific examples of the alkyl group having 1 to 6 carbon atoms include a methyl group, an ethyl group, a propyl group, an isopropyl group, a butyl group, a sec-butyl group, an isobutyl group, and a tert-butyl group.

In General Formulae (G21) to (G28), specific examples of the unsubstituted or substituted cycloalkyl group having 3 to 10 carbon atoms include a cyclopropyl group, a cyclobutyl group, and a cyclohexyl group.

In General Formulae (G21) to (G28), examples of the aryl group having 6 to 30 carbon atoms and the heteroaryl group having 2 to 30 carbon atoms include substituents represented by Structural Formulae (Ar-1) to (Ar-80). Note that the aryl group having 6 to 30 carbon atoms and the heteroaryl group having 2 to 30 carbon atoms that can be used in General Formulae (G21) to (G28) are not limited to the substituents represented by Structural Formulae (Ar-1) to (Ar-80).

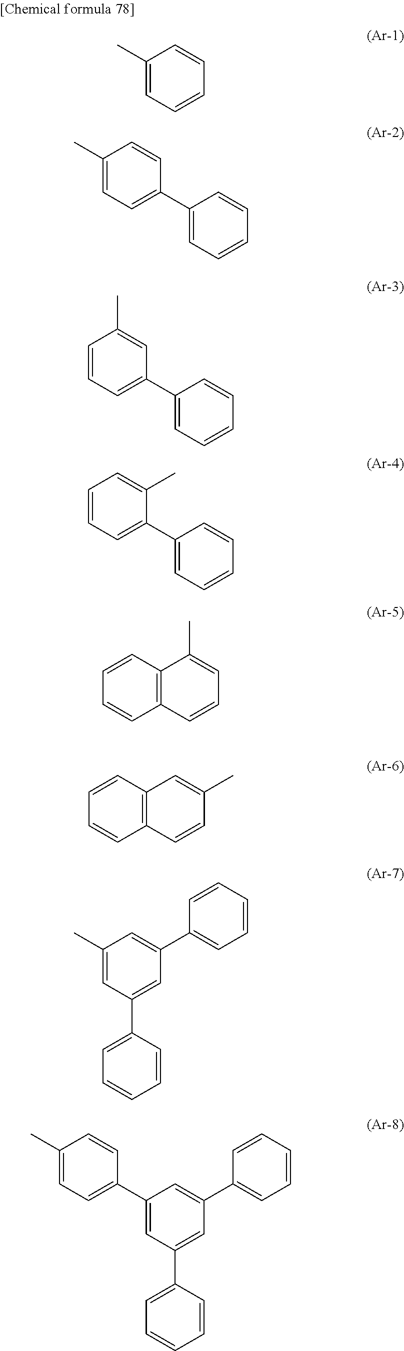

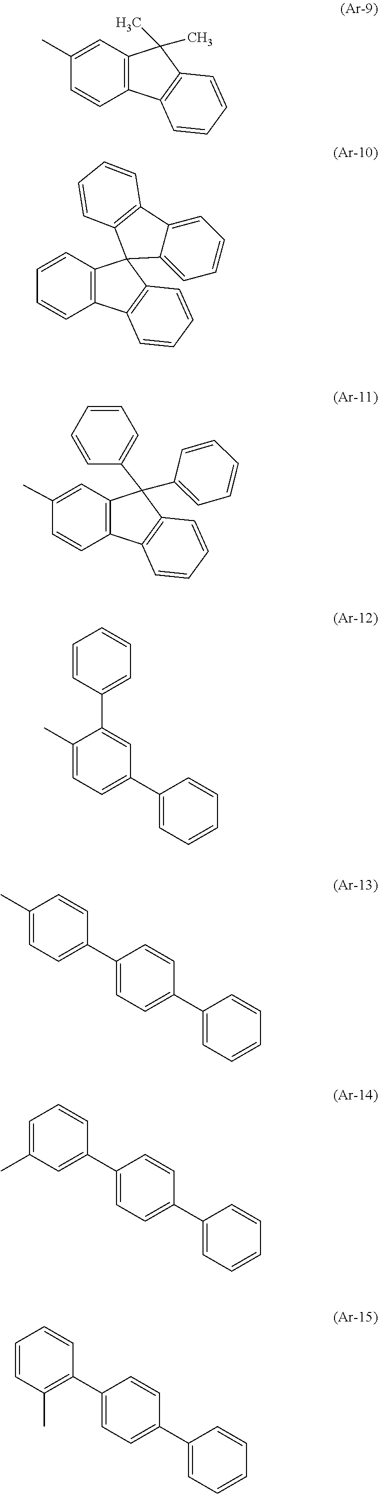

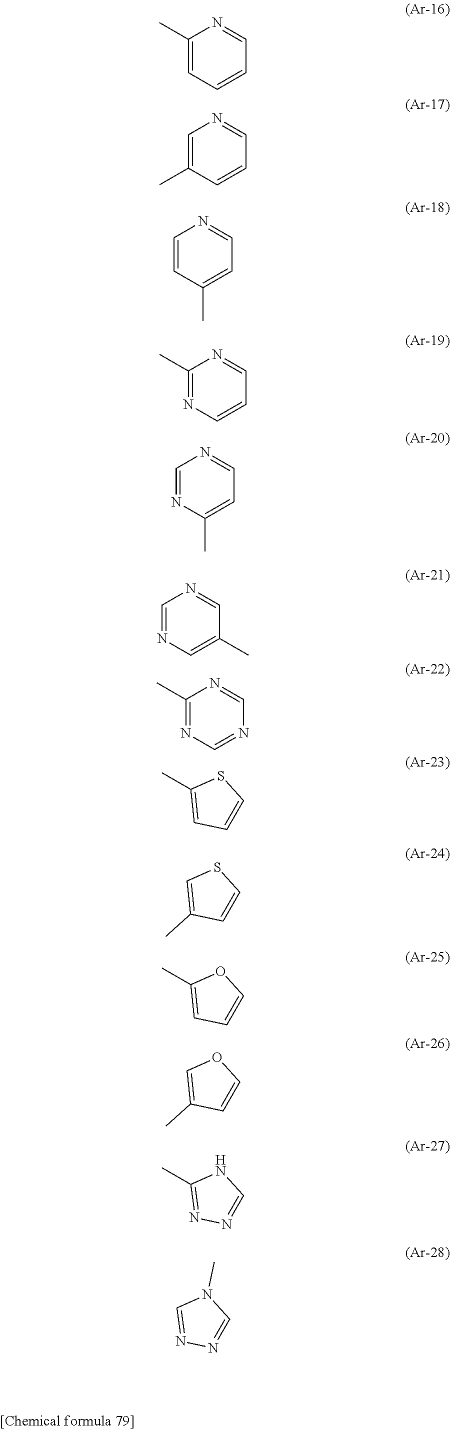

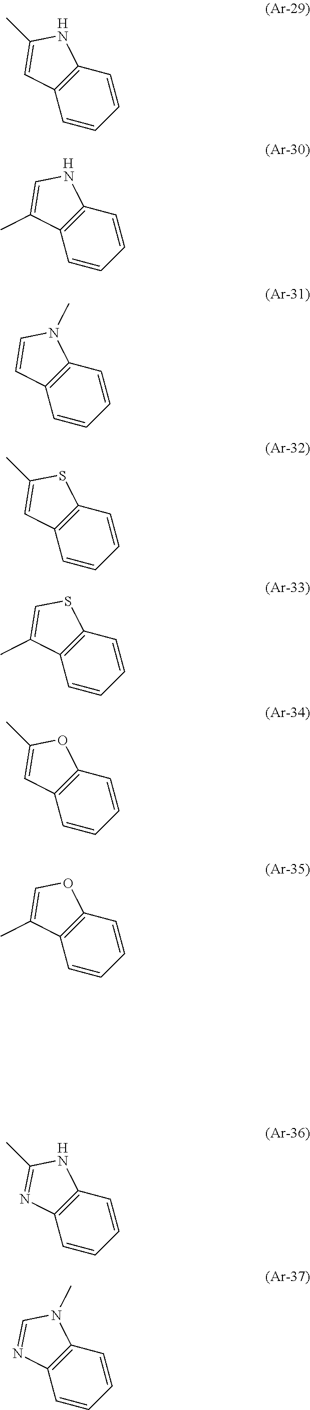

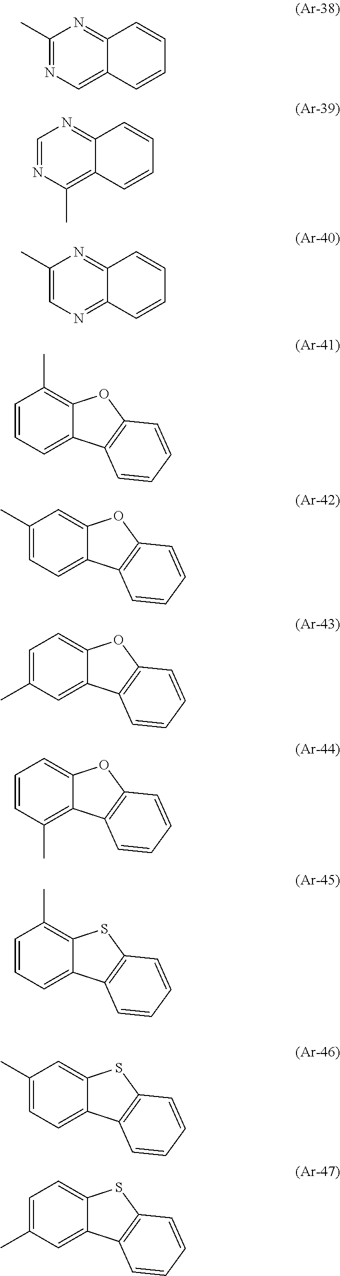

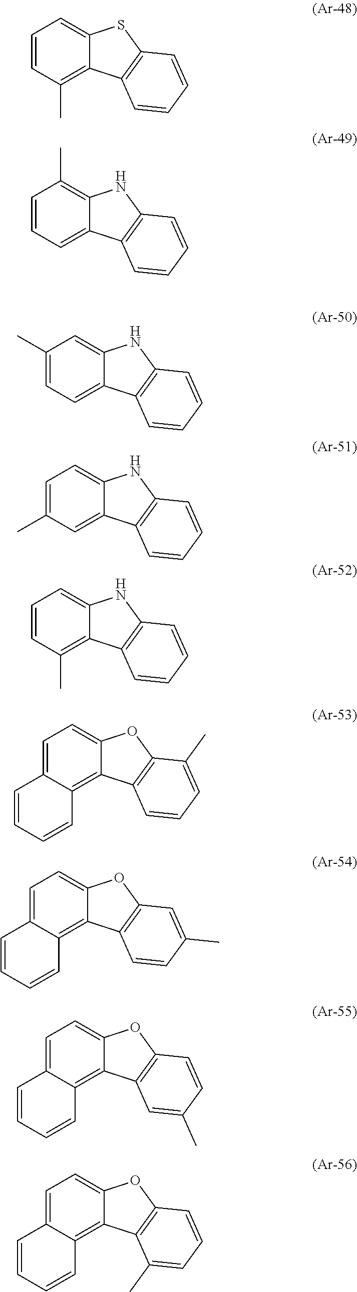

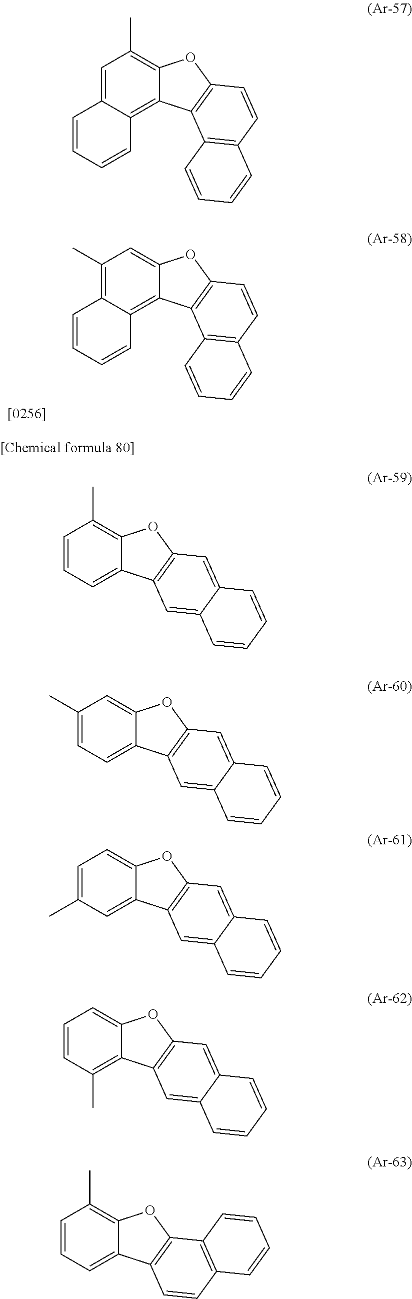

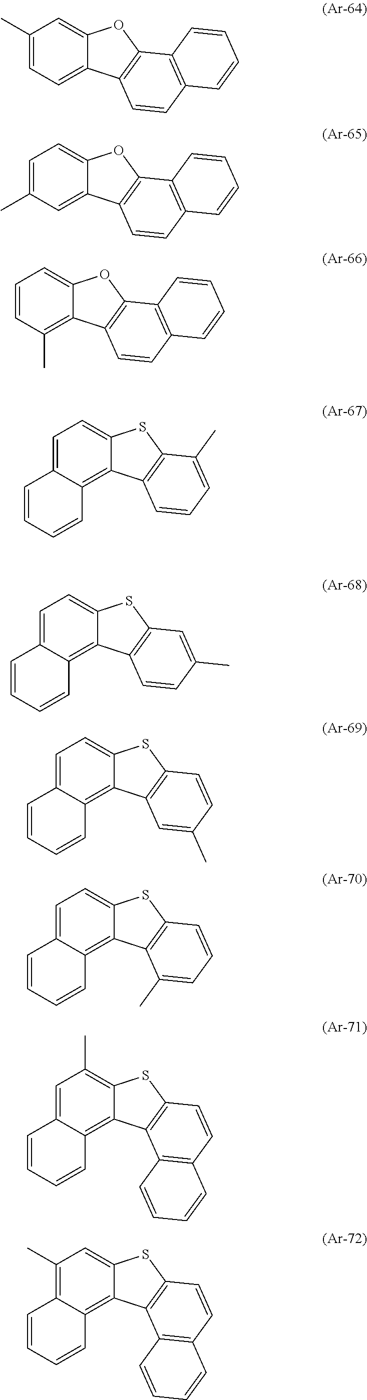

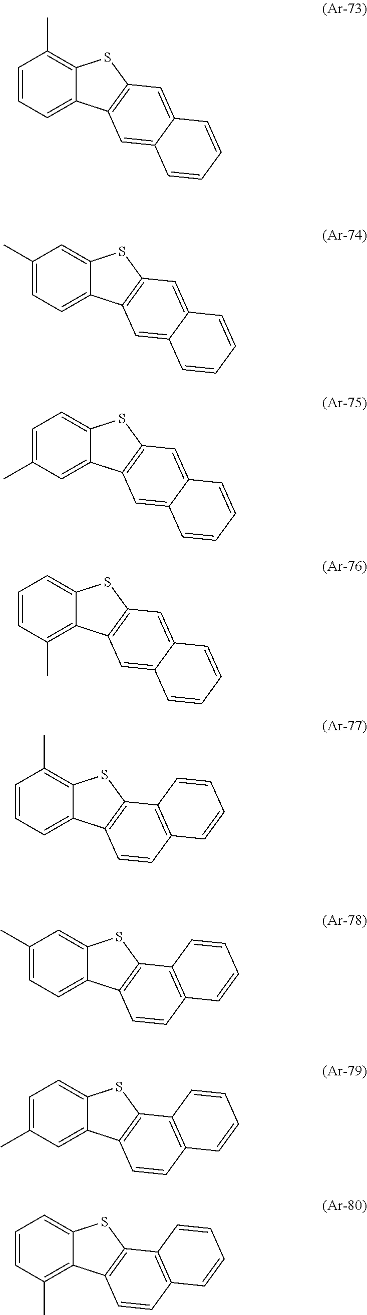

Note that the substituents represented by Structural Formulae (Ar-1) to (Ar-80) are examples of the aryl group having 6 to 30 carbon atoms and the heteroaryl group having 2 to 30 carbon atoms; however, the aryl group having 6 to 30 carbon atoms and the heteroaryl group having 2 to 30 carbon atoms that can be used in General Formulae (G21) to (G28) are not limited to the substituents represented by General Formulae (Ar-1) to (Ar-80).

In the case where the alkyl group having 1 to 6 carbon atoms, the cycloalkyl group having 3 to 10 carbon atoms, the aryl group having 6 to 30 carbon atoms, and the heteroaryl group having 2 to 30 carbon atoms each have a substituent, specific examples of the substituent include an alkyl group having 1 to 7 carbon atoms, such as a methyl group, an ethyl group, a propyl group, an isopropyl group, a butyl group, an isobutyl group, a sec-butyl group, a tert-butyl group, a pentyl group, or a hexyl group; a cycloalkyl group having 5 to 7 carbon atoms, such as a cyclopentyl group, a cyclohexyl group, a cycloheptyl group, or a 8,9,10-trinorbornanyl group; and an aryl group having 6 to 12 carbon atoms, such as a phenyl group, a naphthyl group, or a biphenyl group.

As described above, Ar²¹ and Ar²² may be bonded to form a ring; for example, a spirobifluorenyl group is formed by the bond between the substituents to form one ring (for example, when both Ar²¹ and Ar²² are phenyl groups, a compound in which the two phenyl groups are bonded to each other to form a ring is a spirobifluorenyl group).

Specific examples of structures represented by General Formulae (G21) to (G28) are included in the specific examples of structures represented by General Formulae (G1) to (G4). Specific examples of the structures represented by General Formulae (G21) to (G28) extracted from the specific examples of the structures represented by General Formulae (G1) to (G4) are given below. Note that the organic compounds represented by Structural Formulae (630) to (689) below and the organic compounds represented by Structural Formulae (700) to (759) below are not described above as the specific examples of the structures represented by General Formulae (G1) to (G4); however, the organic compounds are also specific examples of the structures represented by General Formulae (G1) to (G4) and can be used suitably for the first organic compound.

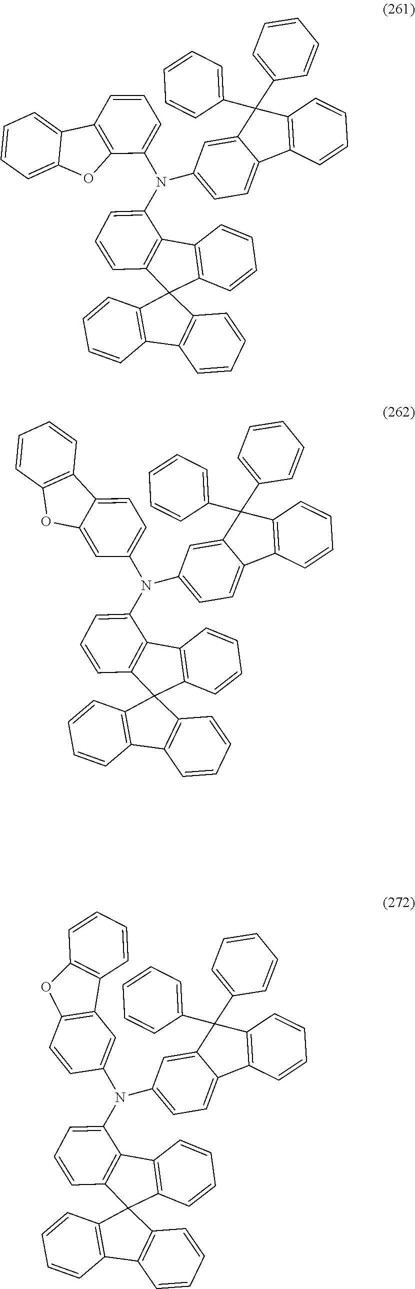

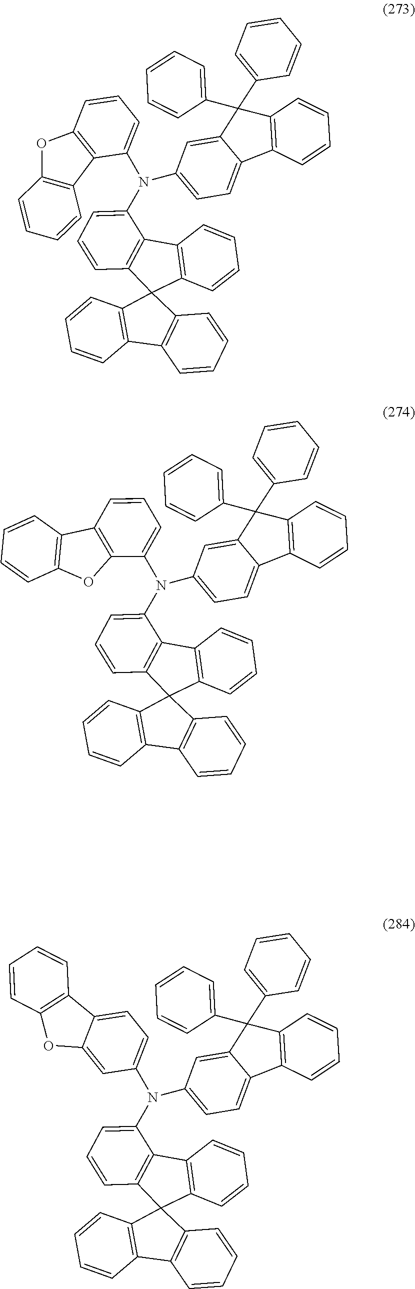

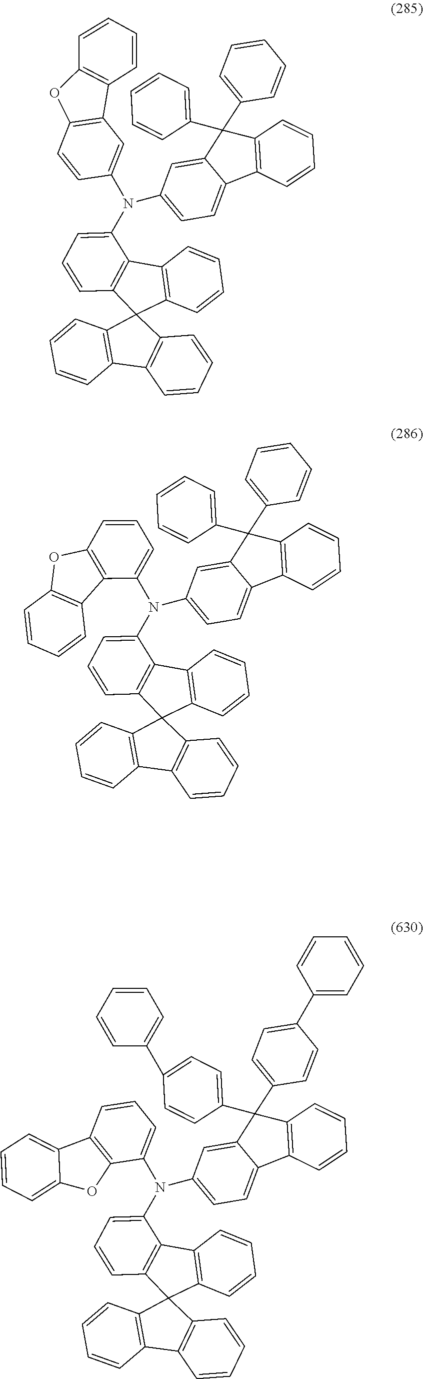

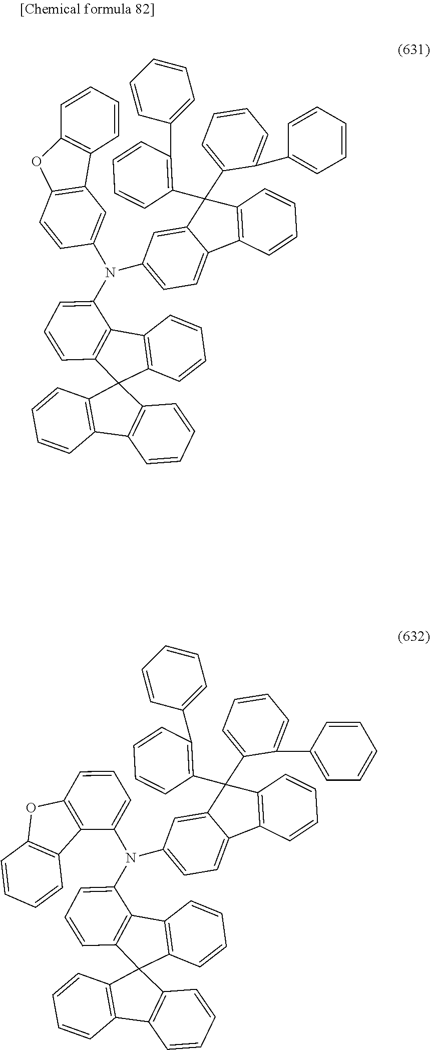

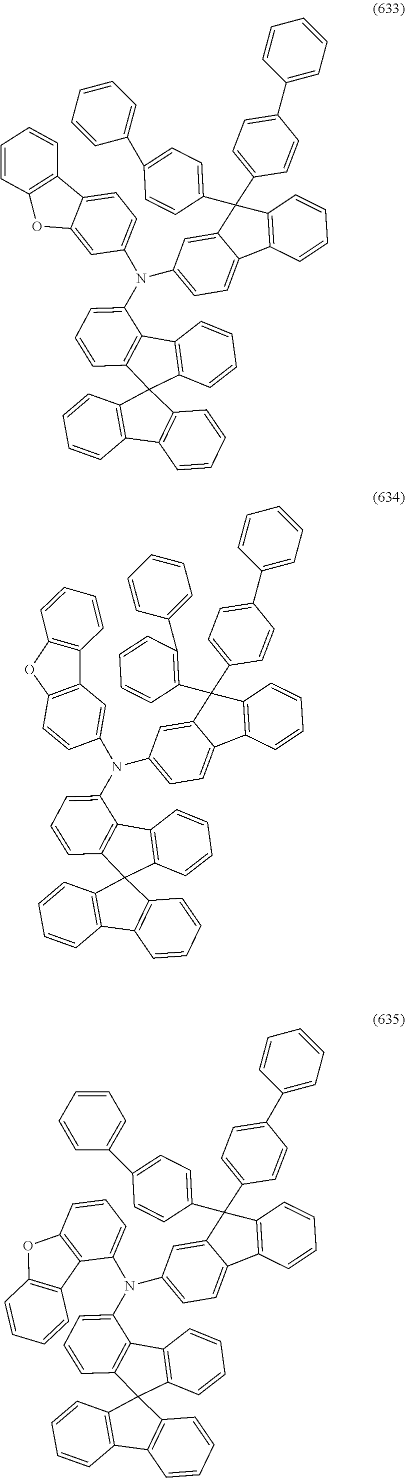

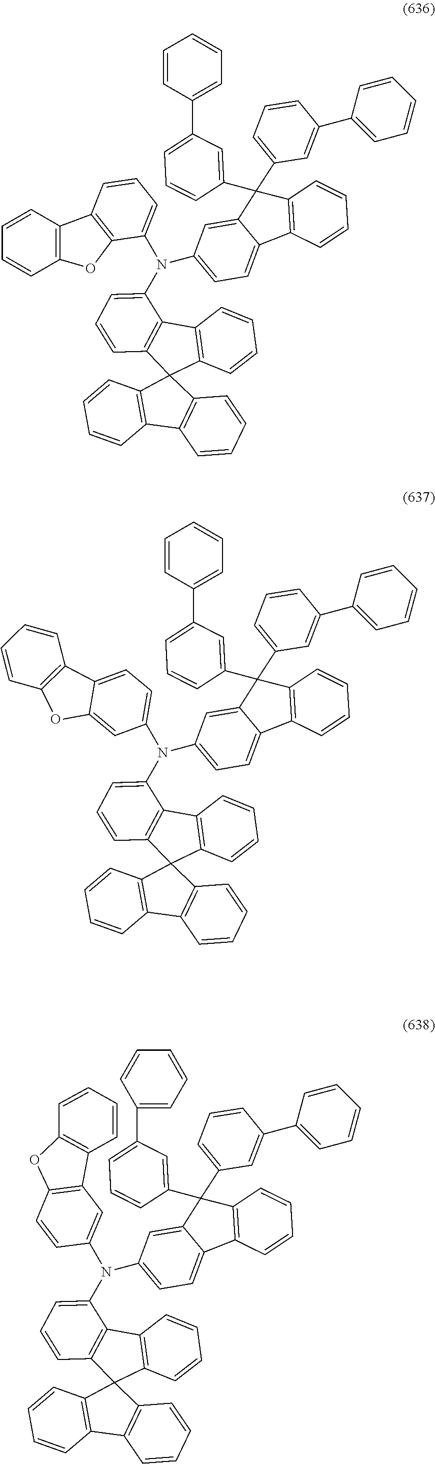

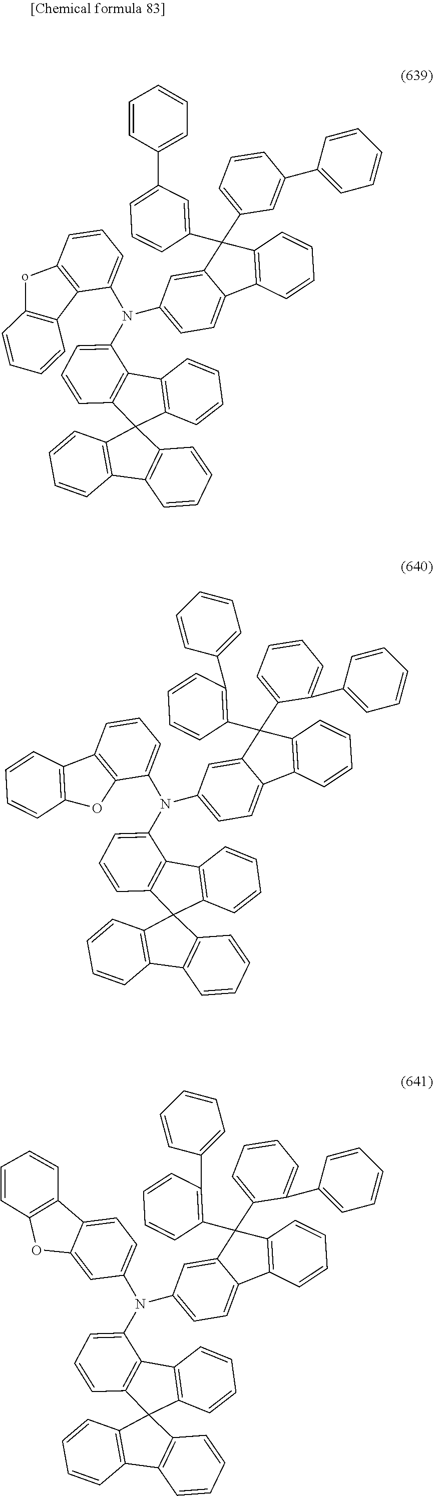

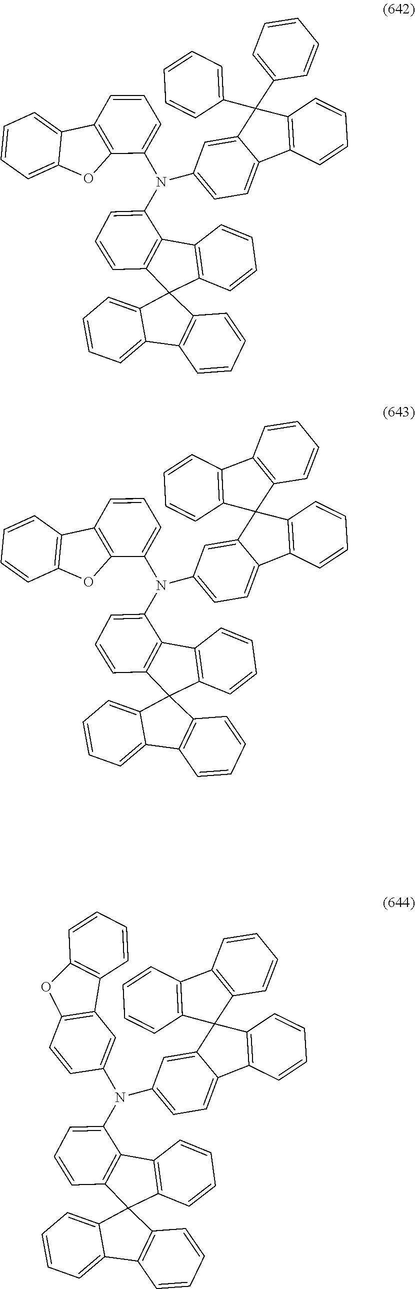

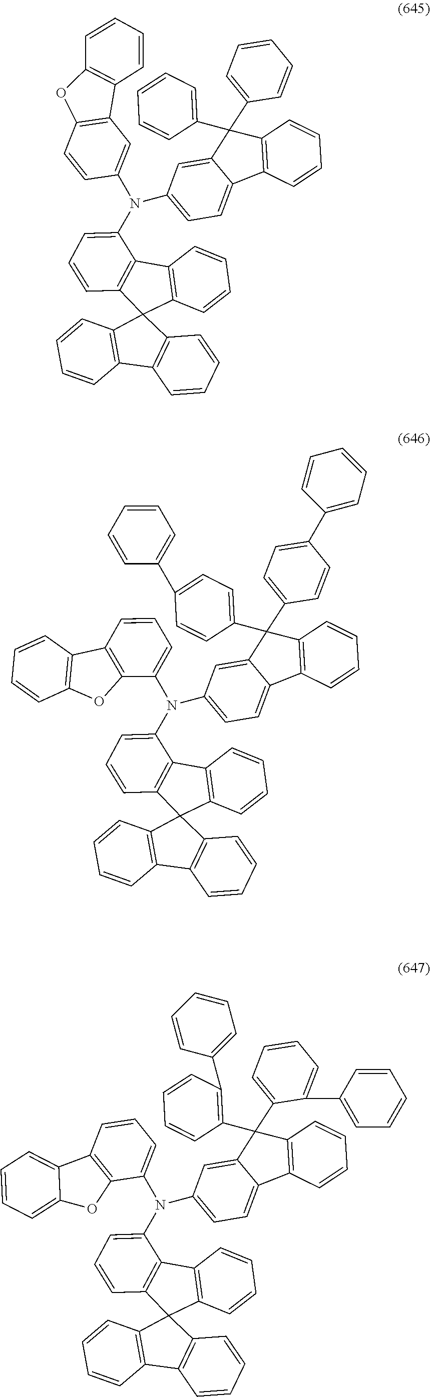

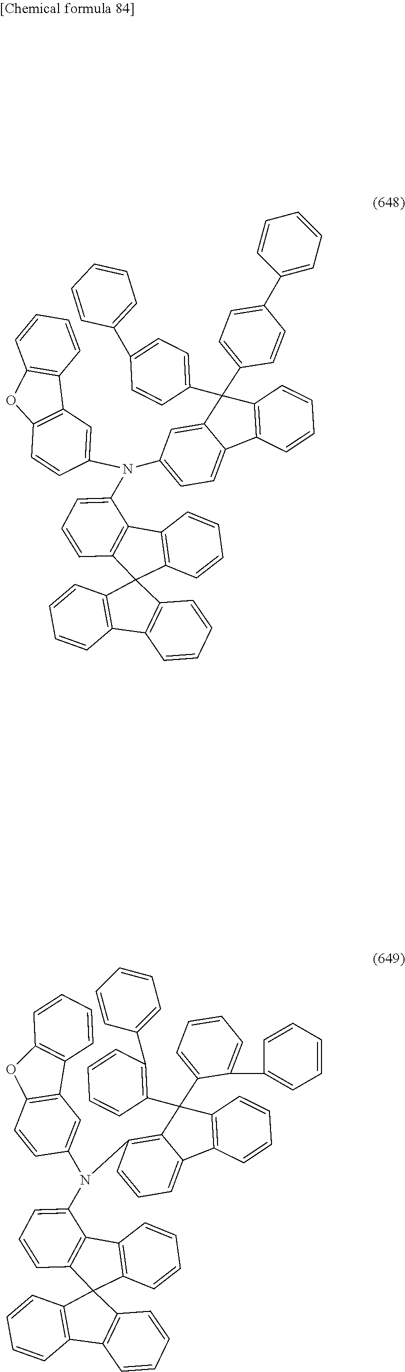

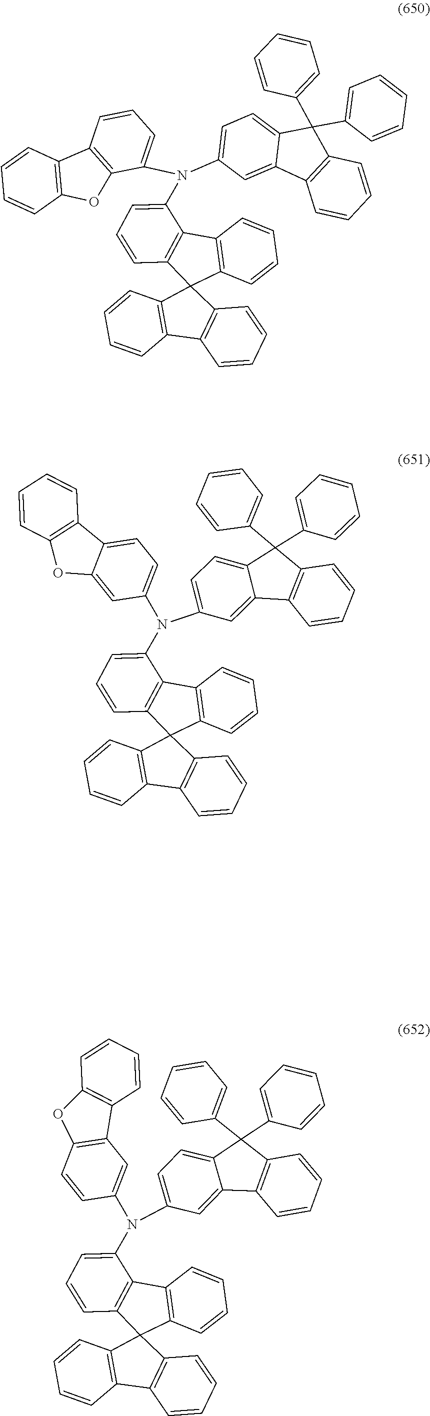

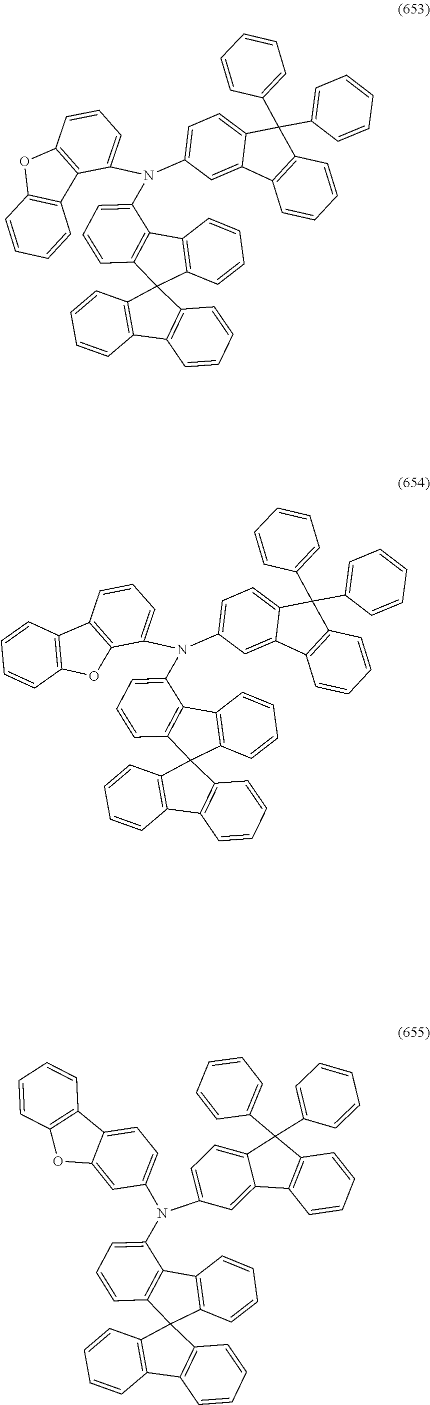

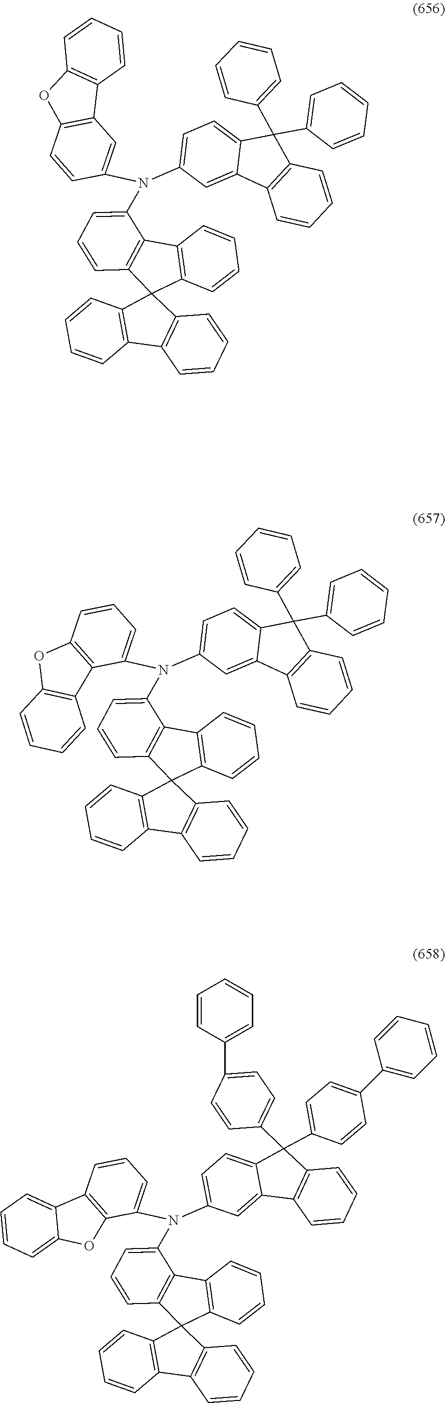

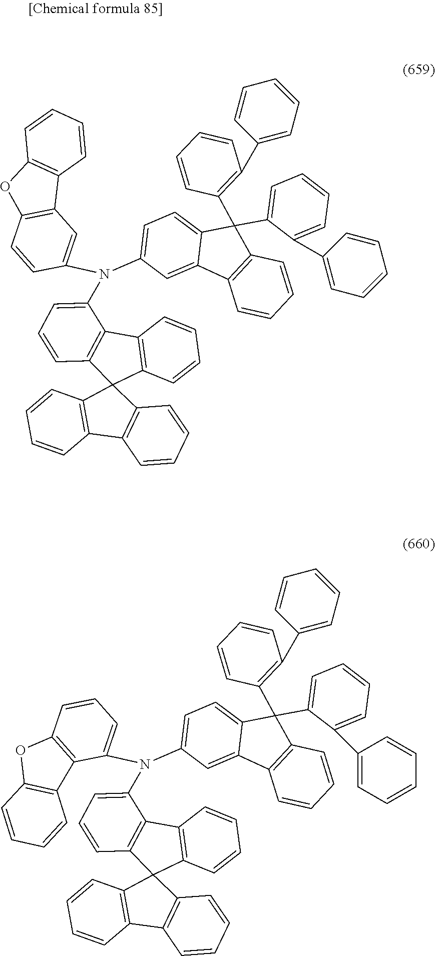

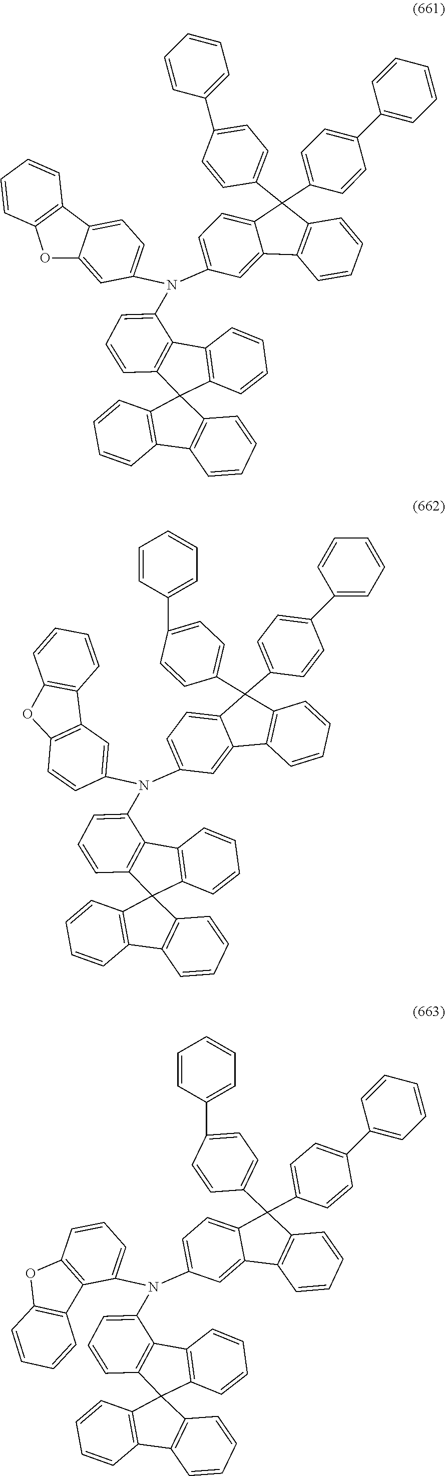

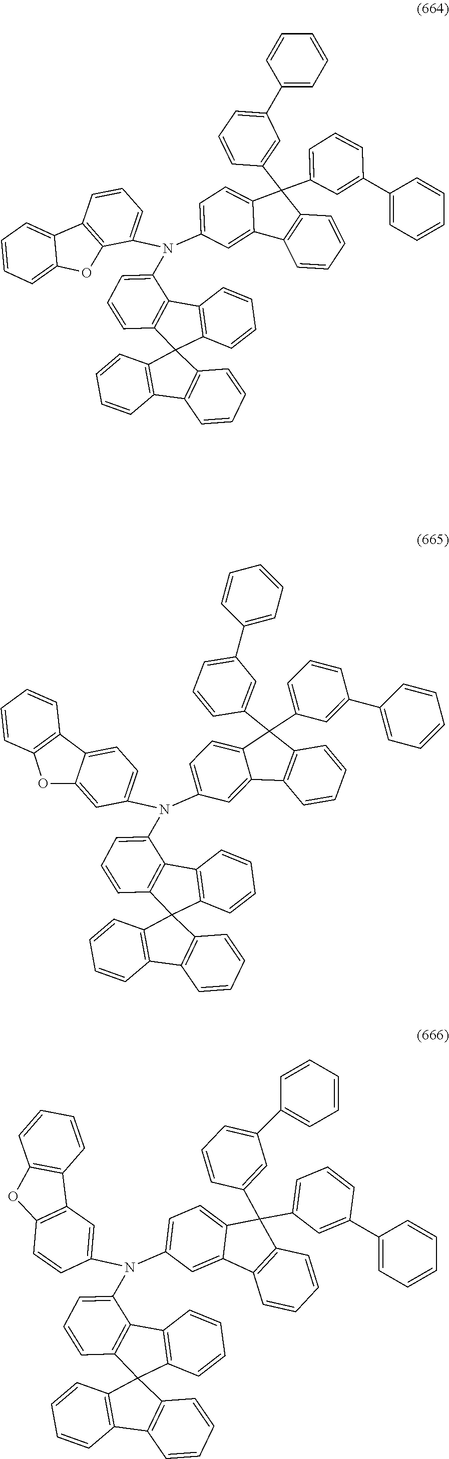

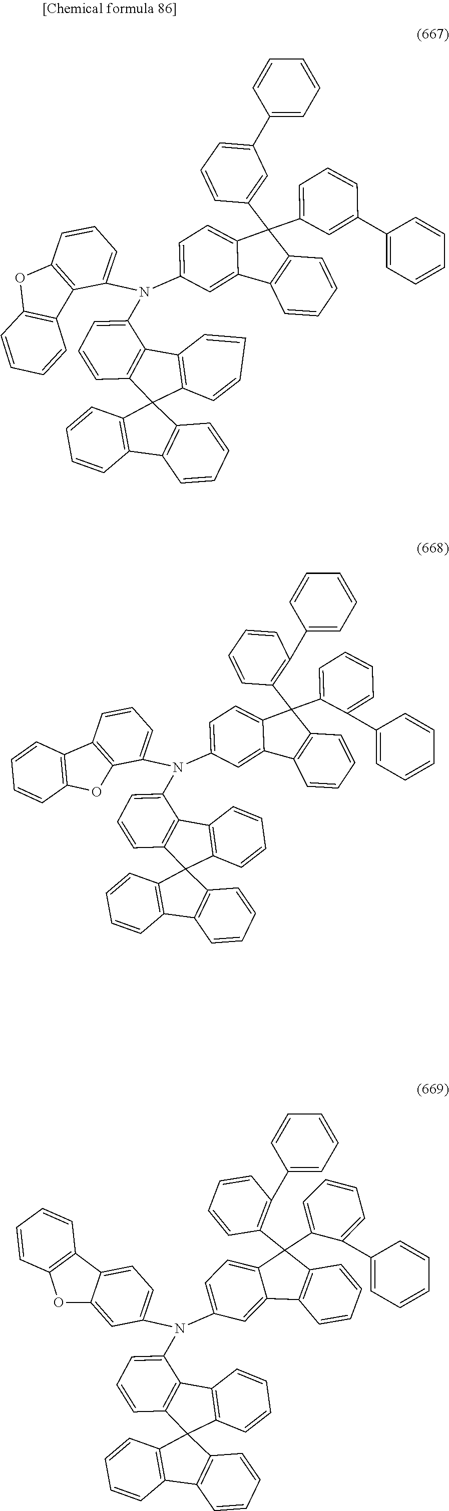

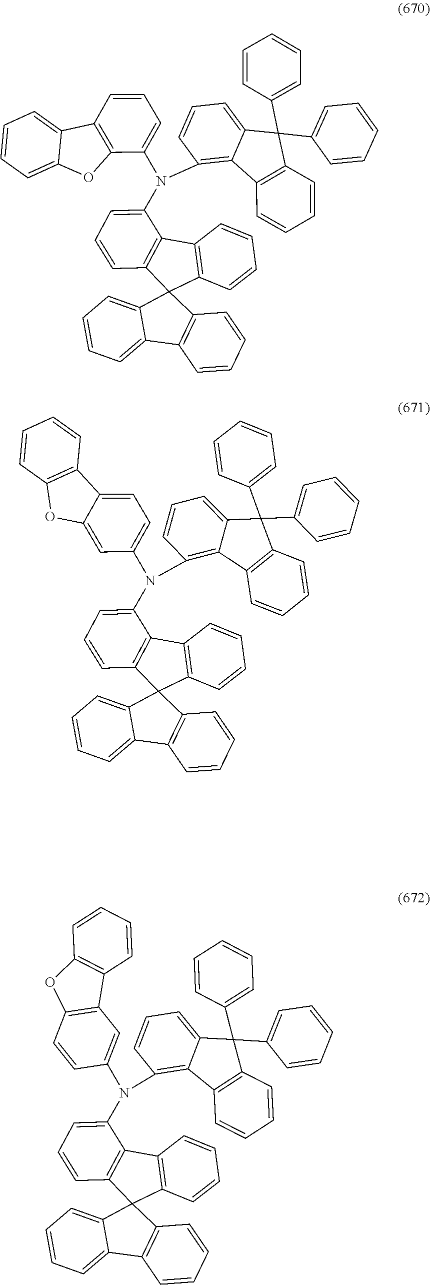

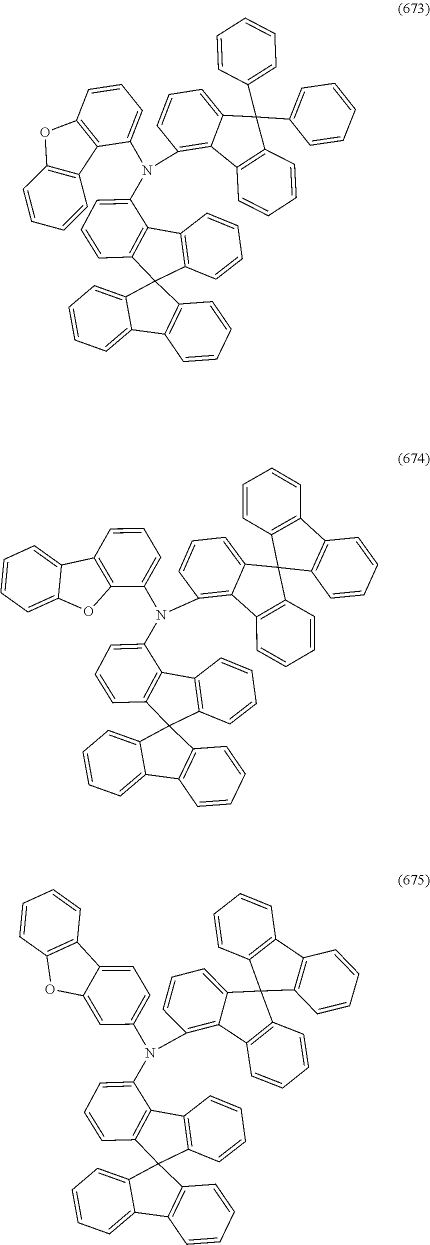

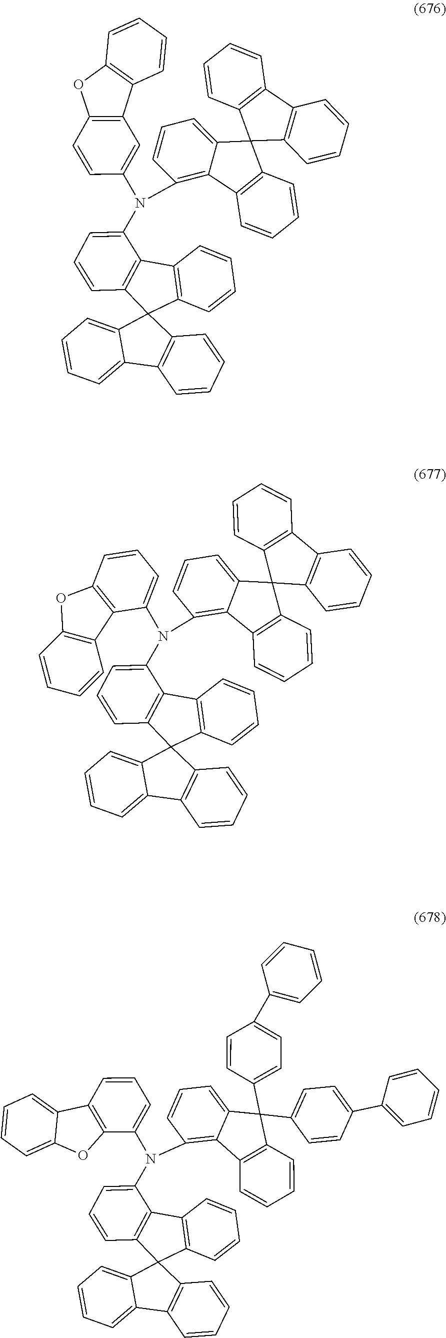

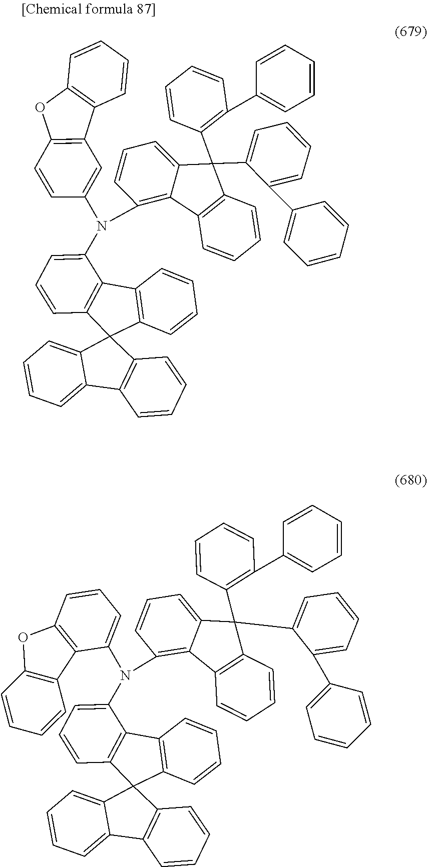

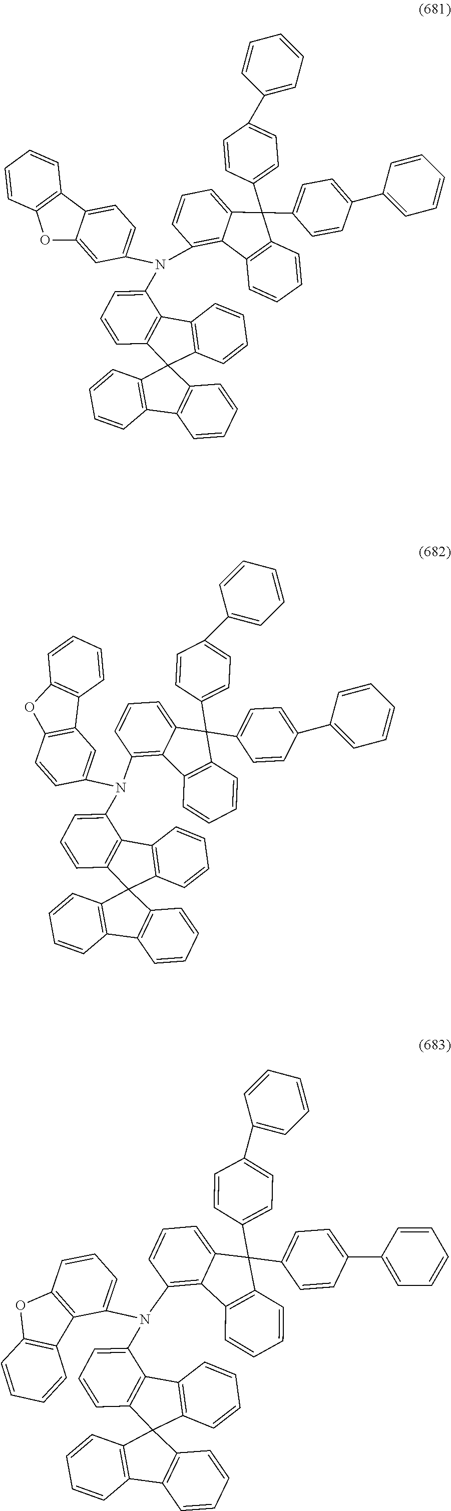

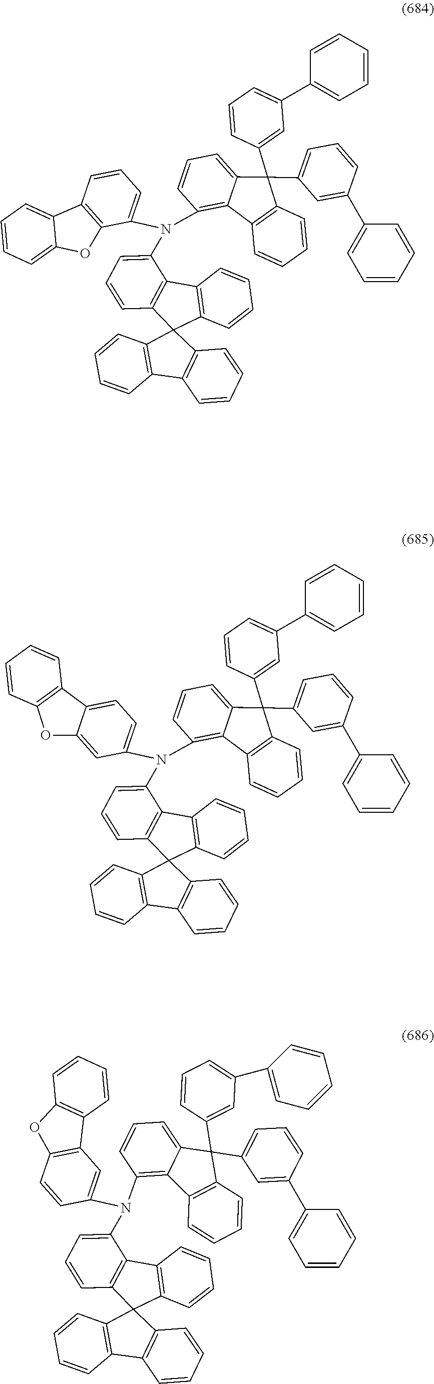

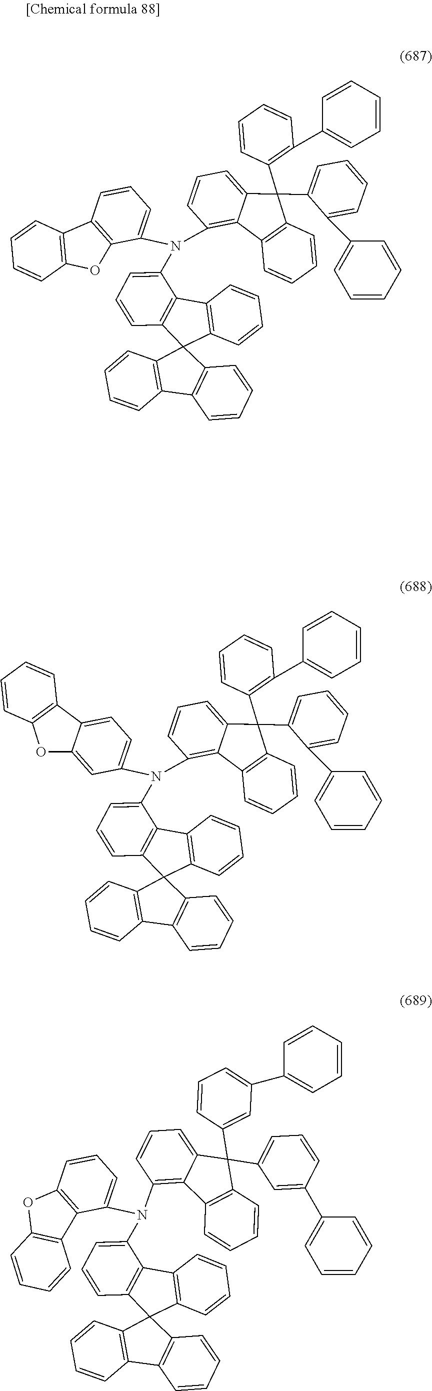

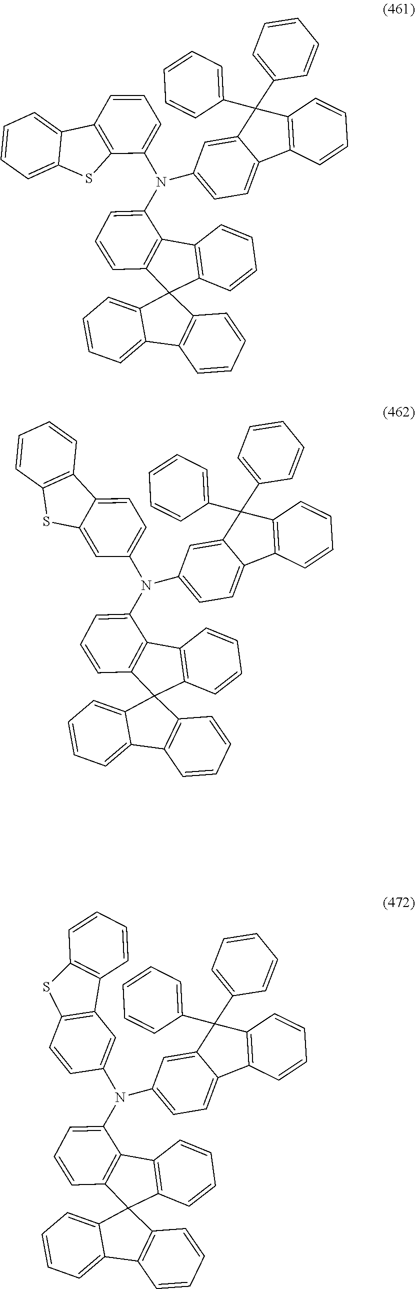

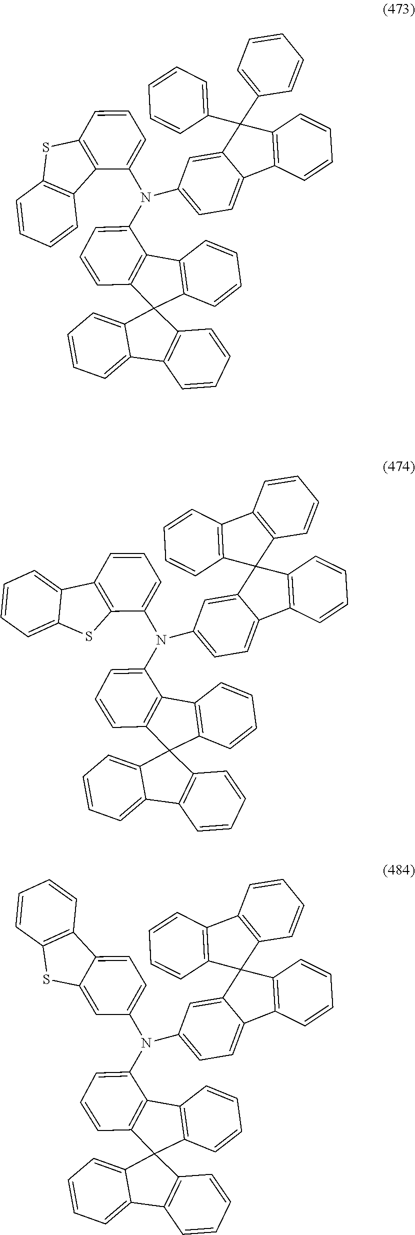

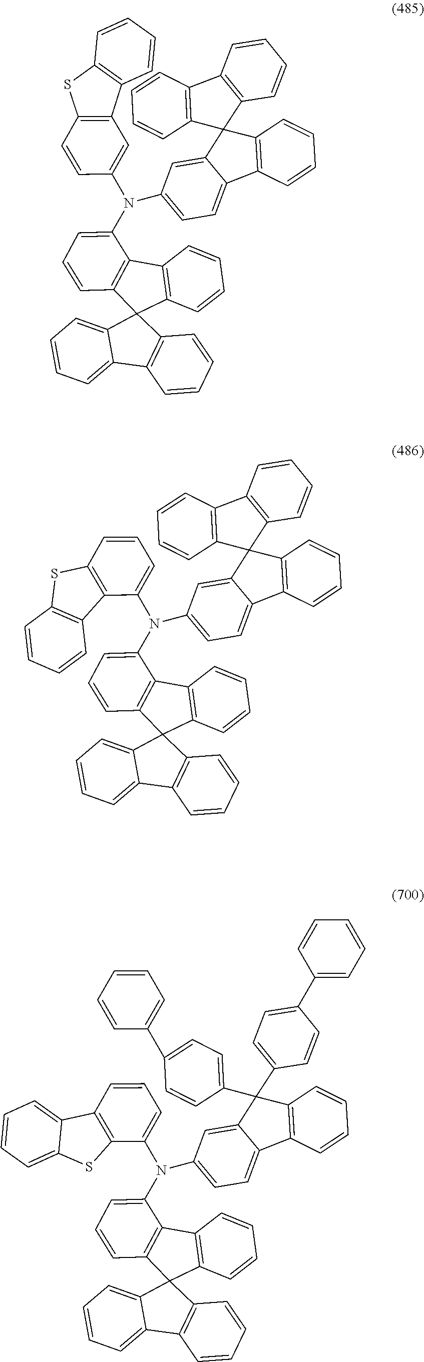

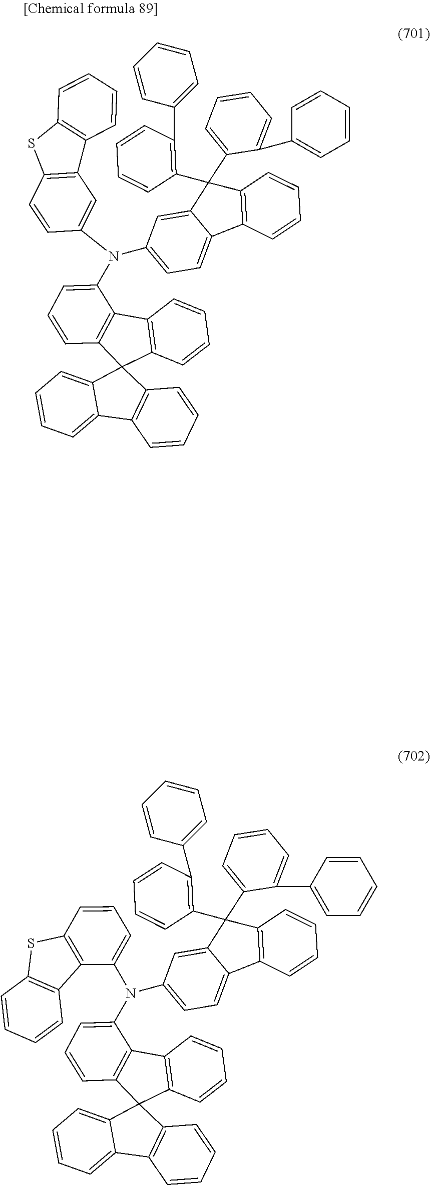

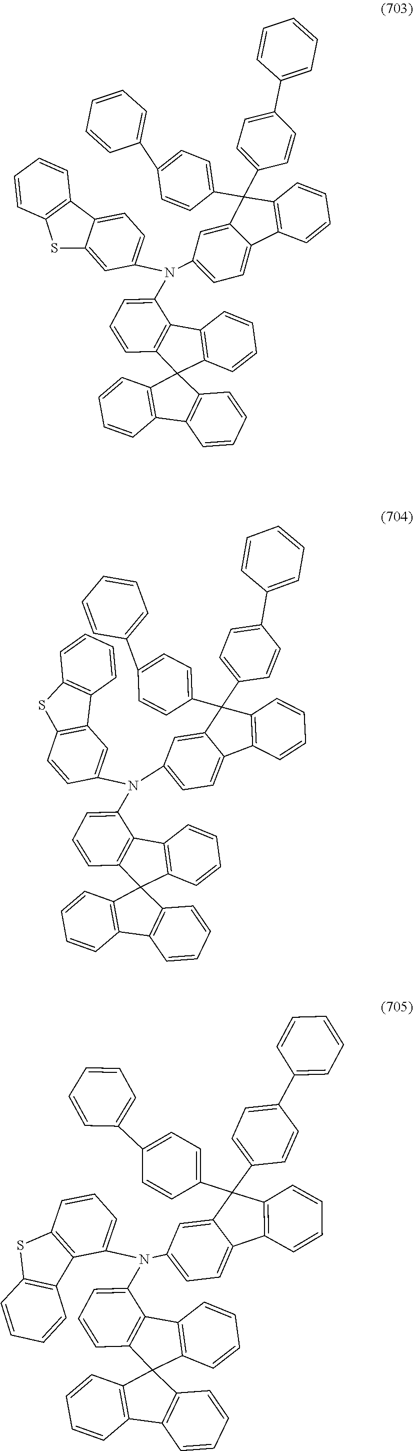

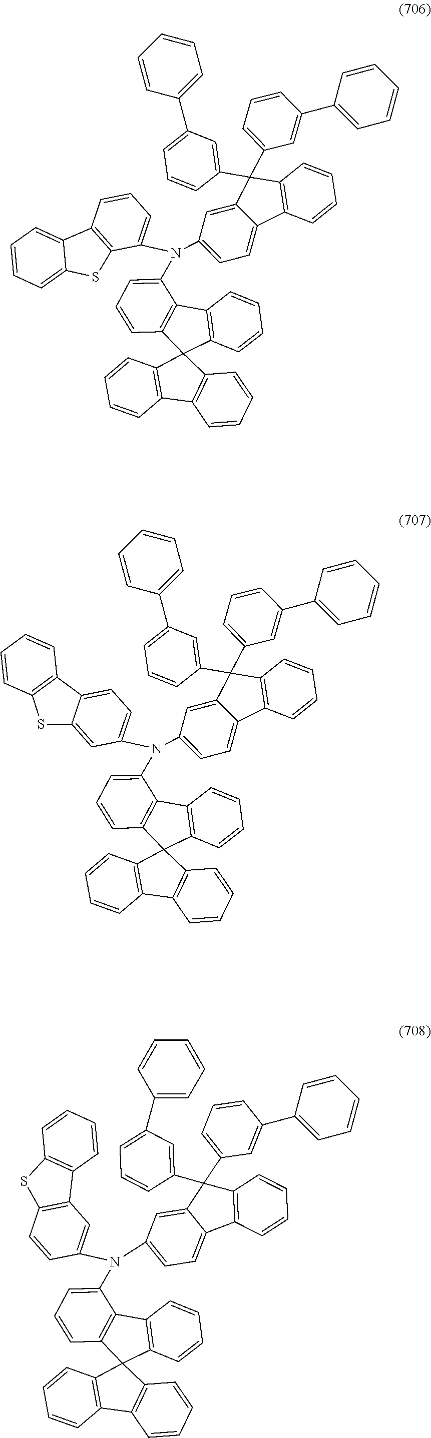

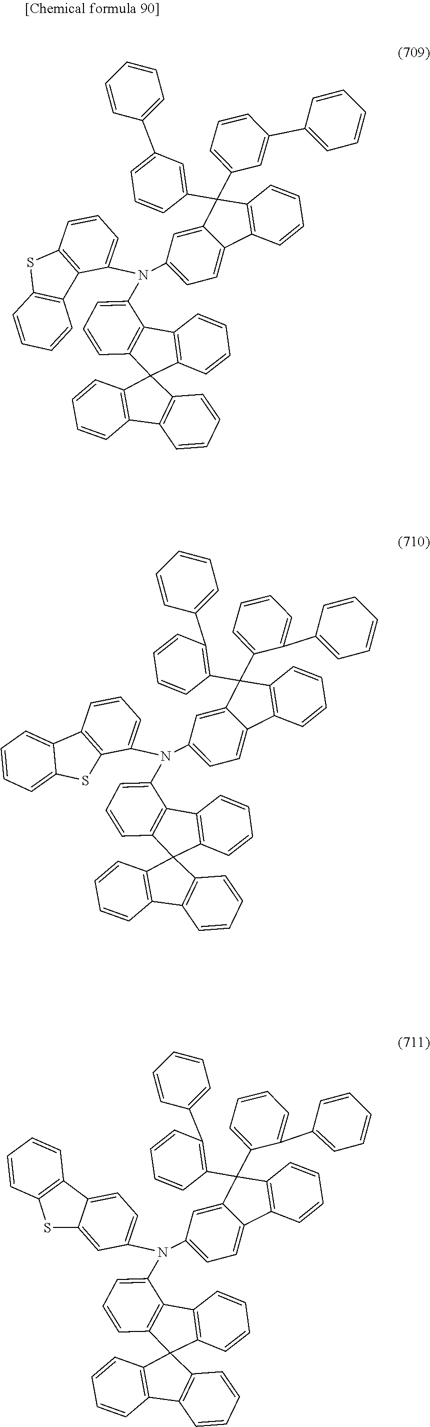

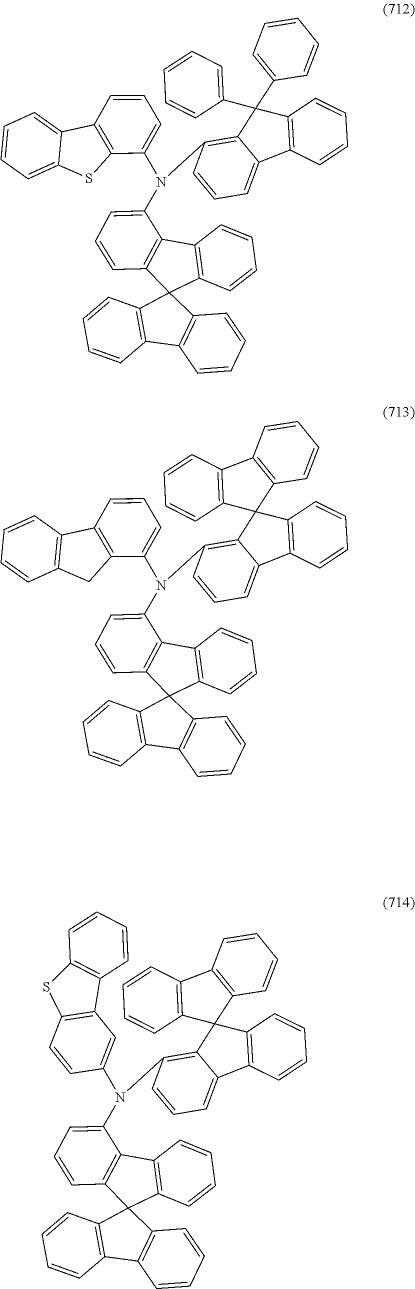

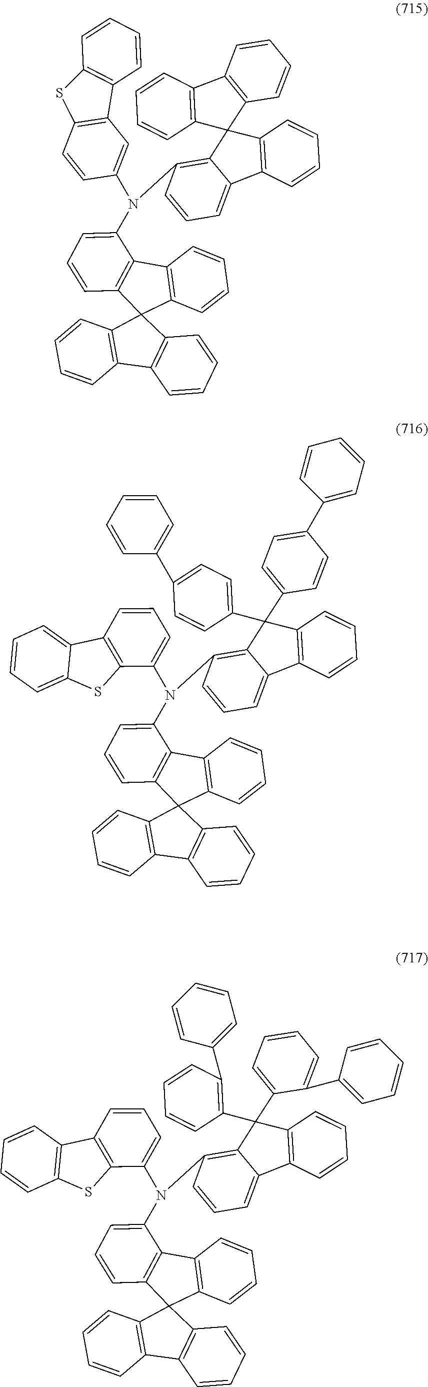

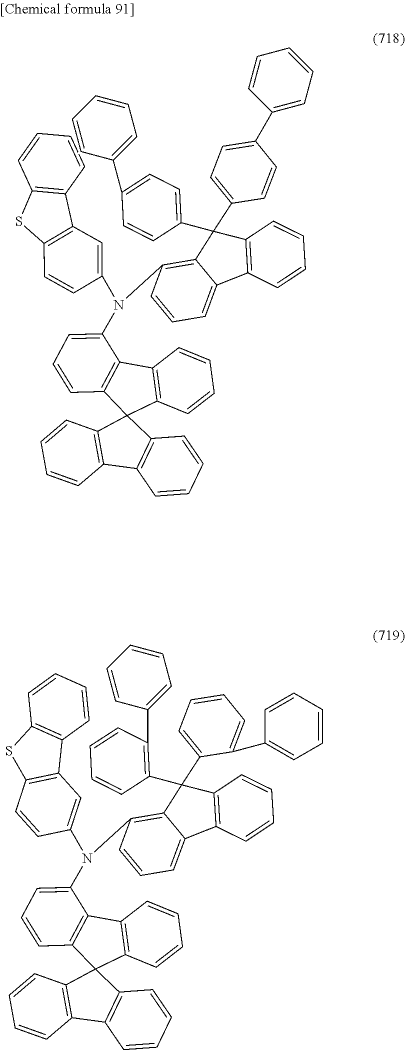

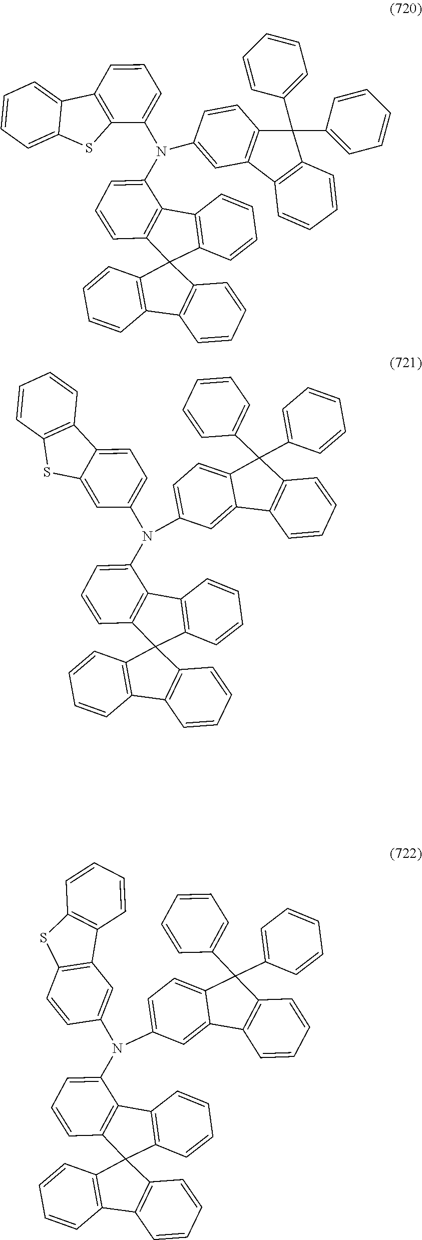

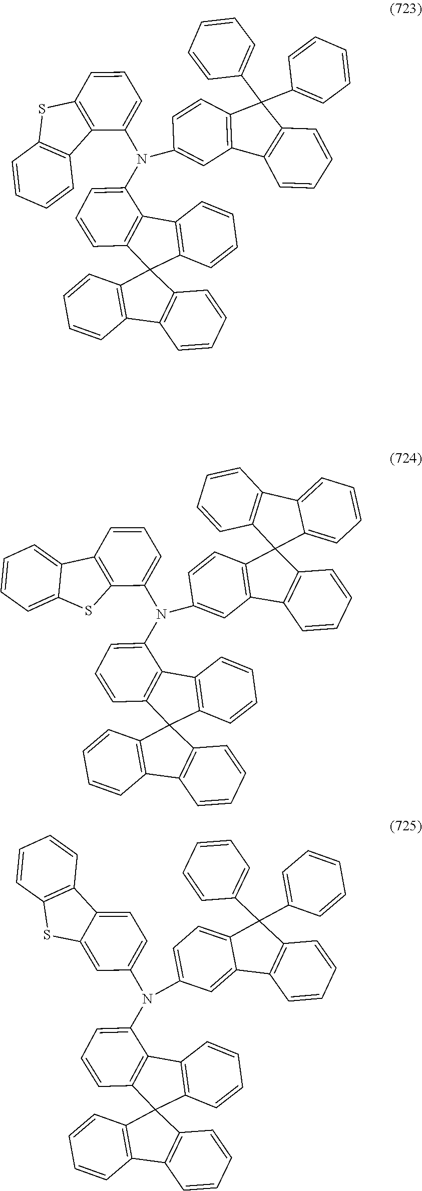

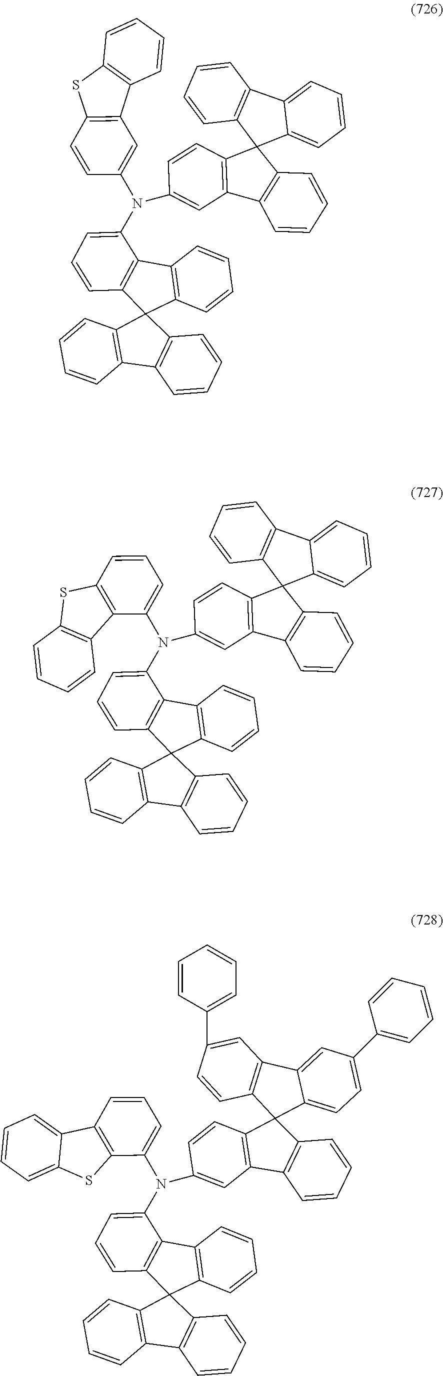

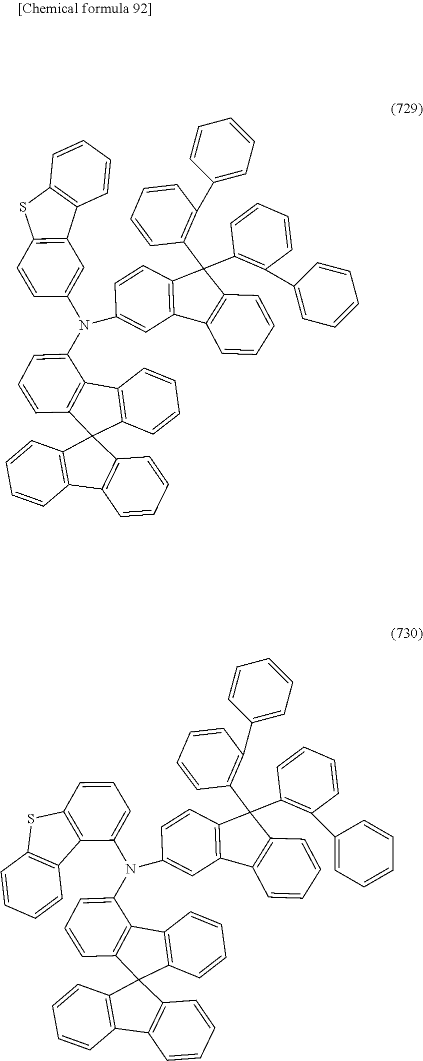

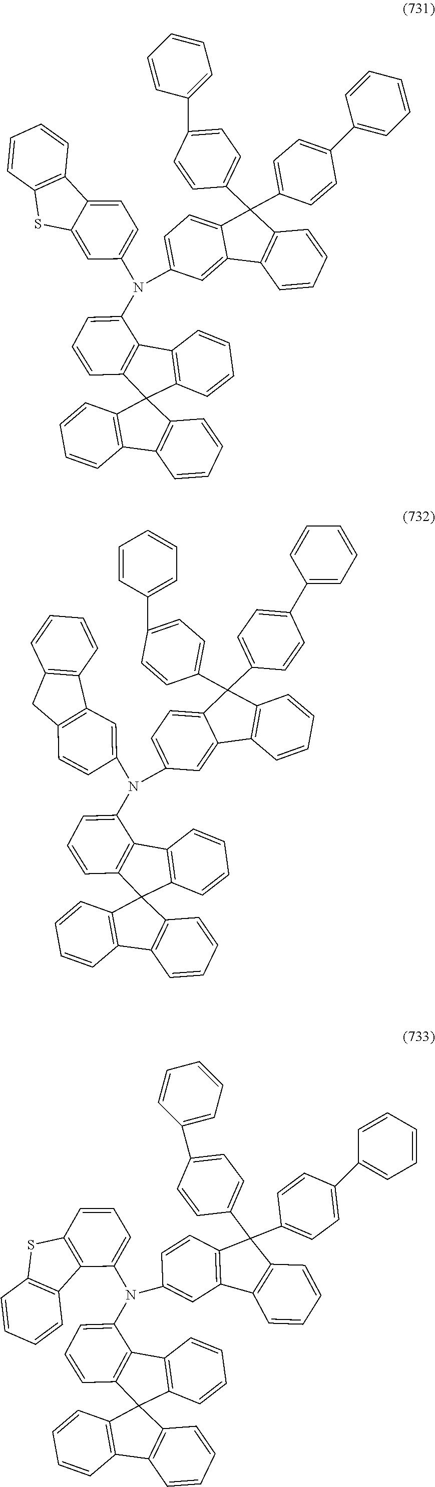

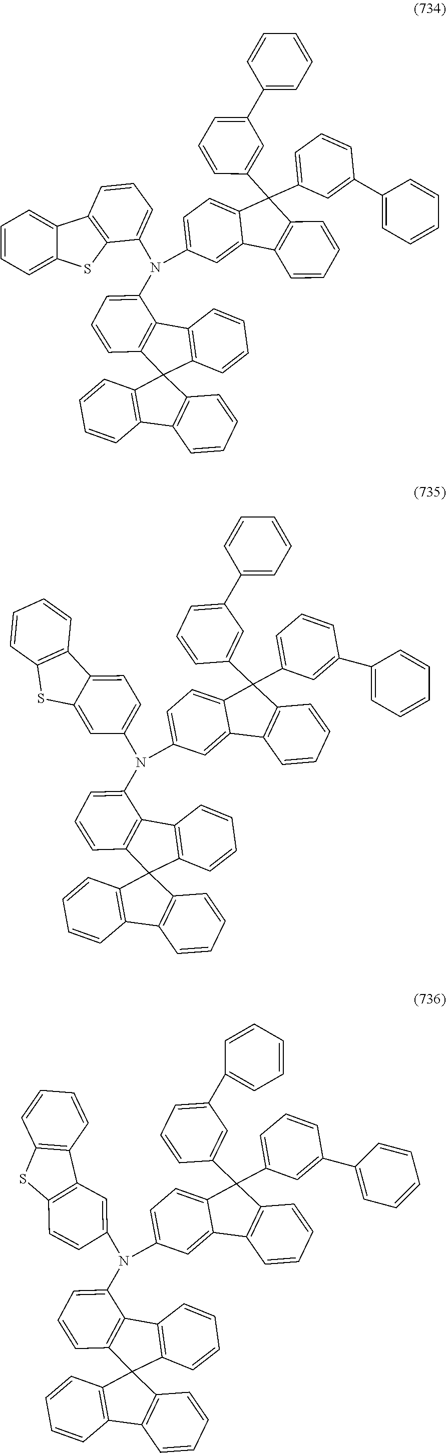

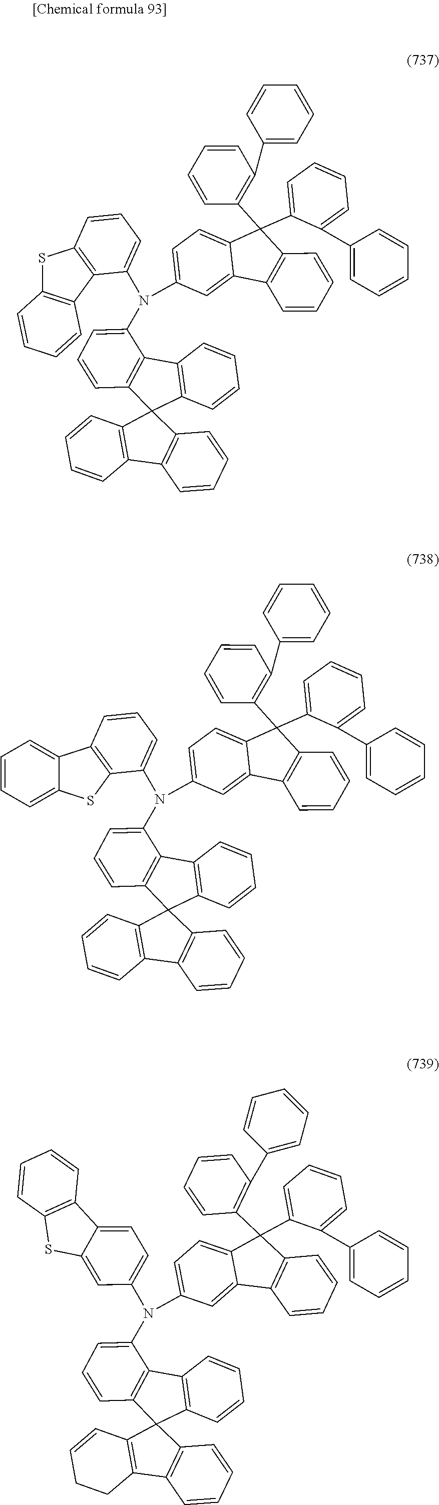

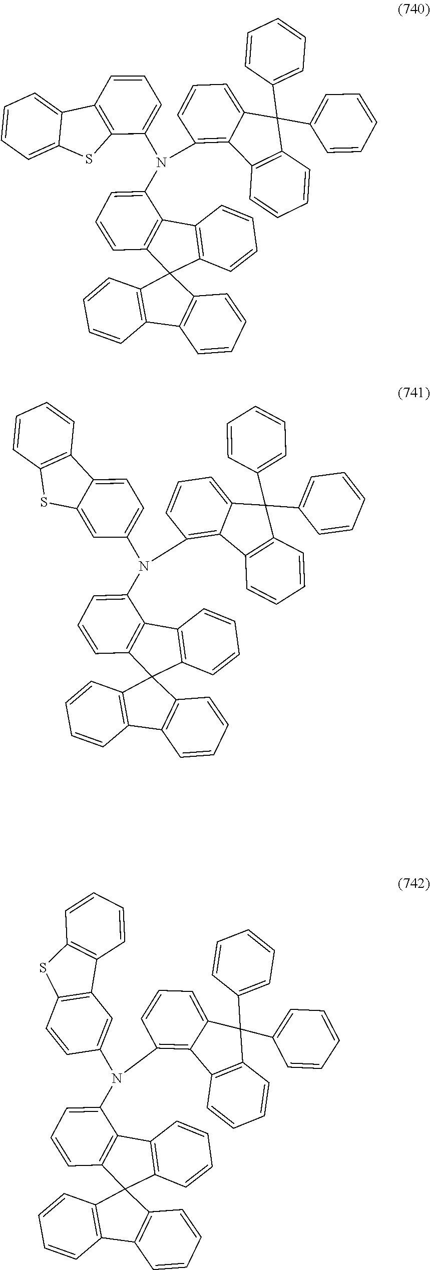

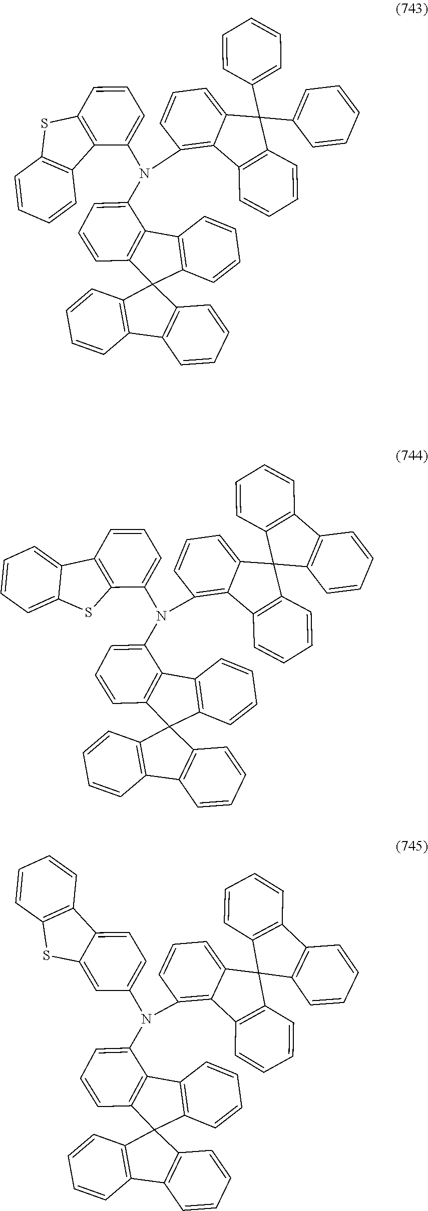

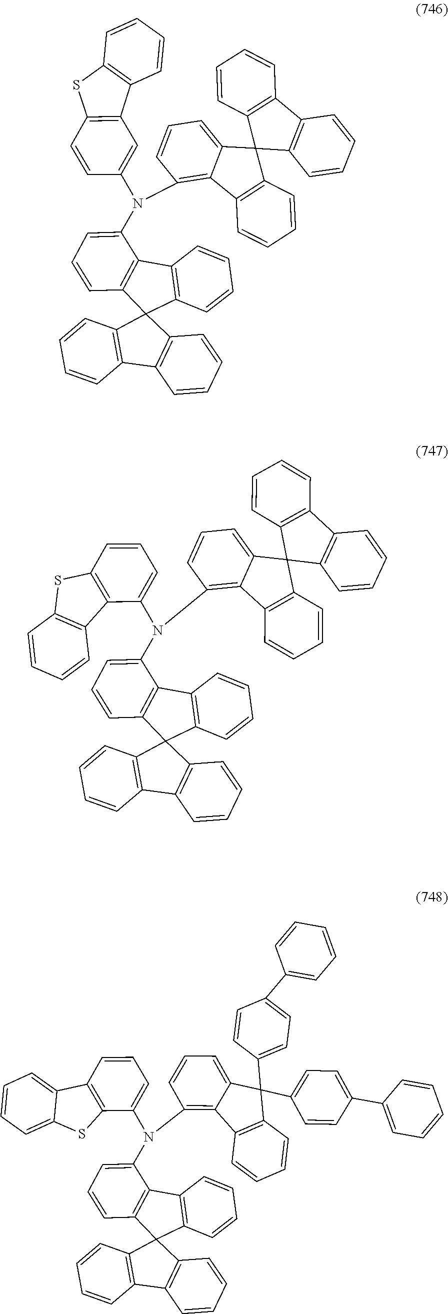

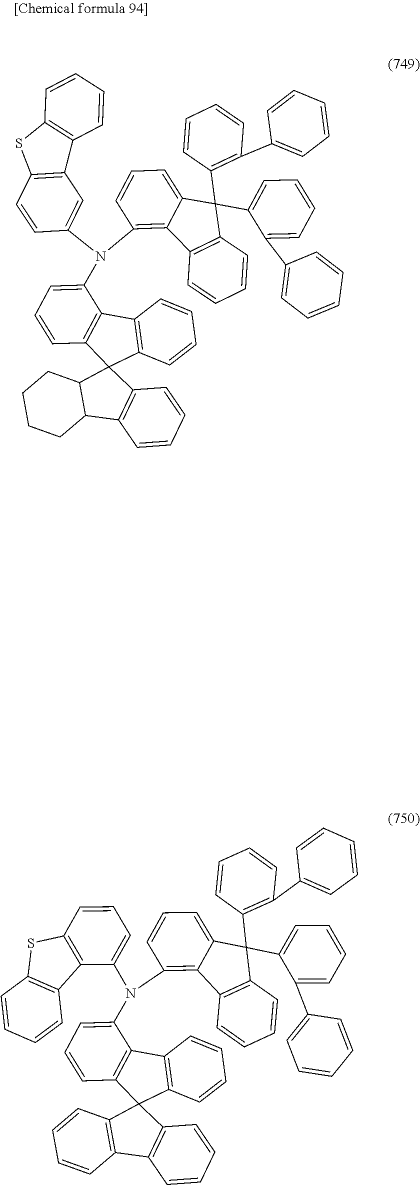

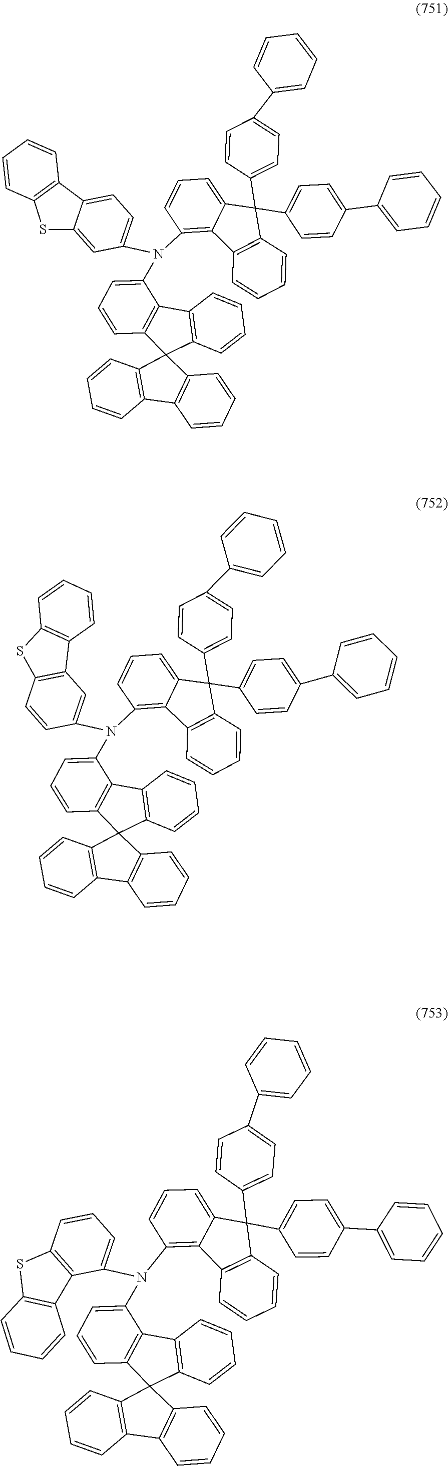

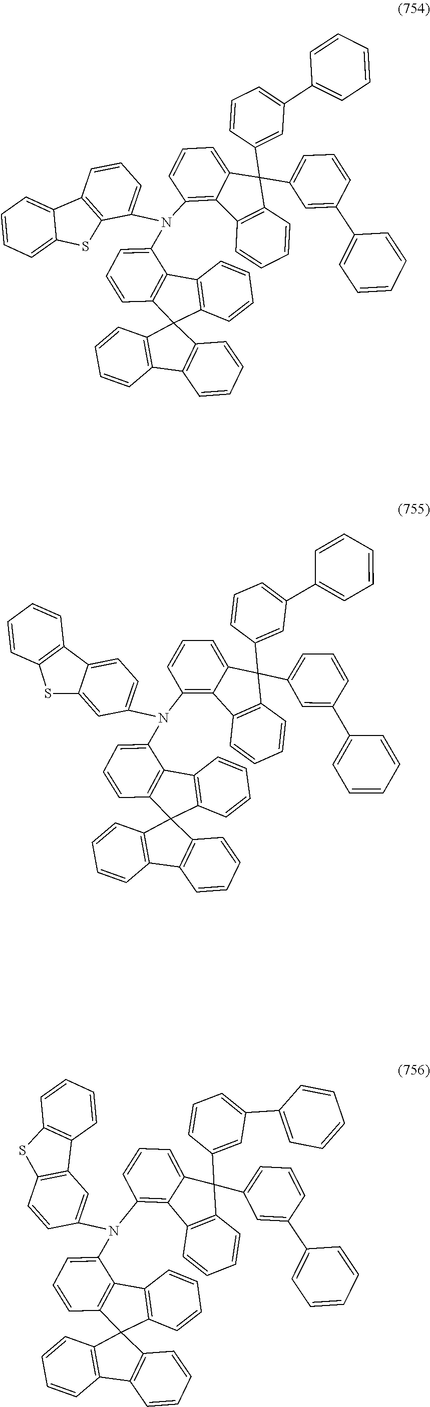

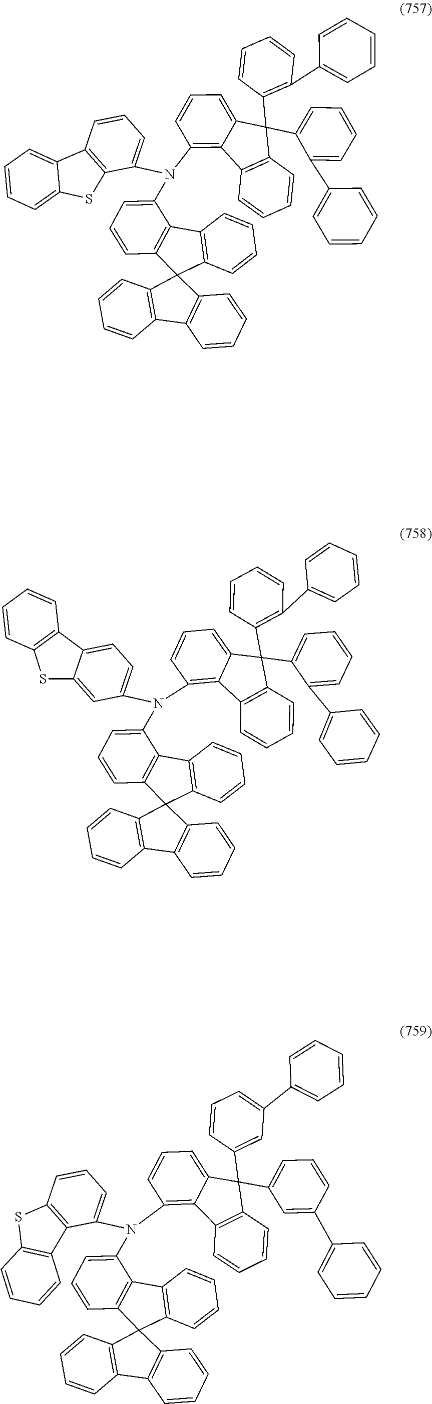

The organic compounds represented by the above Structural Formulae are specific examples of the structures represented by General Formulae (G21) to (G28). The organic compound of one embodiment of the present invention is not limited thereto.

Next, a method for synthesizing the organic compound of one embodiment of the present invention represented by General Formula (G21) will be described.

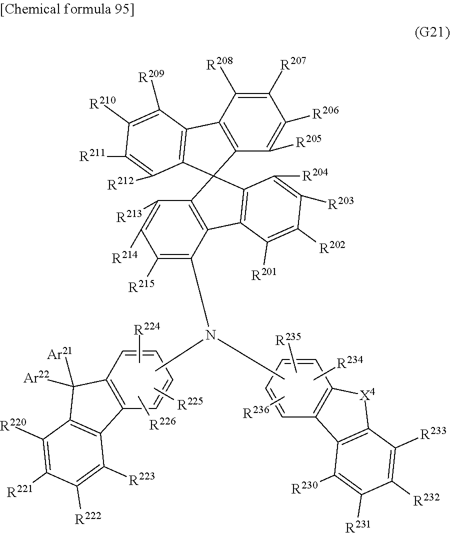

In General Formula (G21), Ar²¹ and Ar²² each independently represent a substituted or unsubstituted aryl group having 6 to 30 carbon atoms, R²⁰¹ to R²¹⁵, R²³⁰ to R²²⁶, and R²³⁰ to R²³⁶ each independently represent hydrogen (including deuterium), a substituted or unsubstituted alkyl group having 1 to 6 carbon atoms, a substituted or unsubstituted cycloalkyl group having 3 to 10 carbon atoms, a substituted or unsubstituted aryl group having 6 to 30 carbon atoms, a substituted or unsubstituted heteroaryl group having 3 to 30 carbon atoms, and X⁴ represents sulfur or oxygen. Note that the substituents represented by Ar²¹ and Ar²² may be bonded to each other to form a ring.

Synthesis Schemes (B-1-1) or (B-1-2) and (B-2), Synthesis Schemes (B-3-1) or (B-3-2) and (B-4), and Synthesis Schemes (B-5-1) or (B-5-2) and (B-6) of the organic compound of one embodiment of the present invention represented by General Formula (G21) are shown below. The organic compound of one embodiment of the present invention represented by General Formula (G21) can be synthesized through any of Synthesis Schemes (B-1-1) or (B-1-2) and (B-2), Synthesis Schemes (B-3-1) or (B-3-2) and (B-4), and Synthesis Schemes (B-5-1) or (B-5-2) and (B-6).

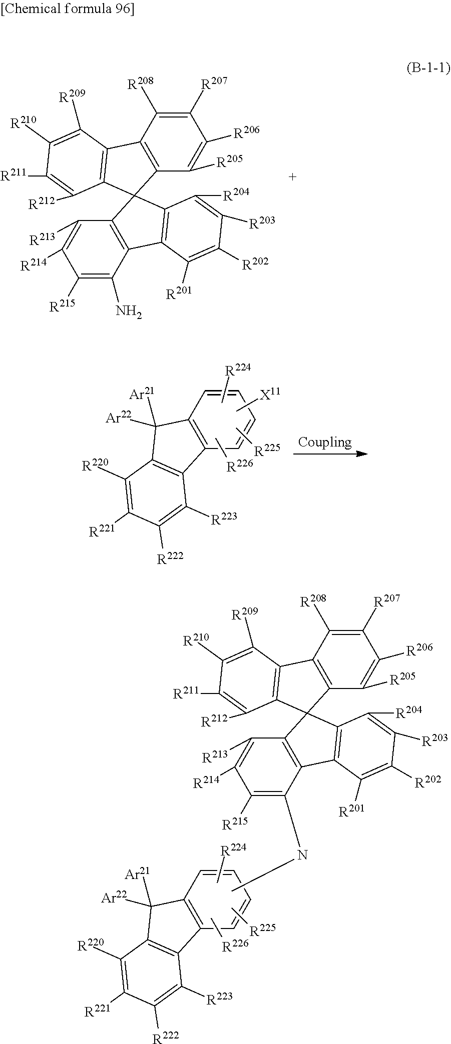

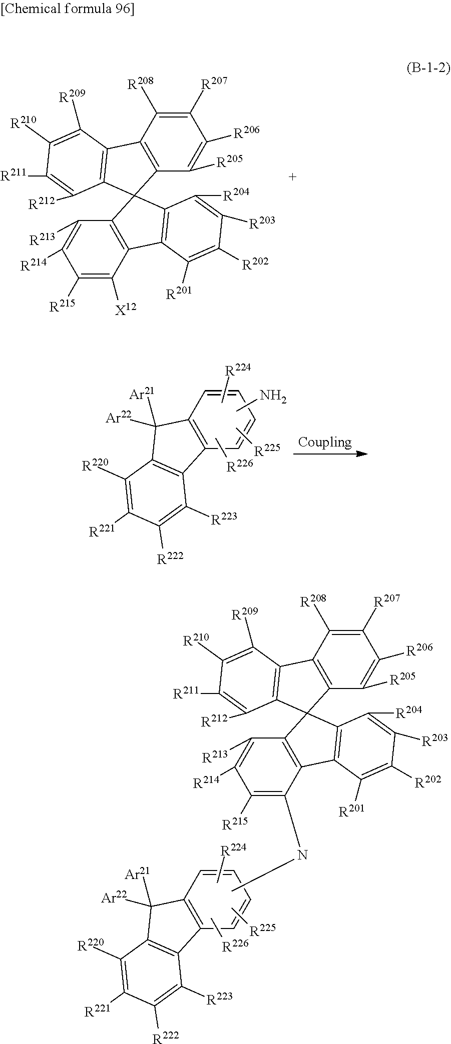

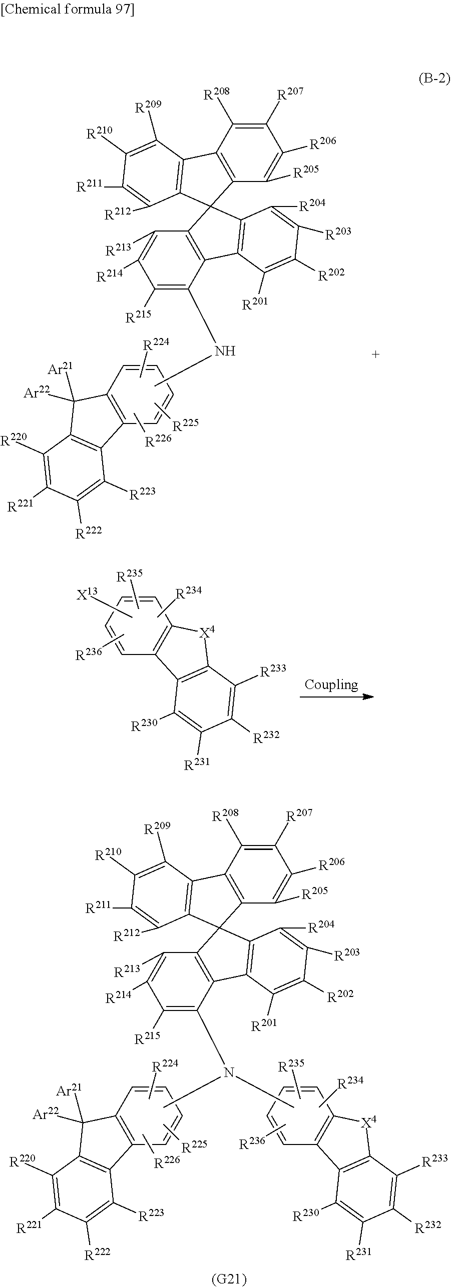

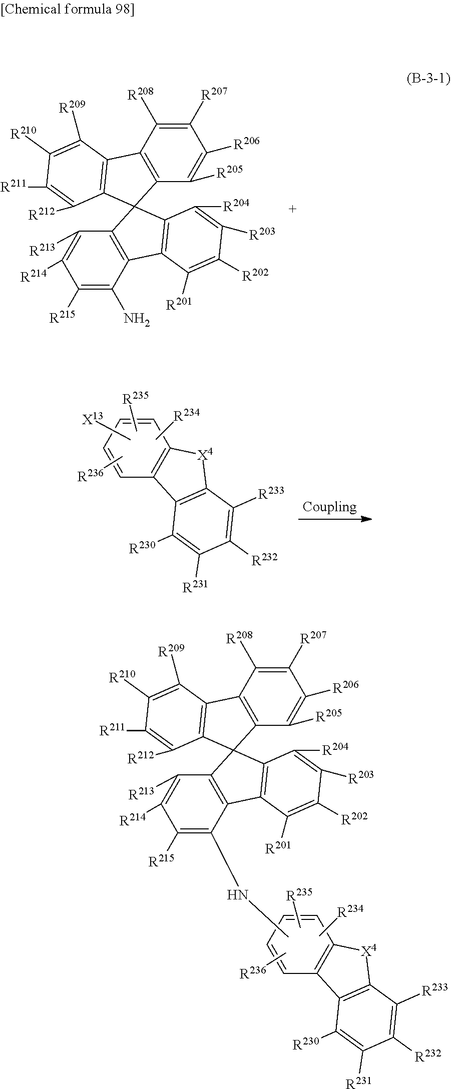

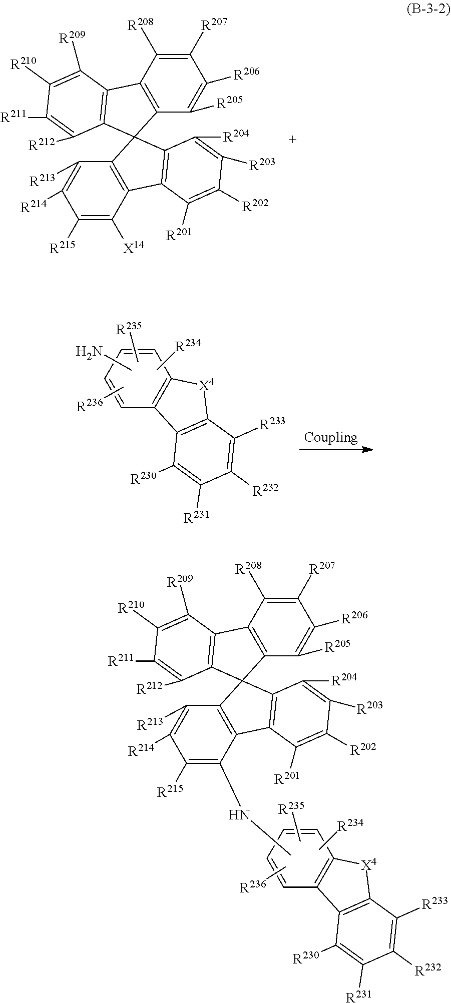

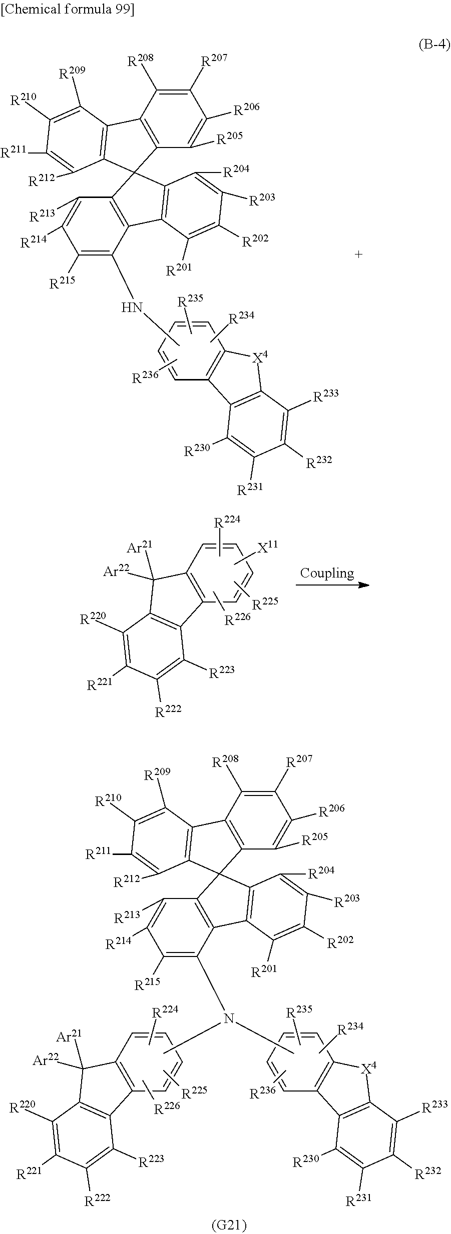

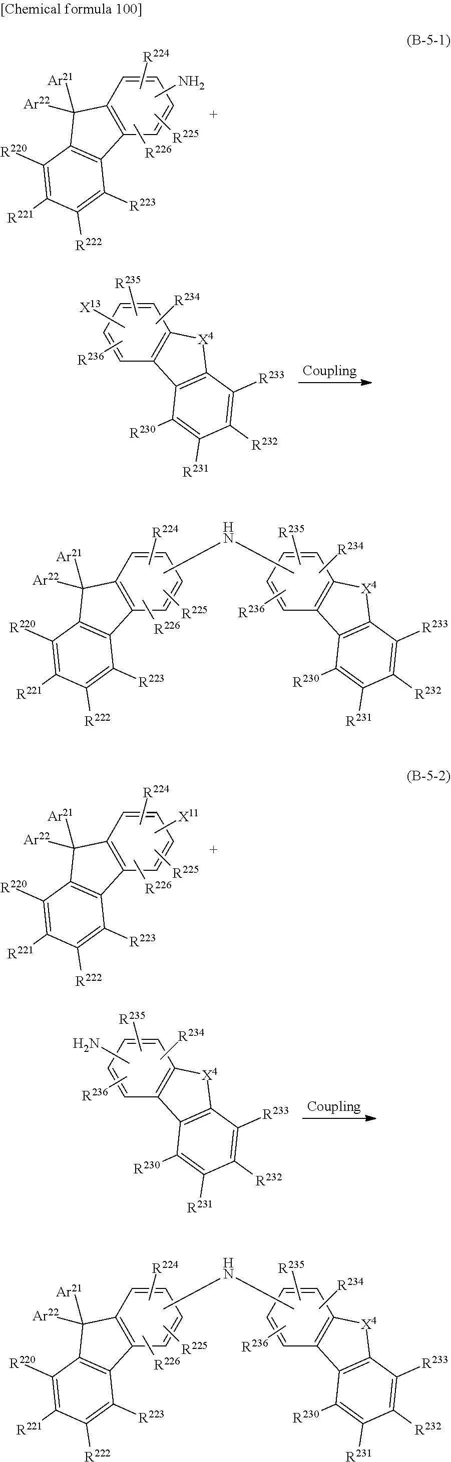

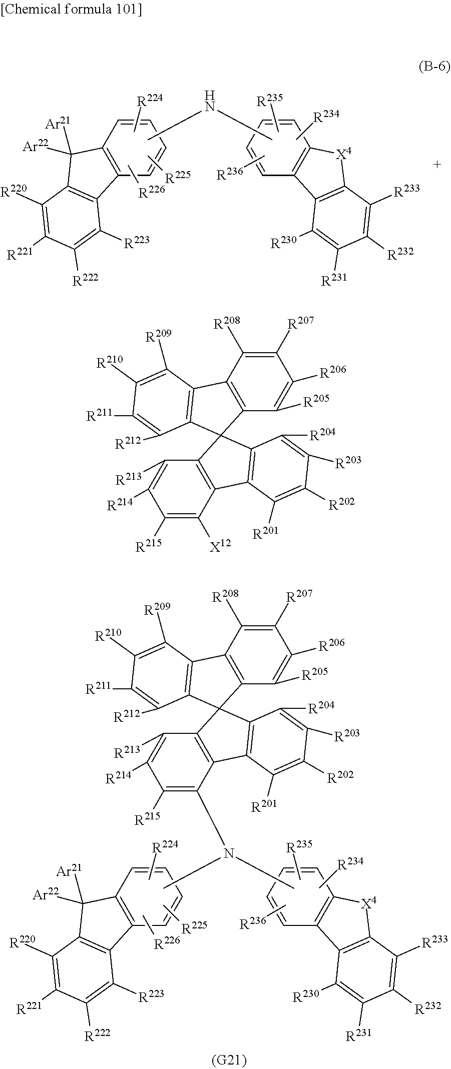

In Synthesis Schemes (B-1-1) or (B-1-2) and (B-2), Synthesis Schemes (B-3-1) or (B-3-2) and (B-4), and Synthesis Schemes (B-5-1) or(B-5-2) and (B-6), Ar²¹ and Ar²² each independently represent a substituted or unsubstituted aryl group having 6 to 30 carbon atoms; R²⁰¹ to R²¹⁵, R²²⁰ to R²²⁶, and R²³⁰ to R²³⁶ each independently represent hydrogen (including deuterium), a substituted or unsubstituted alkyl group having 1 to 6 carbon atoms, a substituted or unsubstituted cycloalkyl group having 3 to 10 carbon atoms, a substituted or unsubstituted aryl group having 6 to 30 carbon atoms, a substituted or unsubstituted heteroaryl group having 3 to 30 carbon atoms, and X⁴ represents sulfur or oxygen. Note that the substituents represented by Ar²¹ and Ar²² may be bonded to each other to form a ring. Furthermore, X¹¹ to X¹³ each represent a halogen or a trifluoromethanesulfonic acid group, and preferably represent chlorine, bromine, or iodine.

As shown in Synthesis Scheme (B-3-1), a coupling reaction between a spirobifluorene compound having an amino group and a fluorene compound having a halogen or a trifluoromethanesulfonic acid group is performed to produce a secondary amine. Alternatively, as shown in Synthesis Scheme (B-3-2), a coupling reaction between a fluorene compound having an amino group and a spirobifluorene compound having a halogen or a trifluoromethanesulfonic acid group is performed to produce a secondary amine. Then, as shown in Synthesis Scheme (B-2), a coupling reaction between the secondary amine compound obtained through Synthesis Scheme (B-3-1) or (B-3-2) and a dibenzofuran compound having a halogen or a trifluoromethanesulfonic acid group or a dibenzothiophene compound having a halogen or a trifluoromethanesulfonic acid group is performed to produce the target substance represented by General Formula (G21).

In addition, as shown in Synthesis Scheme (B-3-1), a coupling reaction between a spirobifluorene compound having an amino group and a dibenzofuran compound having a halogen or a trifluoromethanesulfonic acid group or a dibenzothiophene compound having a halogen or a trifluoromethanesulfonic acid group is performed to produce a secondary amine. Alternatively, as shown in Synthesis Scheme (B-3-2), a coupling reaction between a dibenzofuran compound having an amino group or a dibenzothiophene compound having an amino group and a spirobifluorene compound having a halogen or a trifluoromethanesulfonic acid group is performed to produce a secondary amine. Then, as shown in Synthesis Scheme (B-4), a coupling reaction between the secondary amine compound obtained through Synthesis Scheme (B-3-1) or (B-3-2) and a fluorene compound having a halogen or a trifluoromethanesulfonic acid group is performed to produce the target substance represented by General Formula(G21).

As shown in Synthesis Scheme (B-5-1), a coupling reaction between a fluorene compound having an amino group and a dibenzofuran compound having a halogen or a trifluoromethanesulfonic acid group or a dibenzothiophene compound having a halogen or a trifluoromethanesulfonic acid group is performed to produce a secondary amine. Alternatively, as shown in Synthesis Scheme (B-5-2), a coupling reaction between a dibenzofuran compound having an amino group or a dibenzothiophene compound having an amino group and a fluorene compound having a halogen or a trifluoromethanesulfonic acid group is performed to produce a secondary amine. Then, as shown in Synthesis Scheme (B-6), a coupling reaction between the secondary amine compound obtained through the reaction shown in Synthesis Scheme (B-5-1) or (B-5-2) and a spirobifluorene compound having a halogen or a trifluoromethanesulfonic acid group is performed to produce the target substance represented by General Formula (G21).

As shown in Synthesis Schemes (B-1-1) or (B-1-2) and (B-2), Synthesis Schemes (B-3-1) or (B-3-2) and (B-4), and Synthesis Schemes (B-5-1) or (B-5-2) and (B-6), the target substance can be obtained through coupling reactions in any order, and thus any material can be selected.

In Synthesis Schemes (B-1-1) or (B-1-2) and (B-2), Synthesis Schemes (B-3-1) or (B-3-2) and (B-4), and Synthesis Schemes (B-5-1) or (B-5-2) and (B-6), in the case where the Buchwald-Hartwig reaction using a palladium catalyst is employed, a palladium compound such as bis(dibenzylideneacetone)palladium(0), palladium(II) acetate, [1,1-bis(diphenylphosphino)ferrocene]palladium(II) dichloride, tetrakis(triphenylphosphine)palladium(0), or allylpalladium(II) chloride dimer, and a ligand such as tri(tert-butyl)phosphine, tri(n-hexyl)phosphine, tricyclohexylphosphine, di(1-adamantyl)-n-butylphosphine, 2-dicyclohexylphosphino-2′,6′-dimethoxybiphenyl, tri(ortho-tolyl)phosphine, cBRIDP or 4,5-bis(diphenylphosphino)-9,9-dimethylxanthene can be used. In the reaction, an organic base such as sodium tert-butoxide, an inorganic base such as potassium carbonate, cesium carbonate, or sodium carbonate, or the like can be used. As a solvent in the reaction, toluene, xylene, benzene, tetrahydrofuran, dioxane, or the like can be used. Reagents that can be used for the reaction are not limited to the above reagents. Alternatively, a compound in which an organotin group is bonded to an amino group can be used instead of the compound having an amino group.

In Synthesis Schemes (B-1-1) or (B-1-2) and (B-2), Synthesis Schemes (B-3-1) or (B-3-2) and (B-4), and Synthesis Schemes (B-5-1) or (B-5-2) and (B-6), the Ullmann reaction using copper or a copper compound can be performed. Examples of the base to be used include an inorganic base such as potassium carbonate. Examples of the solvent that can be used in the reaction include 1,3-dimethyl-3,4,5,6-tetrahydro-2(1H)pyrimidinone (DMPU), toluene, xylene, and benzene. In the Ullmann reaction, the target substance can be obtained in a shorter time and in a higher yield when the reaction temperature is 100° C. or higher; therefore, it is preferable to use DMPU or xylene, which have high boiling temperatures. Furthermore, a reaction temperature of 150° C. or higher is further preferred, and accordingly, DMPU is preferred. The reagents that can be used in the reaction are not limited to the above reagents.

As described above, the organic compound of one embodiment of the present invention represented by General Formula (G21) can be synthesized, and an organic compound having an amino group, which is a material for the synthesis, can be synthesized through Synthesis Schemes (B-7) and (B-8).

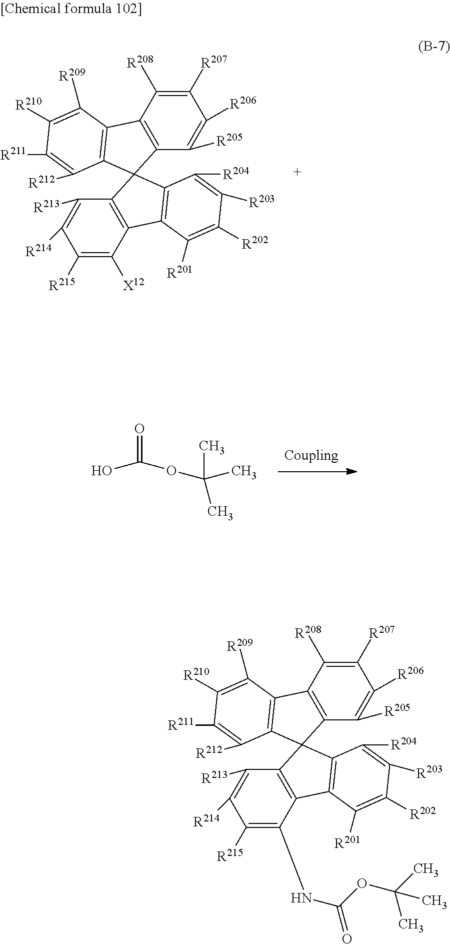

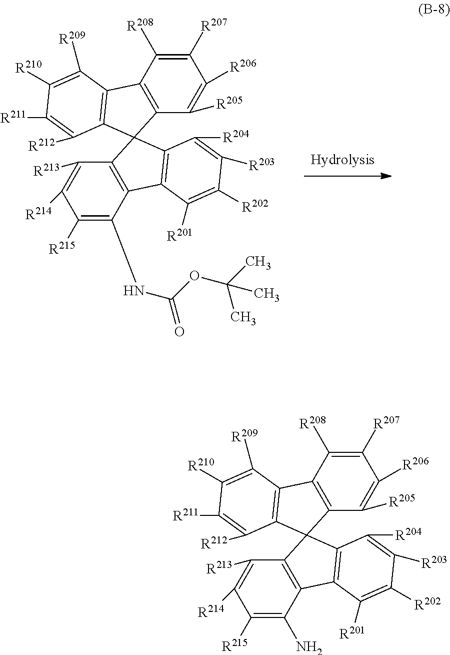

In Synthesis Scheme (B-7) and (B-8), R²⁰¹ to R²¹⁵ are the same as those in General Formula (G21), and X¹² is the same as those in Synthesis Schemes (B-1-1) or (B-1-2) and (B-2), Synthesis Schemes (B-3-1) or (B-3-2) and (B-4), and Synthesis Schemes (B-5-1) or (B-5-2) and (B-6).

In Synthesis Scheme (B-7), in the case where a coupling reaction using a palladium catalyst is employed, a palladium compound such as bis(dibenzylideneacetone)palladium(0), palladium(II) acetate, [1,1-bis(diphenylphosphino)ferrocene]palladium(II) dichloride, tetrakis(triphenylphosphine)palladium(0), or allylpalladium(II) chloride dimer and a ligand such as tri(tert-butyl)phosphine, tri(n-hexyl)phosphine, tricyclohexylphosphine, di(1-adamantyl)-n-butylphosphine, 2-dicyclohexylphosphino-2′,6′-dimethoxybiphenyl, tri(ortho-tolyl)phosphine, cBRIDP, or 4,5-bis(diphenylphosphino)-9,9-dimethylxanthene can be used. In the reaction, an organic base such as sodium tert-butoxide, an inorganic base such as potassium carbonate, cesium carbonate, or sodium carbonate, or the like can be used. As a solvent in the reaction, toluene, xylene, benzene, tetrahydrofuran, dioxane, or the like can be used. Reagents that can be used for the reaction are not limited to the above reagents. In addition, a compound in which an organotin group is bonded to an amino group can be used instead of the compound having an amino group.

Alternatively, in Synthesis Scheme (B-7), the Ullmann reaction using copper or a copper compound can be performed. Examples of the base to be used include an inorganic base such as potassium carbonate. Examples of the solvent that can be used in the reaction include 1,3-dimethyl-3,4,5,6-tetrahydro-2(1H)pyrimidinone (DMPU), toluene, xylene, and benzene. In the Ullmann reaction, the target substance can be obtained in a shorter time and in a higher yield when the reaction temperature is 100° C. or higher; therefore, it is preferable to use DMPU or xylene, which have high boiling temperatures. Furthermore, a reaction temperature of 150° C. or higher is further preferred, and accordingly, DMPU is preferred. The reagents that can be used in the reaction are not limited to the above reagents.

In the case where the hydrolysis reaction shown in Synthesis Scheme (B-8) is performed, a preferred acid is an acid not causing a dehydration reaction, such as trifluoroacetic acid, trifluoromethanesulfonic acid, acetic acid, hydrochloric acid, or hydrobromic acid, or a preferred base is a sodium hydroxide solution, a potassium hydroxide solution, or the like.

The organic compounds having an amino group in Synthesis Schemes (B-1-1) or (B-1-2) and (B-2), Synthesis Schemes (B-3-1) or (B-3-2) and (B-4), and Synthesis Schemes (B-5-1) or (B-5-2) and (B-6) can be synthesized through a reaction similar to Synthesis Schemes (B-7) and (B-8) above, and can undergo amination as shown in Synthesis Schemes (B-9) and (B-10). The amination shown in Synthesis Schemes (B-9) and (B-10) can be performed by a synthesis method similar to the reaction shown in Synthesis Schemes (B-7) and (B-8).

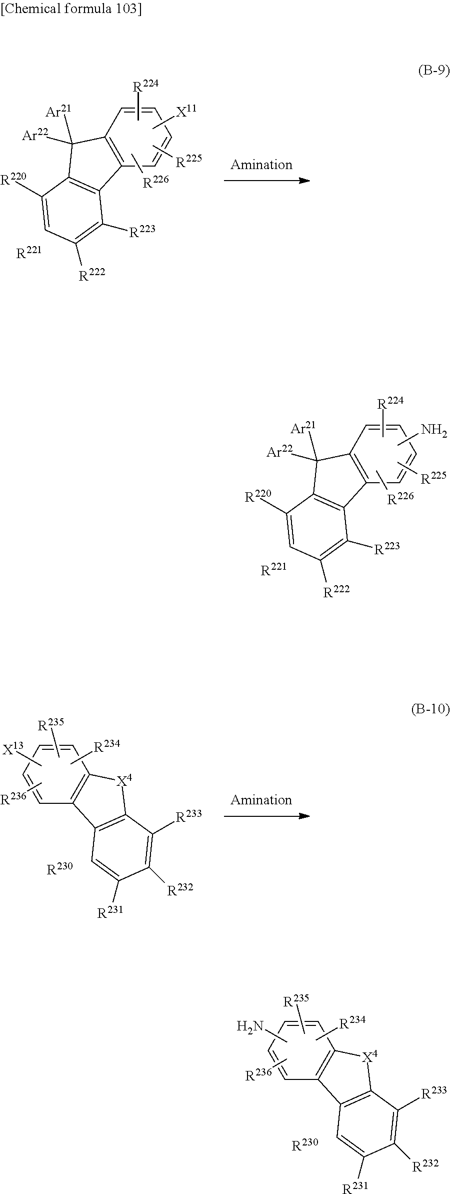

Although an example of a method for synthesizing the organic compound of one embodiment of the present invention is described above, the present invention is not limited thereto and any other synthesis method may be employed.

FIGS. 1B and 1C each illustrate an example of a specific structure of the light-emitting device 100 in FIG. 1A. In FIG. 1B, the hole-injection layer 111, the hole-transport layer 112, the light-emitting layer 113, the electron-transport layer 114, and the electron-injection layer 115 are sequentially stacked over the first electrode 101.

As illustrated in the cross-sectional view in FIG. 1B, end portions (or side surfaces) of the hole-injection layer 111, the hole-transport layer 112, the light-emitting layer 113, and the electron-transport layer 114 are positioned on the inner side than an end portion (or a side surface) of the first electrode 101. In addition, the end portions (or side surfaces) of the hole-injection layer 111, the hole-transport layer 112, the light-emitting layer 113, and the electron-transport layer 114, and part of the top surface and the end portion (or side surface) of the first electrode 101 are in contact with an insulating layer 107.

With the insulating layer 107, the end portions (or side surfaces) of the hole-injection layer 111, the hole-transport layer 112, the light-emitting layer 113, and the electron-transport layer 114 can be protected. This can reduce damage to the layers due to a manufacturing process and prevent an electrical connection caused by contact with another layer.

Although the electron-injection layer 115 is part of the EL layer 103, the shape of the electron-injection layer 115 differs from those of the other layers (the hole-injection layer 111, the hole-transport layer 112, the light-emitting layer 113, and the electron-transport layer 114) of the EL layer 103, as illustrated in FIG. 1B. However, the shape of the electron-injection layer 115 may be the same as that of the second electrode 102. The electron-injection layer 115 and the second electrode 102 can be shared by a plurality of light-emitting devices; hence, the manufacturing process of the light-emitting device 100 can be simplified and the throughput can be improved.

The light-emitting device may have a structure illustrated in FIG. 1C. In this structure, the hole-injection layer 111, the hole-transport layer 112, the light-emitting layer 113, the electron-transport layer 114, and the electron-injection layer 115 are sequentially stacked over the first electrode 101 to cover the first electrode 101. As can be seen from the cross sectional view in FIG. 1C, the end portions of the hole-injection layer 111, the hole-transport layer 112, the light-emitting layer 113, and the electron-transport layer 114 are positioned on the outer side than the end portion (or side surface) of the first electrode 101. The insulating layer 107 is in contact with the end portions of the hole-injection layer 111, the hole-transport layer 112, the light-emitting layer 113, and the electron-transport layer 114.

The insulating layer 107 is in contact with the end portions (or side surfaces) of the hole-injection layer 111, the hole-transport layer 112, the light-emitting layer 113, and the electron-transport layer 114. The insulating layer 107 is positioned between a second insulating layer 140 and the end portions (or side surfaces) of the hole-injection layer 111, the hole-transport layer 112, the light-emitting layer 113, and the electron-transport layer 114. The electron-injection layer 115 is provided over the second insulating layer 140, the insulating layer 107, and the electron-transport layer 114. The second insulating layer 140 can be formed using an organic compound or an inorganic compound.

When the second insulating layer 140 is formed using an organic compound, an acrylic resin, a polyimide resin, an epoxy resin, a polyamide resin, a polyimide-amide resin, a siloxane resin, a benzocyclobutene-based resin, a phenol resin, precursors of these resins, or the like can be used, for example. A photosensitive resin may be used. The photosensitive resin may be a positive-type photosensitive resin or a negative-type photosensitive resin.

When formed using a photosensitive resin, the second insulating layer 140 can be formed through only light-exposure and development steps in the manufacturing process, reducing the influence on other layers by dry etching, wet etching, or the like. A negative photosensitive resin is preferably used, in which case the negative photosensitive resin can also serve as a photomask (a light-exposure mask) in a different step.

With the device structures illustrated in FIGS. 1B and 1C, the light-emitting device might decrease in reliability and luminance due to problems, such as crystallization of the light-emitting layer 113 or the electron-transport layer 114 having a surface to be processed that is sometimes heated or exposed to the air when some layers of the EL layer 103 are patterned into a desired shape in the middle of the manufacturing process. In contrast, in the light-emitting device 100 described in Embodiment 1, patterning is performed after the electron-transport layer 114 is formed and thus a problem such as crystallization of the light-emitting layer 113 can be prevented. Note that in the EL layer 103 in this case, only the structure of the electron-injection layer 115 is different from those of the other layers (the hole-injection layer 111, the hole-transport layer 112, the light-emitting layer 113, and the electron-transport layer 114) because the electron-injection layer 115 is formed after the formation of the electron-transport layer 114.

Note that the light-emitting devices 100 having the shapes illustrated in FIGS. 1B and 1C are examples of a device structure for which patterning is performed by such a manufacturing method, but the shape of the light-emitting device of one embodiment of the present invention is not limited to the shapes. With the device structure of one embodiment of the present invention, reduction in efficiency and reliability in the light-emitting device can be inhibited.

The insulating layer 107 illustrated in each of FIGS. 1B and 1C is not necessarily provided when not needed. For example, when electrical continuity between the electron-injection layer 115 and the hole-injection layer 111 and hole-transport layer 112 is sufficiently low, the light-emitting device 100 does not necessarily include the insulating layer 107.

Materials that can be used for the first electrode 101, the second electrode 102, the hole-injection layer 111, the hole-transport layer 112, the light-emitting layer 113, the electron-injection layer 115, and the insulating layer 107 will be described later in an embodiment below.

The structures described in this embodiment can be used in combination with any of the structures described in the other embodiments as appropriate.

### Embodiment 2

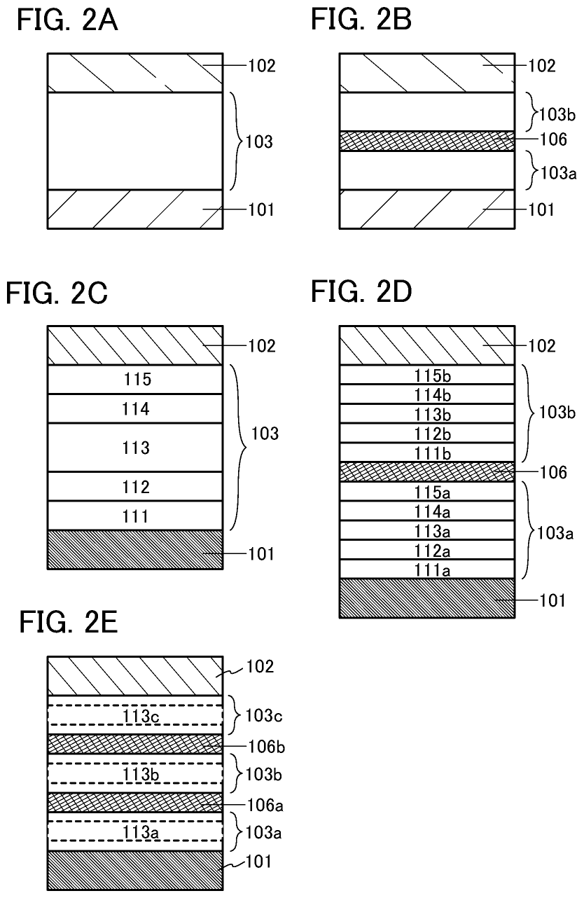

In this embodiment, other structures of the light-emitting devices described in Embodiment 1 are described with reference to FIGS. 2A to 2E.

### <<Basic Structure of Light-Emitting Device>>

A basic structure of a light-emitting device is described. FIG. 2A illustrates a light-emitting device including, between a pair of electrodes, an EL layer including a light-emitting layer. Specifically, the EL layer 103 is positioned between the first electrode 101 and the second electrode 102. The first organic compound described in Embodiment 1 can be used for the EL layer 103.

FIG. 2B illustrates a light-emitting device that has a stacked-layer structure (tandem structure) in which a plurality of EL layers (two EL layers 103a and 103b in FIG. 2B) are provided between a pair of electrodes and a charge-generation layer 106 is provided between the EL layers. A light-emitting device having a tandem structure enables fabrication of a light-emitting apparatus that has high efficiency without changing the amount of current.

The charge-generation layer 106 has a function of injecting electrons into one of the EL layers 103a and 103b and injecting holes into the other of the EL layers 103a and 103b when a potential difference is caused between the first electrode 101 and the second electrode 102. Thus, when voltage is applied in FIG. 2B such that the potential of the first electrode 101 can be higher than that of the second electrode 102, electrons are injected into the EL layer 103a from the charge-generation layer 106 and holes are injected into the EL layer 103b from the charge-generation layer 106.

Note that in terms of light extraction efficiency, the charge-generation layer 106 preferably has a property of transmitting visible light (specifically, the charge-generation layer 106 preferably has a visible light transmittance of 40% or more). The charge-generation layer 106 functions even if it has lower conductivity than the first electrode 101 or the second electrode 102.

FIG. 2C illustrates a stacked-layer structure of the EL layer 103 in the light-emitting device of one embodiment of the present invention. In this case, the first electrode 101 is regarded as functioning as an anode and the second electrode 102 is regarded as functioning as a cathode. The EL layer 103 has a structure in which the hole-injection layer 111, the hole-transport layer 112, the light-emitting layer 113, the electron-transport layer 114, and the electron-injection layer 115 are stacked in this order over the first electrode 101. Note that the light-emitting layer 113 may have a stacked-layer structure of a plurality of light-emitting layers that emit light of different colors. For example, a light-emitting layer containing a light-emitting substance that emits red light, a light-emitting layer containing a light-emitting substance that emits green light, and a light-emitting layer containing a light-emitting substance that emits blue light may be stacked with or without a layer containing a carrier-transport material therebetween. Alternatively, a light-emitting layer containing a light-emitting substance that emits yellow light and a light-emitting layer containing a light-emitting substance that emits blue light may be used in combination. Note that the stacked-layer structure of the light-emitting layer 113 is not limited to the above. For example, the light-emitting layer 113 may have a stacked-layer structure of a plurality of light-emitting layers that emit light of the same color. For example, a first light-emitting layer containing a light-emitting substance that emits blue light and a second light-emitting layer containing a light-emitting substance that emits blue light may be stacked with or without a layer containing a carrier-transport material therebetween. The structure in which a plurality of light-emitting layers that emit light of the same color are stacked can achieve higher reliability than a single-layer structure in some cases. In the case where a plurality of EL layers are provided as in the tandem structure illustrated in FIG. 2B, the layers in each EL layer are sequentially stacked from the anode side as described above. When the first electrode 101 is the cathode and the second electrode 102 is the anode, the stacking order of the layers in the EL layer 103 is reversed. Specifically, the layer 111 over the first electrode 101 serving as the cathode is an electron-injection layer; the layer 112 is an electron-transport layer; the layer 113 is a light-emitting layer; the layer 114 is a hole-transport layer; and the layer 115 is a hole-injection layer.

The light-emitting layer 113 included in each of the EL layers (103, 103a, and 103b) contains an appropriate combination of a light-emitting substance and a plurality of substances, so that fluorescent light of a desired color or phosphorescent light of a desired color can be obtained. The light-emitting layer 113 may have a stacked-layer structure having different emission colors. In that case, light-emitting substances and other substances are different between the stacked light-emitting layers. Alternatively, the plurality of EL layers (103a and 103b) in FIG. 2B may exhibit their respective emission colors. Also in that case, the light-emitting substances and other substances are different between the stacked light-emitting layers.

The light-emitting device of one embodiment of the present invention can have a micro optical resonator (microcavity) structure when, for example, the first electrode 101 is a reflective electrode and the second electrode 102 is a transflective electrode in FIG. 2C. Thus, light from the light-emitting layer 113 in the EL layer 103 can be resonated between the electrodes and light emitted through the second electrode 102 can be intensified.

Note that when the first electrode 101 of the light-emitting device is a reflective electrode having a stacked-layer structure of a reflective conductive material and a light-transmitting conductive material (transparent conductive film), optical adjustment can be performed by adjusting the thickness of the transparent conductive film. Specifically, when the wavelength of light obtained from the light-emitting layer 113 is λ, the optical path length between the first electrode 101 and the second electrode 102 (the product of the thickness and the refractive index) is preferably adjusted to be mλ/2 (m is an integer of 1 or more) or close to mλ/2.

To amplify desired light (wavelength: λ) obtained from the light-emitting layer 113, it is preferable to adjust each of the optical path length from the first electrode 101 to a region where the desired light is obtained in the light-emitting layer 113 (light-emitting region) and the optical path length from the second electrode 102 to the region where the desired light is obtained in the light-emitting layer 113 (light-emitting region) to be (2m′+1)λ/4 (m′ is an integer of 1 or more) or close to (2m′+1)λ/4. Here, the light-emitting region means a region where holes and electrons are recombined in the light-emitting layer 113.

By such optical adjustment, the spectrum of specific monochromatic light obtained from the light-emitting layer 113 can be narrowed and light emission with high color purity can be obtained.

In the above case, the optical path length between the first electrode 101 and the second electrode 102 is, to be exact, the total thickness from a reflective region in the first electrode 101 to a reflective region in the second electrode 102. However, it is difficult to precisely determine the reflective regions in the first electrode 101 and the second electrode 102; thus, it is assumed that the above effect can be sufficiently obtained wherever the reflective regions may be set in the first electrode 101 and the second electrode 102. Furthermore, the optical path length between the first electrode 101 and the light-emitting layer that emits the desired light is, to be exact, the optical path length between the reflective region in the first electrode 101 and the light-emitting region in the light-emitting layer that emits the desired light. However, it is difficult to precisely determine the reflective region in the first electrode 101 and the light-emitting region in the light-emitting layer that emits the desired light; thus, it is assumed that the above effect can be sufficiently obtained wherever the reflective region and the light-emitting region may be set in the first electrode 101 and the light-emitting layer that emits the desired light, respectively.

The light-emitting device illustrated in FIG. 2D is a light-emitting device having a tandem structure. Owing to a microcavity structure, light (monochromatic light) with a desired wavelength derived from any of the light-emitting layers can be extracted when light-emitting layers that emit light of different colors are used in the EL layers (103a and 103b). Thus, it is unnecessary to separately form EL layers for obtaining different emission colors (e.g., R, G, and B) by using such a light-emitting device in the light-emitting apparatus and adjusting the microcavity structure in order to extract light with wavelengths which differ depending on subpixels. Therefore, high resolution can be easily achieved. A combination with coloring layers (color filters) is also possible. Furthermore, the emission intensity of light with a specific wavelength in the front direction can be increased, whereby power consumption can be reduced.

The light-emitting device illustrated in FIG. 2E is an example of the light-emitting device having the tandem structure illustrated in FIG. 2B, and includes three EL layers (103a, 103b, and 103c) stacked with charge-generation layers (106a and 106b) positioned therebetween, as illustrated in FIG. 2E. The three EL layers (103a, 103b, and 103c) include respective light-emitting layers (113a, 113b, and 113c), and the emission colors of the light-emitting layers can be selected freely. For example, the light-emitting layer 113a can emit blue light, the light-emitting layer 113b can emit red light, green light, or yellow light, and the light-emitting layer 113c can emit blue light, or the light-emitting layer 113a can emit red light, the light-emitting layer 113b can emit blue light, green light, or yellow light, and the light-emitting layer 113c can emit red light.

In the light-emitting device of one embodiment of the present invention, at least one of the first electrode 101 and the second electrode 102 is a light-transmitting electrode (e.g., a transparent electrode or a transflective electrode). In the case where the light-transmitting electrode is a transparent electrode, the transparent electrode has a visible light transmittance higher than or equal to 40%. In the case where the light-transmitting electrode is a transflective electrode, the transflective electrode has a visible light reflectance higher than or equal to 20% and lower than or equal to 80%, preferably higher than or equal to 40% and lower than or equal to 70%. These electrodes preferably have a resistivity of 1×10⁻² Ωcm or less.

When one of the first electrode 101 and the second electrode 102 is a reflective electrode in the light-emitting device of one embodiment of the present invention, the visible light reflectance of the reflective electrode is higher than or equal to 40% and lower than or equal to 100%, preferably higher than or equal to 70% and lower than or equal to 100%. This electrode preferably has a resistivity of 1×10⁻² Ωcm or less.

### <<Specific Structure of Light-Emitting Device>>

Next, a specific structure of the light-emitting device of one embodiment of the present invention will be described. Here, the description is made using FIG. 2D illustrating the tandem structure. Note that the structure of the EL layer applies also to the structure of the light-emitting devices having a single structure in FIGS. 2A and 2C. When the light-emitting device in FIG. 2D has a microcavity structure, the first electrode 101 is formed as a reflective electrode and the second electrode 102 is formed as a transflective electrode. Thus, a single-layer structure or a stacked-layer structure can be formed using one or more kinds of desired electrode materials. Note that the second electrode 102 is formed after formation of the EL layer 103b, with the use of a material selected as appropriate.

### <First Electrode and Second Electrode>

As materials for the first electrode 101 and the second electrode 102, any of the following materials can be used in an appropriate combination as long as the above functions of the electrodes can be fulfilled. For example, a metal, an alloy, an electrically conductive compound, a mixture of these, and the like can be used as appropriate. Specifically, an In—Sn oxide (also referred to as ITO), an In—Si—Sn oxide (also referred to as ITSO), an In—Zn oxide, or an In—W—Zn oxide can be used. In addition, it is possible to use a metal such as aluminum (Al), titanium (Ti), chromium (Cr), manganese (Mn), iron (Fe), cobalt (Co), nickel (Ni), copper (Cu), gallium(Ga), zinc (Zn), indium (In), tin (Sn), molybdenum (Mo), tantalum (Ta), tungsten (W), palladium (Pd), gold (Au), platinum (Pt), silver (Ag), yttrium (Y), or neodymium (Nd) or an alloy containing an appropriate combination of any of these metals. It is also possible to use a Group 1 element or a Group 2 element in the periodic table that is not described above (e.g., lithium (Li), cesium (Cs), calcium (Ca), or strontium (Sr)), a rare earth metal such as europium (Eu) or ytterbium (Yb), an alloy containing an appropriate combination of any of these elements, graphene, or the like.

In the light-emitting device in FIG. 2D, when the first electrode 101 is the anode, a hole-injection layer 111a and a hole-transport layer 112a of the EL layer 103a are sequentially stacked over the first electrode 101 by a vacuum evaporation method. After the EL layer 103a and the charge-generation layer 106 are formed, a hole-injection layer 111b and a hole-transport layer 112b of the EL layer 103b are sequentially stacked over the charge-generation layer 106 in a similar manner.

### <Hole-Injection Layer>

The hole-injection layers (111, 111a, and 111b) inject holes from the first electrode 101 serving as the anode and the charge-generation layers (106, 106a, and 106b) to the EL layers (103, 103a, and 103b) and contain an organic acceptor material or a material having a high hole-injection property. The first organic compound described in Embodiment 1 can be used for the hole-injection layer.

The organic acceptor material allows holes to be generated in another organic compound whose HOMO level is close to the lowest unoccupied molecular orbital (LUMO)level of the organic acceptor material when charge separation is caused between the organic acceptor material and the organic compound. Thus, as the organic acceptor material, a compound having an electron-withdrawing group (e.g., a halogen group or a cyano group), such as a quinodimethane derivative, a chloranil derivative, and a hexaazatriphenylene derivative, can be used. Examples of the organic acceptor material include 7,7,8,8-tetracyano-2,3,5,6-tetrafluoroquinodimethane (abbreviation: F₄-TCNQ), 3,6-difluoro-2,5,7,7,8,8-hexacyanoquinodimethane, chloranil, 2,3,6,7,10,11-hexacyano-1,4,5,8,9,12-hexaazatriphenylene (abbreviation: HAT-CN), 1,3,4,5,7,8-hexafluorotetracyano-naphthoquinodimethane (abbreviation: F6-TCNNQ), and 2-(7-dicyanomethylene-1,3,4,5,6,8,9,10-octafluoro-7H-pyren-2-ylidene)malononitrile. Note that among organic acceptor materials, a compound in which electron-withdrawing groups are bonded to fused aromatic rings each having a plurality of heteroatoms, such as HAT-CN, is particularly preferred because it has a high acceptor property and stable film quality against heat. Besides, a [3]radialene derivative having an electron-withdrawing group (particularly a cyano group or a halogen group such as a fluoro group), which has a very high electron-accepting property, is preferred; specific examples include α,α′,α″-1,2,3-cyclopropanetriylidenetris[4-cyano-2,3,5,6-tetrafluorobenzeneacetonitrile], α,α′,α″-1,2,3-cyclopropanetriylidenetris[2,6-dichloro-3,5-difluoro-4-(trifluoromethyl)benzeneacetonitrile], and α,α′,α″-1,2,3-cyclopropanetriylidenetris[2,3,4,5,6-pentafluorobenzeneacetonitrile].

As the material having a high hole-injection property, an oxide of a metal belonging to Group 4 to Group 8 in the periodic table (e.g., a transition metal oxide such as molybdenum oxide, vanadium oxide, ruthenium oxide, tungsten oxide, or manganese oxide) can be used. Specific examples include molybdenum oxide, vanadium oxide, niobium oxide, tantalum oxide, chromium oxide, tungsten oxide, manganese oxide, and rhenium oxide. Among these oxides, molybdenum oxide is preferable because it is stable in the air, has a low hygroscopic property, and is easily handled. Other examples are phthalocyanine (abbreviation: H₂Pc) and a phthalocyanine-based compound such as copper phthalocyanine (abbreviation: CuPc), and the like.

Other examples are aromatic amine compounds, which are low-molecular compounds, such as 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl (abbreviation: DPAB), N,N′-bis{4-[bis(3-methylphenyl)amino]phenyl}-N,N′-diphenyl-(1,1′-biphenyl)-4,4′-diamine (abbreviation: DNTPD), 1,3,5-tris[N-(4-diphenylaminophenyl)-N-phenylamino]benzene (abbreviation: DPA3B), 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), and 3-[N-(1-naphthyl)-N-(9-phenylcarbazol-3-yl)amino]-9-phenylcarbazole (abbreviation: PCzPCN1).

Other examples are high-molecular compounds (e.g., oligomers, dendrimers, and polymers) such as poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl)methacrylanide] (abbreviation: PTPDMA), and poly[N,N′-bis(4-butylphenyl)-N,N′-bis(phenyl)benzidine] (abbreviation: Poly-TPD). Alternatively, it is possible to use a high-molecular compound to which acid is added, such as poly(3,4-ethylenedioxythiophene)/poly(styrenesulfonic acid) (abbreviation: PEDOT/PSS) or polyaniline/poly(styrenesulfonic acid) (abbreviation: PAni/PSS), for example.

As the material having a high hole-injection property, a mixed material containing a hole-transport material and the above-described organic acceptor material (electron-accepting material) can be used. In that case, the organic acceptor material extracts electrons from the hole-transport material, so that holes are generated in the hole-injection layer 111 and the holes are injected into the light-emitting layer 113 through the hole-transport layer 112. Note that the hole-injection layer 111 may be formed to have a single-layer structure using a mixed material containing a hole-transport material and an organic acceptor material (electron-accepting material), or a stacked-layer structure of a layer containing a hole-transport material and a layer containing an organic acceptor material (electron-accepting material).

The hole-transport material preferably has a hole mobility higher than or equal to 1×10⁻⁶ cm²/Vs in the case where the square root of the electric field strength [V/cm] is 600. Note that other substances can also be used as long as the substances have hole-transport properties higher than electron-transport properties.

As the hole-transport material, materials having a high hole-transport property, such as a compound having a π-electron rich heteroaromatic ring (e.g., a carbazole derivative, a furan derivative, and a thiophene derivative) and an aromatic amine (an organic compound having an aromatic amine skeleton), are preferable.

Examples of the carbazole derivative (an organic compound having a carbazole ring) include a bicarbazole derivative (e.g., a 3,3′-bicarbazole derivative) and an aromatic amine having a carbazolyl group.

Specific examples of the bicarbazole derivative (e.g., a 3,3′-bicarbazole derivative) include 3,3′-bis(9-phenyl-9H-carbazole) (abbreviation: PCCP), 9,9′-bis(biphenyl-4-yl)-3,3′-bi-9H-carbazole (abbreviation: BisBPCz), 9,9′-bis(1,1′-biphenyl-3-yl)-3,3′-bi-9H-carbazole (abbreviation: BismBPCz), 9-(1,1′-biphenyl-3-yl)-9′-(1,1′-biphenyl-4-yl)-9H,9′H-3,3′-bicarbazole (abbreviation: mBPCCBP), and 9-(2-naphthyl)-9′-phenyl-9H,9′H-3,3′-bicarbazole (abbreviation: βNCCP).

Specific examples of the aromatic amine having a carbazolyl group include 4-phenyl-4′-(9-phenyl-9H-carbazol-3-yl)triphenylamine (abbreviation: PCBA1BP), N-(4-biphenyl)-N-(9,9-dimethyl-9H-fluoren-2-yl)-9-phenyl-9H-carbazol-3-amine (abbreviation: PCBiF), N-(1,1′-biphenyl-4-yl)-N-[4-(9-phenyl-9H-carbazol-3-yl)phenyl]-9,9-dimethyl-9H-fluoren-2-amine (abbreviation: PCBBiF), N-[4-(9-phenyl-9H-carbazol-3-yl)phenyl]-bis(9,9-dimethyl-9H-fluoren-2-yl)amine (abbreviation: PCBFF), N-(1,1′-biphenyl-4-yl)-N-[4-(9-phenyl-9H-carbazol-3-yl)phenyl]-9,9-dimethyl-9H-fluoren-4-amine, N-[4-(9-phenyl-9H-carbazol-3-yl)phenyl]-(9,9-dimethyl-9H-fluoren-2-yl)-9,9-dimethyl-9H-fluoren-4-amine, N-(1,1′-biphenyl-4-yl)-N-[4-(9-phenyl-9H-carbazol-3-yl)phenyl]-9,9-diphenyl-9H-fluoren-2-amine, N-(1,1′-biphenyl-4-yl)-N-[4-(9-phenyl-9H-carbazol-3-yl)phenyl]-9,9-diphenyl-9H-fluoren-4-amine, N-(1,1′-biphenyl-4-yl)-N-[4-(9-phenyl-9H-carbazol-3-yl)phenyl]-9,9′-spirobi(9H-fluoren)-2-amine, N-(1,1′-biphenyl-4-yl)-N-[4-(9-phenyl-9H-carbazol-3-yl)phenyl]-9,9′-spirobi(9H-fluoren)-4-amine, N-[4-(9-phenyl-9H-carbazol-3-yl)phenyl]-N-[1,1′:3′,1″-terphenyl-4-yl]-9,9-dimethyl-9H-fluoren-2-amine, N-[4-(9-phenyl-9H-carbazol-3-yl)phenyl]-N-[1,1′:4′,1″-terphenyl-4-yl]-9,9-dimethyl-9H-fluoren-2-amine, N-[4-(9-phenyl-9H-carbazol-3-yl)phenyl]-N-[1,1′:3′,1″-terphenyl-4-yl]-9,9-dimethyl-9H-fluoren-4-amine, N-[4-(9-phenyl-9H-carbazol-3-yl)phenyl]-N-[1,1′:4′,1″-terphenyl-4-yl]-9,9-dimethyl-9H-fluoren-4-amine, 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 4-phenyldiphenyl-(9-phenyl-9H-carbazol-3-yl)amine (abbreviation: PCA1BP), N,N′-bis(9-phenylcarbazol-3-yl)-N,N′-diphenylbenzene-1,3-diamine (abbreviation: PCA2B), N,N,N″-triphenyl-N,N′,N″-tris(9-phenylcarbazol-3-yl)benzene-1,3,5-triamine (abbreviation: PCA3B), 9,9-dimethyl-N-phenyl-N-[4-(9-phenyl-9H-carbazol-3-yl)phenyl]fluoren-2-amine (abbreviation: PCBAF), N-phenyl-N-[4-(9-phenyl-9H-carbazol-3-yl)phenyl]spiro-9,9′-bifluoren-2-amine (abbreviation: PCBASF), 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), 3-[N-(1-naphthyl)-N-(9-phenylcarbazol-3-yl)amino]-9-phenylcarbazole (abbreviation: PCzPCN1), 3-[N-(4-diphenylaminophenyl)-N-phenylamino]-9-phenylcarbazole (abbreviation: PCzDPA1), 3,6-bis[N-(4-diphenylaminophenyl)-N-phenylamino]-9-phenylcarbazole (abbreviation: PCzDPA2), 3,6-bis[N-(4-diphenylaminophenyl)-N-(1-naphthyl)amino]-9-phenylcarbazole (abbreviation: PCzTPN2), 2-[N-(9-phenylcarbazol-3-yl)-N-phenylamino]spiro-9,9′-bifluorene (abbreviation: PCASF), N-[4-(9H-carbazol-9-yl)phenyl]-N-(4-phenyl)phenylaniline (abbreviation: YGA1BP), N,N′-bis[4-(carbazol-9-yl)phenyl]-N,N′-diphenyl-9,9-dimethylfluorene-2,7-diamine (abbreviation: YGA2F), and 4,4′,4″-tris(carbazol-9-yl)triphenylamine (abbreviation: TCTA).

Other examples of the carbazole derivative include 3-[4-(9-phenanthryl)-phenyl]-9-phenyl-9H-carbazole (abbreviation: PCPPn), 3-[4-(1-naphthyl)-phenyl]-9-phenyl-9H-carbazole (abbreviation: PCPN), 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), 1,3,5-tris[4-(N-carbazolyl)phenyl]benzene (abbreviation: TCPB), and 9-[4-(10-phenyl-9-anthracenyl)phenyl]-9H-carbazole (abbreviation: CzPA).

Specific examples of the furan derivative (an organic compound having a furan ring) include 4,4′,4″-(benzene-1,3,5-triyl)tri(dibenzofuran) (abbreviation: DBF3P-II) and 4-{3-[3-(9-phenyl-9H-fluoren-9-yl)phenyl]phenyl}dibenzofuran (abbreviation: mmDBFFLBi-II).

Specific examples of the thiophene derivative (an organic compound having a thiophene ring) include organic compounds having a thiophene ring, such as 4,4′,4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), and 4-[4-(9-phenyl-9H-fluoren-9-yl)phenyl]-6-phenyldibenzothiophene (abbreviation: DBTFLP-IV).

Specific examples of the aromatic amine include 4,4′-bis[N-(1-naphthyl)-N-phenylamino]biphenyl (abbreviation: NPB or α-NPD), N,N′-bis(3-methylphenyl)-N,N′-diphenyl-[1,1′-biphenyl]-4,4′-diamine (abbreviation: TPD), 4,4′-bis[N-(spiro-9,9′-bifluoren-2-yl)-N-phenylamino]biphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), N-(9,9-dimethyl-9H-fluoren-2-yl)-N-{9,9-dimethyl-2-[N′-phenyl-N′-(9,9-dimethyl-9H-fluoren-2-yl)amino]-9H-fluoren-7-yl}phenylamine (abbreviation: DFLADFL), N-(9,9-dimethyl-2-diphenylamino-9H-fluoren-7-yl)diphenylamine (abbreviation: DPNF), 2-[N-(4-diphenylaminophenyl)-N-phenylamino]spiro-9,9′-bifluorene (abbreviation: DPASF), 2,7-bis[N-(4-diphenylaminophenyl)-N-phenylamino]spiro-9,9′-bifluorene (abbreviation: DPA2SF), 4,4′,4″-tris[N-(1-naphthyl)-N-phenylamino]triphenylamine (abbreviation: 1′-TNATA), 4,4′,4″-tris(N,N-diphenylamino)triphenylamine (abbreviation: TDATA), 4,4′,4″-tris[N-(3-methylphenyl)-N-phenylamino]triphenylamine (abbreviation: m-MTDATA), N,N′-di(p-tolyl)-N,N′-diphenyl-p-phenylenediamine (abbreviation: DTDPPA), 4,4′-bis[N-(4-diphenylaminophenyl)-N-phenylamino]biphenyl (abbreviation: DPAB), DNTPD, 1,3,5-tris[N-(4-diphenylaminophenyl)-N-phenylamino]benzene (abbreviation: DPA3B), N-(4-biphenyl)-6,N-diphenylbenzo[b]naphtho[1,2-d]furan-8-amine (abbreviation: BnfABP), N,N-bis(4-biphenyl)-6-phenylbenzo[b]naphtho[1,2-d]furan-8-amine (abbreviation: BBABnf), 4,4′-bis(6-phenylbenzo[b]naphtho[1,2-d]furan-8-yl)-4″-phenyltriphenylamine (abbreviation: BnfBB1BP), N,N-bis(4-biphenyl)benzo[b]naphtho[1,2-d]furan-6-amine (abbreviation: BBABnf(6)), N,N-bis(4-biphenyl)benzo[b]naphtho[1,2-d]furan-8-amine (abbreviation: BBABnf(8)), N,N-bis(4-biphenyl)benzo[b]naphtho[2,3-d]furan-4-amine (abbreviation: BBABnf(II)(4)), N,N-bis[4-(dibenzofuran-4-yl)phenyl]-4-amino-p-terphenyl (abbreviation: DBfBB1TP), N-[4-(dibenzothiophen-4-yl)phenyl]-N-phenyl-4-biphenylamine (abbreviation: ThBA1BP), 4-(2-naphthyl)-4′,4″-diphenyltriphenylamine (abbreviation: BBAβNB), 4-[4-(2-naphthyl)phenyl]-4′,4″-diphenyltriphenylamine (abbreviation: BBAβNBi), 4,4′-diphenyl-4″-(6;1′-binaphthyl-2-yl)triphenylamine (abbreviation: BBAαNβNB), 4,4′-diphenyl-4″-(7;1′-binaphthyl-2-yl)triphenylamine (abbreviation: BBAαNβNB-03), 4,4′-diphenyl-4″-(7-phenyl)naphthyl-2-yltriphenylamine (abbreviation: BBAPβNB-03), 4,4′-diphenyl-4″-(6;2′-binaphthyl-2-yl)triphenylamine (abbreviation: BBA(βN2)B), 4,4′-diphenyl-4″-(7;2′-binaphthyl-2-yl)triphenylamine (abbreviation: BBA(βN2)B-03), 4,4′-diphenyl-4″-(4;2′-binaphthyl-1-yl)triphenylamine (abbreviation: BBAβNαNB), 4,4′-diphenyl-4″-(5;2′-binaphthyl-1-yl)triphenylamine (abbreviation: BBAβNαNB-02), 4-(4-biphenylyl)-4′-(2-naphthyl)-4″-phenyltriphenylamine (abbreviation: TPBiAβNB), 4-(3-biphenylyl)-4′-[4-(2-naphthyl)phenyl]-4″-phenyltriphenylamine (abbreviation: mTPBiAβNBi), 4-(4-biphenylyl)-4′-[4-(2-naphthyl)phenyl]-4″-phenyltriphenylamine (abbreviation: TPBiAβNBi), 4-phenyl-4′-(1-naphthyl)-triphenylamine (abbreviation: αNBA1BP), 4,4′-bis(1-naphthyl)triphenylamine (abbreviation: αNBB1BP), 4,4′-diphenyl-4″-[4′-(carbazol-9-yl)biphenyl-4-yl]triphenylamine (abbreviation: YGTBi1BP), 4′-[4-(3-phenyl-9H-carbazol-9-yl)phenyl]tris(1,1′-biphenyl-4-yl)amine (abbreviation: YGTBi1BP-02), 4-[4′-(carbazol-9-yl)biphenyl-4-yl]-4′-(2-naphthyl)-4″-phenyltriphenylamine (abbreviation: YGTBiβNB), N-[4-(9-phenyl-9H-carbazol-3-yl)phenyl]-N-[4-(1-naphthyl)phenyl]-9,9′-spirobi[9H-fluoren]-2-amine (abbreviation: PCBNBSF), N,N-bis([1,1′-biphenyl]-4-yl)-9,9′-spirobi[9H-fluoren]-2-amine (abbreviation: BBASF), N,N-bis([1,1′-biphenyl]-4-yl)-9,9′-spirobi[9H-fluoren]-4-amine (abbreviation: BBASF(4)), N-(1,1′-biphenyl-2-yl)-N-(9,9-dimethyl-9H-fluoren-2-yl)-9,9′-spirobi[9H-fluoren]-4-amine (abbreviation: oFBiSF), N-(4-biphenyl)-N-(9,9-dimethyl-9H-fluoren-2-yl)dibenzofuran-4-amine (abbreviation: FrBiF), N-[4-(1-naphthyl)phenyl]-N-[3-(6-phenyldibenzofuran-4-yl)phenyl]-1-naphthylamine (abbreviation: mPDBfBNBN), 4-phenyl-4′-[4-(9-phenylfluoren-9-yl)phenyl]triphenylamine (abbreviation: BPAFLBi), N,N-bis(9,9-dimethyl-9H-fluoren-2-yl)-9,9′-spirobi-9H-fluoren-4-amine, N,N-bis(9,9-dimethyl-9H-fluoren-2-yl)-9,9′-spirobi-9H-fluoren-3-amine, N,N-bis(9,9-dimethyl-9H-fluoren-2-yl)-9,9′-spirobi-9H-fluoren-2-amine, and N,N-bis(9,9-dimethyl-9H-fluoren-2-yl)-9,9′-spirobi-9H-fluoren-1-amine.

Other examples of the hole-transport material include high-molecular compounds (e.g., oligomers, dendrimers, and polymers) such as poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl)methacrylamide](abbreviation: PTPDMA), and poly[N,N′-bis(4-butylphenyl)-N,N′-bis(phenyl)benzidine](abbreviation: Poly-TPD). Alternatively, it is possible to use a high-molecular compound to which acid is added, such as poly(3,4-ethylenedioxythiophene)/poly(styrenesulfonic acid) (abbreviation: PEDOT/PSS) or polyaniline/poly(styrenesulfonic acid) (abbreviation: PAni/PSS), for example.

Note that the hole-transport material is not limited to the above examples, and any of a variety of known materials may be used alone or in combination as the hole-transport material.

The hole-injection layers (111, 111a, and 111b) can be formed by any of known film formation methods, and for example, a vacuum evaporation method can be employed.

### <Hole-Transport Layer>

The hole-transport layers (112, 112a, and 112b) transport the holes, which are injected from the first electrodes 101 by the hole-injection layers (111, 111a, and 111b), to the light-emitting layers (113, 113a, and 113b). Note that the hole-transport layers (112, 112a, and 112b) each contain a hole-transport material. Thus, the hole-transport layers (112, 112a, and 112b) can be formed using hole-transport materials that can be used for the hole-injection layers (111, 111a, and 111b).

Note that in the light-emitting device of one embodiment of the present invention, the organic compound used for the hole-transport layers (112, 112a, and 112b) can also be used for the light-emitting layers (113, 113a, 113b, and 113c). The use of the same organic compound for the hole-transport layers (112, 112a, and 112b) and the light-emitting layers (113, 113a, 113b, and 113c) is preferable, in which case holes can be efficiently transported from the hole-transport layers (112, 112a, and 112b) to the light-emitting layers (113, 113a, 113b, and 113c).

The first organic compound described in Embodiment 1 can be used for the hole-transport layer.

### <Light-Emitting Layer>

The light-emitting layers (113, 113a, 113b, and 113c) contain a light-emitting substance. Note that as a light-emitting substance that can be used in the light-emitting layers (113, 113a, 113b, and 113c), a substance whose emission color is blue, violet, bluish violet, green, yellowish green, yellow, orange, red, or the like can be used as appropriate. When a plurality of light-emitting layers are provided, the use of different light-emitting substances for the light-emitting layers enables a structure that exhibits different emission colors (e.g., white light emission obtained by a combination of complementary emission colors). Furthermore, one light-emitting layer may have a stacked-layer structure containing different light-emitting substances.

The light-emitting layers (113, 113a, 113b, and 113c) may each contain one or more kinds of organic compounds (e.g., a host material) in addition to a light-emitting substance (a guest material).

In the case where a plurality of host materials are used in the light-emitting layers (113, 113a, 113b, and 113c), a second host material that is additionally used is preferably a substance having a larger energy gap than those of a known guest material and a first host material. Preferably, the lowest singlet excitation level (Si level) of the second host material is higher than that of the first host material, and the lowest triplet excitation level (T1 level) of the second host material is higher than that of the guest material. Preferably, the lowest triplet excitation level (T1 level) of the second host material is higher than that of the first host material. With such a structure, an exciplex can be formed by the two kinds of host materials. To form an exciplex efficiently, it is particularly preferable to combine a compound that easily accepts holes (hole-transport material) and a compound that easily accepts electrons (electron-transport material). With the above structure, high efficiency, low voltage, and along lifetime can be achieved at the same time.

As an organic compound used as the host material (including the first host material and the second host material), organic compounds such as the hole-transport materials usable for the hole-transport layers (112, 112a, and 112b) described above and electron-transport materials usable for electron-transport layers (114, 114a, and 114b) described later can be used as long as they satisfy requirements for the host material used in the light-emitting layer. Another example is an exciplex formed by two or more kinds of organic compounds (the first host material and the second host material). An exciplex whose excited state is formed by two or more kinds of organic compounds has an extremely small difference between the Si level and the T1 level and functions as a TADF material capable of converting triplet excitation energy into singlet excitation energy. In an example of a preferred combination of two or more kinds of organic compounds forming an exciplex, one compound of the two or more kinds of organic compounds has a π-electron deficient heteroaromatic ring and the other compound has a π-electron rich heteroaromatic ring. A phosphorescent substance such as an iridium-, rhodium-, or platinum-based organometallic complex or a metal complex may be used as one compound in the combination of compounds for forming an exciplex.

There is no particular limitation on the light-emitting substances that can be used for the light-emitting layers (113, 113a, 113b, and 113c), and a light-emitting substance that converts singlet excitation energy into light in the visible light range or a light-emitting substance that converts triplet excitation energy into light in the visible light range can be used.

<<Light-Emitting Substance that Converts Singlet Excitation Energy into Light>>

The following substances that emit fluorescent light (fluorescent substances) can be given as examples of the light-emitting substance that converts singlet excitation energy into light and can be used in the light-emitting layers (113, 113a, 113b, and 113c): a pyrene derivative, an anthracene derivative, a triphenylene derivative, a fluorene derivative, a carbazole derivative, a dibenzothiophene derivative, a dibenzofuran derivative, a dibenzoquinoxaline derivative, a quinoxaline derivative, a pyridine derivative, a pyrimidine derivative, a phenanthrene derivative, and a naphthalene derivative. A pyrene derivative is particularly preferable because it has a high emission quantum yield. Specific examples of the pyrene derivative include N,N′-bis(3-methylphenyl)-N,N′-bis[3-(9-phenyl-9H-fluoren-9-yl)phenyl]pyrene-1,6-diamine (abbreviation: 1,6mMemFLPAPm), N,N′-diphenyl-N,N′-bis[4-(9-phenyl-9H-fluoren-9-yl)phenyl]pyrene-1,6-diamine (abbreviation: 1,6FLPAPm), N,N′-bis(dibenzofuran-2-yl)-N,N′-diphenylpyrene-1,6-diamine (abbreviation: 1,6FrAPm), N,N′-bis(dibenzothiophen-2-yl)-N,N′-diphenylpyrene-1,6-diamine (abbreviation: 1,6ThAPm), N,N′-(pyrene-1,6-diyl)bis[(N-phenylbenzo[b]naphtho[1,2-d]furan)-6-amine] (abbreviation: 1,6BnfAPm), N,N′-(pyrene-1,6-diyl)bis[(N-phenylbenzo[b]naphtho[1,2-d]furan)-8-amine](abbreviation: 1,6BnfAPm-02), and N,N′-(pyrene-1,6-diyl)bis[(6,N-diphenylbenzo[b]naphtho[1,2-d]furan)-8-amine] (abbreviation: 1,6BnfAPm-03).

In addition, it is possible to use, for example, 5,6-bis[4-(10-phenyl-9-anthryl)phenyl]-2,2′-bipyridine (abbreviation: PAP2BPy), 5,6-bis[4′-(10-phenyl-9-anthryl)biphenyl-4-yl]-2,2′-bipyridine (abbreviation: PAPP2BPy), N,N′-bis[4-(9H-carbazol-9-yl)phenyl]-N,N′-diphenylstilbene-4,4′-diamine (abbreviation: YGA2S), 4-(9H-carbazol-9-yl)-4′-(10-phenyl-9-anthryl)triphenylamine (abbreviation: YGAPA), 4-(9H-carbazol-9-yl)-4′-(9,10-diphenyl-2-anthryl)triphenylamine (abbreviation: 2YGAPPA), N,9-diphenyl-N-[4-(10-phenyl-9-anthryl)phenyl]-9H-carbazol-3-amine (abbreviation: PCAPA), 4-(10-phenyl-9-anthryl)-4′-(9-phenyl-9H-carbazol-3-yl)triphenylamine (abbreviation: PCBAPA), 4-[4-(10-phenyl-9-anthryl)phenyl]-4′-(9-phenyl-9H-carbazol-3-yl)triphenylamine (abbreviation: PCBAPBA), perylene, 2,5,8,11-tetra-tert-butylperylene (abbreviation: TBP), N,N″-(2-tert-butylanthracene-9,10-diyldi-4,1-phenylene)bis[N,N′,N′-triphenyl-1,4-phenylenediamine] (abbreviation: DPABPA), N,9-diphenyl-N-[4-(9,10-diphenyl-2-anthryl)phenyl]-9H-carbazol-3-amine (abbreviation: 2PCAPPA), and N-[4-(9,10-diphenyl-2-anthryl)phenyl]-N,N′,N′-triphenyl-1,4-phenylenediamine (abbreviation: 2DPAPPA).

It is also possible to use, for example, N-[9,10-bis(1,1′-biphenyl-2-yl)-2-anthryl]-N,9-diphenyl-9H-carbazol-3-amine (abbreviation: 2PCABPhA), N-(9,10-diphenyl-2-anthryl)-N,N′,N′-triphenyl-1,4-phenylenediamine (abbreviation: 2DPAPA), N-[9,10-bis(1,1′-biphenyl-2-yl)-2-anthryl]-N,N′,N′-triphenyl-1,4-phenylenediamine (abbreviation: 2DPABPhA), 9,10-bis(1,1′-biphenyl-2-yl)-N-[4-(9H-carbazol-9-yl)phenyl]-N-phenylanthracen-2-amine (abbreviation: 2YGABPhA), N,N,9-triphenylanthracen-9-amine (abbreviation: DPhAPhA), counarin 545T, N,N′-diphenylquinacridone (abbreviation: DPQd), rubrene, 5,12-bis(1,1′-biphenyl-4-yl)-6,11-diphenyltetracene (abbreviation: BPT), 2-(2-{2-[4-(dimethylamino)phenyl]ethenyl}-6-methyl-4H-pyran-4-ylidene)propanedinitrile (abbreviation: DCM1), 2-{2-methyl-6-[2-(2,3,6,7-tetrahydro-1H,5H-benzo[ij]quinolizin-9-yl)ethenyl]-4H-pyran-4-ylidene}propanedinitrile (abbreviation: DCM2), N,N,N′,M-tetrakis(4-methylphenyl)tetracene-5,11-diamine (abbreviation: p-mPhTD), 7,14-diphenyl-N,N,N′,N′-tetrakis(4-methylphenyl)acenaphtho[1,2-a]fluoranthene-3,10-diamine (abbreviation: p-mPhAFD), 2-{2-isopropyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTI), 2-{2-tert-butyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTB), 2-(2,6-bis{2-[4-(dimethylamino)phenyl]ethenyl}-4H-pyran-4-ylidene)propanedinitrile (abbreviation: BisDCM), 2-{2,6-bis[2-(8-methoxy-1,1,7,7-tetramethyl-2,3,6,7-tetrahydro-1H,5H-benzo[ij]quinolizin-9-yl)ethenyl]-4H-pyran-4-ylidene}propanedinitrile (abbreviation: BisDCJTM), 1,6BnfAPm-03, 3,10-bis[N-(9-phenyl-9H-carbazol-2-yl)-N-phenylamino]naphtho[2,3-b;6,7-b′]bisbenzofuran (abbreviation: 3,10PCA2Nbf(IV)-02), and 3,10-bis[N-(dibenzofuran-3-yl)-N-phenylamino]naphtho[2,3-b;6,7-b′]bisbenzofuran (abbreviation: 3,10FrA2Nbf(IV)-02). In particular, pyrenediamine compounds such as 1,6FLPAPm, 1,6mMemFLPAPm, and 1,6BnfAPm-03 can be used, for example.

<<Light-Emitting Substance that Converts Triplet Excitation Energy into Light>>

Examples of the light-emitting substance that converts triplet excitation energy into light and can be used in the light-emitting layer 113 include substances that emit phosphorescent light (phosphorescent substances) and thermally activated delayed fluorescent (TADF) materials that exhibit thermally activated delayed fluorescence.

A phosphorescent substance is a compound that emits phosphorescent light but does not emit fluorescent light at a temperature higher than or equal to a low temperature (e.g., 77 K) and lower than or equal to room temperature (i.e., higher than or equal to 77 K and lower than or equal to 313 K). The phosphorescent substance preferably contains a metal element with large spin-orbit interaction, and can be an organometallic complex, a metal complex (platinum complex), or a rare earth metal complex, for example. Specifically, the phosphorescent substance preferably contains a transition metal element. It is preferable that the phosphorescent substance contain a platinum group element (ruthenium (Ru), rhodium (Rh), palladium (Pd), osmium (Os), iridium (Ir), or platinum (Pt)), especially iridium, in which case the probability of direct transition between the singlet ground state and the triplet excited state can be increased.

<<Phosphorescent Substance (from 450 nm to 570 nm: Blue or Green)>>

As examples of a phosphorescent substance which emits blue or green light and whose emission spectrum has a peak wavelength in the wavelength ranging from 450 nm to 570 nm, inclusive, the following substances can be given.

Examples include organometallic complexes having a 4H-triazole ring, such as tris{2-[5-(2-methylphenyl)-4-(2,6-dimethylphenyl)-4H-1,2,4-triazol-3-yl-κN²]phenyl-κC}iridium(III) (abbreviation: [Ir(mpptz-dmp)₃]), tris(5-methyl-3,4-diphenyl-4H-1,2,4-triazolato)iridium(III) (abbreviation: [Ir(Mptz)₃]), tris[4-(3-biphenyl)-5-isopropyl-3-phenyl-4H-1,2,4-triazolato]iridium(III) (abbreviation: [Ir(iPrptz-3b)₃]), and tris[3-(5-biphenyl)-5-isopropyl-4-phenyl-4H-1,2,4-triazolato]iridium(III) (abbreviation: [Ir(iPr5btz)₃]); organometallic complexes having a 1H-triazole ring, such as tris[3-methyl-1-(2-methylphenyl)-5-phenyl-1H-1,2,4-triazolato]iridium(III) (abbreviation: [Ir(Mptz1-mp)₃]) and tris(1-methyl-5-phenyl-3-propyl-1H-1,2,4-triazolato)iridium(III) (abbreviation: [Ir(Prptz1-Me)₃]); organometallic complexes having an imidazole ring, such as fac-tris[1-(2,6-diisopropylphenyl)-2-phenyl-1H-imidazole]iridium(III) (abbreviation: [Ir(iPrpmi)₃]) and tris[3-(2,6-dimethylphenyl)-7-methylimidazo[1,2-f]phenanthridinato]iridium(III) (abbreviation: [Ir(dmpimpt-Me)₃]); and organometallic complexes in which a phenylpyridine derivative having an electron-withdrawing group is a ligand, such as bis[2-(4′,6′-difluorophenyl)pyridinato-N,C^{2′}]iridium(III) tetrakis(1-pyrazolyl)borate (abbreviation: FIr6), bis[2-(4′,6′-difluorophenyl)pyridinato-N,C^{2′}]iridium(III) picolinate (abbreviation: FIrpic), bis{2-[3′,5′-bis(trifluoromethyl)phenyl]pyridinato-N,C^{2′}}iridium(III) picolinate (abbreviation: [Ir(CF₃ppy)₂(pic)]), and bis[2-(4′,6′-difluorophenyl)pyridinato-N,C^{2′}]iridium(III) acetylacetonate (abbreviation: FIr(acac)).

<<Phosphorescent Substance (from 495 nm to 590 nm: Green or Yellow)>>

As examples of a phosphorescent substance which emits green or yellow light and whose emission spectrum has a peak wavelength in the wavelength ranging from 495 nm to 590 nm, inclusive, the following substances can be given.

Examples of the phosphorescent substance include organometallic iridium complexes having a pyrimidine ring, such as tris(4-methyl-6-phenylpyrimidinato)iridium(III) (abbreviation: [Ir(mppm)₃]), tris(4-t-butyl-6-phenylpyrimidinato)iridium(III) (abbreviation: [Ir(tBuppm)₃]), (acetylacetonato)bis(6-methyl-4-phenylpyrimidinato)iridium(III) (abbreviation: [Ir(mppm)₂(acac)]), (acetylacetonato)bis(6-tert-butyl-4-phenylpyrimidinato)iridium(III) (abbreviation: [Ir(tBuppm)₂(acac)]), (acetylacetonato)bis[6-(2-norbornyl)-4-phenylpyrimidinato]iridium(III) (abbreviation: [Ir(nbppm)₂(acac)]), (acetylacetonato)bis[5-methyl-6-(2-methylphenyl)-4-phenylpyrimidinato]iridium(III) (abbreviation: [Ir(mpmppm)₂(acac)]), (acetylacetonato)bis{4,6-dimethyl-2-[6-(2,6-dimethylphenyl)-4-pyrimidinyl-κN³]phenyl-κC}iridium(III) (abbreviation: [Ir(dmppm-dmp)₂(acac)]), and (acetylacetonato)bis(4,6-diphenylpyrimidinato)iridium(III) (abbreviation: [Ir(dppm)₂(acac)]); organometallic iridium complexes having a pyrazine ring, such as (acetylacetonato)bis(3,5-dimethyl-2-phenylpyrazinato)iridium(III) (abbreviation: [Ir(mppr-Me)₂(acac)]) and (acetylacetonato)bis(5-isopropyl-3-methyl-2-phenylpyrazinato)iridium(III) (abbreviation: [Ir(mppr-iPr)₂(acac)]); organometallic iridium complexes having a pyridine ring, such as tris(2-phenylpyridinato-N,C^{2′})iridium(III) (abbreviation: [Ir(ppy)₃]), bis(2-phenylpyridinato-N,C^{2′})iridium(III) acetylacetonate (abbreviation: [Ir(ppy)₂(acac)]), bis(benzo[h]quinolinato)iridium(III) acetylacetonate (abbreviation: [Ir(bzq)₂(acac)]), tris(benzo[h]quinolinato)iridium(III) (abbreviation: [Ir(bzq)₃]), tris(2-phenylquinolinato-N,C^{2′})iridium(III) (abbreviation: [Ir(pq)₃]), bis(2-phenylquinolinato-N,C^{2′})iridium(III) acetylacetonate (abbreviation: [Ir(pq)₂(acac)]), bis[2-(2-pyridinyl-κN)phenyl-κC][2-(4-phenyl-2-pyridinyl-κN)phenyl-κC]iridium(III) (abbreviation: [Ir(ppy)₂(4dppy)]), bis[2-(2-pyridinyl-κN)phenyl-κC][2-(4-methyl-5-phenyl-2-pyridinyl-κN)phenyl-κC], [2-d₃-methyl-8-(2-pyridinyl-κN)benzofuro[2,3-b]pyridine-κC]bis[2-(5-d₃-methyl-2-pyridinyl-κN²)phenyl-κC]iridium(III) (abbreviation: Ir(5mppy-d3)₂(mbfpypy-d3)), [2-(methyl-d₃)-8-[4-(1-methylethyl-1-d)-2-pyridinyl-κN]benzofuro[2,3-b]pyridin-7-yl-κC]bis[5-(methyl-d₃)-2-[5-(methyl-d₃)-2-pyridinyl-κN]phenyl-κC]iridium(III) (abbreviation: Ir(5mtpy-d6)₂(mbfpypy-iPr-d4)), [2-d₃-methyl-(2-pyridinyl-κN)benzofuro[2,3-b]pyridine-κC]bis[2-(2-pyridinyl-κN)phenyl-κC]iridium(III) (abbreviation: Ir(ppy)₂(mbfpypy-d3)), and [2-(4-methyl-5-phenyl-2-pyridinyl-κN)phenyl-κC]bis[2-(2-pyridinyl-κN)phenyl-κC]iridium(III) (abbreviation: Ir(ppy)₂(mdppy)); organometallic complexes such as bis(2,4-diphenyl-1,3-oxazolato-N,C^{2′})iridium(III) acetylacetonate (abbreviation: [Ir(dpo)₂(acac)]), bis{2-[4′-(perfluorophenyl)phenyl]pyridinato-N,C^{2′}}iridium(III) acetylacetonate (abbreviation: [Ir(p-PF-ph)₂(acac)]), and bis(2-phenylbenzothiazolato-N,C^{2′})iridium(III) acetylacetonate (abbreviation: [Ir(bt)₂(acac)]); and a rare earth metal complex such as tris(acetylacetonato) (monophenanthroline)terbium(III) (abbreviation: [Tb(acac)₃(Phen)]).

<<Phosphorescent Substance (from 570 nm to 750 nm: Yellow or Red)>>

As examples of a phosphorescent substance which emits yellow or red light and whose emission spectrum has a peak wavelength in the wavelength ranging from 570 nm to 750 nm, inclusive, the following substances can be given.

Examples of a phosphorescent substance include organometallic complexes having a pyrimidine ring, such as (diisobutyrylmethanato)bis[4,6-bis(3-methylphenyl)pyrimidinato]iridium(III) (abbreviation: [Ir(5mdppm)₂(dibm)]), bis[4,6-bis(3-methylphenyl)pyrimidinato](dipivaloylmethanato)iridium(III) (abbreviation: [Ir(5mdppm)₂(dpm)]), and (dipivaloylmethanato)bis[4,6-di(naphthalen-1-yl)pyrimidinato]iridium(III) (abbreviation: [Ir(d1npm)₂(dpm)]); organometallic complexes having a pyrazine ring, such as (acetylacetonato)bis(2,3,5-triphenylpyrazinato)iridium(III) (abbreviation: [Ir(tppr)₂(acac)]), bis(2,3,5-triphenylpyrazinato)(dipivaloylmethanato)iridium(III) (abbreviation: [Ir(tppr)₂(dpm)]), bis{4,6-dimethyl-2-[3-(3,5-dimethylphenyl)-5-phenyl-2-pyrazinyl-κN]phenyl-κC}(2,6-dimethyl-3,5-heptanedionato-κ²O,O′)iridium(III) (abbreviation: [Ir(dmdppr-P)₂(dibm)]), bis{4,6-dimethyl-2-[5-(4-cyano-2,6-dimethylphenyl)-3-(3,5-dimethylphenyl)-2-pyrazinyl-κN]phenyl-κC}(2,2,6,6-tetramethyl-3,5-heptanedionato-κ²O,O′)iridium(III) (abbreviation: [Ir(dmdppr-dmCP)₂(dpm)]), bis[2-(5-(2,6-dimethylphenyl)-3-(3,5-dimethylphenyl)-2-pyrazinyl-κN)-4,6-dimethylphenyl-κC](2,2′,6,6′-tetramethyl-3,5-heptanedionato-κ²O,O′)iridium(III) (abbreviation: [Ir(dmdppr-dmp)₂(dpm)]), (acetylacetonato)bis[2-methyl-3-phenylquinoxalinato-N,C^{2′}]iridium(III) (abbreviation: [Ir(mpq)₂(acac)]), (acetylacetonato)bis(2,3-diphenylquinoxalinato-N,C^{2′})iridium(III) (abbreviation: [Ir(dpq)₂(acac)]), and (acetylacetonato)bis[2,3-bis(4-fluorophenyl)quinoxalinato]iridium(III) (abbreviation: [Ir(Fdpq)₂(acac)]); organometallic complexes having a pyridine ring, such as tris(1-phenylisoquinolinato-N,C^{2′})iridium(III)(abbreviation: [Ir(piq)₃]), bis(1-phenylisoquinolinato-N,C^{2′})iridium(III) acetylacetonate (abbreviation: [Ir(piq)₂(acac)]), and bis[4,6-dimethyl-2-(2-quinolinyl-κN)phenyl-κC](2,4-pentanedionato-κ²O,O′)iridium(III) (abbreviation: [Ir(dmpqn)₂(acac)]); a platinum complex such as 2,3,7,8,12,13,17,18-octaethyl-21H,23H-porphyrin platinum(II) (abbreviation: [PtOEP]); and rare earth metal complexes such as tris(1,3-diphenyl-1,3-propanedionato)(monophenanthroline)europium(III) (abbreviation: [Eu(DBM)₃(Phen)]) and tris[1-(2-thenoyl)-3,3,3-trifluoroacetonato](monophenanthroline)europium(III) (abbreviation: [Eu(TTA)₃(Phen)]).

### <<TADF Material>>

Any of materials described below can be used as the TADF material. The TADF material is a material that has a small difference between its S1 and T1 levels (preferably less than or equal to 0.2 eV), enables up-conversion of a triplet excited state into a singlet excited state (i.e., reverse intersystem crossing) using a little thermal energy, and efficiently exhibits light (fluorescent light) from the singlet excited state. The thermally activated delayed fluorescence is efficiently obtained under the condition where the difference in energy between the triplet excitation level and the singlet excitation level is greater than or equal to 0 eV and less than or equal to 0.2 eV, preferably greater than or equal to 0 eV and less than or equal to 0.1 eV. Note that delayed fluorescent light by the TADF material refers to light emission having a spectrum similar to that of normal fluorescent light and an extremely long lifetime. The lifetime is longer than or equal to 1×10⁻⁶ seconds, preferably longer than or equal to 1×10⁻³ seconds.

Examples of the TADF material include fullerene, a derivative thereof, an acridine derivative such as proflavine, and eosin. Other examples thereof include a metal-containing porphyrin, containing a metal such as a porphyrin containing magnesium (Mg), zinc (Zn), cadmium (Cd), tin (Sn), platinum (Pt), indium (In), or palladium (Pd). Examples of the metal-containing porphyrin include a protoporphyrin-tin fluoride complex (abbreviation: SnF₂(Proto IX)), a mesoporphyrin-tin fluoride complex (abbreviation: SnF₂(Meso IX)), a hematoporphyrin-tin fluoride complex (abbreviation: SnF₂(Hemato IX)), a coproporphyrin tetramethyl ester-tin fluoride complex (abbreviation: SnF₂(Copro III-4Me)), an octaethylporphyrin-tin fluoride complex (abbreviation: SnF₂(OEP)), an etioporphyrin-tin fluoride complex (abbreviation: SnF₂(Etio I)), and an octaethylporphyrin-platinum chloride complex (abbreviation: PtCl₂OEP).

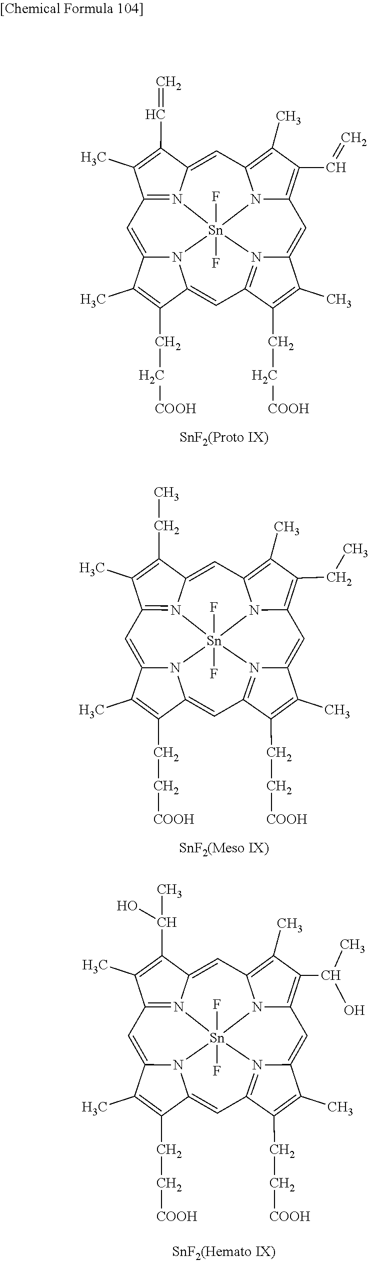

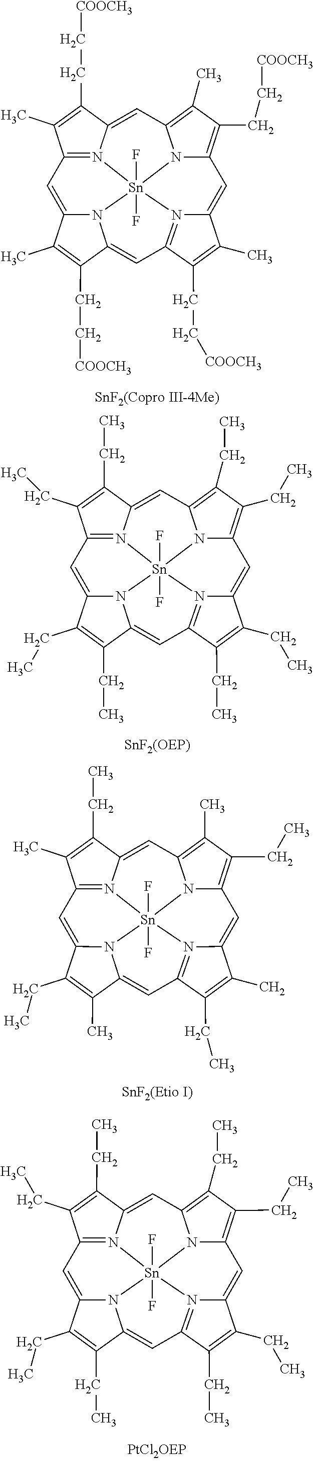

Additionally, a heteroaronmatic compound having a π-electron rich heteroaromatic compound and a π-electron deficient heteroaromatic compound may be used, such as 2-(biphenyl-4-yl)-4,6-bis(12-phenylindolo[2,3-a]carbazol-11-yl)-1,3,5-triazine (abbreviation: PIC-TRZ), 2-{4-[3-(N-phenyl-9H-carbazol-3-yl)-9H-carbazol-9-yl]phenyl}-4,6-diphenyl-1,3,5-triazine (abbreviation: PCCzPTzn), 2-[4-(10H-phenoxazin-10-yl)phenyl]-4,6-diphenyl-1,3,5-triazine (abbreviation: PXZ-TRZ), 3-[4-(5-phenyl-5,10-dihydrophenazin-10-yl)phenyl]-4,5-diphenyl-1,2,4-triazole (abbreviation: PPZ-3TPT), 3-(9,9-dimethyl-9H-acridin-10-yl)-9H-xanthen-9-one (abbreviation: ACRXTN), bis[4-(9,9-dimethyl-9,10-dihydroacridine)phenyl]sulfone (abbreviation: DMAC-DPS), 10-phenyl-10H,10′H-spiro[acridin-9,9′-anthracen]-10′-one (abbreviation: ACRSA), 4-(9′-phenyl-3,3′-bi-9H-carbazol-9-yl)benzofuro[3,2-d]pyrimidine (abbreviation: 4PCCzBfpm), 4-[4-(9′-phenyl-3,3′-bi-9H-carbazol-9-yl)phenyl]benzofuro[3,2-d]pyrimidine (abbreviation: 4PCCzPBfpm), or 9-[3-(4,6-diphenyl-1,3,5-triazin-2-yl)phenyl]-9′-phenyl-2,3′-bi-9H-carbazole (abbreviation: mPCCzPTzn-02).

Note that a substance in which a π-electron rich heteroaromatic compound is directly bonded to a π-electron deficient heteroaromatic compound is particularly preferable because both the donor property of the π-electron rich heteroaromatic compound and the acceptor property of the π-electron deficient heteroaromatic compound are improved and the energy difference between the singlet excited state and the triplet excited state becomes small. As the TADF material, a TADF material in which the singlet and triplet excited states are in thermal equilibrium (TADF100) may be used. Since such a TADF material enables a short emission lifetime (excitation lifetime), the efficiency of a light-emitting device in a high-luminance region can be less likely to decrease.

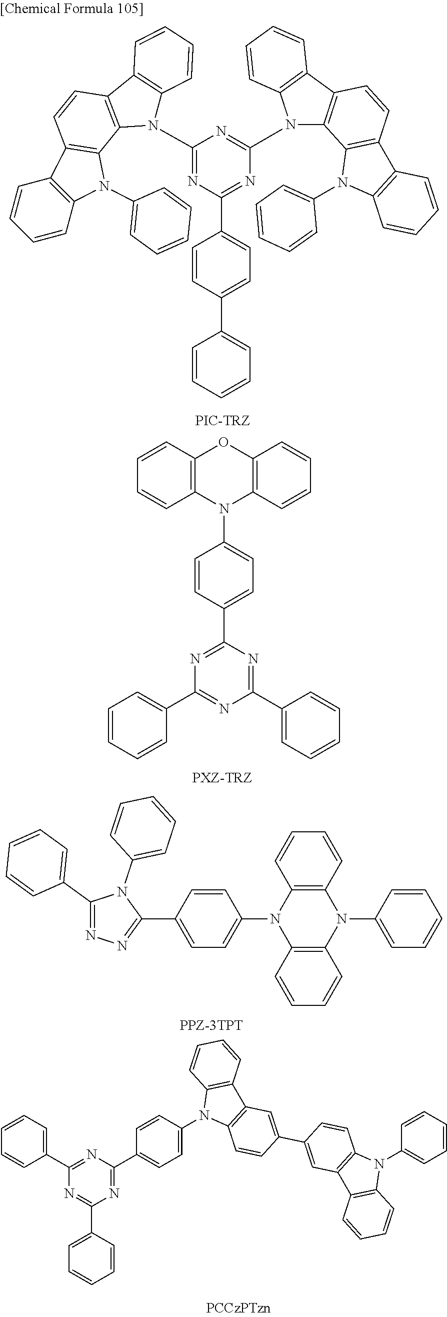

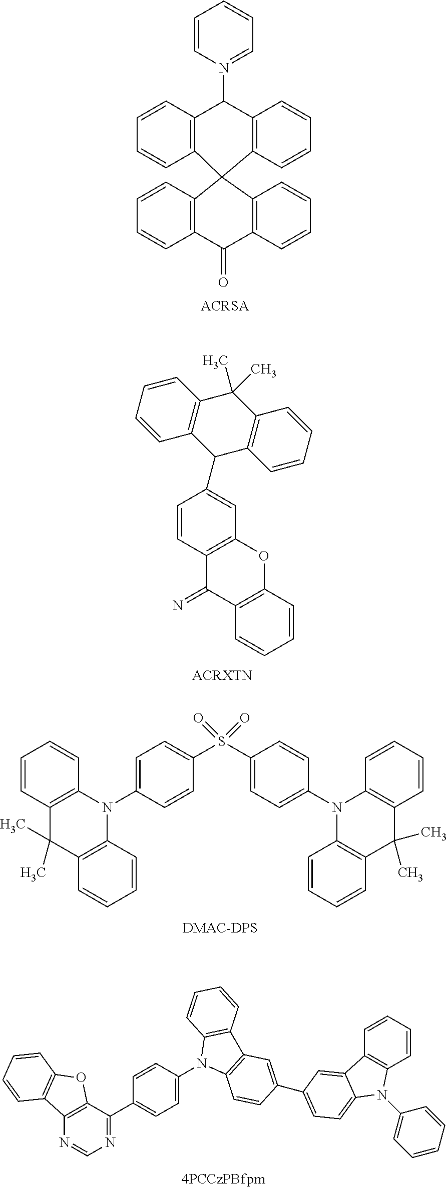

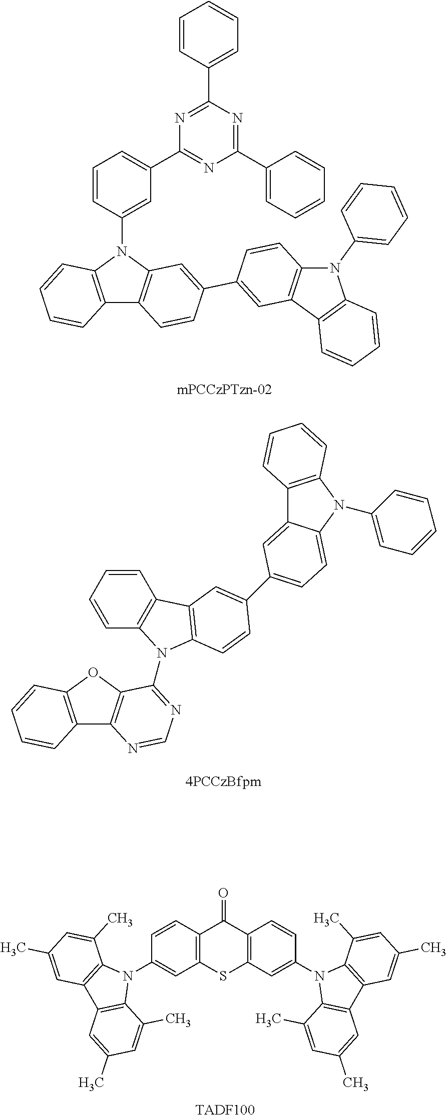

In addition to the above, another example of a material having a function of converting triplet excitation energy into light is a nano-structure of a transition metal compound having a perovskite structure. In particular, a nano-structure of a metal halide perovskite material is preferable. The nano-structure is preferably a nanoparticle or a nanorod.

As the organic compound (e.g., the host material) used in combination with the above-described light-emitting substance (guest material) in the light-emitting layers (113, 113a, 113b, and 113c), one or more kinds selected from substances having a larger energy gap than the light-emitting substance (guest material) can be used.

### <<Host Material for Fluorescent Light>>

In the case where the light-emitting substance used in the light-emitting layers (113, 113a, 113b, and 113c) is a fluorescent substance, an organic compound (a host material) used in combination with the fluorescent substance is preferably an organic compound that has a high energy level in a singlet excited state and has a low energy level in a triplet excited state or an organic compound having a high fluorescence quantum yield. Therefore, the hole-transport material (described above) and the electron-transport material (described below) shown in this embodiment, for example, can be used as long as they are organic compounds that satisfy such a condition.

In terms of a preferred combination with the light-emitting substance (fluorescent substance), examples of the organic compound (host material), some of which are mentioned in the above specific examples, include fused polycyclic aromatic compounds such as an anthracene derivative, a tetracene derivative, a phenanthrene derivative, a pyrene derivative, a chrysene derivative, and a dibenzo[g,p]chrysene derivative.

Specific examples of the organic compound (host material) that is preferably used in combination with the fluorescent substance include 9-phenyl-3-[4-(10-phenyl-9-anthryl)phenyl]-9H-carbazole (abbreviation: PCzPA), 3,6-diphenyl-9-[4-(10-phenyl-9-anthryl)phenyl]-9H-carbazole (abbreviation: DPCzPA), 3-[4-(1-naphthyl)-phenyl]-9-phenyl-9H-carbazole (abbreviation: PCPN), 9,10-diphenylanthracene (abbreviation: DPAnth), N,N-diphenyl-9-[4-(10-phenyl-9-anthryl)phenyl]-9H-carbazol-3-amine (abbreviation: CzA1PA), 4-(10-phenyl-9-anthryl)triphenylamine (abbreviation: DPhPA), YGAPA, PCAPA, N,9-diphenyl-N-{4-[4-(10-phenyl-9-anthryl)phenyl]phenyl}-9H-carbazol-3-amine (abbreviation: PCAPBA), N-(9,10-diphenyl-2-anthryl)-N,9-diphenyl-9H-carbazol-3-amine (abbreviation: 2PCAPA), 6,12-dimethoxy-5,11-diphenylchrysene, N,N,N′,N′,N″,N″,N′″,N′″-octaphenyldibenzo[g,p]chrysene-2,7,10,15-tetraamine (abbreviation: DBC1), 9-[4-(10-phenyl-9-anthracenyl)phenyl]-9H-carbazole (abbreviation: CzPA), 7-[4-(10-phenyl-9-anthryl)phenyl]-7H-dibenzo[c,g]carbazole (abbreviation: cgDBCzPA), 6-[3-(9,10-diphenyl-2-anthryl)phenyl]-benzo[b]naphtho[1,2-d]furan (abbreviation: 2mBnfPPA), 9-phenyl-10-(4-(9-phenyl-9H-fluoren-9-yl)biphenyl-4′-yl)anthracene (abbreviation: FLPPA), 9,10-bis(3,5-diphenylphenyl)anthracene (abbreviation: DPPA), 9,10-di(2-naphthyl)anthracene (abbreviation: DNA), 2-tert-butyl-9,10-di(2-naphthyl)anthracene (abbreviation: t-BuDNA), 9-(1-naphthyl)-10-(2-naphthyl)anthracene (abbreviation: α,β-ADN), 2-(10-phenylanthracen-9-yl)dibenzofuran, 2-(10-phenyl-9-anthracenyl)-benzo[b]naphtho[2,3-d]furan (abbreviation: Bnf(II)PhA), 9-(1-naphthyl)-10-[4-(2-naphthyl)phenyl]anthracene (abbreviation: αN-βNPAnth), 2,9-di(1-naphthyl)-10-phenylanthracene (abbreviation: 2αN-αNPhA), 9-(1-naphthyl)-10-[3-(1-naphthyl)phenyl]anthracene (abbreviation: αN-mαNPAnth), 9-(2-naphthyl)-10-[3-(1-naphthyl)phenyl]anthracene (abbreviation: βN-mαNPAnth), 9-(1-naphthyl)-10-[4-(1-naphthyl)phenyl]anthracene (abbreviation: αN-αNPAnth), 9-(2-naphthyl)-10-[4-(2-naphthyl)phenyl]anthracene (abbreviation: βN-βNPAnth), 2-(1-naphthyl)-9-(2-naphthyl)-10-phenylanthracene (abbreviation: 2αN-βNPhA), 9-(2-naphthyl)-10-[3-(2-naphthyl)phenyl]anthracene (abbreviation: βN-mβNPAnth), 1-[4-(10-[1,1′-biphenyl]-4-yl-9-anthracenyl)phenyl]-2-ethyl-1H-benzimidazole (abbreviation: EtBImPBPhA), 9,9′-bianthryl (abbreviation: BANT), 9,9′-(stilbene-3,3′-diyl)diphenanthrene (abbreviation: DPNS), 9,9′-(stilbene-4,4′-diyl)diphenanthrene (abbreviation: DPNS2), 1,3,5-tri(1-pyrenyl)benzene (abbreviation: TPB3), 5,12-diphenyltetracene, and 5,12-bis(biphenyl-2-yl)tetracene.

### <<Host Material for Phosphorescent Light>>

In the case where the light-emitting substance used in the light-emitting layers (113, 113a, 113b, and 113c) is a phosphorescent substance, an organic compound having triplet excitation energy (an energy difference between a ground state and a triplet excited state) which is higher than that of the light-emitting substance is preferably selected as the organic compound (host material) used in combination with the phosphorescent substance. Note that when a plurality of organic compounds (e.g., a first host material and a second host material (or an assist material)) are used in combination with a light-emitting substance so that an exciplex is formed, the plurality of organic compounds are preferably mixed with the phosphorescent substance.

With such a structure, light emission can be efficiently obtained by exciplex-triplet energy transfer (ExTET), which is energy transfer from an exciplex to a light-emitting substance. Note that a combination of the plurality of organic compounds that easily forms an exciplex is preferred, and it is particularly preferable to combine a compound that easily accepts holes (hole-transport material) and a compound that easily accepts electrons (electron-transport material).

In terms of a preferred combination with the light-emitting substance (phosphorescent substance), examples of the organic compounds (the host material and the assist material), some of which are mentioned in the above specific examples, include an aromatic amine (an organic compound having an aromatic amine skeleton), a carbazole derivative (an organic compound having a carbazole ring), a dibenzothiophene derivative (an organic compound having a dibenzothiophene ring), a dibenzofuran derivative (an organic compound having a dibenzofuran ring), an oxadiazole derivative (an organic compound having an oxadiazole ring), a triazole derivative (an organic compound having a triazole ring), a benzimidazole derivative (an organic compound having a benzimidazole ring), a quinoxaline derivative (an organic compound having a quinoxaline ring), a dibenzoquinoxaline derivative (an organic compound having a dibenzoquinoxaline ring), a pyrimidine derivative (an organic compound having a pyrimidine ring), a triazine derivative (an organic compound having a triazine ring), a pyridine derivative (an organic compound having a pyridine ring), a bipyridine derivative (an organic compound having a bipyridine ring), a phenanthroline derivative (an organic compound having a phenanthroline ring), a furodiazine derivative (an organic compound having a furodiazine ring), and zinc- or aluminum-based metal complexes.

Among the above organic compounds, specific examples of the aromatic amine and the carbazole derivative, which are organic compounds having a high hole-transport property, are the same as the specific examples of the hole-transport materials described above, and those materials are preferable as the host material.

Among the above organic compounds, specific examples of the dibenzothiophene derivative and the dibenzofuran derivative, which are organic compounds having a high hole-transport property, include 4-{3-[3-(9-phenyl-9H-fluoren-9-yl)phenyl]phenyl}dibenzofuran (abbreviation: mmDBFFLBi-II), 4,4′,4″-(benzene-1,3,5-triyl)tri(dibenzofuran) (abbreviation: DBF3P-II), DBT3P-II, 2,8-diphenyl-4-[4-(9-phenyl-9H-fluoren-9-yl)phenyl]dibenzothiophene (abbreviation: DBTFLP-III), 4-[4-(9-phenyl-9H-fluoren-9-yl)phenyl]-6-phenyldibenzothiophene (abbreviation: DBTFLP-IV), and 4-[3-(triphenylen-2-yl)phenyl]dibenzothiophene (abbreviation: mDBTPTp-II). Such derivatives are preferable as the host material.

Other examples of preferred host materials include metal complexes having an oxazole-based or thiazole-based ligand, such as bis[2-(2-benzoxazolyl)phenolato]zinc(II) (abbreviation: ZnPBO) and bis[2-(2-benzothiazolyl)phenolato]zinc(II) (abbreviation: ZnBTZ).

Among the above organic compounds, specific examples of the oxadiazole derivative, the triazole derivative, the benzimidazole derivative, the quinoxaline derivative, the dibenzoquinoxaline derivative, the quinazoline derivative, and the phenanthroline derivative, which are organic compounds having a high electron-transport property, include: an organic compound containing a heteroaromatic ring having a polyazole ring such as 2-(4-biphenylyl)-5-(4-tert-butylphenyl)-1,3,4-oxadiazole (abbreviation: PBD), 1,3-bis[5-(p-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: CO11), 3-(4-biphenylyl)-4-phenyl-5-(4-tert-butylphenyl)-1,2,4-triazole (abbreviation: TAZ), 2,2′,2″-(1,3,5-benzenetriyl)tris(1-phenyl-1H-benzimidazole) (abbreviation: TPBI), 2-[3-(dibenzothiophen-4-yl)phenyl]-1-phenyl-1H-benzimidazole (abbreviation: mDBTBIm-II), or 4,4′-bis(5-methylbenzoxazol-2-yl)stilbene (abbreviation: BzOs); an organic compound containing a heteroaromatic ring having a pyridine ring such as bathophenanthroline (abbreviation: BPhen), bathocuproine (abbreviation: BCP), 2,9-di(naphthalen-2-yl)-4,7-diphenyl-1,10-phenanthroline (abbreviation: NBPhen), or 2,2-(1,3-phenylene)bis[9-phenyl-1,10-phenanthroline](abbreviation: mPPhen2P); 2-[3-(dibenzothiophen-4-yl)phenyl]dibenzo[f,h]quinoxaline (abbreviation: 2mDBTPDBq-II); 2-[3′-(dibenzothiophen-4-yl)biphenyl-3-yl]dibenzo[f,h]quinoxaline (abbreviation: 2mDBTBPDBq-II); 2-[3′-(9H-carbazol-9-yl)biphenyl-3-yl]dibenzo[f,h]quinoxaline (abbreviation: 2mCzBPDBq); 2-[4-(3,6-diphenyl-9H-carbazol-9-yl)phenyl]dibenzo[f,h]quinoxaline (abbreviation: 2CzPDBq-III); 7-[3-(dibenzothiophen-4-yl)phenyl]dibenzo[f,h]quinoxaline (abbreviation: 7mDBTPDBq-II); 6-[3-(dibenzothiophen-4-yl)phenyl]dibenzo[f,h]quinoxaline (abbreviation: 6mDBTPDBq-II); 2-{4-[9,10-di(2-naphthyl)-2-anthryl]phenyl}-1-phenyl-1H-benzimidazole (abbreviation: ZADN); and 2-4′-(9-phenyl-9H-carbazol-3-yl)-3,1′-biphenyl-1-yl]dibenzo[f,h]quinoxaline (abbreviation: 2mpPCBPDBq). Such organic compounds are preferable as the host material.

Among the above organic compounds, specific examples of the pyridine derivative, the diazine derivative (e.g., the pyrimidine derivative, the pyrazine derivative, and the pyridazine derivative), the triazine derivative, the furodiazine derivative, which are organic compounds having a high electron-transport property, include organic compounds containing a heteroaromatic ring having a diazine ring such as 4,6-bis[3-(phenanthren-9-yl)phenyl]pyrimidine (abbreviation: 4,6mPnP2Pm), 4,6-bis[3-(4-dibenzothienyl)phenyl]pyrimidine (abbreviation: 4,6mDBTP2Pm-II), 4,6-bis[3-(9H-carbazol-9-yl)phenyl]pyrimidine (abbreviation: 4,6mCzP2Pm), 2-{4-[3-(N-phenyl-9H-carbazol-3-yl)-9H-carbazol-9-yl]phenyl}-4,6-diphenyl-1,3,5-triazine (abbreviation: PCCzPTzn), 9-[3-(4,6-diphenyl-1,3,5-triazin-2-yl)phenyl]-9′-phenyl-2,3′-bi-9H-carbazole (abbreviation: mPCCzPTzn-02), 3,5-bis[3-(9H-carbazol-9-yl)phenyl]pyridine (abbreviation: 35DCzPPy), 1,3,5-tri[3-(3-pyridyl)phenyl]benzene (abbreviation: TmPyPB), 9,9′-[pyrimidine-4,6-diylbis(biphenyl-3,3′-diyl)]bis(9H-carbazole) (abbreviation: 4,6mCzBP2Pm), 2-[3′-(9,9-dimethyl-9H-fluoren-2-yl)-1,1′-biphenyl-3-yl]-4,6-diphenyl-1,3,5-triazine (abbreviation: mFBPTzn), 8-(1,1′-biphenyl-4-yl)-4-[3-(dibenzothiophen-4-yl)phenyl]-[1]benzofuro[3,2-d]pyrimidine (abbreviation: 8BP-4mDBtPBfpm), 9-[3′-(dibenzothiophen-4-yl)biphenyl-3-yl]naphtho[1′,2′:4,5]furo[2,3-b]pyrazine (abbreviation: 9mDBtBPNfpr), 9-[3′-(dibenzothiophen-4-yl)biphenyl-4-yl]naphtho[1′,2′:4,5]furo[2,3-b]pyrazine (abbreviation: 9pmDBtBPNfpr), 11-[3′-(dibenzothiophen-4-yl)biphenyl-3-yl]phenanthro[9′,10′:4,5]furo[2,3-b]pyrazine (abbreviation: 11mDBtBPPnfpr), 11-[3′-(dibenzothiophen-4-yl)biphenyl-4-yl]phenanthro[9′,10′:4,5]furo[2,3-b]pyrazine, 11-[3′-(9H-carbazol-9-yl)biphenyl-3-yl]phenanthro[9′,10′:4,5]furo[2,3-b]pyrazine, 12-(9′-phenyl-3,3′-bi-9H-carbazol-9-yl)phenanthro[9′,10′:4,5]furo[2,3-b]pyrazine (abbreviation: 12PCCzPnfpr), 9-[(3′-9-phenyl-9H-carbazol-3-yl)biphenyl-4-yl]naphtho[1′,2′:4,5]furo[2,3-b]pyrazine (abbreviation: 9pmPCBPNfpr), 9-(9′-phenyl-3,3′-bi-9H-carbazol-9-yl)naphtho[1′,2′:4,5]furo[2,3-b]pyrazine (abbreviation: 9PCCzNfpr), 10-(9′-phenyl-3,3′-bi-9H-carbazol-9-yl)naphtho[1′,2′:4,5]furo[2,3-b]pyrazine (abbreviation: 10PCCzNfpr), 9-[3′-(6-phenylbenzo[b]naphtho[1,2-d]furan-8-yl)biphenyl-3-yl]naphtho[1′,2′:4,5]furo[2,3-b]pyrazine (abbreviation: 9mBnfBPNfpr), 9-{3-[6-(9,9-dimethylfluoren-2-yl)dibenzothiophen-4-yl]phenyl}naphtho[1′,2′:4,5]furo[2,3-b]pyrazine (abbreviation: 9mFDBtPNfpr), 9-[3′-(6-phenyldibenzothiophen-4-yl)biphenyl-3-yl]naphtho[1′,2′:4,5]furo[2,3-b]pyrazine (abbreviation: 9mDBtBPNfpr-02), 9-[3-(9′-phenyl-3,3′-bi-9H-carbazol-9-yl)phenyl]naphtho[1′,2′:4,5]furo[2,3-b]pyrazine (abbreviation: 9mPCCzPNfpr), 9-{(3′-[2,8-diphenyldibenzothiophen-4-yl)biphenyl-3-yl}naphtho[1′,2′:4,5]furo[2,3-b]pyrazine, 11-((3′-[2,8-diphenyldibenzothiophen-4-yl)biphenyl-3-yl]phenanthro[9′,10′:4,5]furo[2,3-b]pyrazine, 5-[3-(4,6-diphenyl-1,3,5-triazin-2-yl)phenyl]-7,7-dimethyl-5H,7H-indeno[2,1-b]carbazole (abbreviation: mINc(II)PTzn), 2-[3′-(triphenylen-2-yl)-1,1′-biphenyl-3-yl]-4,6-diphenyl-1,3,5-triazine (abbreviation: mTpBPTzn), 2-[(1,1′-biphenyl)-4-yl]-4-phenyl-6-[9,9′-spirobi(9H-fluoren)-2-yl]-1,3,5-triazine (abbreviation: BP-SFTzn), 2,6-bis(4-naphthalen-1-ylphenyl)-4-[4-(3-pyridyl)phenyl]pyrimidine (abbreviation: 2,4NP-6PyPPm), 3-[9-(4,6-diphenyl-1,3,5-triazin-2-yl)-2-dibenzofuranyl]-9-phenyl-9H-carbazole (abbreviation: PCDBfTzn), 2-[1,1′-biphenyl]-3-yl-4-phenyl-6-(8-[1,1′:4′,1″-terphenyl]-4-yl-1-dibenzofuranyl)-1,3,5-triazine (abbreviation: mBP-TPDBfTzn), 6-(1,1′-biphenyl-3-yl)-4-[3,5-bis(9H-carbazol-9-yl)phenyl)-2-phenylpyrimidine (abbreviation: 6mBP-4Cz2PPm), and 4-[3,5-bis(9H-carbazol-9-yl)phenyl]-2-phenyl-6-(1,1′-biphenyl-4-yl)pyrimidine (abbreviation: 6BP-4Cz2PPm), and those materials are preferable as the host material.

Among the above organic compounds, specific examples of metal complexes that are organic compounds having a high electron-transport property include zinc- or aluminum-based metal complexes, such as tris(8-quinolinolato)aluminum(III) (abbreviation: Alq), tris(4-methyl-8-quinolinolato)aluminum(III) (abbreviation: Almq₃), bis(10-hydroxybenzo[h]quinolinato)beryllium(II) (abbreviation: BeBq₂), bis(2-methyl-8-quinolinolato)(4-phenylphenolato)aluminum(III) (abbreviation: BAlq), and bis(8-quinolinolato)zinc(II) (abbreviation: Znq), and metal complexes having a quinoline ring or a benzoquinoline ring. Such metal complexes are preferable as the host material.

Moreover, high-molecular compounds such as poly(2,5-pyridinediyl) (abbreviation: PPy), poly[(9,9-dihexylfluorene-2,7-diyl)-co-(pyridine-3,5-diyl)](abbreviation: PF-Py), and poly[(9,9-dioctylfluorene-2,7-diyl)-co-(2,2′-bipyridine-6,6′-diyl)] (abbreviation: PF-BPy) are preferable as the host material.

Furthermore, the following organic compounds having a diazine ring, which have bipolar properties, a high hole-transport property and a high electron-transport property, can be used as the host material: 9-phenyl-9′-(4-phenyl-2-quinazolinyl)-3,3′-bi-9H-carbazole (abbreviation: PCCzQz), 2-[4′-(9-phenyl-9H-carbazol-3-yl)-3,1′-biphenyl-1-yl]dibenzo[f,h]quinoxaline (abbreviation: 2mpPCBPDBq), 5-[3-(4,6-diphenyl-1,3,5-triazin-2-yl)phenyl]-7,7-dimethyl-5H,7H-indeno[2,1-b]carbazole (abbreviation: mINc(II)PTzn), 11-(4-[1,1′-biphenyl]-4-yl-6-phenyl-1,3,5-triazin-2-yl)-11,12-dihydro-12-phenyl-indolo[2,3-a]carbazole (abbreviation: BP-Icz(II)Tzn), and 7-[4-(9-phenyl-9H-carbazol-2-yl)quinazolin-2-yl]-7H-dibenzo[c,g]carbazole (abbreviation: PC-cgDBCzQz).

### <Electron-Transport Layer>

The electron-transport layers (114, 114a, and 114b) transport electrons, which are injected from the second electrode 102 and the charge-generation layers (106, 106a, and 106b) by electron-injection layers (115, 115a, and 115b) described later, to the light-emitting layers (113, 113a, and 113b). The heat resistance of the light-emitting device of one embodiment of the present invention can be improved by including a stacked structure of electron-transport layers. The electron-transport material used in the electron-transport layers (114, 114a, and 114b) is preferably a substance having an electron mobility of 1×10⁻⁶ cm²/Vs or higher in the case where the square root of the electric field strength [V/cm] is 600. Note that any other substance can also be used as long as the substance has an electron-transport property higher than a hole-transport property. The electron-transport layers (114, 114a, and 114b) can function even with a single-layer structure and may have a stacked-layer structure including two or more layers. When a photolithography process is performed over the electron-transport layer containing the above-described mixed material, which has heat resistance, an adverse effect of the thermal process on the device characteristics can be reduced.

### <<Electron-Transport Material>>

As the electron-transport material that can be used for the electron-transport layers (114, 114a, and 114b), an organic compound having a high electron-transport property can be used, and for example, a heteroaromatic compound can be used. The term heteroaromatic compound refers to a cyclic compound containing at least two different kinds of elements in a ring. Examples of cyclic structures include a three-membered ring, a four-membered ring, a five-membered ring, a six-membered ring, and the like, among which a five-membered ring and a six-membered ring are particularly preferred. The elements contained in the heteroaromatic compound are preferably one or more of nitrogen, oxygen, and sulfur, in addition to carbon. In particular, a heteroaromatic compound containing nitrogen (a nitrogen-containing heteroaromatic compound) is preferred, and any of materials having a high electron-transport property (electron-transport materials), such as a nitrogen-containing heteroaromatic compound and a π-electron deficient heteroaromatic compound including the nitrogen-containing heteroaromatic compound, is preferably used. Note that the electron-transport material is preferably different from the materials used in the light-emitting layer. Not all excitons formed by recombination of carriers in the light-emitting layer can contribute to light emission and some excitons are diffused into a layer in contact with the light-emitting layer or a layer in the vicinity of the light-emitting layer. In order to avoid this phenomenon, the energy level (the lowest singlet excitation level or the lowest triplet excitation level) of a material used for the layer in contact with the light-emitting layer or the layer in the vicinity of the light-emitting layer is preferably higher than that of at least one of the materials used for the light-emitting layer. Thus, in order to obtain a highly efficient device, the electron-transport material is preferably different from the materials used in the light-emitting layer.

The heteroaromatic compound is an organic compound including at least one heteroaromatic ring.

The heteroaromatic ring includes any one of a pyridine ring, a diazine ring, a triazine ring, a polyazole ring, an oxazole ring, a thiazole ring, and the like. A heteroaromatic ring having a diazine ring includes a heteroaromatic ring having a pyrimidine ring, a pyrazine ring, a pyridazine ring, or the like. A heteroaromatic ring having a polyazole ring includes a heteroaromatic ring having an imidazole ring, a triazole ring, or an oxadiazole ring.

The heteroaromatic ring includes a fused heteroaromatic ring having a fused ring structure. Examples of the fused heteroaromatic ring include a quinoline ring, a benzoquinoline ring, a quinoxaline ring, a dibenzoquinoxaline ring, a quinazoline ring, a benzoquinazoline ring, a dibenzoquinazoline ring, a phenanthroline ring, a furodiazine ring, and a benzimidazole ring.

Examples of the heteroaromatic compound having a five-membered ring structure, which is a heteroaromatic compound containing carbon and one or more of nitrogen, oxygen, sulfur, and the like, include a heteroaromatic compound having an imidazole ring, a heteroaromatic compound having a triazole ring, a heteroaromatic compound having an oxazole ring, a heteroaromatic compound having an oxadiazole ring, a heteroaromatic compound having a thiazole ring, and a heteroaromatic compound having a benzimidazole ring.

Examples of the heteroaromatic compound having a six-membered ring structure, which is a heteroaromatic compound containing carbon and one or more of nitrogen, oxygen, sulfur, and the like, include a heteroaromatic compound having a heteroaromatic ring, such as a pyridine ring, a diazine ring (a pyrimidine ring, a pyrazine ring, a pyridazine ring, or the like), a triazine ring, or a polyazole ring. Other examples include a heteroaromatic compound having a bipyridine structure, a heteroaromatic compound having a terpyridine structure, and the like, which are included in examples of a heteroaromatic compound in which pyridine rings are connected.

Examples of the heteroaromatic compound having a fused ring structure partly including the above six-membered ring structure include a heteroaromatic compound having a fused heteroaromatic ring such as a quinoline ring, a benzoquinoline ring, a quinoxaline ring, a dibenzoquinoxaline ring, a phenanthroline ring, a furodiazine ring (including a structure in which an aromatic ring is fused to a furan ring of a furodiazine ring), or a benzimidazole ring.

Specific examples of the above-described heteroaromatic compound having a five-membered ring structure (a polyazole ring (including an imidazole ring, a triazole ring, or an oxadiazole ring), an oxazole ring, a thiazole ring, or a benzimidazole ring) include 2-(4-biphenylyl)-5-(4-tert-butylphenyl)-1,3,4-oxadiazole (abbreviation: PBD), 1,3-bis[5-(p-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: CO11), 3-(4-biphenylyl)-4-phenyl-5-(4-tert-butylphenyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), 2,2′,2″-(1,3,5-benzenetriyl)tris(1-phenyl-1H-benzimidazole) (abbreviation: TPBI), 2-[3-(dibenzothiophen-4-yl)phenyl]-1-phenyl-1H-benzimidazole (abbreviation: mDBTBIm-II), and 4,4′-bis(5-methylbenzoxazol-2-yl)stilbene (abbreviation: BzOs).

Specific examples of the above-described heteroaromatic compound having a six-membered ring structure (including a heteroaromatic ring having a pyridine ring, a diazine ring, a triazine ring, or the like) include: a heteroaromatic compound including a heteroaromatic ring having a pyridine ring, such as 3,5-bis[3-(9H-carbazol-9-yl)phenyl]pyridine (abbreviation: 35DCzPPy) or 1,3,5-tri[3-(3-pyridyl)phenyl]benzene (abbreviation: TmPyPB); a heteroaromatic compound including a heteroaromatic ring having a triazine ring, such as 2-{4-[3-(N-phenyl-9H-carbazol-3-yl)-9H-carbazol-9-yl]phenyl}-4,6-diphenyl-1,3,5-triazine (abbreviation: PCCzPTzn), 9-[3-(4,6-diphenyl-1,3,5-triazin-2-yl)phenyl]-9′-phenyl-2,3′-bi-9H-carbazole (abbreviation: mPCCzPTzn-02), 5-[3-(4,6-diphenyl-1,3,5-triazin-2-yl)phenyl]-7,7-dimethyl-5H,7H-indeno[2,1-b]carbazole (abbreviation: mINc(II)PTzn), 2-[3′-(triphenylen-2-yl)-1,1′-biphenyl-3-yl]-4,6-diphenyl-1,3,5-triazine (abbreviation: mTpBPTzn), 2-[(1,1′-biphenyl)-4-yl]-4-phenyl-6-[9,9′-spirobi(9H-fluoren)-2-yl]-1,3,5-triazine (abbreviation: BP-SFTzn), 2,6-bis(4-naphthalen-1-ylphenyl)-4-[4-(3-pyridyl)phenyl]pyrimidine (abbreviation: 2,4NP-6PyPPm), 3-[9-(4,6-diphenyl-1,3,5-triazin-2-yl)-2-dibenzofuranyl]-9-phenyl-9H-carbazole (abbreviation: PCDBfTzn), 2-[1,1′-biphenyl]-3-yl-4-phenyl-6-(8-[1,1′:4′,1″-terphenyl]-4-yl-1-dibenzofuranyl)-1,3,5-triazine (abbreviation: mBP-TPDBfTzn), 2-{3-[3-(dibenzothiophen-4-yl)phenyl]phenyl}-4,6-diphenyl-1,3,5-triazine (abbreviation: mDBtBPTzn), or mFBPTzn; and a heteroaromatic compound including a heteroaromatic ring having a diazine (pyrimidine) ring, such as 4,6-bis[3-(phenanthren-9-yl)phenyl]pyrimidine (abbreviation: 4,6mPnP2Pm), 4,6-bis[3-(4-dibenzothienyl)phenyl]pyrimidine (abbreviation: 4,6mDBTP2Pm-II), 4,6-bis[3-(9H-carbazol-9-yl)phenyl]pyrimidine (abbreviation: 4,6mCzP2Pm), 4,6mCzBP2Pm, 6-(1,1′-biphenyl-3-yl)-4-[3,5-bis(9H-carbazol-9-yl)phenyl]-2-phenylpyrimidine (abbreviation: 6mBP-4Cz2PPm), 4-[3,5-bis(9H-carbazol-9-yl)phenyl]-2-phenyl-6-(1,1′-biphenyl-4-yl)pyrimidine (abbreviation: 6BP-4Cz2PPm), 4-[3-(dibenzothiophen-4-yl)phenyl]-8-(naphthalen-2-yl)-[1]benzofuro[3,2-d]pyrimidine (abbreviation: 8βN-4mDBtPBfpm), 8BP-4mDBtPBfpm, 9mDBtBPNfpr, 9pmDBtBPNfpr, 3,8-bis[3-(dibenzothiophen-4-yl)phenyl]benzofuro[2,3-b]pyrazine (abbreviation: 3,8mDBtP2Bfpr), 4,8-bis[3-(dibenzothiophen-4-yl)phenyl]-[1]benzofuro[3,2-d]pyrimidine (abbreviation: 4,8mDBtP2Bfpm), 8-[3′-(dibenzothiophen-4-yl)(1,1′-biphenyl-3-yl)]naphtho[1′,2′: 4,5]furo[3,2-d]pyrimidine (abbreviation: 8mDBtBPNfpm), or 8-[(2,2′-binaphthalen)-6-yl]-4-[3-(dibenzothiophen-4-yl)phenyl]-[1]benzofuro[3,2-d]pyrimidine (abbreviation: 8(βN2)-4mDBtPBfpm). Note that the above aromatic compounds including a heteroaromatic ring include a heteroaromatic compound having a fused heteroaromatic ring.

Other examples include heteroaromatic compounds including a heteroaromatic ring having a diazine (pyrimidine) ring, such as 2,2′-(pyridine-2,6-diyl)bis(4-phenylbenzo[h]quinazoline) (abbreviation: 2,6(P-Bqn)2Py), 2,2′-(2,2′-bipyridine-6,6′-diyl)bis(4-phenylbenzo[h]quinazoline) (abbreviation: 6,6′(P-Bqn)2BPy), 2,2′-(pyridine-2,6-diyl)bis{4-[4-(2-naphthyl)phenyl]-6-phenylpyrimidine} (abbreviation: 2,6(NP-PPm)2Py), or 6-(1,1′-biphenyl-3-yl)-4-[3,5-bis(9H-carbazol-9-yl)phenyl]-2-phenylpyrimidine (abbreviation: 6mBP-4Cz2PPm), and a heteroaromatic compound including a heteroaromatic ring having a triazine ring, such as 2,4,6-tris(3′-(pyridin-3-yl)biphenyl-3-yl)-1,3,5-triazine (abbreviation: TmPPPyTz), 2,4,6-tris(2-pyridyl)-1,3,5-triazine (abbreviation: 2Py3Tz), or 2-[3-(2,6-dimethyl-3-pyridinyl)-5-(9-phenanthrenyl)phenyl]-4,6-diphenyl-1,3,5-triazine (abbreviation: mPn-mDMePyPTzn).

Specific examples of the above-described heteroaromatic compound having a fused ring structure partly including a six-membered ring structure (the heteroaromatic compound having a fused ring structure) include a heteroaromatic compound having a quinoxaline ring, such as bathophenanthroline (abbreviation: BPhen), bathocuproine (abbreviation: BCP), 2,9-di(naphthalen-2-yl)-4,7-diphenyl-1,10-phenanthroline (abbreviation: NBPhen), 2,2-(1,3-phenylene)bis[9-phenyl-1,10-phenanthroline](abbreviation: mPPhen2P), 2,2′-(pyridin-2,6-diyl)bis(4-phenylbenzo[h]quinazoline) (abbreviation: 2,6(P-Bqn)2Py), 2-[3-(dibenzothiophen-4-yl)phenyl]dibenzo[f,h]quinoxaline (abbreviation: 2mDBTPDBq-II), 2-[3′-(dibenzothiophen-4-yl)biphenyl-3-yl]dibenzo[f,h]quinoxaline (abbreviation: 2mDBTBPDBq-II), 2-[3′-(9H-carbazol-9-yl)biphenyl-3-yl]dibenzo[f,h]quinoxaline (abbreviation: 2mCzBPDBq), 2-[4-(3,6-diphenyl-9H-carbazol-9-yl)phenyl]dibenzo[f,h]quinoxaline (abbreviation: 2CzPDBq-III), 7-[3-(dibenzothiophen-4-yl)phenyl]dibenzo[f,h]quinoxaline (abbreviation: 7mDBTPDBq-II), 6-[3-(dibenzothiophen-4-yl)phenyl]dibenzo[f,h]quinoxaline (abbreviation: 6mDBTPDBq-II), or 2mpPCBPDBq.

For the electron-transport layers (114, 114a, and 114b), any of the metal complexes given below can be used as well as the heteroaromatic compounds described above. Examples of the metal complexes include a metal complex having a quinoline ring or a benzoquinoline ring, such as tris(8-quinolinolato)aluminum(III) (abbreviation: Alq₃), Almq₃, 8-quinolinolatolithium(I) (abbreviation: Liq), BeBq₂, bis(2-methyl-8-quinolinolato)(4-phenylphenolato)aluminum(III) (abbreviation: BAlq), or bis(8-quinolinolato)zinc(II) (abbreviation: Znq), and a metal complex having an oxazole ring or a thiazole ring, such as bis[2-(2-benzoxazolyl)phenolato]zinc(II) (abbreviation: ZnPBO) or bis[2-(2-benzothiazolyl)phenolato]zinc(II) (abbreviation: ZnBTZ).

High-molecular compounds such as poly(2,5-pyridinediyl) (abbreviation: PPy), poly[(9,9-dihexylfluorene-2,7-diyl)-co-(pyridine-3,5-diyl)] (abbreviation: PF-Py), and poly[(9,9-dioctylfluorene-2,7-diyl)-co-(2,2′-bipyridine-6,6′-diyl)] (abbreviation: PF-BPy) can be used as the electron-transport material.

Each of the electron-transport layers (114, 114a, and 114b) is not limited to a single layer and may be a stack of two or more layers each containing any of the above substances.

### <Electron-Injection Layer>

The electron-injection layers (115, 115a, and 115b) contain a substance having a high electron-injection property. The electron-injection layers (115, 115a, and 115b) are layers for increasing the efficiency of electron injection from the second electrode 102 and are preferably formed using a material whose value of the LUMO level has a small difference (0.5 eV or less) from the work function of a material used for the second electrode 102. Thus, the electron-injection layer 115 can be formed using an alkali metal, an alkaline earth metal, or a compound thereof, such as lithium, cesium, lithium fluoride (LiF), cesium fluoride (CsF), calcium fluoride (CaF₂), Liq, 2-(2-pyridyl)phenolatolithium (abbreviation: LiPP), 2-(2-pyridyl)-3-pyridinolatolithium (abbreviation: LiPPy), 4-phenyl-2-(2-pyridyl)phenolatolithium (abbreviation: LiPPP), an oxide of lithium (LiOₓ), or cesium carbonate. A rare earth metal such as ytterbium (Yb) and a compound of a rare earth metal such as erbium fluoride (ErF₃) can also be used. For the electron-injection layers (115, 115a, and 115b), a plurality of kinds of materials given above may be mixed or stacked as films. Electrode may also be used for the electron-injection layers (115, 115a, and 115b). Examples of the electrode include a substance in which electrons are added at high concentration to calcium oxide-aluminum oxide. Any of the substances used for the electron-transport layers (114, 114a, and 114b), which are given above, can also be used.

A mixed material in which an organic compound and an electron donor (donor) are mixed may also be used for the electron-injection layers (115, 115a, and 115b). Such a mixed material is excellent in an electron-injection property and an electron-transport property because electrons are generated in the organic compound by the electron donor. The organic compound here is preferably a material excellent in transporting the generated electrons; specifically, for example, the above-described electron-transport materials used for the electron-transport layers (114, 114a, and 114b), such as a metal complex and a heteroaromatic compound, can be used. As the electron donor, a substance showing an electron-donating property with respect to an organic compound is preferably used. Specifically, an alkali metal, an alkaline earth metal, and a rare earth metal are preferable, and lithium, cesium, magnesium, calcium, erbium, ytterbium, and the like are given. In addition, an alkali metal oxide and an alkaline earth metal oxide are preferable, and lithium oxide, calcium oxide, barium oxide, and the like are given. Alternatively, a Lewis base such as magnesium oxide can be used. Further alternatively, an organic compound such as tetrathiafulvalene (abbreviation: TTF) can be used. Alternatively, a stack of two or more of these materials may be used.

A mixed material in which an organic compound and a metal are mixed may also be used for the electron-injection layers (115, 115a, and 115b). The organic compound used here preferably has a LUMO level higher than or equal to −3.6 eV and lower than or equal to −2.3 eV. Moreover, a material having an unshared electron pair is preferable.

Thus, as the organic compound used in the above mixed material, a mixed material obtained by mixing a metal and the heteroaromatic compound given above as the material that can be used for the electron-transport layer may be used. Preferred examples of the heteroaromatic compound include materials having an unshared electron pair, such as a heteroaromatic compound having a five-membered ring structure (e.g., an imidazole ring, a triazole ring, an oxazole ring, an oxadiazole ring, a thiazole ring, or a benzimidazole ring), a heteroaromatic compound having a six-membered ring structure (e.g., a pyridine ring, a diazine ring (including a pyrimidine ring, a pyrazine ring, a pyridazine ring, or the like), a triazine ring, a bipyridine ring, or a terpyridine ring), and a heteroaromatic compound having a fused ring structure partly including a six-membered ring structure (e.g., a quinoline ring, a benzoquinoline ring, a quinoxaline ring, a dibenzoquinoxaline ring, or a phenanthroline ring). Since the materials are specifically described above, description thereof is omitted here.

As a metal used for the above mixed material, a transition metal that belongs to Group 5, Group 7, Group 9, or Group 11 or a material that belongs to Group 13 in the periodic table is preferably used, and examples thereof include Ag, Cu, Al, and In. Here, the organic compound forms a singly occupied molecular orbital (SOMO) with the transition metal.

To amplify light obtained from the light-emitting layer 113b, for example, the optical path length between the second electrode 102 and the light-emitting layer 113b is preferably less than one fourth of the wavelength λ of light emitted from the light-emitting layer 113b. In that case, the optical path length can be adjusted by changing the thickness of the electron-transport layer 114b or the electron-injection layer 115b.

When the charge-generation layer 106 is provided between the two EL layers (103a and 103b) as in the light-emitting device in FIG. 2D, a structure in which a plurality of EL layers are stacked between the pair of electrodes (the structure is also referred to as a tandem structure) can be obtained.

### <Charge-Generation Layer>

The charge-generation layer 106 has a function of injecting electrons into the EL layer 103a and injecting holes into the EL layer 103b when voltage is applied between the first electrode (anode) 101 and the second electrode (cathode) 102. The charge-generation layer 106 may have either a structure in which an electron acceptor (acceptor) is added to a hole-transport material or a structure in which an electron donor (donor) is added to an electron-transport material. Alternatively, both of these structures may be stacked. Note that forming the charge-generation layer 106 with the use of any of the above materials can inhibit an increase in driving voltage in the stack of the EL layers.

The first organic compound described in Embodiment 1 can be used for the charge-generation layer 106.

In the case where the charge-generation layer 106 has a structure in which an electron acceptor is added to a hole-transport material, which is an organic compound, any of the materials described in this embodiment can be used as the hole-transport material. Examples of the electron acceptor include 7,7,8,8-tetracyano-2,3,5,6-tetrafluoroquinodimethane (abbreviation: F₄-TCNQ) and chloranil. Other examples include oxides of metals that belong to Group 4 to Group 8 of the periodic table. Specific examples include vanadium oxide, niobium oxide, tantalum oxide, chromium oxide, molybdenum oxide, tungsten oxide, manganese oxide, and rhenium oxide.

In the case where the charge-generation layer 106 has a structure in which an electron donor is added to an electron-transport material, any of the materials described in this embodiment can be used as the electron-transport material. As the electron donor, it is possible to use an alkali metal, an alkaline earth metal, a rare earth metal, a metal belonging to Group 2 or Group 13 of the periodic table, or an oxide or a carbonate thereof. Specifically, lithium (Li), cesium (Cs), magnesium (Mg), calcium (Ca), ytterbium (Yb), indium (In), lithium oxide, cesium carbonate, or the like is preferably used. An organic compound such as tetrathianaphthacene may be used as the electron donor.

Although FIG. 2D illustrates the structure in which two EL layers 103 are stacked, three or more EL layers may be stacked with charge-generation layers each provided between two adjacent EL layers.

### <Substrate>

The light-emitting device described in this embodiment can be formed over a variety of substrates. Note that the type of substrate is not limited to a certain type. Examples of the substrate include semiconductor substrates (e.g., a single crystal substrate or a silicon substrate), an SOI substrate, a glass substrate, a quartz substrate, a plastic substrate, a metal substrate, a stainless steel substrate, a substrate including stainless steel foil, a tungsten substrate, a substrate including tungsten foil, a flexible substrate, an attachment film, paper including a fibrous material, and a base material film.

Examples of the glass substrate include a barium borosilicate glass substrate, an aluminoborosilicate glass substrate, and a soda lime glass substrate. Examples of the flexible substrate, the attachment film, and the base material film include plastics typified by polyethylene terephthalate (PET), polyethylene naphthalate (PEN), and polyether sulfone (PES), a synthetic resin such as acrylic resin, polypropylene, polyester, polyvinyl fluoride, polyvinyl chloride, polyamide, polyimide, aramid, epoxy resin, an inorganic vapor deposition film, and paper.

For fabrication of the light-emitting device in this embodiment, a gas phase method such as an evaporation method or a liquid phase method such as a spin coating method or an ink-jet method can be used. When an evaporation method is used, a physical vapor deposition method (PVD method) such as a sputtering method, an ion plating method, an ion beam evaporation method, a molecular beam evaporation method, or a vacuum evaporation method, a chemical vapor deposition method (CVD method), or the like can be used. Specifically, the layers having various functions (the hole-injection layer 111, the hole-transport layer 112, the light-emitting layer 113, the electron-transport layer 114, and the electron-injection layer 115) included in the EL layers of the light-emitting device can be formed by an evaporation method (e.g., a vacuum evaporation method), a coating method (e.g., a dip coating method, a die coating method, a bar coating method, a spin coating method, or a spray coating method), a printing method (e.g., an ink-jet method, screen printing (stencil), offset printing (planography), flexography (relief printing), gravure printing, or micro-contact printing), or the like.

In the case where a film formation method such as the coating method or the printing method is employed, a high-molecular compound (e.g., an oligomer, a dendrimer, or a polymer), a middle-molecular compound (a compound between a low-molecular compound and a high-molecular compound with a molecular weight of 400 to 4000), an inorganic compound (e.g., a quantum dot material), or the like can be used. The quantum dot material can be a colloidal quantum dot material, an alloyed quantum dot material, a core-shell quantum dot material, a core quantum dot material, or the like.

Materials that can be used for the layers (the hole-injection layer 111, the hole-transport layer 112, the light-emitting layer 113, the electron-transport layer 114, and the electron-injection layer 115) included in the EL layer 103 of the light-emitting device described in this embodiment are not limited to the materials described in this embodiment, and other materials can be used in combination as long as the functions of the layers are fulfilled.

Note that in this specification and the like, the terms “layer” and “film” can be interchanged with each other as appropriate.

The structures described in this embodiment can be used in combination with any of the structures described in the other embodiments as appropriate.

### Embodiment 3

This embodiment will describe a light-emitting and light-receiving apparatus 700 as a specific example of a light-emitting apparatus of one embodiment of the present invention and an example of the manufacturing method. The light-emitting and light-receiving apparatus 700 includes a light-emitting device and thus can be regarded as a light-emitting apparatus; includes a light-receiving device and thus can be regarded as a light-receiving apparatus; and can be used in a display unit in an electronic appliance and thus can be regarded as a display panel or a display device.

### <Structure Example of Light-Emitting and Light-Receiving Apparatus 700>

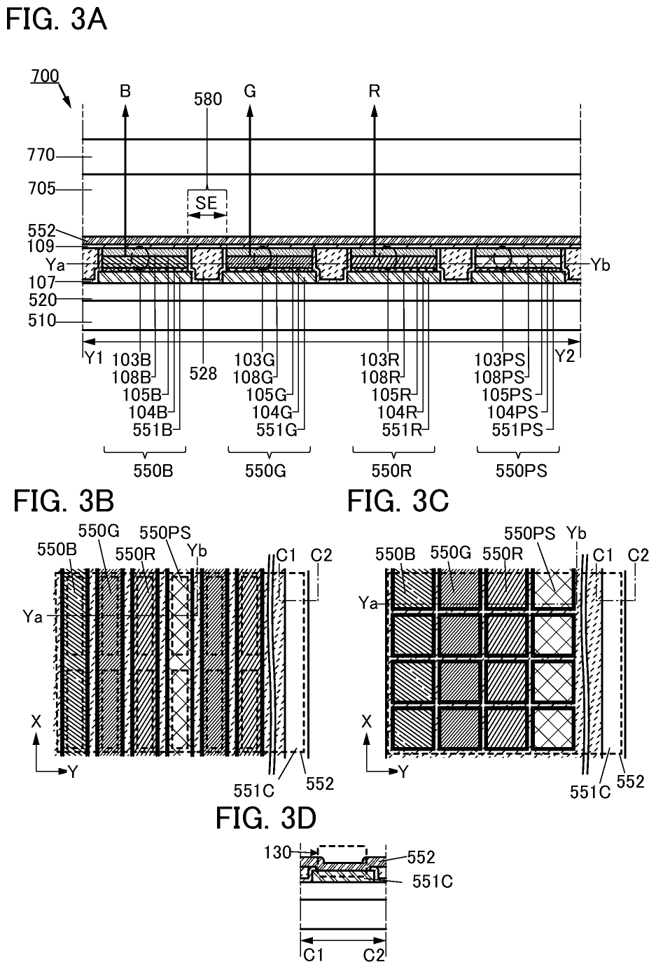

A light-emitting and light-receiving apparatus 700 illustrated in FIG. 3A includes a light-emitting device 550B, a light-emitting device 550G, a light-emitting device 550R, and a light-receiving device 550PS. The light-emitting device 550B, the light-emitting device 550G, the light-emitting device 550R, and the light-receiving device 550PS are formed over a functional layer 520 provided over a first substrate 510. The functional layer 520 includes, for example, circuits such as a driver circuit composed of a plurality of transistors, and wirings that electrically connect these circuits. Note that these driver circuits are electrically connected to the light-emitting device 550B, the light-emitting device 550G, the light-emitting device 550R, and the light-receiving device 550PS, for example, to drive them. The light-emitting and light-receiving apparatus 700 includes an insulating layer 705 over the functional layer 520 and the devices (the light-emitting devices and the light-receiving device), and the insulating layer 705 has a function of attaching a second substrate 770 and the functional layer 520.

The light-emitting device 550B, the light-emitting device 550G, and the light-emitting device 550R include the device structure described in Embodiment 1, and the light-receiving device 550PS has a device structure described later in Embodiment 8. Note that this embodiment describes a non-limiting example in which all the devices (the light-emitting devices and the light-receiving device) are formed to be separated from each other.

In this specification and the like, a structure in which light-emitting layers in light-emitting devices of different colors (for example, blue (B), green (G), and red (R)) and a light-receiving layer in a light-receiving device are separately formed or separately patterned is sometimes referred to as a side-by-side (SBS) structure. Although the light-emitting device 550B, the light-emitting device 550G, the light-emitting device 550R, and the light-receiving device 550PS are arranged in this order in the light-emitting and light-receiving apparatus 700 illustrated in FIG. 3A, one embodiment of the present invention is not limited to this structure. For example, in the light-emitting and light-receiving apparatus 700, these devices may be arranged in the order of the light-emitting device 550R, the light-emitting device 550G, the light-emitting device 550B, and the light-receiving device 550PS.

In FIG. 3A, the light-emitting device 550B includes an electrode 551B, the electrode 552, and an EL layer 103B. The light-emitting device 550G includes an electrode 551G, the electrode 552, and an EL layer 103G. The light-emitting device 550R includes an electrode 551R, the electrode 552, and an EL layer 103R. The light-receiving device 550PS includes an electrode 551PS, the electrode 552, and a light-receiving layer 103PS. Note that a specific structure of each layer of the light-receiving device is as described in Embodiment 8. In addition, a specific structure of each layer of the light-emitting device is as described in Embodiment 2. The EL layer 103B, the EL layer 103G, and the EL layer 103R each have a stacked-layer structure of layers having different functions including their respective light-emitting layers (105B, 105G, and 105R). The light-receiving layer 103PS has a stacked-layer structure of layers having different functions including an active layer 105PS. FIG. 3A illustrates a case where the EL layer 103B includes a hole-injection/transport layer 104B, the light-emitting layer 105B, an electron-transport layer 108B, and an electron-injection layer 109; the EL layer 103G includes a hole-injection/transport layer 104G, the light-emitting layer 105G, an electron-transport layer 108G, and the electron-injection layer 109; the EL layer 103R includes a hole-injection/transport layer 104R, the light-emitting layer 105R, an electron-transport layer 108R, and the electron-injection layer 109; and the light-receiving layer 103PS includes a first transport layer 104PS, the active layer 105PS, a second transport layer 108PS, and the electron-injection layer 109. However, the present invention is not limited thereto. Note that each of the hole-injection/transport layers (104B, 104G, and 104R) represents a layer having the functions of the hole-injection layer and the hole-transport layer described in Embodiment 2, and may have a stacked-layer structure.

Note that the electron-transport layers (108B, 108G, and 108R) and the second transport layer 108PS may have a function of blocking holes moving from the anode side to the cathode side through the EL layers (103B, 103G, and 103R) and the light-receiving layer 103PS. The electron-injection layer 109 may have a stacked-layer structure in which some or all of layers are formed using different materials.

As illustrated in FIG. 3A, the insulating layer 107 may be formed on side surfaces (or end portions) of the hole-injection/transport layers (104B, 104G, and 104R), the light-emitting layers (105B, 105G, and 105R), and the electron-transport layers (108B, 108G, and 108R) included in the EL layers (103B, 103G, and 103R), and side surfaces (or end portions) of the first transport layer 104PS, the active layer 105PS, and the second transport layer 108PS included in the light-receiving layer 103PS. The insulating layer 107 is formed in contact with the side surfaces (or the end portions) of the EL layers (103B, 103G, and 103R) and the light-receiving layer 103PS. This can inhibit entry of oxygen, moisture, or constituent elements thereof into the inside through the side surfaces of the EL layers (103B, 103G, and 103R) and the light-receiving layer 103PS. For the insulating layer 107, aluminum oxide, magnesium oxide, hafnium oxide, gallium oxide, indium gallium zinc oxide, silicon nitride, or silicon nitride oxide can be used, for example. Some of the above-described materials may be stacked to form the insulating layer 107. The insulating layer 107 can be formed by a sputtering method, a CVD method, an MBE method, a PLD method, an ALD method, or the like and is formed preferably by an ALD method, which achieves favorable coverage. Note that the insulating layer 107 continuously covers the side surfaces (or the end portions) of parts of the EL layers (103B, 103G, and 103R) and parts of the light-receiving layer 103PS of adjacent devices. For example, in FIG. 3A, the side surfaces of parts of the EL layer 103B of the light-emitting device 550B and the EL layer 103G of the light-emitting device 550G are covered with the insulating layer 107. In regions covered with the insulating layer 107, partition walls 528 formed using an insulating material are preferably formed, as illustrated in FIG. 3A.

In addition, the electron-injection layer 109 is formed over the electron-transport layers (108B, 108G, and 108R) that are parts of the EL layers (103B, 103G, and 103R), the second transport layer 108PS that is part of the light-receiving layer 103PS, and the insulating layer 107. Note that the electron-injection layer 109 may have a stacked-layer structure of two or more layers (for example, stacked layers having different electric resistances).

The electrode 552 is formed over the electron-injection layer 109. Note that the electrodes (551B, 551G, and 551R) and the electrode 552 include overlap regions. The light-emitting layer 105B is provided between the electrode 551B and the electrode 552, the light-emitting layer 105G is provided between the electrode 551G and the electrode 552, the light-emitting layer 105R is provided between the electrode 551R and the electrode 552, and the light-receiving layer 103PS is provided between the electrode 551PS and the electrode 552.

The EL layers (103B, 103G, and 103R) illustrated in FIG. 3A each have a structure similar to that of the EL layer 103 described in Embodiments 1 and 2. The light-receiving layer 103PS has a structure similar to that of a light-receiving layer described later in Embodiment 8. The light-emitting layer 105B can emit blue light, the light-emitting layer 105G can emit green light, and the light-emitting layer 105R can emit red light, for example.

The partition walls 528 are provided in regions surrounded by the electron-injection layer 109 and the insulating layer 107. As illustrated in FIG. 3A, the partition walls 528 are in contact with the side surfaces (or the end portions) of parts of the electrodes (551B, 551G, 551R, and 551PS), the EL layers (103B, 103G, and 103R), and the light-receiving layer 103PS with the insulating layer 107 therebetween.

In each of the EL layers and the light-receiving layer, particularly the hole-injection layer, which is included in the hole-transport region between the anode and the light-emitting layer and between the anode and the active layer, often has high conductivity; thus, a hole-injection layer formed as a layer shared by adjacent devices might cause crosstalk. Thus, as described in this structure example, the partition walls 528 formed using an insulating material are provided between the EL layers and between the EL layer and the light-receiving layer, which can inhibit occurrence of crosstalk between adjacent devices.

In the manufacturing method described in this embodiment, side surfaces (or end portions) of the EL layer and the light-receiving layer are exposed in the patterning step. This may promote deterioration of the EL layer and the light-receiving layer by allowing the entry of oxygen, water, or the like through the side surfaces (or the end portions) of the EL layer and the light-receiving layer. Hence, providing the partition wall 528 can inhibit the deterioration of the EL layer and the light-receiving layer in the manufacturing process.

Providing the partition wall 528 can flatten the surface by reducing a depressed portion formed between adjacent devices. When the depressed portion is reduced, disconnection of the electrode 552 formed over the EL layers and the light-receiving layer can be inhibited. Examples of an insulating material used to form the partition wall 528 include organic materials such as an acrylic resin, a polyimide resin, an epoxy resin, an imide resin, a polyamide resin, a polyimide-amide resin, a silicone resin, a siloxane resin, a benzocyclobutene-based resin, a phenol resin, and precursors of these resins. Other examples include organic materials such as polyvinyl alcohol (PVA), polyvinyl butyral, polyvinyl pyrrolidone, polyethylene glycol, polyglycerin, pullulan, water-soluble cellulose, and alcohol-soluble polyamide resin. A photosensitive resin such as a photoresist can also be used. Examples of the photosensitive resin include positive-type materials and negative-type materials.

With the use of the photosensitive resin, the partition wall 528 can be fabricated by only light exposure and developing steps. The partition wall 528 may be fabricated using a negative photosensitive resin (e.g., a resist material). In the case where an insulating layer containing an organic material is used as the partition wall 528, a material absorbing visible light is suitably used. When such a material absorbing visible light is used for the partition wall 528, light emission from the EL layer can be absorbed by the partition wall 528, leading to a reduction in light leakage (stray light) to an adjacent EL layer or light-receiving layer. Accordingly, a display panel with high display quality can be provided.

For example, the difference between the top-surface level of the partition wall 528 and the top-surface level of any of the EL layer 103B, the EL layer 103G, the EL layer 103R, and the light-receiving layer 103PS is preferably 0.5 times or less, further preferably 0.3 times or less the thickness of the partition wall 528. The partition wall 528 may be provided such that the top-surface level of any of the EL layer 103B, the EL layer 103G, the EL layer 103R, and the light-receiving layer 103PS is higher than the top-surface level of the partition wall 528, for example. Alternatively, the partition wall 528 may be provided such that the top-surface level of the partition wall 528 is higher than the top-surface level of any of the EL layer 103B, the EL layer 103G, the EL layer 103R, and the light-receiving layer 103PS, for example.

When electrical continuity is established between the EL layer 103B, the EL layer 103G, the EL layer 103R, and the light-receiving layer 103PS in a light-emitting and light-receiving apparatus (display panel) with a high resolution more than 1000 ppi, crosstalk occurs, resulting in a narrower color gamut that the light-emitting and light-receiving apparatus is capable of reproducing. Providing the partition wall 528 in a high-resolution display panel with more than 1000 ppi, preferably more than 2000 ppi, or further preferably in an ultrahigh-resolution display panel with more than 5000 ppi allows the display panel to express vivid colors.

FIGS. 3B and 3C are each a schematic top view of the light-emitting and light-receiving apparatus 700 taken along the dashed-dotted line Ya-Yb in the cross-sectional view of FIG. 3A. Specifically, the light-emitting device 550B, the light-emitting device 550G, and the light-emitting device 550R are arranged in a matrix. Note that FIG. 3B illustrates what is called a stripe arrangement, in which the light-emitting devices of the same color are arranged in the X-direction. FIG. 3C illustrates a structure in which the light-emitting devices of the same color are arranged in the X-direction and separated by patterning for each pixel. Note that the arrangement method of the light-emitting devices is not limited thereto; another method such as a delta, zigzag, PenTile, or diamond arrangement may also be used.

The EL layers (103B, 103G, and 103R) and the light-receiving layer 103PS are processed to be separated by patterning using a photolithography method; hence, a light-emitting and light-receiving apparatus (display panel) with a high resolution can be fabricated. The side surfaces (end portions) of the layers of the EL layer processed by patterning using a photolithography method have substantially the same surface (or are positioned on substantially the same plane). In addition, the side surfaces (end portions) of the layers of the light-receiving layer processed by patterning using a photolithography method have substantially the same surface (or are positioned on substantially the same plane). In this case, the widths (SE) of spaces 580 between the EL layers and between the EL layer and the light-receiving layer are each preferably 5 μm or less, further preferably 1 μm or less.

In the EL layer, particularly the hole-injection layer, which is included in the hole-transport region between the anode and the light-emitting layer, often has high conductivity; thus, a hole-injection layer formed as a layer shared by adjacent light-emitting devices might cause crosstalk. Therefore, processing the EL layers to be separated by patterning using a photolithography method as described in this structure example can suppress occurrence of crosstalk between adjacent light-emitting devices.

FIG. 3D is a schematic cross-sectional view taken along the dashed-dotted line C1-C2 in FIGS. 3B and 3C. FIG. 3D illustrates a connection portion 130 where a connection electrode 551C and the electrode 552 are electrically connected to each other. In the connection portion 130, the electrode 552 is provided over and in contact with the connection electrode 551C. The partition wall 528 is provided to cover an end portion of the connection electrode 551C.

### <Example of Method for Manufacturing Light-Emitting and Light-Receiving Apparatus>

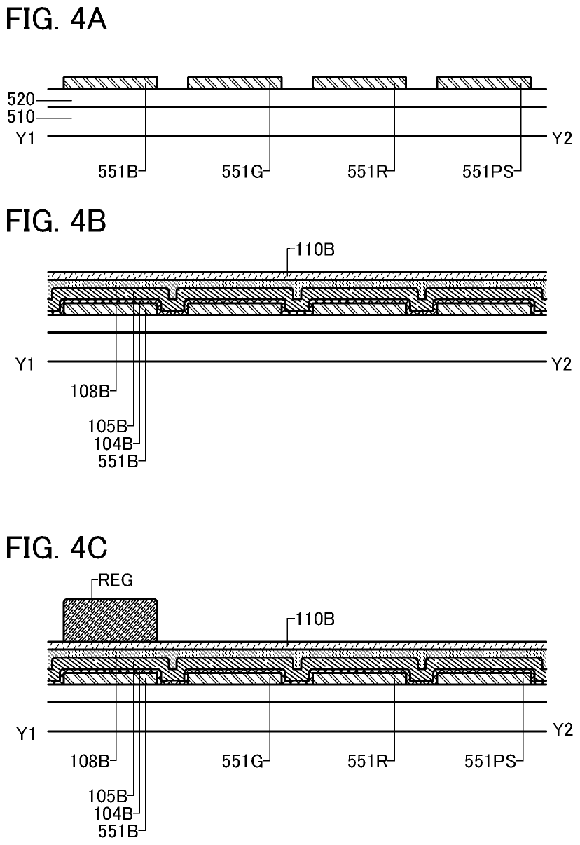

The electrode 551B, the electrode 551G, the electrode 551R, and the electrode 551PS are formed as illustrated in FIG. 4A. For example, a conductive film is formed over the functional layer 520 over the first substrate 510 and processed into predetermined shapes by a photolithography method.

The conductive film can be formed by any of a sputtering method, a chemical vapor deposition (CVD) method, a molecular beam epitaxy (MBE) method, a vacuum evaporation method, a pulsed laser deposition (PLD) method, an atomic layer deposition (ALD) method, and the like. Examples of the CVD method include a plasma-enhanced chemical vapor deposition (PECVD) method and a thermal CVD method. An example of a thermal CVD method is a metal organic CVD (MOCVD) method.

The conductive film may be processed by a nanoimprinting method, a sandblasting method, a lift-off method, or the like as well as a photolithography method described above. Alternatively, island-shaped thin films may be directly formed by a film formation method using a shielding mask such as a metal mask.

There are two typical examples of photolithography methods. In one of the methods, a resist mask is formed over a thin film to be processed, the thin film is processed by etching or the like, and then the resist mask is removed. In the other method, a photosensitive thin film is formed and then processed into a desired shape by light exposure and development. The former method involves heat treatment steps such as pre-applied bake (PAB) after resist application and post-exposure bake (PEB) after light exposure. In one embodiment of the present invention, a lithography method is used not only for processing of a conductive film but also for processing of a thin film used for formation of an EL layer (a film made of an organic compound or a film partly containing an organic compound).

As light for exposure in a photolithography method, it is possible to use light with the i-line (wavelength: 365 nm), light with the g-line (wavelength: 436 nm), light with the h-line (wavelength: 405 nm), or light in which the i-line, the g-line, and the h-line are mixed. Alternatively, ultraviolet light, KrF laser light, ArF laser light, or the like can be used. Exposure may be performed by liquid immersion exposure technique. As the light for exposure, extreme ultraviolet (EUV) light or X-rays may also be used. Instead of the light for exposure, an electron beam can be used. It is preferable to use EUV, X-rays, or an electron beam because extremely minute processing can be performed. Note that a photomask is not needed when light exposure is performed by scanning with a beam such as an electron beam.

For etching of a thin film using a resist mask, a dry etching method, a wet etching method, a sandblasting method, or the like can be used.

Subsequently, as illustrated in FIG. 4B, the hole-injection/transport layer 104B, the light-emitting layer 105B, and the electron-transport layer 108B are formed over the electrode 551B, the electrode 551G, the electrode 551R, and the electrode 551PS. Note that the hole-injection/transport layer 104B, the light-emitting layer 105B, and the electron-transport layer 108B can be formed using a vacuum evaporation method, for example. Furthermore, a sacrifice layer 110B is formed over the electron-transport layer 108B. For the formation of the hole-injection/transport layer 104B, the light-emitting layer 105B, and the electron-transport layer 108B, any of the materials described in Embodiment 2 can be used.

For the sacrifice layer 110B, it is preferable to use a film highly resistant to etching treatment performed on the hole-injection/transport layer 104B, the light-emitting layer 105B, and the electron-transport layer 108B, i.e., a film having high etching selectivity with respective to the hole-injection/transport layer 104B, the light-emitting layer 105B, and the electron-transport layer 108B. The sacrifice layer 110B preferably has a stacked-layer structure of a first sacrifice layer and a second sacrifice layer which have different etching selectivities. For the sacrifice layer 110B, it is possible to use a film that can be removed by a wet etching method, which causes less damage to the EL layer 103B. In wet etching, oxalic acid or the like can be used as an etching material.

For the sacrifice layer 110B, an inorganic film such as a metal film, an alloy film, a metal oxide film, a semiconductor film, or an inorganic insulating film can be used, for example. The sacrifice layer 110B can be formed by any of a variety of film formation methods such as a sputtering method, an evaporation method, a CVD method, and an ALD method.

For the sacrifice layer 110B, a metal material such as gold, silver, platinum, magnesium, nickel, tungsten, chromium, molybdenum, iron, cobalt, copper, palladium, titanium, aluminum, yttrium, zirconium, or tantalum or an alloy material containing the metal material can be used, for example. It is particularly preferable to use a low-melting-point material such as aluminum or silver.

A metal oxide such as indium gallium zinc oxide (also referred to as In—Ga—Zn oxide or IGZO) can be used for the sacrifice layer 110B. It is also possible to use indium oxide, indium zinc oxide (In—Zn oxide), indium tin oxide (In—Sn oxide), indium titanium oxide (In—Ti oxide), indium tin zinc oxide (In—Sn—Zn oxide), indium titanium zinc oxide (In—Ti—Zn oxide), indium gallium tin zinc oxide (In—Ga—Sn—Zn oxide), or the like. Alternatively, indium tin oxide containing silicon can also be used, for example.

An element M (M is one or more of aluminum, silicon, boron, yttrium, copper, vanadium, beryllium, titanium, iron, nickel, germanium, zirconium, molybdenum, lanthanum, cerium, neodymium, hafnium, tantalum, tungsten, and magnesium) may be used instead of gallium. In particular, M is preferably one or more of gallium, aluminum, and yttrium.

For the sacrifice layer 110B, an inorganic insulating material such as aluminum oxide, hafnium oxide, or silicon oxide can be used.

The sacrifice layer 110B is preferably formed using a material that can be dissolved in a solvent chemically stable with respect to the electron-transport layer 108B that is in the uppermost position. Specifically, a material that can be dissolved in water or alcohol can be suitably used for the sacrifice layer 110B. In formation of the sacrifice layer 110B, preferably, application of such a material dissolved in a solvent such as water or alcohol is performed by a wet process, followed by heat treatment for evaporating the solvent. At this time, the heat treatment is preferably performed under a reduced-pressure atmosphere, in which case the solvent can be removed at a low temperature in a short time and thermal damage to the hole-injection/transport layer 104B, the light-emitting layer 105B, and the electron-transport layer 108B can be accordingly reduced.

In the case where the sacrifice layer 110B having a stacked-layer structure is formed, the stacked-layer structure can include the first sacrifice layer formed using any of the above-described materials and the second sacrifice layer thereover.

The second sacrifice layer in that case is a film used as a hard mask for etching of the first sacrifice layer. In processing the second sacrifice layer, the first sacrifice layer is exposed. Thus, a combination of films having greatly different etching rates is selected for the first sacrifice layer and the second sacrifice layer. Thus, a film that can be used for the second sacrifice layer can be selected in accordance with the etching conditions of the first sacrifice layer and those of the second sacrifice layer.

For example, in the case where the second sacrifice layer is etched by dry etching using a fluorine-containing gas (also referred to as a fluorine-based gas), the second sacrifice layer can be formed using silicon, silicon nitride, silicon oxide, tungsten, titanium, molybdenum, tantalum, tantalum nitride, an alloy containing molybdenum and niobium, an alloy containing molybdenum and tungsten, or the like. Here, a film of a metal oxide such as IGZO or ITO can be given as an example of a film having a high etching selectivity to the second sacrifice layer (i.e., a film with a low etching rate) in the dry etching using the fluorine-based gas, and can be used for the first sacrifice layer.

Note that the material for the second sacrifice layer is not limited to the above and can be selected from a variety of materials in accordance with the etching conditions of the first sacrifice layer and those of the second sacrifice layer. For example, any of the films that can be used for the first sacrifice layer can be used for the second sacrifice layer.

For the second sacrifice layer, a nitride film can be used, for example. Specifically, it is possible to use a nitride such as silicon nitride, aluminum nitride, hafnium nitride, titanium nitride, tantalum nitride, tungsten nitride, gallium nitride, or germanium nitride.

Alternatively, an oxide film can be used for the second sacrifice layer. Typically, it is possible to use a film of an oxide or an oxynitride such as silicon oxide, silicon oxynitride, aluminum oxide, aluminum oxynitride, hafnium oxide, or hafnium oxynitride.

Next, as illustrated in FIG. 4C, a resist is applied onto the sacrifice layer 110B, and the resist having a desired shape (a resist mask REG) is formed by a photolithography method. Such a method involves heat treatment steps such as pre-applied bake (PAB) after the resist application and post-exposure bake (PEB) after light exposure. The temperature reaches approximately 100° C. during the PAB, and approximately 120° C. during the PEB, for example. Therefore, the light-emitting device should be resistant to such high treatment temperatures.

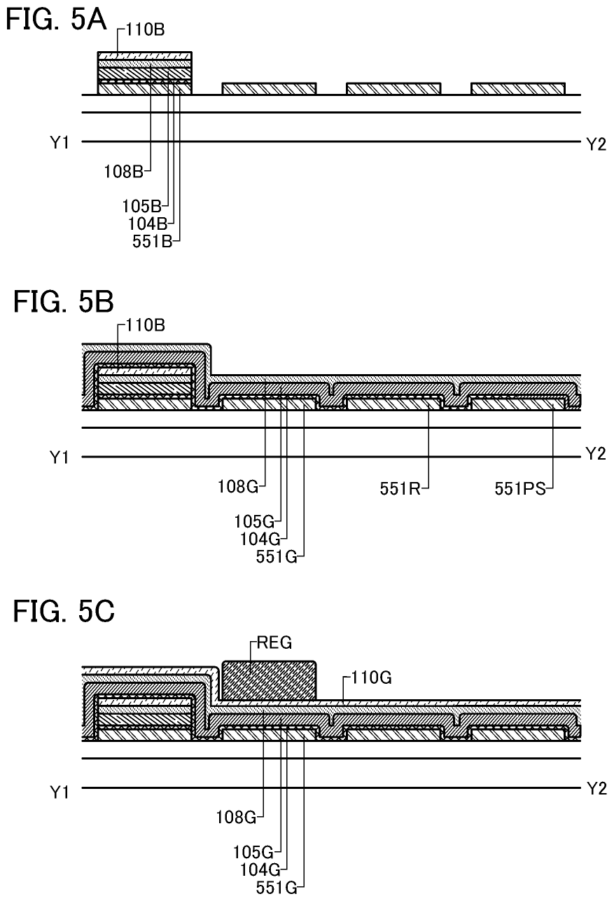

Next, part of the sacrifice layer 110B that is not covered with the resist mask REG is removed by etching using the resist mask REG, the resist mask REG is removed, and then the hole-injection/transport layer 104B, the light-emitting layer 105B, and the electron-transport layer 108B that are not covered with the sacrifice layer 110B are partly removed by etching, so that the hole-injection/transport layer 104B, the light-emitting layer 105B, and the electron-transport layer 108B are processed to have side surfaces (or have their side surfaces exposed) over the electrode 551B or have belt-like shapes extending in the direction intersecting the sheet of the diagram. Note that dry etching is preferably employed for the etching. Note that in the case where the sacrifice layer 110B has the aforementioned stacked-layer structure of the first sacrifice layer and the second sacrifice layer, the hole-injection/transport layer 104B, the light-emitting layer 105B, and the electron-transport layer 108B may be processed into a predetermined shape in the following manner: part of the second sacrifice layer is etched using the resist mask REG, the resist mask REG is then removed, and part of the first sacrifice layer is etched using the second sacrifice layer as a mask. The structure illustrated in FIG. 5A is obtained through these etching steps.

Subsequently, as illustrated in FIG. 5B, the hole-injection/transport layer 104G, the light-emitting layer 105G, and the electron-transport layer 108G are formed over the sacrifice layer 110B, the electrode 551G, the electrode 551R, and the electrode 551PS. The hole-injection/transport layer 104G, the light-emitting layer 105G, and the electron-transport layer 108G can be formed using any of the materials described in Embodiment 2. Note that the hole-injection/transport layer 104G, the light-emitting layer 105G, and the electron-transport layer 108G can be formed by a vacuum evaporation method, for example.

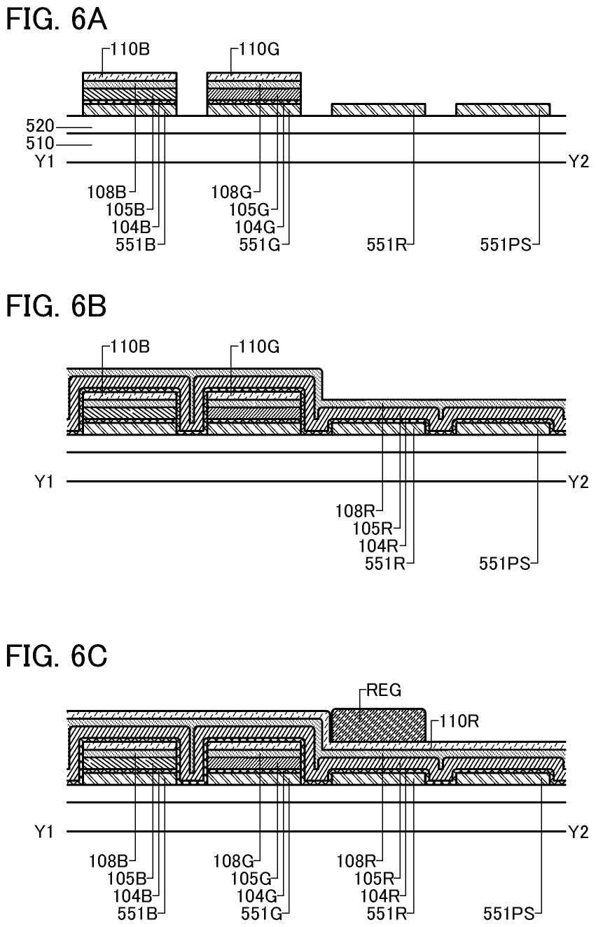

Next, as illustrated in FIG. 5C, the sacrifice layer 110G is formed over the electron-transport layer 108G, a resist is applied onto the sacrifice layer 110G, and the resist having a desired shape (the resist mask REG) is formed by a photolithography method. Part of the sacrifice layer 110G that is not covered with the obtained resist mask REG is removed by etching, the resist mask REG is removed, and then parts of the hole-injection/transport layer 104G, the light-emitting layer 105G, and the electron-transport layer 108G that are not covered with the sacrifice layer 110G are removed by etching. Thus, the hole-injection/transport layer 104G, the light-emitting layer 105G, and the electron-transport layer 108G are processed to have side surfaces (or have their side surfaces exposed) over the electrode 551G or have belt-like shapes extending in the direction perpendicular to the sheet of the diagram. Note that dry etching is preferably employed for the etching. Note that the sacrifice layer 110G can be formed using a material similar to that for the sacrifice layer 110B. In the case where the sacrifice layer 110G has the aforementioned stacked-layer structure of the first sacrifice layer and the second sacrifice layer, the hole-injection/transport layer 104G, the light-emitting layer 105G, and the electron-transport layer 108G may be processed into a predetermined shape in the following manner: part of the second sacrifice layer is etched using the resist mask REG, the resist mask REG is then removed, and part of the first sacrifice layer is etched using the second sacrifice layer as a mask. The structure illustrated in FIG. 6A is obtained through these etching steps.

Next, as illustrated in FIG. 6B, the hole-injection/transport layer 104R, the light-emitting layer 105R, and the electron-transport layer 108R are formed over the sacrifice layer 110B, the sacrifice layer 110G, the electrode 551R, and the electrode 551PS. The hole-injection/transport layer 104R, the light-emitting layer 105R, and the electron-transport layer 108R can be formed using any of the materials described in Embodiment 2. The hole-injection/transport layer 104R, the light-emitting layer 105R, and the electron-transport layer 108R can be formed by a vacuum evaporation method, for example.

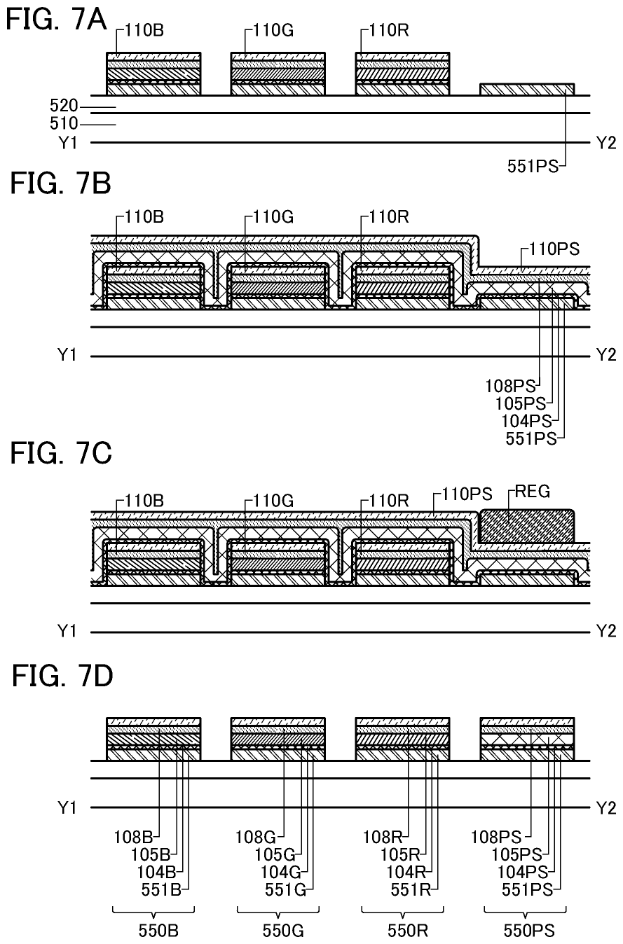

Next, as illustrated in FIG. 6C, the sacrifice layer 110R is formed over the electron-transport layer 108R, a resist is applied onto the sacrifice layer 110R, and the resist having a desired shape (the resist mask REG) is formed by a photolithography method. Part of the sacrifice layer 110R that is not covered with the obtained resist mask REG is removed by etching, the resist mask REG is removed, and then parts of the hole-injection/transport layer 104R, the light-emitting layer 105R, and the electron-transport layer 108R that are not covered with the sacrifice layer 110R are removed by etching. Thus, the hole-injection/transport layer 104R, the light-emitting layer 105R, and the electron-transport layer 108R are processed to have side surfaces (or have their side surfaces exposed) over the electrode 551R or have belt-like shapes extending in the direction intersecting the sheet of the diagram. Note that dry etching is preferably employed for the etching. Note that the sacrifice layer 110R can be formed using a material similar to that for the sacrifice layer 110B. In the case where the sacrifice layer 110R has the aforementioned stacked-layer structure of the first sacrifice layer and the second sacrifice layer, the hole-injection/transport layer 104R, the light-emitting layer 105R, and the electron-transport layer 108R may be processed into a predetermined shape in the following manner: part of the second sacrifice layer is etched using the resist mask REG, the resist mask REG is then removed, and part of the first sacrifice layer is etched using the second sacrifice layer as a mask. The structure illustrated in FIG. 7A is obtained through these etching steps.

Next, as illustrated in FIG. 7B, the first transport layer 104PS, the active layer 105PS, and the second transport layer 108PS are formed over the sacrifice layer 110B, the sacrifice layer 110G, the sacrifice layer 110R, and the electrode 551PS. The first transport layer 104PS, the active layer 105PS, and the second transport layer 108PS can be formed using any of the materials described in Embodiment 1. Note that the first transport layer 104PS, the active layer 105PS, and the second transport layer 108PS can be formed by a vacuum evaporation method, for example.

Next, as illustrated in FIG. 7C, the sacrifice layer 110PS is formed over the second transport layer 108PS, a resist is applied onto the sacrifice layer 110PS, and the resist having a desired shape (the resist mask REG) is formed by a photolithography method. Part of the sacrifice layer 110PS that is not covered with the obtained resist mask REG is removed by etching, the resist mask REG is removed, and then the first transport layer 104PS, the active layer 105PS, and the second transport layer 108PS that are not covered with the sacrifice layer 110PS are partly removed by etching. Thus, the first transport layer 104PS, the active layer 105PS, and the second transport layer 108PS are processed to have side surfaces (or have their side surfaces exposed) over the electrode 551PS or have belt-like shapes extending in the direction intersecting the sheet of the diagram. Note that dry etching is preferably employed for the etching. Note that the sacrifice layer 110PS can be formed using a material similar to that for the sacrifice layer 110B. In the case where the sacrifice layer 110PS has the aforementioned stacked-layer structure of the first sacrifice layer and the second sacrifice layer, the first transport layer 104PS, the active layer 105PS, and the second transport layer 108PS may be processed into a predetermined shape in the following manner: part of the second sacrifice layer is etched using the resist mask REG, the resist mask REG is then removed, and part of the first sacrifice layer is etched using the second sacrifice layer as a mask. The structure illustrated in FIG. 7D is obtained through these etching steps.

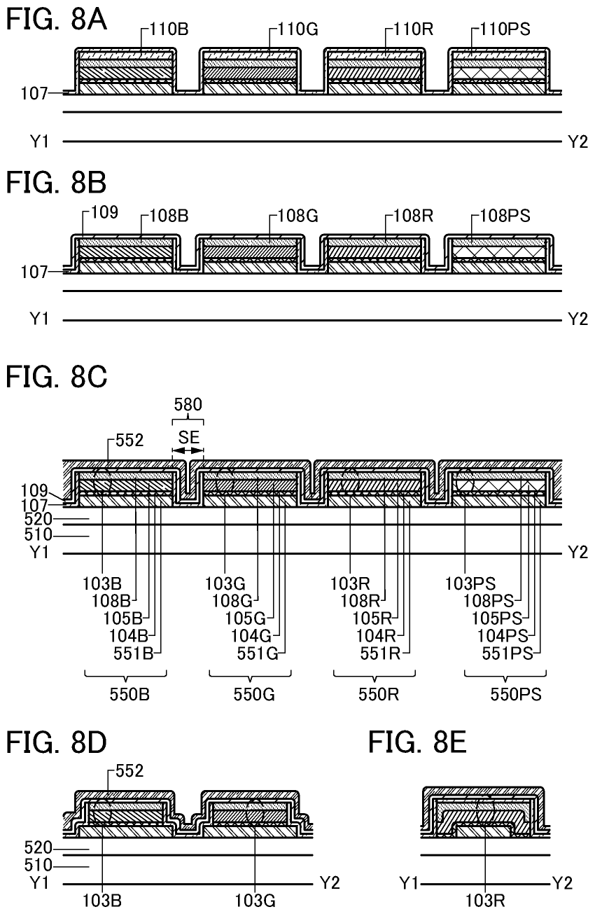

Next, as illustrated in FIG. 8A, the insulating layer 107 is formed over the sacrifice layers 110B, 110G, 110R, and 110PS.

Note that the insulating layer 107 can be formed by an ALD method, for example. In this case, as illustrated in FIG. 8A, the insulating layer 107 is formed to be in contact with the side surfaces (end portions) of the hole-injection/transport layers (104B, 104G, and 104R), the light-emitting layers (105B, 105G, and 105R), and the electron-transport layers (108B, 108G, and 108R) of the light-emitting devices and the first transport layer 104PS, the active layer 105PS, and the second transport layer 108PS of the light-receiving device. This can inhibit entry of oxygen, moisture, or constituent elements thereof into the inside through the side surfaces of the layers. Examples of the material used for the insulating layer 107 include aluminum oxide, magnesium oxide, hafnium oxide, gallium oxide, indium gallium zinc oxide, silicon nitride, and silicon nitride oxide.

Then, as illustrated in FIG. 8B, after part of the insulating layer 107 and the sacrifice layers (110B, 110G, 110R, and 110PS) are removed, the electron-injection layer 109 is formed over the insulating layer 107, the electron-transport layers (108B, 108G, and 108R), and the second transport layer 108PS. The electron-injection layer 109 can be formed using any of the materials described in Embodiment 2. The electron-injection layer 109 is formed by a vacuum evaporation method, for example. Note that the electron-injection layer 109 is in contact with the side surfaces (end portions) of the hole-injection/transport layers (104B, 104G, and 104R), the light-emitting layers (105B, 105G, and 105R), and the electron-transport layers (108B, 108G, and 108R) of the light-emitting devices and the first transport layer 104PS, the active layer 105PS, and the second transport layer 108PS of the light-receiving device with the insulating layer 107 therebetween.

Next, as illustrated in FIG. 8C, the electrode 552 is formed. The electrode 552 is formed by a vacuum evaporation method, for example. The electrode 552 is formed over the electron-injection layer 109. Note that the electrode 552 is in contact with the side surfaces (end portions) of the hole-injection/transport layers (104B, 104G, and 104R), the light-emitting layers (105B, 105G, and 105R), and the electron-transport layers (108B, 108G, and 108R) of the light-emitting devices and the first transport layer 104PS, the active layer 105PS, and the second transport layer 108PS of the light-receiving device with the electron-injection layer 109 and the insulating layer 107 therebetween. This can prevent electrical short circuits between the electrode 552 and each of the following layers: the hole-injection/transport layers (104B, 104G, and 104R), the light-emitting layers (105B, 105G, and 105R), and the electron-transport layers (108B, 108G, and 108R) of the light-emitting devices and the first transport layer 104PS, the active layer 105PS, and the second transport layer 108PS of the light-receiving device.

Through the above steps, the EL layer 103B, the EL layer 103G, the EL layer 103R, and the light-receiving layer 103PS in the light-emitting device 550B, the light-emitting device 550G, the light-emitting device 550R, and the light-receiving device 550PS can be processed to be separated from each other.

The EL layers (103B, 103G, and 103R) and the light-receiving layer 103PS are processed to be separated by patterning using a photolithography method; hence, a light-emitting and light-receiving apparatus (display panel) with a high resolution can be fabricated. Side surfaces (end portions) of the layers of the EL layer processed by patterning using a photolithography method have substantially the same surface (or are positioned on substantially the same plane). In addition, the side surfaces (end portions) of the layers of the light-receiving layer processed by patterning using a photolithography method have substantially the same surface (or are positioned on substantially the same plane).

Each of the hole-injection/transport layers (104B, 104G, and 104R) of the EL layers and the first transport layer 104PS of the light-receiving layer often has high conductivity, and thus might cause crosstalk when formed as a layer shared by adjacent devices. Therefore, processing the layers to be separated by patterning using a photolithography method as described in this structure example can inhibit occurrence of crosstalk between adjacent devices.

In this structure example, the hole-injection/transport layers (104B, 104G, and 104R), the light-emitting layers (105B, 105G, and 105R), and the electron-transport layers (108B, 108G, and 108R) of the EL layers (103B, 103G, and 103R) included in the light-emitting devices and the first transport layer 104PS, the active layer 105PS, and the second transport layer 108PS of the light-receiving layer 103PS included in the light-receiving device are processed to be separated by patterning using a photolithography method; thus, the side surfaces (end portions) of the layers of the processed EL layer have substantially the same surface (or are positioned on substantially the same plane). In addition, the side surfaces (end portions) of the layers of the light-receiving layer processed by patterning using a photolithography method have substantially the same surface (or are positioned on substantially the same plane).

In addition, the hole-injection/transport layers (104B, 104G, and 104R), the light-emitting layers (105B, 105G, and 105R), and the electron-transport layers (108B, 108G, and 108R) of the EL layers (103B, 103G, and 103R) included in the light-emitting devices and the first transport layer 104PS, the active layer 105PS, and the second transport layer 108PS of the light-receiving layer 103PS included in the light-receiving device are processed to be separated by patterning using a photolithography method. Thus, the space 580 is provided between the processed side surfaces (end portions) of adjacent devices. In FIG. 8C, when the space 580 is denoted by a distance SE between the EL layers or between the EL layer and the light-receiving layer of adjacent devices, decreasing the distance SE increases the aperture ratio and the resolution. By contrast, as the distance SE is increased, the effect of the difference in the fabrication process between the adjacent devices becomes permissible, which leads to an increase in manufacturing yield. Since the light-emitting device and the light-receiving device fabricated according to this specification are suitable for a miniaturization process, the distance SE between the EL layers or between the EL layer and the light-receiving layer of adjacent devices can be longer than or equal to 0.5 μm and shorter than or equal to 5 μm, preferably longer than or equal to 1 μm and shorter than or equal to 3 μm, further preferably longer than or equal to 1 μm and shorter than or equal to 2.5 μm, and still further preferably longer than or equal to 1 μm and shorter than or equal to 2 sm. Typically, the distance SE is preferably longer than or equal to 1 μm and shorter than or equal to 2 μm (e.g., 1.5 μm or a neighborhood thereof).

In this specification and the like, a device formed using a metal mask or a fine metal mask (FMM) is sometimes referred to as a device having a metal mask (MM) structure. In this specification and the like, a device formed without using a metal mask or an FMM is sometimes referred to as a device having a metal maskless (MML) structure. Since a light-emitting and light-receiving apparatus having the MML structure is formed without using a metal mask, the pixel arrangement, the pixel shape, and the like can be designed more flexibly than in a light-emitting and light-receiving apparatus having the FMM structure or the MM structure.

Note that the island-shaped EL layers of the light-emitting and light-receiving apparatus having the MML structure are formed by not patterning using a metal mask but processing after deposition of an EL layer. Thus, a light-emitting and light-receiving apparatus with a higher resolution or a higher aperture ratio than a conventional one can be achieved. Moreover, EL layers can be formed separately for each color, which enables extremely clear images; thus, a light-emitting and light-receiving apparatus with a high contrast and high display quality can be achieved. Furthermore, provision of a sacrifice layer over an EL layer can reduce damage on the EL layer during the manufacturing process and increase the reliability of the light-emitting device.

In FIG. 3A and FIG. 8C, the widths of the EL layers (103B, 103G, and 103R) are substantially equal to those of the electrodes (551B, 551G, and 551R) in the light-emitting device 550B, the light-emitting device 550G, and the light-emitting device 550R, and the width of the light-receiving layer 103PS is substantially equal to that of the electrode 551PS in the light-receiving device 550PS; however, one embodiment of the present invention is not limited thereto.

In the light-emitting device 550B, the light-emitting device 550G, and the light-emitting device 550R, the widths of the EL layers (103B, 103G, and 103R) may be smaller than those of the electrodes (551B, 551G, and 551R). In the light-receiving device 550PS, the width of the light-receiving layer 103PS may be smaller than that of the electrode 551PS. FIG. 8D illustrates an example in which the widths of the EL layers (103B and 103G) are smaller than those of the electrodes (551B and 551G) in the light-emitting devices 550B and 550G.

In the light-emitting devices 550B, 550G, and 550R, the widths of the EL layers (103B, 103G, and 103R) may be larger than those of the electrodes (551B, 551G, and 551R). In the light-receiving device 550PS, the width of the light-receiving layer 103PS may be larger than that of the electrode 551PS. FIG. 8E illustrates an example in which the width of the EL layer 103R is larger than that of the electrode 551R in the light-emitting device 550R.

The structures described in this embodiment can be used in combination with any of the structures described in the other embodiments as appropriate.

### Embodiment 4

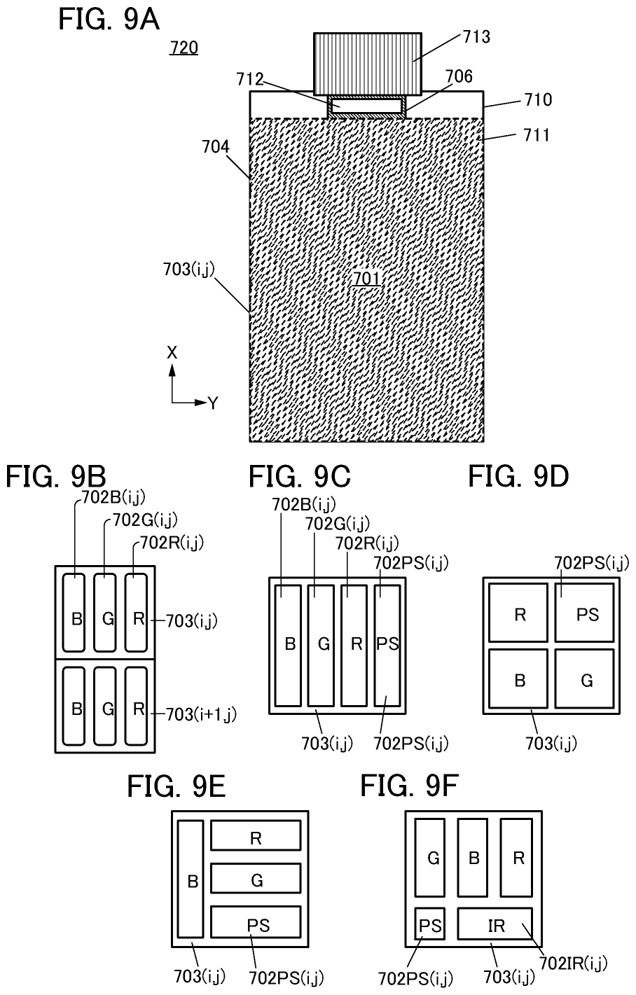

In this embodiment, an apparatus 720 is described with reference to FIGS. 9A to 9F, FIGS. 10A to 10C, and FIG. 11. The apparatus 720 illustrated in FIGS. 9A to 9F, FIGS. 10A to 10C, and FIG. 11 includes any of the light-emitting devices described in Embodiments 1 and 2 and therefore is a light-emitting apparatus. Furthermore, the apparatus 720 described in this embodiment can be used in a display unit of an electronic appliance or the like and therefore can also be referred to as a display panel or a display device. Moreover, when the apparatus 720 includes the light-emitting device as a light source and a light-receiving device that can receive light from the light-emitting device, the apparatus 720 can be referred to as a light-emitting and light-receiving apparatus. Note that the light-emitting apparatus, the display panel, the display device, and the light-emitting and light-receiving apparatus each include at least a light-emitting device.

Furthermore, the light-emitting apparatus, the display panel, the display device, and the light-emitting and light-receiving apparatus of this embodiment can each have high definition or a large size. Therefore, the light-emitting apparatus, the display panel, the display device, and the light-emitting and light-receiving apparatus of this embodiment can be used, for example, in display units of electronic appliances such as a digital camera, a digital video camera, a digital photo frame, a mobile phone, a portable game console, a smart phone, a wristwatch terminal, a tablet terminal, a portable information terminal, and an audio reproducing apparatus, in addition to display units of electronic appliances with a relatively large screen, such as a television device, a desktop or laptop personal computer, a monitor of a computer or the like, digital signage, and a large game machine such as a pachinko machine.

FIG. 9A is a top view of the apparatus 720 (e.g., the light-emitting apparatus, the display panel, the display device, and the light-emitting and light-receiving apparatus).

In FIG. 9A, the apparatus 720 has a structure in which a substrate 710 and a substrate 711 are attached to each other. In addition, the apparatus 720 includes a display region 701, a circuit 704, a wiring 706, and the like. Note that the display region 701 includes a plurality of pixels. As illustrated in FIG. 9B, a pixel 703(i,j) illustrated in FIG. 9A and a pixel 703(i+1,j) are adjacent to each other.

Furthermore, in the example of the apparatus 720 illustrated in FIG. 9A, the substrate 710 is provided with an integrated circuit (IC) 712 by a chip on glass (COG) method, a chip on film (COF) method, or the like. As the IC 712, an IC including a scan line driver circuit, a signal line driver circuit, or the like can be used, for example. In the example illustrated in FIG. 9A, an IC including a signal line driver circuit is used as the IC 712, and a scan line driver circuit is used as the circuit 704.

The wiring 706 has a function of supplying signals and power to the display region 701 and the circuit 704. The signals and power are input to the wiring 706 from the outside through a flexible printed circuit (FPC) 713 or to the wiring 706 from the IC 712. Note that the apparatus 720 is not necessarily provided with the IC. The IC may be mounted on the FPC by a COF method or the like.

FIG. 9B illustrates the pixel 703(i, j) and the pixel 703(i+1, j) of the display region 701. A plurality of kinds of subpixels including light-emitting devices that emit different color light from each other can be included in the pixel 703(i,j). Alternatively, a plurality of subpixels including light-emitting devices that emit the same color light may be included. In the case where a plurality of kinds of subpixels including light-emitting devices that emit different color light from each other are included in the pixel, three kinds of subpixels can be included, for example. The three subpixels can be of three colors of red (R), green (G), and blue (B) or of three colors of yellow (Y), cyan (C), and magenta (M), for example. Alternatively, the pixel can include four kinds of subpixels. The four subpixels can be of four colors of R, G, B, and white (W) or of four colors of R, G, B, and Y, for example. Specifically, the pixel 703(i, j) can consist of a subpixel 702B(i,j) for blue display, a subpixel 702G(i,j) for green display, and a subpixel 702R(i, j) for red display.

The 720 includes not only a subpixel including a light-emitting device, but also a subpixel including a light-receiving device.

FIGS. 9C to 9E illustrate various layout examples of the pixel 703(i,j)including a subpixel 702PS(i,j) including a light-receiving device. The pixel arrangement in FIG. 9C is stripe arrangement, and the pixel arrangement in FIG. 9D is matrix arrangement. The pixel arrangement in FIG. 9E has a structure where three subpixels (the subpixels R, G, and PS) are vertically arranged next to one subpixel (the subpixel B).

Furthermore, as illustrated in FIG. 9F, a subpixel 702IR(i,j) that emits infrared rays may be added to any of the above-described sets of subpixels in the pixel 703(i, j). In the pixel arrangement in FIG. 9F, the vertically oriented three subpixels G, B, and R are arranged laterally, and the subpixel PS and the horizontally oriented subpixel IR are arranged laterally below the three subpixels. Specifically, the subpixel 702IR(i, j) that emits light including light with a wavelength ranging from 650 nm to 1000 nm, inclusive, may be used in the pixel 703(i, j). Note that the wavelength of light detected by the subpixel 702PS(i, j) is not particularly limited; however, the light-receiving device included in the subpixel 702PS(i,j) preferably has sensitivity to light emitted by the light-emitting device included in the subpixel 702R(i, j), the subpixel 702G(i, j), the subpixel 702B(i, j), or the subpixel 702IR(i, j). For example, the light-receiving device preferably detects one or more kinds of light in blue, violet, bluish violet, green, yellowish green, yellow, orange, red, and infrared wavelength ranges, for example.

Note that the arrangement of subpixels is not limited to the structures illustrated in FIGS. 9B to 9F and a variety of arrangement methods can be employed. The arrangement of subpixels may be stripe arrangement, S stripe arrangement, matrix arrangement, delta arrangement, Bayer arrangement, or pentile arrangement, for example.

Furthermore, top surfaces of the subpixels may have a triangular shape, a quadrangular shape (including a rectangular shape and a square shape), a polygonal shape such as a pentagonal shape, a polygonal shape with rounded corners, an elliptical shape, or a circular shape, for example. The top surface shape of a subpixel herein refers to a top surface shape of a light-emitting region of a light-emitting device.

Furthermore, in the case where not only a light-emitting device but also a light-receiving device is included in a pixel, the pixel has a light-receiving function and thus can detect a contact or approach of an object while displaying an image. For example, an image can be displayed by using all the subpixels included in a light-emitting apparatus; or light can be emitted by some of the subpixels as a light source and an image can be displayed by using the remaining subpixels.

Note that the light-receiving area of the subpixel 702PS(i, j) is preferably smaller than the light-emitting areas of the other subpixels. A smaller light-receiving area leads to a narrower image-capturing range, inhibits a blur in a captured image, and improves the definition. Thus, by using the subpixel 702PS(i, j), high-resolution or high-definition image capturing is possible. For example, image capturing for personal authentication with the use of a fingerprint, a palm print, the iris, the shape of a blood vessel (including the shape of a vein and the shape of an artery), a face, or the like is possible by using the subpixel 702PS(i,j).

Moreover, the subpixel 702PS(i, j) can be used in a touch sensor (also referred to as a direct touch sensor), a near touch sensor (also referred to as a hover sensor, a hover touch sensor, a contactless sensor, or a touchless sensor), or the like. For example, the subpixel 702PS(i, j) preferably detects infrared light. Thus, touch sensing is possible even in a dark place.

Here, the touch sensor or the near touch sensor can detect an approach or contact of an object (e.g., a finger, a hand, or a pen). The touch sensor can detect the object when the light-emitting and light-receiving apparatus and the object come in direct contact with each other. Furthermore, the near touch sensor can detect the object even when the object is not in contact with the light-emitting and light-receiving apparatus. For example, the light-emitting and light-receiving apparatus can preferably detect the object when the distance between the light-emitting and light-receiving apparatus and the object is more than or equal to 0.1 mm and less than or equal to 300 mm, preferably more than or equal to 3 mm and less than or equal to 50 mm. With this structure, the light-emitting and light-receiving apparatus can be controlled without the object directly contacting with the light-emitting and light-receiving apparatus. In other words, the light-emitting and light-receiving apparatus can be controlled in a contactless (touchless) manner. With the above-described structure, the light-emitting and light-receiving apparatus can be controlled with a reduced risk of being dirty or damaged, or without direct contact between the object and a dirt (e.g., dust, bacteria, or a virus) attached to the light-emitting and light-receiving apparatus.

For high-resolution image capturing, the subpixel 702PS(i, j) is preferably provided in every pixel included in the light-emitting and light-receiving apparatus. Meanwhile, in the case where the subpixel 702PS(i, j) is used in a touch sensor, a near touch sensor, or the like, high accuracy is not required as compared to the case of capturing an image of a fingerprint or the like; accordingly, the subpixel 702PS(i, j) is provided in some subpixels in the light-emitting and light-receiving apparatus. When the number of subpixels 702PS(i, j) included in the light-emitting and light-receiving apparatus is smaller than the number of subpixels 702R(i, j) or the like, higher detection speed can be achieved.

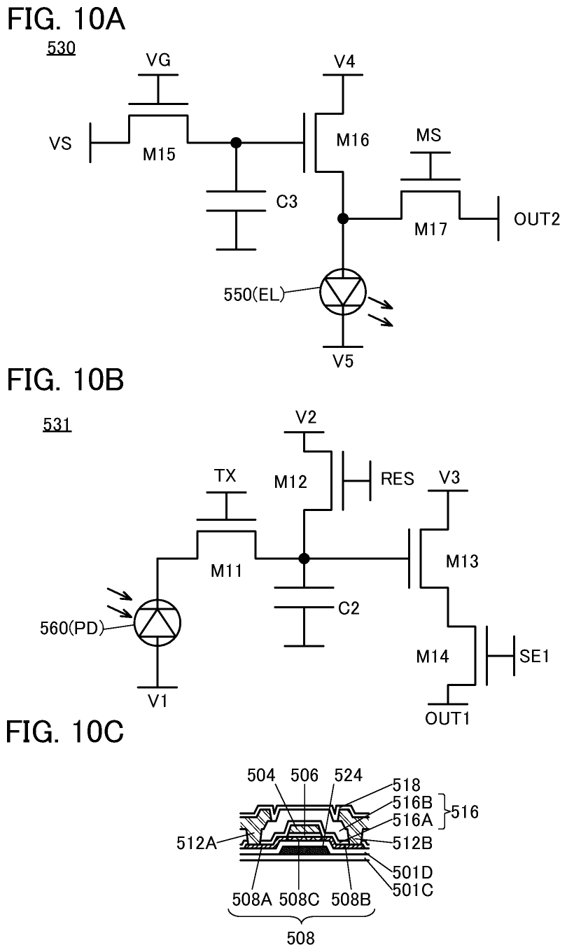

Next, an example of a pixel circuit of a subpixel included in the light-emitting device is described with reference to FIG. 10A A pixel circuit 530 illustrated in FIG. 10A includes a light-emitting device (EL) 550, a transistor M15, a transistor M16, a transistor M17, and a capacitor C3. Note that a light-emitting diode can be used as the light-emitting device 550. In particular, any of the light-emitting devices described in Embodiments 1 and 2 is preferably used as the light-emitting device 550.

In FIG. 10A, a gate of the transistor M15 is electrically connected to a wiring VG, one of a source and a drain of the transistor M15 is electrically connected to a wiring VS, and the other of the source and the drain of the transistor M15 is electrically connected to one electrode of the capacitor C3 and agate of the transistor M16. One of a source and a drain of the transistor M16 is electrically connected to a wiring V4, and the other is electrically connected to an anode of the light-emitting device 550 and one of a source and a drain of the transistor M17. A gate of the transistor M17 is electrically connected to a wiring MS, and the other of the source and the drain of the transistor M17 is electrically connected to a wiring OUT2. A cathode of the light-emitting device 550 is electrically connected to a wiring V5.

A constant potential is supplied to the wiring V4 and the wiring V5. In the light-emitting device 550, the anode side can have a high potential and the cathode side can have a lower potential than the anode side. The transistor M15 is controlled by a signal supplied to the wiring VG and functions as a selection transistor for controlling a selection state of the pixel circuit 530. The transistor M16 functions as a driving transistor that controls a current flowing through the light-emitting device 550 in accordance with a potential supplied to the gate of the transistor M16. When the transistor M15 is on, a potential supplied to the wiring VS is supplied to the gate of the transistor M16, and the luminance of the light-emitting device 550 can be controlled in accordance with the potential. The transistor M17 is controlled by a signal supplied to the wiring MS and has a function of outputting a potential between the transistor M16 and the light-emitting device 550 to the outside through the wiring OUT2.

Here, a transistor in which a metal oxide (an oxide semiconductor) is used as a semiconductor layer where a channel is formed is preferably used as transistors M15, M16, and M17 included in a pixel circuit 530 in FIG. 10A and the transistors M11, M12, M13, and M14 included in the pixel circuit 531 in FIG. 10B.

A transistor using a metal oxide having a wider band gap and a lower carrier density than silicon can achieve an extremely low off-state current. Such a low off-state current enables retention of charges accumulated in a capacitor that is connected in series to the transistor for along time. Therefore, it is particularly preferable to use a transistor containing an oxide semiconductor as the transistors M11, M12, and M15 each of which is connected in series to a capacitor C2 or the capacitor C3. When each of the other transistors also includes an oxide semiconductor, manufacturing cost can be reduced.

Alternatively, transistors using silicon as a semiconductor in which a channel is formed can be used as the transistors M11 to M17. In particular, it is preferable to use silicon with high crystallinity such as single crystal silicon or polycrystalline silicon because high field-effect mobility can be achieved and higher-speed operation can be performed.

Alternatively, a transistor containing an oxide semiconductor may be used as at least one of the transistors M11 to M17, and transistors containing silicon may be used as the other transistors.

Next, an example of a pixel circuit of a subpixel including a light-receiving device is described with reference to FIG. 10B. The pixel circuit 531 illustrated in FIG. 10B includes a light-receiving device (PD) 560, the transistor M11, the transistor M12, the transistor M13, the transistor M14, and the capacitor C2. For example, a photodiode is used as the light-receiving device (PD) 560.

In FIG. 10B, an anode of the light-receiving device (PD) 560 is electrically connected to a wiring V1, and a cathode of the light-receiving device (PD) 560 is electrically connected to one of a source and a drain of the transistor M11. A gate of the transistor M11 is electrically connected to a wiring TX, and the other of the source and the drain of the transistor M11 is electrically connected to one electrode of the capacitor C2, one of a source and a drain of the transistor M12, and a gate of the transistor M13. A gate of the transistor M12 is electrically connected to a wiring RES, and the other of the source and the drain of the transistor M12 is electrically connected to a wiring V2. One of a source and a drain of the transistor M13 is electrically connected to a wiring V3, and the other of the source and the drain of the transistor M13 is electrically connected to one of a source and a drain of the transistor M14. A gate of the transistor M14 is electrically connected to a wiring SE1, and the other of the source and the drain of the transistor M14 is electrically connected to a wiring OUT1.

A constant potential is supplied to the wiring V1, the wiring V2, and the wiring V3. When the light-receiving device (PD) 560 is driven with a reverse bias, the wiring V2 is supplied with a potential higher than the potential of the wiring V1. The transistor M12 is controlled by a signal supplied to the wiring RES and has a function of resetting the potential of a node connected to the gate of the transistor M13 to a potential supplied to the wiring V2. The transistor M11 is controlled by a signal supplied to the wiring TX and has a function of controlling the timing at which the potential of the node changes, in accordance with a current flowing through the light-receiving device (PD) 560. The transistor M13 functions as an amplifier transistor for outputting a signal corresponding to the potential of the node. The transistor M14 is controlled by a signal supplied to the wiring SE1 and functions as a selection transistor for reading an output corresponding to the potential of the node by an external circuit connected to the wiring OUT1.

Although n-channel transistors are illustrated in FIGS. 10A and 10B, p-channel transistors can be used instead.

The transistors included in the pixel circuit 530 and the transistors included in the pixel circuit 531 are preferably formed side by side over the same substrate. Preferably, the transistors included in the pixel circuit 530 and the transistors included in the pixel circuit 531 are periodically arranged in one region, in particular.

One or more layers including the transistor and/or the capacitor are preferably provided to overlap with the light-receiving device (PD) 560 or the light-emitting device (EL) 550. Thus, the effective area of each pixel circuit can be reduced, and a high-resolution light-receiving unit or display unit can be achieved.

FIG. 10C illustrates an example of a specific structure of a transistor that can be used in the pixel circuit described with reference to FIGS. 10A and 10B. As the transistor, a bottom-gate transistor, a top-gate transistor, or the like can be used as appropriate.

The transistor illustrated in FIG. 10C includes a semiconductor film 508, a conductive film 504, an insulating film 506, a conductive film 512A, and a conductive film 512B. The transistor is formed over an insulating film 501C, for example. The transistor also includes an insulating film 516 (an insulating film 516A and an insulating film 516B) and an insulating film 518.

The semiconductor film 508 includes a region 508A electrically connected to the conductive film 512A and a region 508B electrically connected to the conductive film 512B. The semiconductor film 508 includes a region 508C between the region 508A and the region 508B.

The conductive film 504 includes a region overlapping with the region 508C and has a function of a gate electrode.

The insulating film 506 includes a region positioned between the semiconductor film 508 and the conductive film 504. The insulating film 506 has a function of a first gate insulating film.

The conductive film 512A has one of a function of a source electrode and a function of a drain electrode, and the conductive film 512B has the other thereof.

A conductive film 524 can be used in the transistor. The semiconductor film 508 is sandwiched between the conductive film 504 and a region included in the conductive film 524. The conductive film 524 has a function of a second gate electrode. An insulating film 501D is positioned between the semiconductor film 508 and the conductive film 524 and has a function of a second gate insulating film.

The insulating film 516 functions as, for example, a protective film covering the semiconductor film 508. Specifically, a film including a silicon oxide film, a silicon oxynitride film, a silicon nitride oxide film, a silicon nitride film, an aluminum oxide film, a hafnium oxide film, an yttrium oxide film, a zirconium oxide film, a gallium oxide film, a tantalum oxide film, a magnesium oxide film, a lanthanum oxide film, a cerium oxide film, or a neodymium oxide film can be used as the insulating film 516, for example.

For the insulating film 518, a material that has a function of inhibiting diffusion of oxygen, hydrogen, water, an alkali metal, an alkaline earth metal, and the like is preferably used. Specifically, the insulating film 518 can be formed using silicon nitride, silicon oxynitride, aluminum nitride, or aluminum oxynitride, for example. In each of silicon oxynitride and aluminum oxynitride, the number of nitrogen atoms contained is preferably larger than the number of oxygen atoms contained.

Note that in a step of forming the semiconductor film used in the transistor of the pixel circuit, the semiconductor film used in the transistor of the driver circuit can be formed. A semiconductor film having the same composition as the semiconductor film used in the transistor of the pixel circuit can be used in the driver circuit, for example.

The semiconductor film 508 preferably contains indium, M (M is one or more of gallium, aluminum, silicon, boron, yttrium, tin, copper, vanadium, beryllium, titanium, iron, nickel, germanium, zirconium, molybdenum, lanthanum, cerium, neodymium, hafnium, tantalum, tungsten, and magnesium), and zinc, for example. Specifically, M is preferably one or more of aluminum, gallium, yttrium, and tin.

In particular, an oxide containing indium (In), gallium (Ga), and zinc (Zn) (also referred to as IGZO) is preferably used as the semiconductor film 508. Alternatively, it is preferable to use an oxide containing indium, tin, and zinc. Further alternatively, it is preferable to use an oxide containing indium, gallium, tin, and zinc. Further alternatively, it is preferable to use an oxide containing indium (In), aluminum (Al), and zinc (Zn) (also referred to as IAZO). Further alternatively, it is preferable to use an oxide containing indium (In), aluminum (Al), gallium (Ga), and zinc (Zn) (also referred to as IAGZO).

When the semiconductor film is an In-M-Zn oxide, the atomic ratio of In is preferably greater than or equal to the atomic ratio of M in the In-M-Zn oxide. Examples of the atomic ratio of the metal elements in such an In-M-Zn oxide are In:M:Zn=1:1:1, 1:1:1.2, 1:3:2, 1:3:4, 2:1:3, 3:1:2, 4:2:3, 4:2:4.1, 5:1:3, 5:1:6, 5:1:7, 5:1:8, 6:1:6, and 5:2:5 and a composition in the vicinity of any of the above atomic ratios. Note that the vicinity of the atomic ratio includes ±30% of an intended atomic ratio.

For example, in the case of describing an atomic ratio of In:Ga:Zn=4:2:3 or a composition in the vicinity thereof, the case is included in which with the atomic proportion of In being 4, the atomic proportion of Ga is greater than or equal to 1 and less than or equal to 3 and the atomic proportion of Zn is greater than or equal to 2 and less than or equal to 4. In the case of describing an atomic ratio of In:Ga:Zn=5:1:6 or a composition in the vicinity thereof, the case is included in which with the atomic proportion of In being 5, the atomic proportion of Ga is greater than 0.1 and less than or equal to 2 and the atomic proportion of Zn is greater than or equal to 5 and less than or equal to 7. In the case of describing an atomic ratio of In:Ga:Zn=1:1:1 or a composition in the vicinity thereof, the case is included in which with the atomic proportion of In being 1, the atomic proportion of Ga is greater than 0.1 and less than or equal to 2 and the atomic proportion of Zn is greater than 0.1 and less than or equal to 2.

There is no particular limitation on the crystallinity of a semiconductor material used in the transistor, and an amorphous semiconductor or a semiconductor having crystallinity (a microcrystalline semiconductor, a polycrystalline semiconductor, a single crystal semiconductor, or a semiconductor partly including crystal regions) can be used. It is preferable to use a semiconductor having crystallinity, in which case deterioration of transistor characteristics can be suppressed.

A semiconductor layer of a transistor preferably contains a metal oxide (also referred to as an oxide semiconductor). As an oxide semiconductor having crystallinity, a c-axis aligned crystalline oxide semiconductor (CAAC-OS), a nanocrystalline oxide semiconductor (nc-OS), and the like are given.

Alternatively, a transistor using silicon in its channel formation region (a Si transistor) may be used. Examples of silicon include single crystal silicon (single crystal Si), polycrystalline silicon, and amorphous silicon. In particular, a transistor containing low-temperature polysilicon (LTPS) in its semiconductor layer (hereinafter also referred to as an LTPS transistor) can be used. The LTPS transistor has high field-effect mobility and excellent frequency characteristics.

With the use of Si transistors such as LTPS transistors, a circuit required to be driven at a high frequency (e.g., a source driver circuit) can be formed on the same substrate as the display unit. This allows simplification of an external circuit mounted on the light-emitting apparatus and a reduction in costs of parts and mounting costs.

A transistor containing a metal oxide (hereinafter also referred to as an oxide semiconductor) in a semiconductor where a channel is formed (hereinafter such a transistor is also referred to as an OS transistor) has much higher field-effect mobility than a transistor containing amorphous silicon. In addition, the OS transistor has an extremely low leakage current between a source and a drain in an off state (hereinafter also referred to as off-state current), and charge accumulated in a capacitor that is connected in series to the transistor can be held for a long period. Furthermore, the power consumption of the light-emitting apparatus can be reduced with the OS transistor.

The off-state current per micrometer of channel width of the OS transistor at room temperature can be lower than or equal to 1 aA (1×10⁻¹⁸ A), lower than or equal to 1 zA (1×10⁻²¹ A), or lower than or equal to 1 yA (1×10⁻²⁴ A). Note that the off-state current per micrometer of channel width of a Si transistor at room temperature is higher than or equal to 1 fA (1×10⁻¹⁵ A) and lower than or equal to 1 pA (1×10⁻¹² A). In other words, the off-state current of the OS transistor is lower than that of the Si transistor by approximately ten orders of magnitude.

To increase the luminance of the light-emitting device included in the pixel circuit, the amount of current fed through the light-emitting device needs to be increased. To increase the current amount, the source-drain voltage of a driving transistor included in the pixel circuit needs to be increased. An OS transistor has a higher withstand voltage between a source and a drain than a Si transistor; hence, high voltage can be applied between the source and the drain of the OS transistor. Thus, with use of an OS transistor as a driving transistor included in the pixel circuit, the amount of current flowing through the light-emitting device can be increased, resulting in an increase in emission luminance of the light-emitting device.

When transistors operate in a saturation region, a change in source-drain current relative to a change in gate-source voltage can be smaller in an OS transistor than in a Si transistor. Accordingly, when an OS transistor is used as the driving transistor in the pixel circuit, a current flowing between the source and the drain can be set minutely in accordance with a change in gate-source voltage; hence, the amount of current flowing through the light-emitting device can be controlled. Consequently, the number of gray levels expressed by the pixel circuit can be increased.

Regarding saturation characteristics of current flowing when transistors operates in a saturation region, even in the case where the source-drain voltage of an OS transistor increases gradually, a more stable current (saturation current) can be fed through the OS transistor than through a Si transistor. Thus, by using an OS transistor as the driving transistor, a stable current can be fed through light-emitting devices even when the current-voltage characteristics of the light-emitting devices vary, for example. In other words, when the OS transistor operates in the saturation region, the source-drain current hardly changes with an increase in the source-drain voltage; hence, the luminance of the light-emitting device can be stable.

As described above, with use of an OS transistor as a driving transistor included in the pixel circuit, it is possible to achieve “inhibition of black floating”, “increase in emission luminance”, “increase in gray level”, “inhibition of variation in light-emitting devices”, and the like.

The semiconductor film used in the transistor of the driver circuit can be formed in the same step as the semiconductor film used in the transistor of the pixel circuit. The driver circuit can be formed over a substrate where the pixel circuit is formed. The number of components of an electronic appliance can be reduced.

Alternatively, silicon may be used for the semiconductor film 508. Examples of silicon include single crystal silicon, polycrystalline silicon, and amorphous silicon. In particular, a transistor containing low-temperature polysilicon (LTPS) in its semiconductor layer (hereinafter also referred to as an LTPS transistor) is preferably used. The LTPS transistor has high field-effect mobility and excellent frequency characteristics.

With the use of transistors using silicon such as LTPS transistors, a circuit required to be driven at a high frequency (e.g., a source driver circuit) can be formed on the same substrate as the display unit. This allows simplification of an external circuit mounted on the light-emitting apparatus and a reduction in costs of parts and mounting costs.

It is preferable to use an OS transistor as at least one of the transistors included in the pixel circuit. The OS transistor has much higher field-effect mobility than a transistor containing amorphous silicon. In addition, the OS transistor has an extremely low leakage current between a source and a drain in an off state (hereinafter also referred to as off-state current), and charge accumulated in a capacitor that is connected in series to the transistor can be held for a long period. Furthermore, the power consumption of the light-emitting apparatus can be reduced with the OS transistor.

When an LTPS transistor is used as one or more of the transistors included in the pixel circuit and an OS transistor is used as the rest, the light-emitting apparatus can have low power consumption and high driving capability. As a favorable example, it is preferable that an OS transistor be used as a transistor functioning as a switch for controlling electrical continuity between wirings and an LTPS transistor be used as a transistor for controlling current, for instance. A structure where an LTPS transistor and an OS transistor are used in combination may be referred to as LTPO. The use of LTPO enables the display panel to have low power consumption and high drive capability.

For example, one of the transistors included in the pixel circuit functions as a transistor for controlling a current flowing through the light-emitting device and can be referred to as a driving transistor. One of a source and a drain of the driving transistor is electrically connected to the pixel electrode of the light-emitting device. An LTPS transistor is preferably used as the driving transistor. Accordingly, the amount of current flowing through the light-emitting device can be increased in the pixel circuit.

Another transistor included in the pixel circuit functions as a switch for controlling selection and non-selection of the pixel and can be referred to as a selection transistor. A gate of the selection transistor is electrically connected to a gate line, and one of a source and a drain thereof is electrically connected to a source line (signal line). An OS transistor is preferably used as the selection transistor. Accordingly, the gray level of the pixel can be maintained even with an extremely low frame frequency (e.g., 1 fps or less); thus, power consumption can be reduced by stopping the driver in displaying a still image.

In the case of using an oxide semiconductor in a semiconductor film, the apparatus 720 includes a light-emitting device including an oxide semiconductor in its semiconductor film and having a metal maskless (MML) structure. With this structure, the leakage current that might flow through the transistor and the leakage current that might flow between adjacent light-emitting devices (also referred to as a lateral leakage current, a side leakage current, or the like) can become extremely low. With the structure, a viewer can observe any one or more of the image crispness, the image sharpness, a high chroma, and a high contrast ratio in an image displayed on the display device. When the leakage current that might flow through the transistor and the lateral leakage current that might flow between light-emitting devices are extremely low, display with little leakage of light at the time of black display (what is called black floating) (such display is also referred to as deep black display) can be achieved.

In particular, in the case where a light-emitting device having an MML structure employs the above-described SBS structure, a layer provided between light-emitting devices (for example, also referred to as an organic layer or a common layer which is shared by the light-emitting devices) is disconnected; accordingly, display with no or extremely small lateral leakage can be achieved.

The structure of the transistors used in the display panel may be selected as appropriate depending on the size of the screen of the display panel. For example, single crystal Si transistors can be used in the display panel with a screen diagonal greater than or equal to 0.1 inches and less than or equal to 3 inches. In addition, LTPS transistors can be used in the display panel with a screen diagonal greater than or equal to 0.1 inches and less than or equal to 30 inches, preferably greater than or equal to 1 inch and less than or equal to 30 inches. In addition, an LTPO structure (where an LTPS transistor and an OS transistor are used in combination) can be used for the display panel with a screen diagonal greater than or equal to 0.1 inches and less than or equal to 50 inches, preferably greater than or equal to 1 inch and less than or equal to 50 inches. In addition, OS transistors can be used in the display panel with a screen diagonal greater than or equal to 0.1 inches and less than or equal to 200 inches, preferably greater than or equal to 50 inches and less than or equal to 100 inches.

With the use of single crystal Si transistors, an increase in screen size is extremely difficult due to the size of a single crystal Si substrate. Furthermore, since a laser crystallization apparatus is used in the fabrication process, LTPS transistors are unlikely to respond to an increase in screen size (typically to a screen diagonal greater than 30 inches). By contrast, since the fabrication process does not necessarily require a laser crystallization apparatus or the like or can be performed at a relatively low temperature (typically, lower than or equal to 450° C.), OS transistors can be applied to a display panel with a relatively large area (typically, a screen diagonal greater than or equal to 50 inches and less than or equal to 100 inches). In addition, LTPO can be applied to a display panel with a size (typically, a screen diagonal greater than or equal to 1 inch and less than or equal to 50 inches) midway between the size of a display panel using LTPS transistors and the size of a display panel using OS transistors.

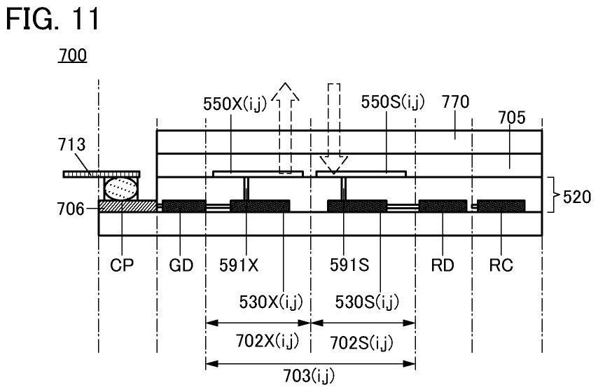

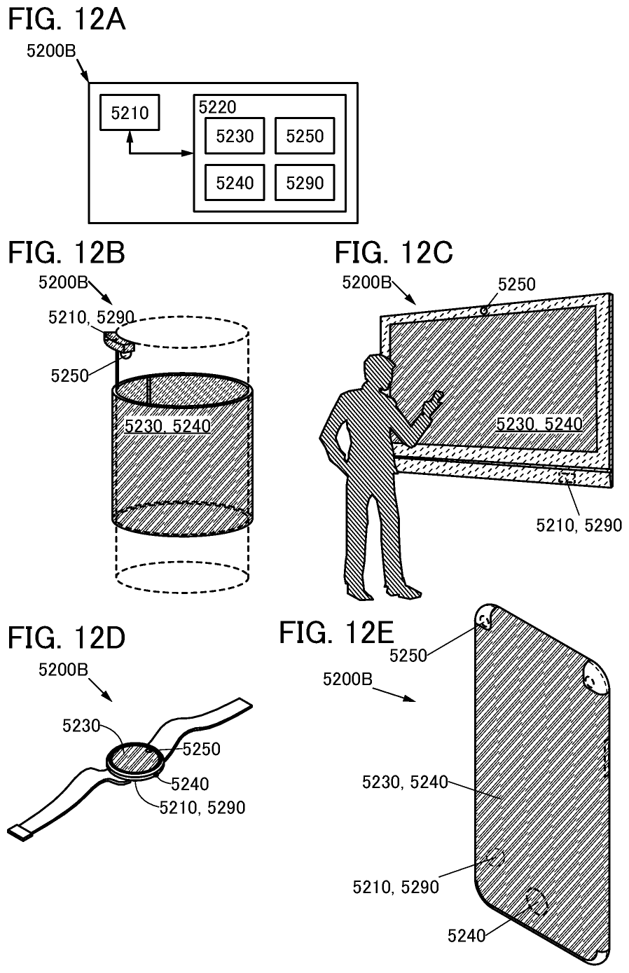

Next, a cross-sectional view of a light-emitting and light-receiving apparatus is shown. FIG. 11 is a cross-sectional view of the light-emitting and light-receiving apparatus illustrated in FIG. 9A.

FIG. 11 is a cross-sectional view of part of a region including the FPC 713 and the wiring 706 and part of the display region 701 including the pixel 703(i,j).

In FIG. 11, the light-emitting and light-receiving apparatus 700 includes the functional layer 520 between the first substrate 510 and the second substrate 770. The functional layer 520 includes, as well as the above-described transistors (M11, M12, M13, M14, M15, M16, and M17), the capacitors (C2 and C3), and the like described with reference to FIGS. 10A to 10C, wirings (VS, VG, V1, V2, V3, V4, and V5) electrically connected to these components, for example. FIG. 11 illustrates a non-limiting example of the functional layer 520 that includes a pixel circuit 530X(i, j), a pixel circuit 530S(i, j), and a driver circuit GD.

Furthermore, each pixel circuit included in the functional layer 520 is electrically connected to a light-emitting device or a light-receiving device. For example, in FIG. 11, the pixel circuit 530X(i, j) and the pixel circuit 530S(i, j) included in the functional layer 520 are electrically connected to a light-emitting device 550X(i,j) and a light-receiving device 550S(i,j), respectively, in FIG. 11 formed over the functional layer 520. Concretely, the light-emitting device 550X(i, j) is electrically connected to the pixel circuit 530X(i,j) through a wiring 591X, and the light-receiving device 550S(i, j) is electrically connected to the pixel circuit 530S(i, j) through a wiring 591S. The insulating layer 705 is provided over the functional layer 520, the light-emitting devices, and the light-receiving device, and has a function of attaching the second substrate 770 and the functional layer 520.

As the second substrate 770, a substrate where touch sensors are arranged in a matrix can be used. For example, a substrate provided with capacitive touch sensors or optical touch sensors can be used as the second substrate 770. Thus, the light-emitting and light-receiving apparatus of one embodiment of the present invention can be used as a touch panel.

The structures described in this embodiment can be used in appropriate combination with any of the structures described in the other embodiments.

### Embodiment 5

This embodiment will describe structures of electronic appliances of embodiments of the present invention with reference to FIGS. 12A to 12E, FIGS. 13A to 13E, and FIGS. 14A and 14B.

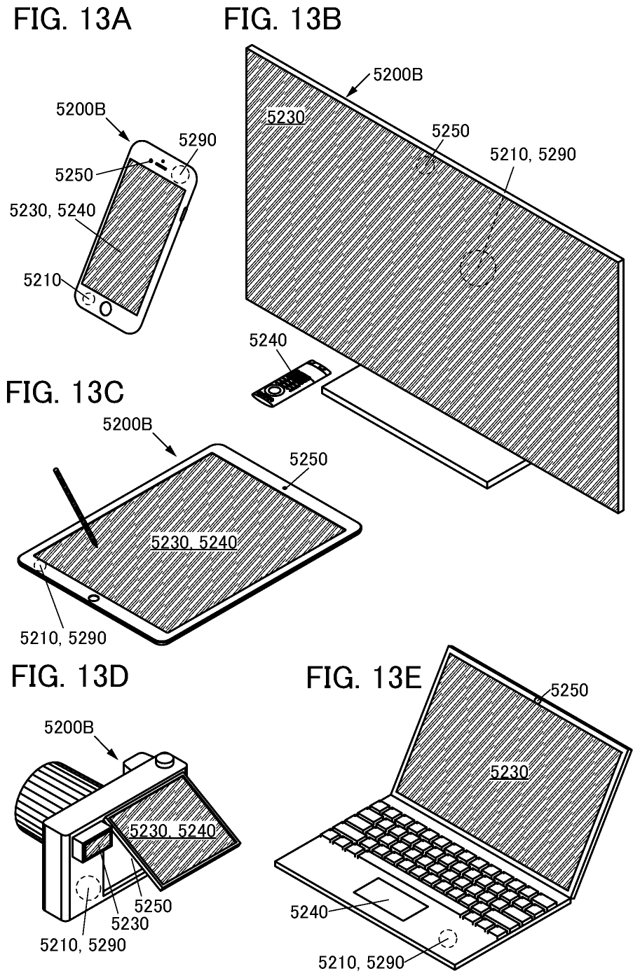

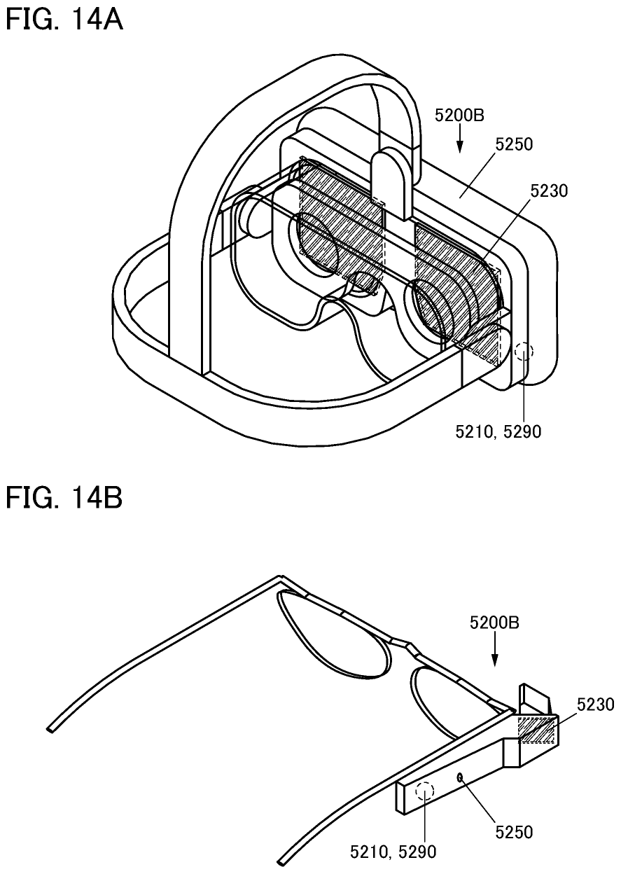

FIGS. 12A to 12E, FIGS. 13A to 13E, and FIGS. 14A and 14B each illustrate a structure of an electronic appliance of one embodiment of the present invention. FIG. 12A is a block diagram of an electronic appliance and FIGS. 12B to 12E are perspective views illustrating structures of electronic appliances. FIGS. 13A to 13E are perspective views illustrating structures of electronic appliances. FIGS. 14A and 14B are perspective views illustrating structures of electronic appliances.

An electronic appliance 5200B described in this embodiment includes an arithmetic device 5210 and an input/output device 5220 (see FIG. 12A).

The arithmetic device 5210 has a function of receiving handling data and a function of supplying image data on the basis of the handling data.

The input/output device 5220 includes a display unit 5230, an input unit 5240, a sensor unit 5250, and a communication unit 5290, and has a function of supplying handling data and a function of receiving image data. The input/output device 5220 also has a function of supplying sensing data, a function of supplying communication data, and a function of receiving communication data.

The input unit 5240 has a function of supplying handling data. For example, the input unit 5240 supplies handling data on the basis of handling by a user of the electronic appliance 5200B.

Specifically, a keyboard, a hardware button, a pointing device, a touch sensor, an illuminance sensor, an imaging device, an audio input device, an eye-gaze input device, an attitude sensing device, or the like can be used as the input unit 5240.

The display unit 5230 includes a display panel and has a function of displaying image data. For example, the display panel described in Embodiment 3 can be used for the display unit 5230.

The sensor unit 5250 has a function of supplying sensing data. For example, the sensor unit 5250 has a function of sensing a surrounding environment where the electronic appliance is used and supplying the sensing data.

Specifically, an illuminance sensor, an imaging device, an attitude sensing device, a pressure sensor, a human motion sensor, or the like can be used as the sensor unit 5250.

The communication unit 5290 has a function of receiving and supplying communication data. For example, the communication unit 5290 has a function of being connected to another electronic appliance or a communication network by wireless communication or wired communication. Specifically, the communication unit 5290 has a function of wireless local area network communication, telephone communication, near field communication, or the like.

FIG. 12B illustrates an electronic appliance having an outer shape along a cylindrical column or the like. An example of such an electronic appliance is digital signage. The display panel of one embodiment of the present invention can be used for the display unit 5230. The electronic appliance may have a function of changing its display method in accordance with the illuminance of a usage environment. The electronic appliance has a function of changing the displayed content when sensing the existence of a person. Thus, for example, the electronic appliance can be provided on a column of a building. The electronic appliance can display advertising, guidance, or the like.

FIG. 12C illustrates an electronic appliance having a function of generating image data on the basis of the path of a pointer used by the user. Examples of such an electronic appliance include an electronic blackboard, an electronic bulletin board, and digital signage. Specifically, a display panel with a diagonal size of 20 inches or longer, preferably 40 inches or longer, further preferably 55 inches or longer can be used. A plurality of display panels can be arranged and used as one display region. Alternatively, a plurality of display panels can be arranged and used as a multiscreen.

FIG. 12D illustrates an electronic appliance that is capable of receiving data from another device and displaying the data on the display unit 5230. An example of such an electronic appliance is a wearable electronic appliance. Specifically, the electronic appliance can display several options, and the user can choose some from the options and send a reply to the data transmitter. As another example, the electronic appliance has a function of changing its display method in accordance with the illuminance of a usage environment. Thus, for example, power consumption of the wearable electronic appliance can be reduced. As another example, the wearable electronic appliance can display an image so as to be suitably used even in an environment under strong external light, e.g., outdoors in fine weather.

FIG. 12E illustrates an electronic appliance including the display unit 5230 having a surface gently curved along a side surface of a housing. An example of such an electronic appliance is a mobile phone. The display unit 5230 includes a display panel that has a function of displaying images on the front surface, the side surfaces, the top surface, and the rear surface, for example. Thus, a mobile phone can display data on not only its front surface but also its side surfaces, top surface, and rear surface, for example.

FIG. 13A illustrates an electronic appliance that is capable of receiving data via the Internet and displaying the data on the display unit 5230. An example of such an electronic appliance is a smartphone. For example, the user can check a created message on the display unit 5230 and send the created message to another device. As another example, the electronic appliance has a function of changing its display method in accordance with the illuminance of a usage environment. Thus, power consumption of the smartphone can be reduced. As another example, it is possible to obtain a smartphone which can display an image such that the smartphone can be suitably used in an environment under strong external light, e.g., outdoors in fine weather.

FIG. 13B illustrates an electronic appliance that can use a remote controller as the input unit 5240. An example of such an electronic appliance is a television system. For example, data received from a broadcast station or via the Internet can be displayed on the display unit 5230. The electronic appliance can take an image of the user with the sensor unit 5250 and transmit the image of the user. The electronic appliance can acquire a viewing history of the user and provide it to a cloud service. The electronic appliance can acquire recommendation data from a cloud service and display the data on the display unit 5230. A program or a moving image can be displayed on the basis of the recommendation data. As another example, the electronic appliance has a function of changing its display method in accordance with the illuminance of a usage environment. Accordingly, for example, it is possible to obtain a television system which can display an image such that the television system can be suitably used even under strong external light entering the room from the outside in fine weather.

FIG. 13C illustrates an electronic appliance that is capable of receiving an educational material via the Internet and displaying it on the display unit 5230. An example of such an electronic appliance is a tablet computer. The user can input an assignment with the input unit 5240 and send it via the Internet. The user can obtain a corrected assignment or the evaluation from a cloud service and have it displayed on the display unit 5230. The user can select a suitable educational material on the basis of the evaluation and have it displayed.

For example, an image signal can be received from another electronic appliance and displayed on the display unit 5230. When the electronic appliance is placed on a stand or the like, the display unit 5230 can be used as a sub-display. Thus, for example, it is possible to obtain a tablet computer which can display an image such that the tablet computer is favorably used even in an environment under strong external light, e.g., outdoors in fine weather.

FIG. 13D illustrates an electronic appliance including a plurality of display units 5230. An example of such an electronic appliance is a digital camera. For example, the display unit 5230 can display an image that the sensor unit 5250 is capturing. A captured image can be displayed on the sensor unit. A captured image can be decorated using the input unit 5240. A message can be attached to a captured image. A captured image can be transmitted via the Internet. The electronic appliance has a function of changing shooting conditions in accordance with the illuminance of a usage environment. Accordingly, for example, it is possible to obtain a digital camera that can display a subject such that an image is favorably viewed even in an environment under strong external light, e.g., outdoors in fine weather.

FIG. 13E illustrates an electronic appliance in which the electronic appliance of this embodiment is used as a master to control another electronic appliance used as a slave. An example of such an electronic appliance is a portable personal computer. For example, part of image data can be displayed on the display unit 5230 and another part of the image data can be displayed on a display unit of another electronic appliance. Image signals can be supplied. Data written from an input unit of another electronic appliance can be obtained with the communication unit 5290. Thus, a large display region can be utilized in the case of using a portable personal computer, for example.

FIG. 14A illustrates an electronic appliance including the sensor unit 5250 that senses an acceleration or a direction. An example of such an electronic appliance is a goggles-type electronic appliance. The sensor unit 5250 can supply data on the position of the user or the direction in which the user faces. The electronic appliance can generate image data for the right eye and image data for the left eye in accordance with the position of the user or the direction in which the user faces. The display unit 5230 includes a display region for the right eye and a display region for the left eye. Thus, a virtual reality image that gives the user a sense of immersion can be displayed on the goggles-type electronic appliance, for example.

FIG. 14B illustrates an electronic appliance including an imaging device and the sensor unit 5250 that senses an acceleration or a direction. An example of such an electronic appliance is a glasses-type electronic appliance. The sensor unit 5250 can supply data on the position of the user or the direction in which the user faces. The electronic appliance can generate image data in accordance with the position of the user or the direction in which the user faces. Accordingly, the data can be shown together with a real-world scene, for example. Alternatively, an augmented reality image can be displayed on the glasses-type electronic appliance.

Note that this embodiment can be combined with any of the other embodiments in this specification as appropriate.

### Embodiment 6

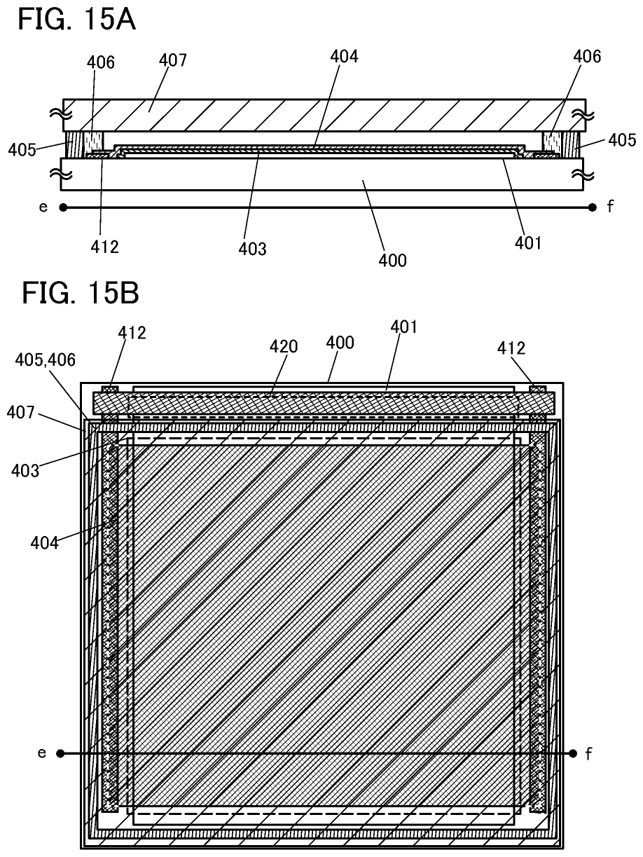

This embodiment will describe a structure in which any of the light-emitting devices described in Embodiments 1 and 2 is used as a lighting device with reference to FIGS. 15A and 15B. FIG. 15A illustrates a cross section taken along the line e-f in a top view of the lighting device in FIG. 15B.

In the lighting device in this embodiment, a first electrode 401 is formed over a substrate 400 that is a support and has a light-transmitting property. The first electrode 401 corresponds to the first electrode 101 in Embodiments 1 and 2. When light is extracted from the first electrode 401 side, the first electrode 401 is formed using a material having a light-transmitting property.

A pad 412 for applying voltage to a second electrode 404 is provided over the substrate 400.

An EL layer 403 is formed over the first electrode 401. The structure of the EL layer 403 corresponds to the structure of the EL layer 103 in Embodiments 1 and 2. Refer to the corresponding description for these structures.

The second electrode 404 is formed to cover the EL layer 403. The second electrode 404 corresponds to the second electrode 102 in Embodiments 1 and 2. The second electrode 404 is formed using a material having high reflectance when light is extracted from the first electrode 401 side. The second electrode 404 is connected to the pad 412 so that voltage is applied to the second electrode 404.

As described above, the lighting device described in this embodiment includes a light-emitting device including the first electrode 401, the EL layer 403, and the second electrode 404. Since the light-emitting device has high emission efficiency, the lighting device in this embodiment can have low power consumption.

The substrate 400 provided with the light-emitting device having the above structure and a sealing substrate 407 are fixed and sealed with sealing materials 405 and 406, whereby the lighting device is completed. It is possible to use only either the sealing material 405 or the sealing material 406. In addition, the inner sealing material 406 (not illustrated in FIG. 15B) can be mixed with a desiccant that enables moisture to be adsorbed, increasing the reliability.

When parts of the pad 412 and the first electrode 401 are extended to the outside of the sealing materials 405 and 406, the extended parts can serve as external input terminals. An IC chip 420 mounted with a converter or the like may be provided over the external input terminals.

### Embodiment 7

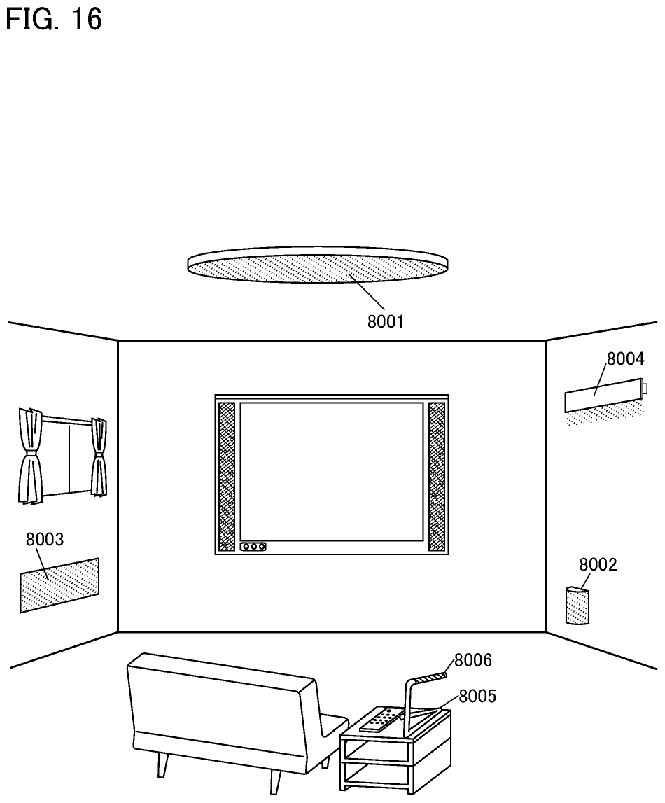

This embodiment will describe application examples of lighting devices fabricated using the light-emitting apparatus of one embodiment of the present invention or the light-emitting device, which is part of the light-emitting apparatus with reference to FIG. 16.

A ceiling light 8001 can be used as an indoor lighting device. Examples of the ceiling light 8001 include a direct-mount light and an embedded light. Such lighting devices are fabricated using the light-emitting apparatus and a housing and a cover in combination. Application to a cord pendant light (light that is suspended from a ceiling by a cord) is also possible.

A foot light 8002 lights a floor so that safety on the floor can be improved. For example, it can be effectively used in a bedroom, on a staircase, and on a passage. In such cases, the size and shape of the foot light can be changed in accordance with the dimensions and structure of a room. The foot light can be a stationary lighting device using the light-emitting apparatus and a support in combination.

A sheet-like lighting 8003 is a thin sheet-like lighting device. The sheet-like lighting, which is attached to a wall when used, is space-saving and thus can be used for a wide variety of uses. Furthermore, the area of the sheet-like lighting can be easily increased. The sheet-like lighting can also be used on an object such as a wall or a housing that has a curved surface.

A lighting device 8004 in which the direction of light from a light source is controlled to be only a desired direction can be used.

A desk lamp 8005 includes a light source 8006. As the light source 8006, the light-emitting apparatus of one embodiment of the present invention or the light-emitting device, which is part of the light-emitting apparatus, can be used.

Besides the above examples, when the light-emitting apparatus of one embodiment of the present invention or the light-emitting device, which is part of the light-emitting apparatus, is used as part of furniture in a room, a lighting device that functions as the furniture can be obtained.

As described above, a variety of lighting devices that include the light-emitting apparatus can be obtained. Note that these lighting devices are also embodiments of the present invention.

The structures described in this embodiment can be used in appropriate combination with any of the structures described in the other embodiments.

### Embodiment 8

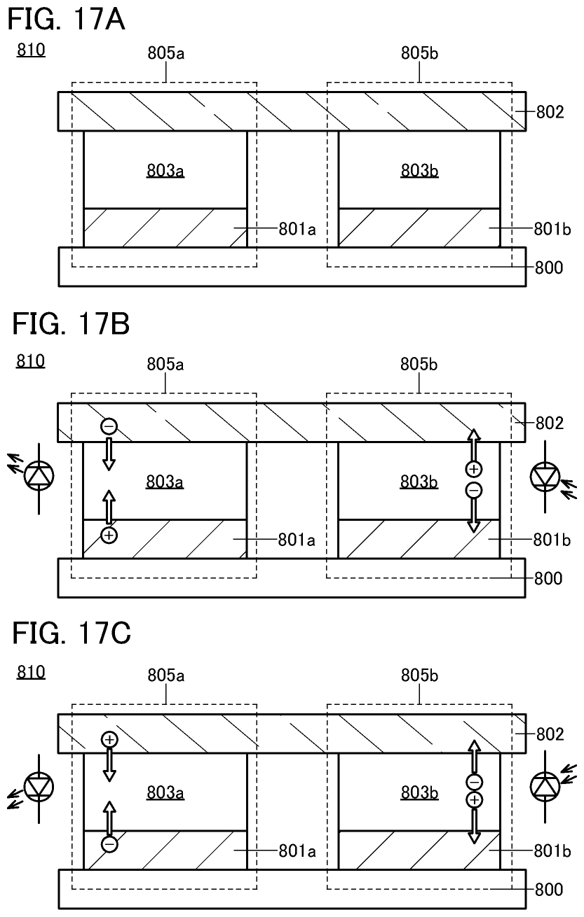

This embodiment will describe a light-emitting and light-receiving apparatus 810 with reference to FIGS. 17A to 17C, for description of a light-emitting device and a light-receiving device that can be used in a light-emitting apparatus of one embodiment of the present invention. The light-emitting and light-receiving apparatus 810 includes a light-emitting device and thus can be regarded as a light-emitting apparatus; includes a light-receiving device and thus can be regarded as a light-receiving apparatus; and can be used in a display unit in an electronic appliance and thus can be regarded as a display panel or a display device.

FIG. 17A is a schematic cross-sectional view of a light-emitting device 805a and a light-receiving device 805b included in the light-emitting and light-receiving apparatus 810 of one embodiment of the present invention.

The light-emitting device 805a has a function of emitting light (hereinafter, also referred to as a light-emitting function). The light-emitting device 805a includes an electrode 801a, an EL layer 803a, and an electrode 802. The light-emitting device 805a is preferably a light-emitting device utilizing organic EL (an organic EL device) described in Embodiments 1 and 2. Thus, the EL layer 803a interposed between the electrode 801a and the electrode 802 includes at least a light-emitting layer. The light-emitting layer contains a light-emitting substance. The EL layer 803a emits light when voltage is applied between the electrode 801a and the electrode 802. The EL layer 803a may include any of a variety of layers such as a hole-injection layer, a hole-transport layer, an electron-transport layer, an electron-injection layer, a carrier-blocking (hole-blocking or electron-blocking) layer, and a charge-generation layer, in addition to the light-emitting layer. The first organic compound described in Embodiment 1 can be used for any layer in the EL layer 803a.

The light-receiving device 805b has a function of sensing light (hereinafter, also referred to as a light-receiving function). As the light-receiving device 805b, a PN photodiode or a PIN photodiode can be used, for example. The light-receiving device 805b includes an electrode 801b, a light-receiving layer 803b, and the electrode 802. Thus, the light-receiving layer 803b interposed between the electrode 801b and the electrode 802 includes at least an active layer. Note that for the light-receiving layer 803b, any of materials that are used for the variety of layers (e.g., the hole-injection layer, the hole-transport layer, the light-emitting layer, the electron-transport layer, the electron-injection layer, the carrier-blocking (hole-blocking or electron-blocking) layer, and the charge-generation layer) included in the above-described EL layer 803a can be used. The first organic compound described in Embodiment 1 can be used for any layer in the light-receiving layer 803b. The light-receiving device 805b functions as a photoelectric conversion device. When light is incident on the light-receiving layer 803b, electric charge can be generated and extracted as a current. At this time, voltage may be applied between the electrode 801b and the electrode 802. The amount of generated electric charge depends on the amount of the light incident on the light-receiving layer 803b.

The light-receiving device 805b has a function of sensing visible light. The light-receiving device 805b has sensitivity to visible light. The light-receiving device 805b further preferably has a function of sensing visible light and infrared light. The light-receiving device 805b preferably has sensitivity to visible light and infrared light.

In this specification and the like, a blue (B) wavelength region ranges from 400 nm to less than 490 nm, and blue (B) light has at least one emission spectrum peak in the wavelength region. A green (G) wavelength region ranges from 490 nm to less than 580 nm, and green (G) light has at least one emission spectrum peak in the wavelength region. A red (R) wavelength region ranges from 580 nm to less than 700 nm, and red (R) light has at least one emission spectrum peak in the wavelength region. In this specification and the like, a visible wavelength region ranges from 400 nm to less than 700 nm, and visible light has at least one emission spectrum peak in the wavelength region. An infrared (IR) wavelength region ranges from 700 nm to less than 900 nm, and infrared (IR) light has at least one emission spectrum peak in the wavelength region.

The active layer in the light-receiving device 805b includes a semiconductor. Examples of the semiconductor are inorganic semiconductors such as silicon and organic semiconductors such as organic compounds. As the light-receiving device 805b, an organic semiconductor device (or an organic photodiode) including an organic semiconductor in the active layer is preferably used. An organic photodiode, which is easily made thin, lightweight, and large in area and has a high degree of freedom for shape and design, can be used in a variety of display devices. An organic semiconductor is preferably used, in which case the EL layer 803a included in the light-emitting device 805a and the light-receiving layer 803b included in the light-receiving device 805b can be formed by the same method (e.g., a vacuum evaporation method) with the same manufacturing apparatus. Note that any of the organic compounds of one embodiment of the present invention can be used for the light-receiving layer 803b in the light-receiving device 805b.

In the display device of one embodiment of the present invention, an organic EL device and an organic photodiode can be suitably used as the light-emitting device 805a and the light-receiving device 805b, respectively. The organic EL device and the organic photodiode can be formed over one substrate. Thus, the organic photodiode can be incorporated into the display device including the organic EL device. A display device of one embodiment of the present invention has one or both of an image capturing function and a sensing function in addition to a function of displaying an image.

The electrode 801a and the electrode 801b are provided on the same plane. In FIG. 17A, the electrodes 801a and 801b are provided over a substrate 800. The electrodes 801a and 801b can be formed by processing a conductive film formed over the substrate 800 into island shapes, for example. In other words, the electrodes 801a and 801b can be formed through the same process.

As the substrate 800, a substrate having heat resistance high enough to withstand the formation of the light-emitting device 805a and the light-receiving device 805b can be used. When an insulating substrate is used, a glass substrate, a quartz substrate, a sapphire substrate, a ceramic substrate, an organic resin substrate, or the like can be used as the substrate 800. Alternatively, a semiconductor substrate can be used. For example, a single crystal semiconductor substrate or a polycrystalline semiconductor substrate of silicon, silicon carbide, or the like; a compound semiconductor substrate of silicon germanium or the like; an SOI substrate; or the like can be used.

In particular, it is preferable to use, as the substrate 800, the insulating substrate or the semiconductor substrate over which a semiconductor circuit including a semiconductor element such as a transistor is formed. The semiconductor circuit preferably constitutes part of a pixel circuit, a gate line driver circuit (a gate driver), a source line driver circuit (a source driver), or the like. In addition to the above, the semiconductor circuit may constitute part of an arithmetic circuit, a memory circuit, or the like.

The electrode 802 is formed of a layer shared by the light-emitting device 805a and the light-receiving device 805b. As the electrode through which light enters or exits, a conductive film that transmits visible light and infrared light is used. As the electrode through which light neither enters nor exits, a conductive film that reflects visible light and infrared light is preferably used.

The electrode 802 in the display device of one embodiment of the present invention functions as one of the electrodes in each of the light-emitting device 805a and the light-receiving device 805b.

In FIG. 17B, the electrode 801a of the light-emitting device 805a has a potential higher than the electrode 802. In this case, the electrode 801a and the electrode 802 function as an anode and a cathode, respectively, in the light-emitting device 805a. The electrode 801b of the light-receiving device 805b has a potential lower than the electrode 802. For easy understanding of the direction of current flow, FIG. 17B illustrates a circuit symbol of a light-emitting diode on the left of the light-emitting device 805a and a circuit symbol of a photodiode on the right of the light-receiving device 805b. The flow directions of carriers (electrons and holes) in each device are also schematically indicated by arrows.

In the structure illustrated in FIG. 17B, when a first potential is supplied to the electrode 801a through a first wiring, a second potential is supplied to the electrode 802 through a second wiring, and a third potential is supplied to the electrode 801b through a third wiring, the following relationship is satisfied: the first potential >the second potential >the third potential.

In FIG. 17C, the electrode 801a of the light-emitting device 805a has a potential lower than the electrode 802. In this case, the electrode 801a and the electrode 802 function as a cathode and an anode, respectively, in the light-emitting device 805a. The electrode 801b of the light-receiving device 805b has a potential lower than the potential of the electrode 802 and a potential higher than the potential of the electrode 801a. For easy understanding of the direction of current flow, FIG. 17C illustrates a circuit symbol of a light-emitting diode on the left of the light-emitting device 805a and a circuit symbol of a photodiode on the right of the light-receiving device 805b. The flow directions of carriers (electrons and holes) in each device are also schematically indicated by arrows.

In the structure illustrated in FIG. 17C, when a first potential is supplied to the electrode 801a through a first wiring, a second potential is supplied to the electrode 802 through a second wiring, and a third potential is supplied to the electrode 801b through a third wiring, the following relationship is satisfied: the second potential >the third potential >the first potential.

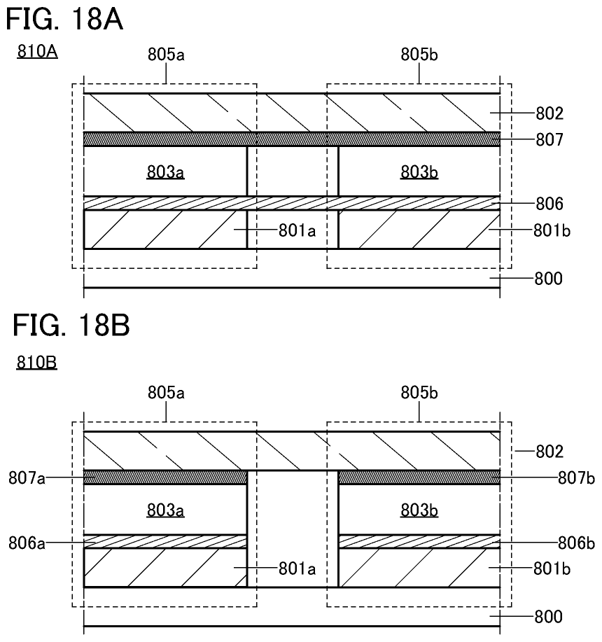

FIG. 18A illustrates a light-emitting and light-receiving apparatus 810A that is a variation example of the light-emitting and light-receiving apparatus 810. The light-emitting and light-receiving apparatus 810A is different from the light-emitting and light-receiving apparatus 810 in including a common layer 806 and a common layer 807. In the light-emitting device 805a, the common layers 806 and 807 function as part of the EL layer 803a. In the light-receiving device 805b, the common layers 806 and 807 function as part of the light-receiving layer 803b. The common layer 806 includes a hole-injection layer and a hole-transport layer, for example. The common layer 807 includes an electron-transport layer and an electron-injection layer, for example.

With the common layers 806 and 807, a light-receiving device can be incorporated without a significant increase in the number of times of separate formation of devices, whereby the light-emitting and light-receiving apparatus 810A can be manufactured with a high throughput. The first organic compound described in Embodiment 1 can be used for any of the common layer 806 and the common layer 807.

FIG. 18B illustrates a light-emitting and light-receiving apparatus 810B that is a variation example of the light-emitting and light-receiving apparatus 810. The light-emitting and light-receiving apparatus 810B is different from the light-emitting and light-receiving apparatus 810 in that the EL layer 803a includes a layer 806a and a layer 807a and the light-receiving layer 803b includes a layer 806b and a layer 807b. The layers 806a and 806b are formed using different materials, and each include a hole-injection layer and a hole-transport layer, for example. Note that the layers 806a and 806b may be formed using a common material. The layers 807a and 807b are formed using different materials, and each include an electron-transport layer and an electron-injection layer, for example. Note that the layers 807a and 807b may be formed using a common material.

An optimum material for forming the light-emitting device 805a is selected for the layers 806a and 807a and an optimum material for forming the light-receiving device 805b is selected for the layers 806b and 807b, whereby the light-emitting device 805a and the light-receiving device 805b can have higher performance in the light-emitting and light-receiving apparatus 810B.

The resolution of the light-receiving device 805b described in this embodiment can be 100 ppi or higher, preferably 200 ppi or higher, further preferably 300 ppi or higher, still further preferably 400 ppi or higher, and still further preferably 500 ppi or higher, and 2000 ppi or lower, 1000 ppi or lower, or 600 ppi or lower, for example. In particular, when the resolution of the light-receiving device 805b is 200 ppi or higher and 600 ppi or lower, preferably 300 ppi or higher and 600 ppi or lower, the display device of one embodiment of the present invention can be suitably applied to image capturing of fingerprints. In fingerprint authentication with the display device of one embodiment of the present invention, the increased resolution of the light-receiving device 805b enables, for example, high accuracy extraction of the minutiae of fingerprints; thus, the accuracy of the fingerprint authentication can be increased. The resolution is preferably 500 ppi or higher, in which case the authentication conforms to the standard by the National Institute of Standards and Technology (NIST) or the like. On the assumption that the resolution of the light-receiving device is 500 ppi, the size of each pixel is 50.8 μm, which is adequate for image capturing of a fingerprint ridge distance (typically, greater than or equal to 300 μm and less than or equal to 500 μm).

The structures described in this embodiment can be used in appropriate combination with any of the structures described in the other embodiments.

### Example 1

### Synthesis Example 1

This example will describe a method for synthesizing N-(9,9-dimethyl-9H-fluoren-2-yl)-N-(9,9-dimethyl-9H-fluoren-4-yl)dibenzothiophen-4-amine (abbreviation: ThFAF(4)), which is the organic compound represented by Structural Formula (400) in Embodiment 1. The structure of ThFAF(4) is shown below.

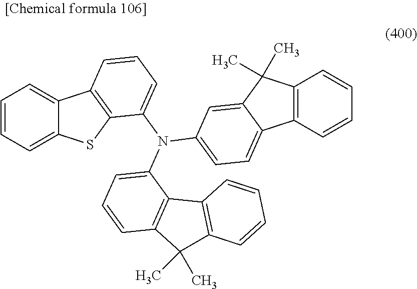 [SMILES: CC1(C)c2ccccc2-c2ccc(N(c3cccc4c3-c3ccccc3C4(C)C)c3cccc4c3sc3ccccc34)cc21]

### Step 1: Synthesis of N-(9,9-dimethyl-9H-fluoren-4-yl)-9-,9-dimethyl-9H-fluoren-2-amine

Into a 200 mL three-neck flask were put 6.13 g (29.3 mmol) of 2-amino-9,9-dimethyl-9H-fluorene, 7.55 g (27.7 mmol) of 2-bromo-9,9-dimethyl-9H-fluorene, and 8.21 g (85.5 mmol) of sodium-tert-butoxide. The air in the flask was replaced with nitrogen. Then, 145.0 mL of toluene and 0.5 mL of a 10% hexane solution of tri(tert-butyl)phosphine were added to this mixture. The temperature of the mixture was set to 60° C., 73.0 mg (0.1 mmol) of bis(dibenzylideneacetone)palladium(0) was added, and the mixture was stirred at 80° C. for 3.3 hours. After the stirring, toluene was added to the mixture, and the mixture was suction-filtered through Florisil, Celite, and alumina to give a filtrate. The obtained filtrate was concentrated to give an oily substance. The obtained oily substance was purified by silica gel column chromatography (developing solvent, hexane:toluene=4:1) to give 8.31 g of a target compound in a yield of 72%. The synthesis scheme of Step 1 is shown in (a-1) below.

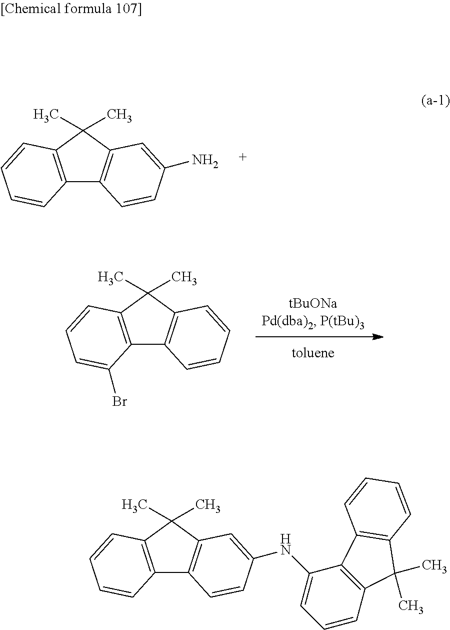 [SMILES: CC1(C)c2ccccc2-c2ccc(N)cc21 ; CC1(C)c2ccccc2-c2c(Br)cccc21 ; CC1(C)c2ccccc2-c2ccc(Nc3cccc4c3-c3ccccc3C4(C)C)cc21]

### Step 2: Synthesis of ThFAF(4)

Into a 200-mL three-neck flask was put 2.01 g (5.0 mmol) of N-(9, 9-dimethyl-9H-fluoren-4-yl)-9,9-dimethyl-9H-fluoren-2-amine, 1.34 g (5.1 mmol) of 4-bromo-dibenzothiophene, and 1.48 g (15.4 mmol) of sodium-tert-butoxide. The air in the flask was replaced with nitrogen. To this mixture were added 25.6 mL of xylene and 0.5 mL of a 10% hexane solution of tri(tert-butyl)phosphine. The temperature of this mixture was set to 60° C., and 60.7 mg (0.1 mmol) of bis(dibenzylideneacetone)palladium(0) was added to the mixture. The temperature of this mixture was raised to 90° C., followed by stirring for 2.7 hours. After the stirring, the mixture was suction-filtered through Florisil, Celite, and alumina to give a filtrate. The obtained filtrate was concentrated to give a solid. The obtained solid was purified by silica gel column chromatography (developing solvent, hexane:toluene=4:1) to give 2.27 g of a target solid in a yield of 78%.

Then, 2.2 g of the obtained solid was purified by a train sublimation method. In the purification by sublimation, the solid was heated at 240° C. under a pressure of 3.0 Pa with an argon gas flow rate of 12.0 mL/min. After the purification by sublimation, 1.9 g of a target solid, was obtained at a collection rate of 87%. Then, 1.8 g of the obtained solid was purified again by train sublimation. In the purification by sublimation, the solid was heated at 220° C. under a pressure of 3.0 Pa with an argon gas flow rate of 12.0 mL/min. After the sublimation purification, 1.7 g of a target solid was obtained at a collection rate of 94%. The synthesis scheme of Step 2 is shown in (a-2) below.

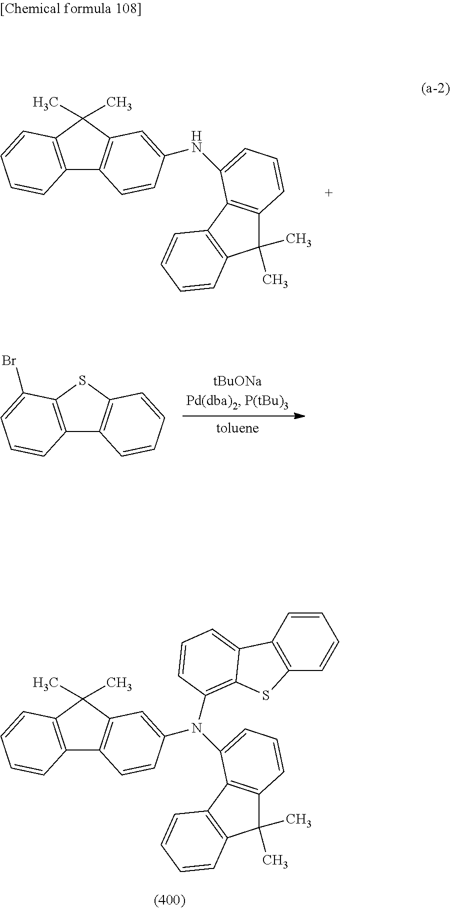 [SMILES: CC1(C)c2ccccc2-c2ccc(Nc3cccc4c3-c3ccccc3C4(C)C)cc21 ; Brc1cccc2c1sc1ccccc12 ; CC1(C)c2ccccc2-c2ccc(N(c3cccc4c3-c3ccccc3C4(C)C)c3cccc4c3sc3ccccc34)cc21]

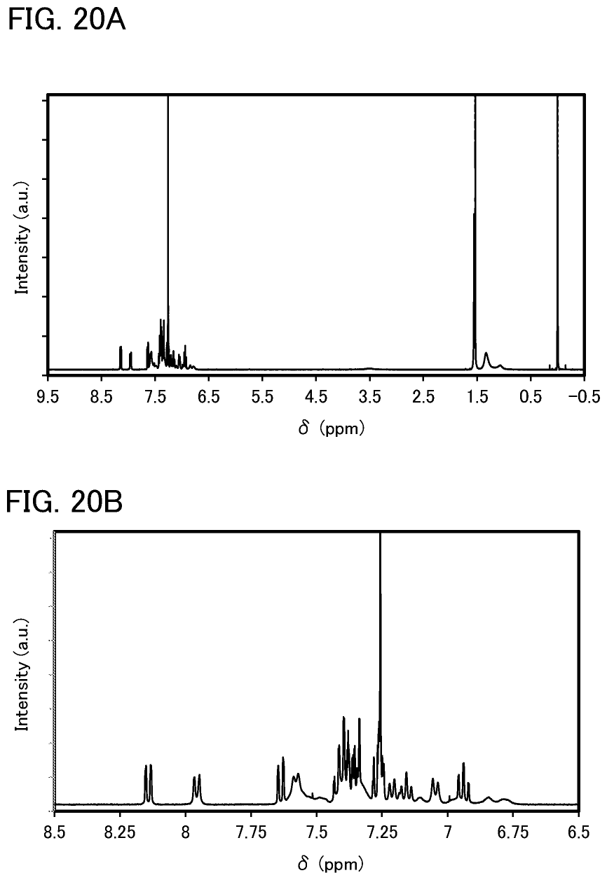

Analysis results by nuclear magnetic resonance spectroscopy (¹H-NMR) of the yellow solid obtained in Step 2 are shown below. FIGS. 20A and 20B show the ¹H-NMR chart. The result revealed that ThFAF(4) (Structural formula (400)) was obtained in this synthesis example.

¹H-NMR (CDCl₃, 400 MHz): δ=1.07-1.56 (m, 12H), 6.78-7.65 (m, 19H), 7.96 (d, J=7.6 Hz, 1H), 8.14 (d, J=7.5 Hz, 1H).

### Example 2

### Synthesis Example 2

This example will describe a method for synthesizing N-(9,9-dimethyl-9H-fluoren-2-yl)-N-(9,9-dimethyl-9H-fluoren-4-yl)-dibenzofuran-4-amine (abbreviation: FrFAF(4)), which is the organic compound represented by Structural Formula (200) in Embodiment 1. The structure of FrFAF(4) is shown below.

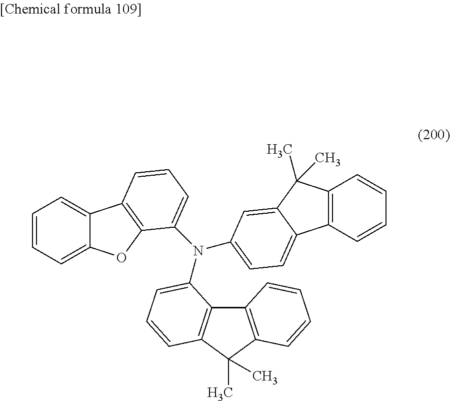 [SMILES: CC1(C)c2ccccc2-c2ccc(N(c3cccc4c3-c3ccccc3C4(C)C)c3cccc4c3oc3ccccc34)cc21]

### Step 2: Synthesis of FrFAF(4)

Into a 200-mL three-neck flask was put 2.12 g (5.28 mmol) of N-(9, 9-dimethyl-9H-fluoren-4-yl)-9,9-dimethyl-9H-fluoren-2-amine, 1.30 g (5.28 mmol) of 4-bromodibenzofuran, 1.52 g (15.8 mmol) of sodium-tert-butoxide. The air in the flask was replaced with nitrogen. To this mixture were added 27 mL of toluene and 0.3 mL of a 10% hexane solution of tri-t-butylphosphine, and degassing was performed while the mixture in the flask was stirred under reduced pressure. The temperature of this mixture was stirred at 60° C., and 0.04 g (0.066 mmol) of bis(dibenzylideneacetone)palladium(0) (abbreviation: Pd(dba)₂) was added to the mixture and the obtained mixture was stirred at 90° C. for three hours. After a predetermined time elapsed, the obtained mixture was filtered through a filter aid in which Celite, alumina, Celite, Florisil, and Celite were stacked in this order and the solvent was distilled off. The given residue was purified by flash column chromatography in which a hexane-toluene mixed solvent was used as a developing solvent. The resulting fraction was concentrated to give 2.93 g of the target yellow oily substance. The obtained yellow oily substance was recrystallized with toluene/ethanol and washed with hexane. In this manner, 2.21 g of a target yellowish white solid was obtained in a yield of 74%.

By a train sublimation method, 2.21 g of the obtained yellowish white solid was purified. In the purification by sublimation, the solid was heated at 215° C. for 20 hours and then at 225° C. for 22 hours under a pressure of 2.54 Pa and with an argon gas flow rate of 15 mL/min. After the sublimation purification, 1.43 g of a target yellow solid was obtained at a collection rate of 65%. The synthesis scheme of Step 1 is shown in (b-1) below.

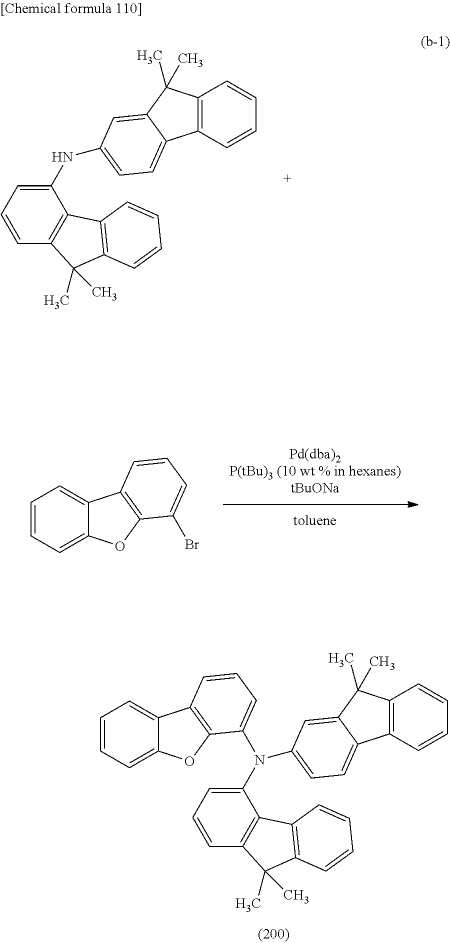 [SMILES: CC1(C)c2ccccc2-c2ccc(Nc3cccc4c3-c3ccccc3C4(C)C)cc21 ; Brc1cccc2c1oc1ccccc12 ; CC1(C)c2ccccc2-c2ccc(N(c3cccc4c3-c3ccccc3C4(C)C)c3cccc4c3oc3ccccc34)cc21]

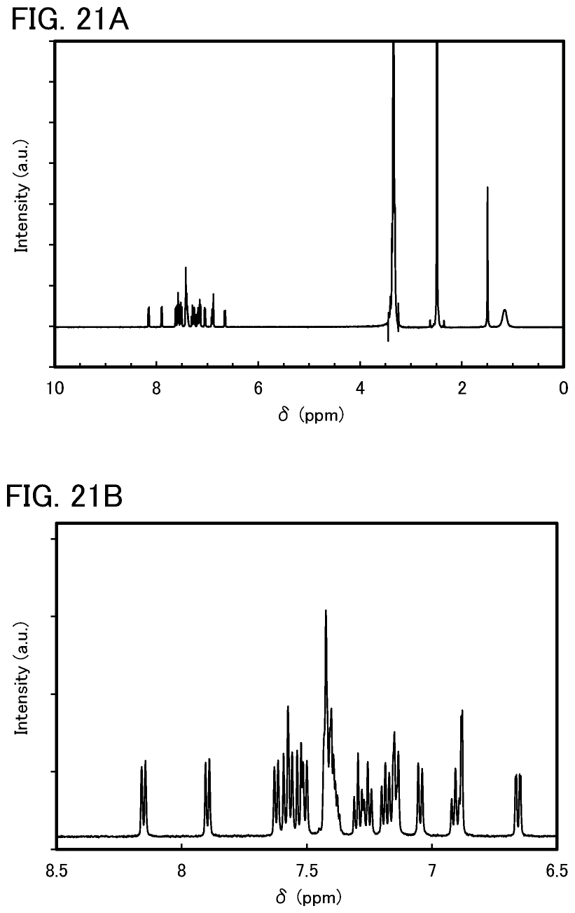

Analysis results by nuclear magnetic resonance (¹H-NMR) spectroscopy of the yellow solid obtained in Step 1 are shown below. The ¹H-NMR chart is shown in FIGS. 21A and 21B. The result revealed that FrFAF(4) (Structural formula (200)) was obtained in this synthesis example.

¹H-NMR. δ (DMSO-d₆, 500 MHz): 1.16 (s, 6H), 1.50 (s, 6H), 6.66 (dd, J1=8.59 Hz, J2=2.29 Hz, 1H), 6.88-6.92 (m, 2H), 7.05 (d, J=8.02 Hz, 1H), 7.13-7.20 (m, 3H), 7.24-7.31 (m, 2H), 7.37-7.43 (m, 5H), 7.50-7.63 (m, 5H), 7.90 (d, J=7.45 Hz, 1H), 8.15 (d, J=7.45 Hz, 1H).

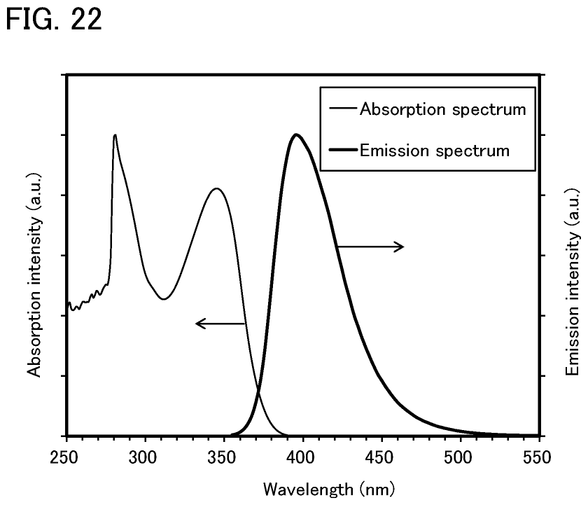

Next, an ultraviolet-visible absorption spectrum (hereinafter, simply referred to as an absorption spectrum) and an emission spectrum of FrFAF(4) in a toluene solution were measured. The absorption spectrum was measured with an ultraviolet-visible spectrophotometer (V-550, produced by JASCO Corporation). The emission spectrum was measured with a spectrofluorometer (FP-8600DS, produced by JASCO Corporation). FIG. 22 shows measurement results of the absorption spectrum and the emission spectrum. The horizontal axis represents the wavelength and the vertical axes represent the absorption intensity and the emission intensity. Note that the measurement was performed at room temperature. The absorption intensity shown in FIG. 22 is a result obtained by subtraction of an absorption spectrum of only toluene in a quartz cell from the measured absorption spectrum of the toluene solution in the quartz cell.

As shown in FIG. 22, the absorption peak and the emission peak of FrFAF(4) were observed at 345 nm and at 396 nm, respectively.

### Example 3

In this example, light-emitting devices 1 to 3, which are fluorescent light-emitting devices of embodiments of the present invention, were fabricated, and comparison results of the device characteristics are shown. Structural formulae of organic compounds used for the light-emitting devices 1 to 3 are shown below. In addition, device structures of the light-emitting devices 1 to 3 are shown.

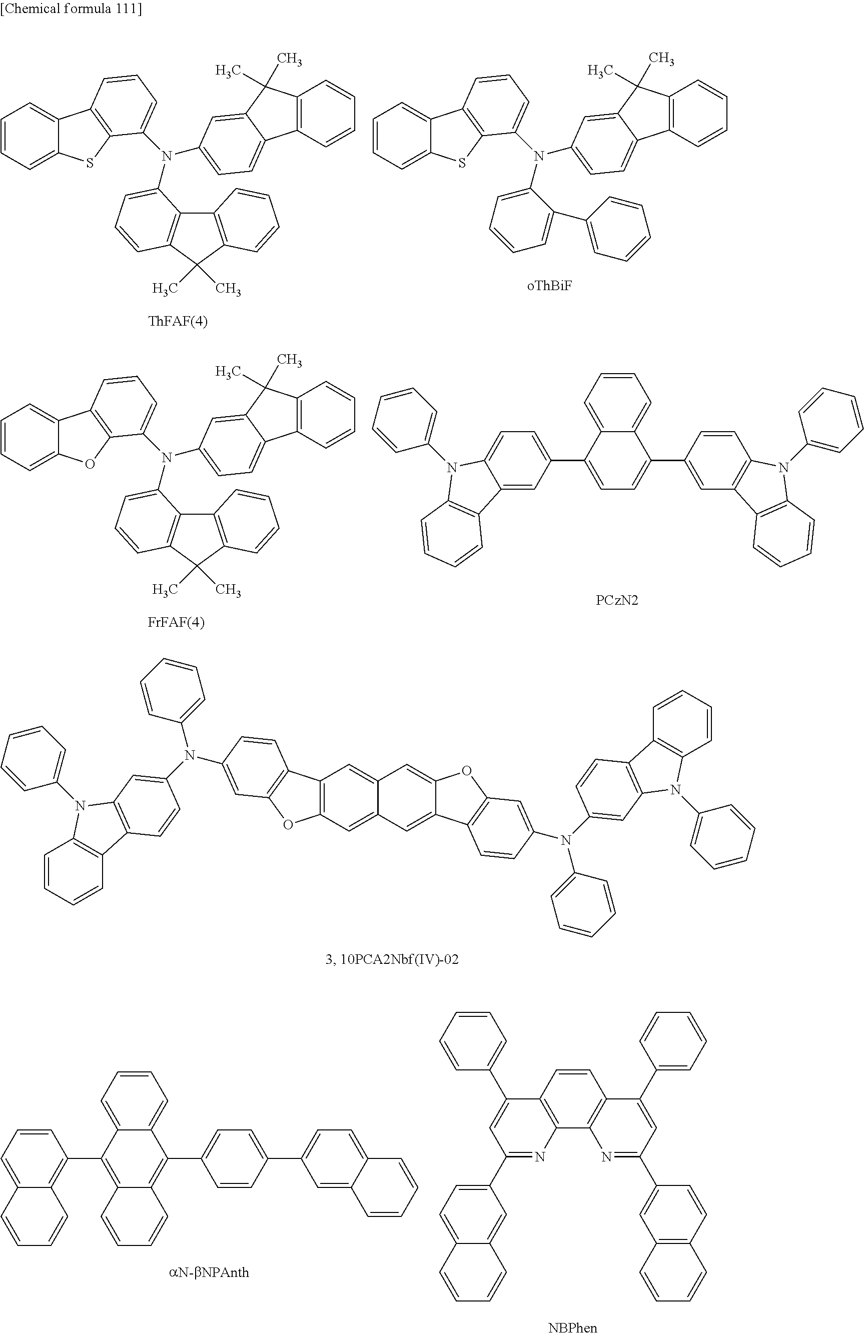

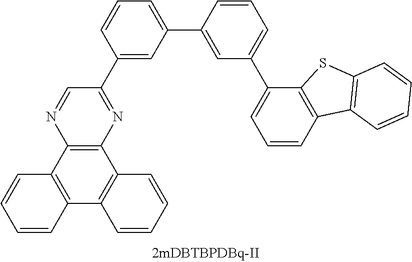 [SMILES: c1cc(-c2cccc(-c3cccc4c3sc3ccccc34)c2)cc(-c2cnc3c4ccccc4c4ccccc4c3n2)c1]

TABLE 1

| | | | Light-emitting | Light-emitting | Light-emitting |
|---|---|---|---|---|---|
| | | Thickness | device 1 | device 2 | device 3 |
| Second | | 150 nm | Al | | |
| electrode | | | | | |
| Electron- | | 1 nm | LiF | | |
| injection layer | | | | | |
| Electron- | 2 | 10 nm | NBPhen | | |
| transport | 1 | 15 nm | 2mDBTBPDBq-II | | |
| layer | | | | | |
| Light-emitting | | 25 nm | αN-β NPAnth:3,10PCA2Nbf(IV)-02 (1:0.015) | | |
| layer | | | | | |
| Hole- | 2 | 10 nm | PCzN2 | | |
| transport | 1 | 90 nm | ThFAF(4) | oThBiF | FrFAF(4) |
| layer | | | | | |

| | | | | |
|---|---|---|---|---|
| Hole-injection | 10 nm | ThFAF(4):OCHD-003 | oThBiF:OCHD-003 | FrFAF(4):OCHD-003 |
| layer | | (1:0.1) | (1:0.1) | (1:0.1) |
| First electrode | 110 nm | | | |

### <<Fabrication of Light-Emitting Device 1>>

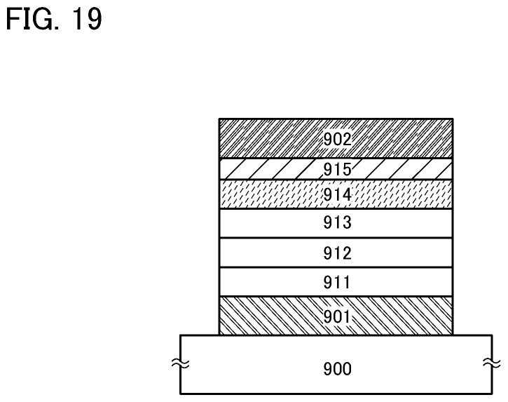

In the light-emitting device 1 described in this example, as illustrated in FIG. 19, a hole-injection layer 911, a hole-transport layer 912, a light-emitting layer 913, an electron-transport layer 914, and an electron-injection layer 915 are stacked in this order over a first electrode 901 formed over a substrate 900, and a second electrode 902 is stacked over the electron-injection layer 915.

First, the first electrode 901 was formed over the substrate 900. The electrode area was set to 4 mm² (2 mm×2 mm). A glass substrate was used as the substrate 900. As the first electrode 901, 110-nm-thick indium tin oxide containing silicon oxide (ITSO) were deposited by a sputtering method. In this example, the first electrode 901 functions as an anode.

For pretreatment, a surface of the substrate was washed with water, baking was performed at 200° C. for one hour, and then UV ozone treatment was performed for 370 seconds. After that, the substrate was transferred into a vacuum evaporation apparatus whose interior pressure was reduced to approximately 10⁻⁴ Pa, and was subjected to vacuum baking at 170° C. for 60 minutes in a heating chamber of the vacuum evaporation apparatus, and then the substrate was cooled down for approximately 30 minutes.

Next, the hole-injection layer 911 was formed over the first electrode 901. After the pressure in the vacuum evaporation chamber reduced to 10⁻⁴ Pa, the hole-injection layer 911 was formed to a thickness of 10 nm by co-evaporation of N-(9,9-dimethyl-9H-fluoren-2-yl)-N-(9,9-dimethyl-9H-fluoren-4-yl)dibenzothiophen-4-amine (abbreviation: ThFAF(4)) (Structural Formula (400)) and an electron acceptor material containing fluorine with a molecular weight of 672 (OCHD-003) at weight ratio of 1:0.1 (=ThFAF(4): OCHD-003).

Then, the hole-transport layer 912 was formed over the hole-injection layer 911. The hole-transport layer 912 was formed by evaporation of ThFAF(4) to a thickness of 90 nm and then evaporation of 3,3′-(naphthalene-1,4-diyl)bis(9-phenyl-9H-carbazole) (abbreviation: PCzN2) to a thickness of 10 nm.

Next, the light-emitting layer 913 was formed over the hole-transport layer 912.

The light-emitting layer 913 was formed to a thickness of 25 nm by co-evaporation of 9-(1-naphthyl)-10-[4-(2-naphthyl)phenyl]anthracene (abbreviation: αN-βNPAnth) and 3,10-bis[N-(9-phenyl-9H-carbazol-2-yl)-N-phenylamino]naphtho[2,3-b;6,7-b′]bisbenzofuran (abbreviation: 3,10PCA2Nbf(IV)-02) as a fluorescent substance such that the weight ratio of αN-βNPAnth to 3,10PCA2Nbf(IV)-02 was 1:0.015.

Next, the electron-transport layer 914 was formed over the light-emitting layer 913. Next, as the electron-transport layer 914, 2-[3′-(dibenzothiophen-4-yl)biphenyl-3-yl]dibenzo[f,h]quinoxaline (abbreviation: 2mDBTBPDBq-II) was formed to a thickness of 15 nm by evaporation and then 2,9-di(2-naphthyl)-4,7-diphenyl-1,10-phenanthroline (abbreviation: NBPhen) was formed to a thickness of 10 nm by evaporation.

The electron-injection layer 915 was formed over the electron-transport layer 914. The electron-injection layer 915 was formed to a thickness of 1 nm by evaporation of lithium fluoride (LiF).

After that, the second electrode 902 was formed over the electron-injection layer 915. The second electrode 902 was formed to a thickness of 150 nm by evaporation of aluminum (Al). In this example, the second electrode 902 functions as a cathode.

Through the above process, the light-emitting device 1 was fabricated. Next, methods for fabricating the light-emitting devices 2 and 3 are described.

### <<Fabrication of Light-Emitting Device 2>>

The light-emitting device 2 is different from the light-emitting device 1 in that ThFAF(4) used for the hole-injection layer 911 and the hole-transport layer 912 was replaced with N-(2-biphenyl)-N-(9,9-dimethyl-9H-fluoren-2-yl)-dibenzothiophen-4-amine (abbreviation: oThBiF) (Structural Formula (136)). The layers other than the hole-injection layer 911 and the hole-transport layer 912 were formed in a manner similar to that of the light-emitting device 1.

### <<Fabrication of Light-Emitting Device 3>>

The light-emitting device 3 is different from the light-emitting device 1 in that ThFAF(4) used for the hole-injection layer 911 and the hole-transport layer 912 was replaced with N-(9,9-dimethyl-9H-fluoren-2-yl)-N-(9,9-dimethyl-9H-fluoren-4-yl)-dibenzofuran-4-amine (abbreviation: FrFAF(4)) (Structural Formula (200)). The layers other than the hole-injection layer 911 and the hole-transport layer 912 were formed in a manner similar to that of the light-emitting device 1.

The light-emitting devices 1 to 3 were sealed using a glass substrate in a glove box containing a nitrogen atmosphere so as not to be exposed to the air (a sealing material was applied to surround the device and UV treatment and heat treatment at 80° C. for 1 hour were performed at the time of sealing). Then, the initial characteristics of the light-emitting devices were measured.

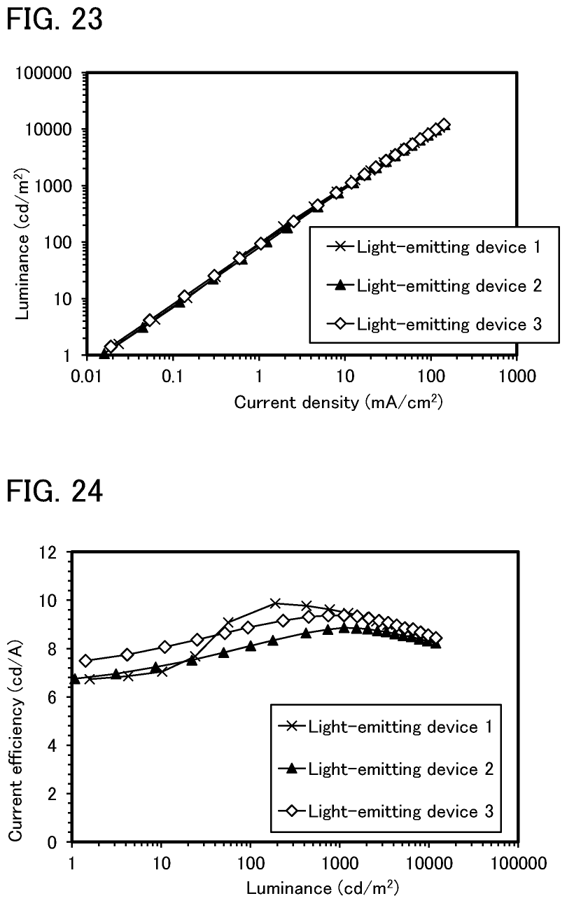

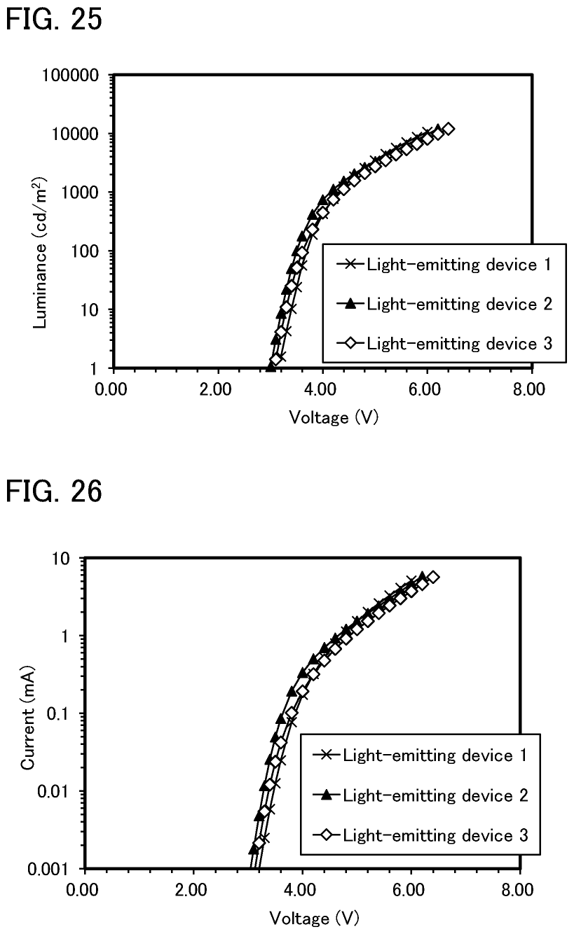

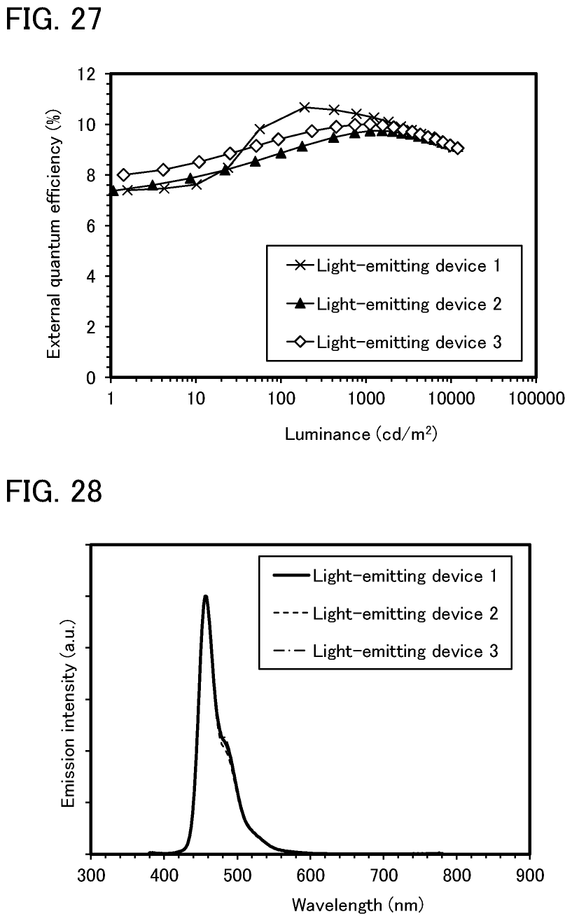

FIG. 23 shows the luminance-current density characteristics of the light-emitting devices 1 to 3. FIG. 24 shows the current efficiency-luminance characteristics thereof. FIG. 25 shows the luminance-voltage characteristics thereof. FIG. 26 shows the current-voltage characteristics thereof. FIG. 27 shows the external quantum efficiency-luminance characteristics thereof. FIG. 28 shows the emission spectrum thereof. Table 2 shows the main characteristics of the light-emitting devices at a luminance of approximately 1000 cd/m². Note that luminance, CIE chromaticity, and emission spectra were measured with a spectroradiometer (SR-UL1R manufactured by TOPCON TECHNOHOUSE CORPORATION). The external quantum efficiency was calculated from the luminance and the emission spectra measured with the spectroradiometer, on the assumption that the light-emitting devices had Lambertian light-distribution characteristics.

TABLE 2

| | | | | | | | External |
|---|---|---|---|---|---|---|---|
| | | | Current | | | Current | quantum |
| | Voltage | Current | density | Chromaticity | Chromaticity | efficiency | efficiency |
| | (V) | (mA) | (mA/cm²) | x | y | (cd/A) | (%) |
| Light-emitting | 4.2 | 0.32 | 8.0 | 0.138 | 0.105 | 9.6 | 10.4 |
| device 1 | | | | | | | |
| Light-emitting | 4.2 | 0.50 | 12.6 | 0.139 | 0.102 | 8.9 | 9.7 |
| device 2 | | | | | | | |
| Light-emitting | 4.4 | 0.48 | 11.9 | 0.138 | 0.107 | 9.4 | 10.0 |
| device 3 | | | | | | | |

FIG. 23 to FIG. 28 show that the light-emitting devices 1 to 3 containing any one of ThFAF(4), oThBiF, and FrFAF(4) in the EL layer have a low driving voltage of 4.4 V at a luminance of 1000 cd/m², and can emit a deep blue light with a chromaticity y of 0.12 or less with a high external quantum efficiency of 10%. In other words, the light-emitting devices 1 to 3 are found to be light-emitting devices having favorable characteristics.

### Example 4

In this example, light-emitting devices 4 to 7, which are fluorescent light-emitting devices of embodiments of the present invention, were fabricated, and comparison results of the device characteristics are shown. Structural formulae of organic compounds used for the light-emitting devices 4 to 7 are shown below. Furthermore, device structures of the light-emitting devices 4 to 7 are shown.

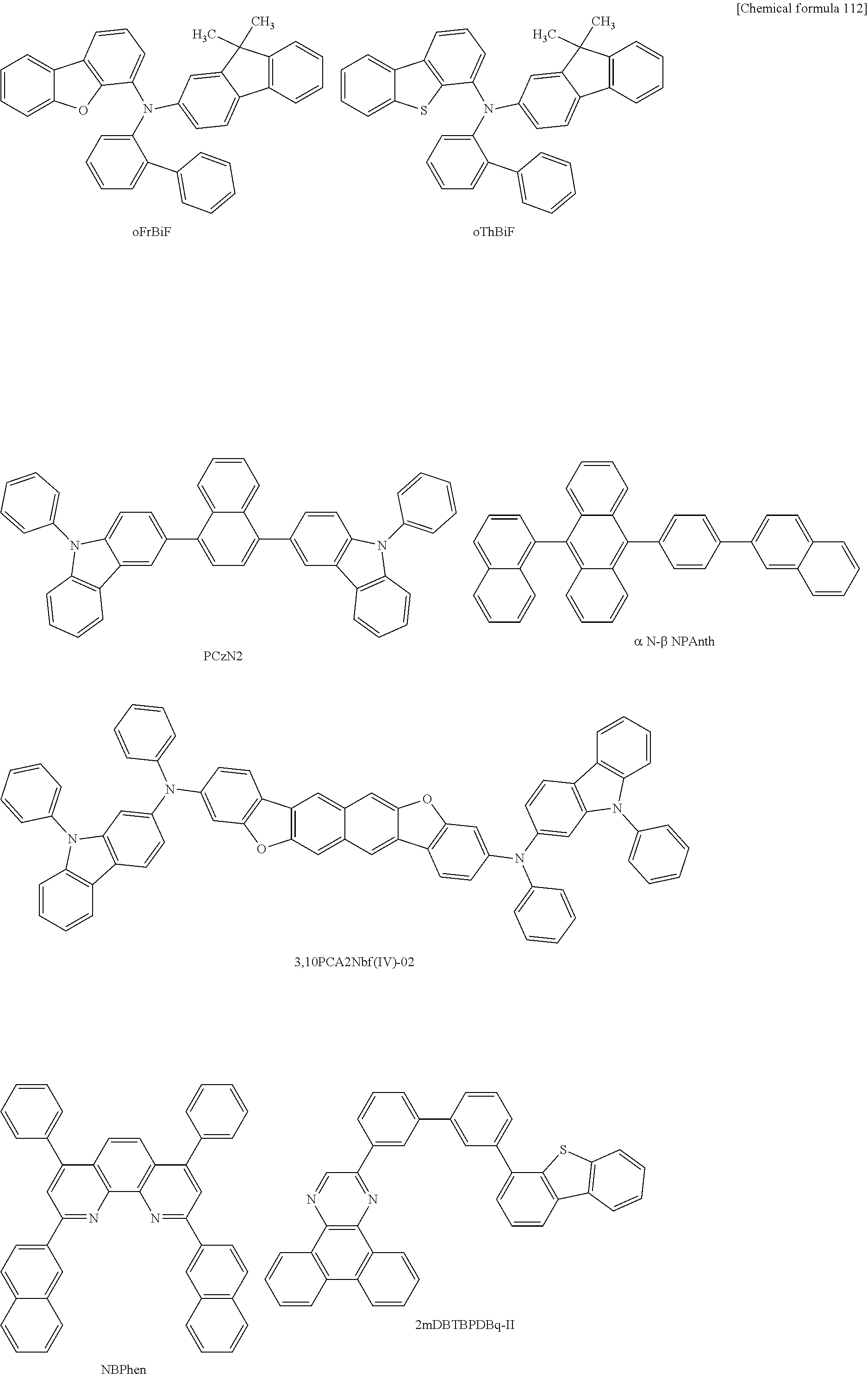

TABLE 3

| | | | Light-emitting | Light-emitting | Light-emitting | Light-emitting |
|---|---|---|---|---|---|---|
| | | Thickness | device 4 | device 5 | device 6 | device 7 |
| Second | | 150 nm | Al | | | |
| electrode | | | | | | |
| Electron- | | 1 nm | LiF | | | |
| injection layer | | | | | | |
| Electron- | 2 | 10 nm | NBPhen | | | |
| layer | | | | | | |
| transport | 1 | 15 nm | 2mDBTBPDBq-II | | | |
| Light-emitting | | 25 nm | αN-β NPAnth: 3,10PCA2Nbf(IV)-02 (1:0.015) | | | |
| layer | | | | | | |
| Hole-layer | 2 | 10 nm | PCzN2 | oFrBiF | PCzN2 | oThBiF |

| | | | | |
|---|---|---|---|---|
| transport | 1 | 90 nm | oFrBiF | oThBiF |

| | | | |
|---|---|---|---|
| Hole-injection | 10 nm | oFrBiF:OCHD-003 | oThBiF:OCHD-003 |
| layer | | (1 :0.05) | (1 :0.05) |

| | | |
|---|---|---|
| First electrode | 110 nm | ITSO |

### <<Fabrication of Light-Emitting Device 4>>

The light-emitting device 4 described in this example has the stacked structure illustrated in FIG. 19, as in Example 3.

First, the first electrode 901 was formed over the substrate 900. The electrode area was set to 4 mm² (2 mm×2 mm). A glass substrate was used as the substrate 900. The first electrode 901 was formed in such a manner that and 110-nm-thick indium tin oxide containing silicon oxide (ITSO) were deposited by a sputtering method. In this example, the first electrode 901 functions as an anode.

For pretreatment, a surface of the substrate was washed with water, baking was performed at 200° C. for one hour, and then UV ozone treatment was performed for 370 seconds. After that, the substrate was transferred into a vacuum evaporation apparatus whose interior pressure was reduced to approximately 10⁻⁴ Pa, and was subjected to vacuum baking at 170° C. for 60 minutes in a heating chamber of the vacuum evaporation apparatus, and then the substrate was cooled down for approximately 30 minutes.

Next, the hole-injection layer 911 was formed over the first electrode 901. After the interior pressure of the vacuum evaporation apparatus was reduced to 10⁻⁴ Pa, the hole-injection layer 911 was formed to a thickness of 10 nm by co-evaporation of N-(2-biphenyl)-N-(9,9-dimethyl-9H-fluoren-2-yl)dibenzofuran-4-amine (abbreviation: oFrBiF) (Structural Formula (105)) and OCHD-003 at a weight ratio of 1:0.05 (=oFrBiF: OCHD-003).

Then, the hole-transport layer 912 was formed over the hole-injection layer 911. As the hole-transport layer 912, oFrBiF was evaporated to a thickness of 90 nm and then PCzN2 was evaporated to a thickness of 10 nm.

Next, the light-emitting layer 913 was formed over the hole-transport layer 912.

The light-emitting layer 913 was formed to a thickness of 25 nm by co-evaporation of αN-βNPAnth and a fluorescent substance, 3,10PCA2Nbf(IV)-02 at a weight ratio of αN-βNPAnth: 3,10PCA2Nbf(IV)-02=1:0.015.

Next, the electron-transport layer 914 was formed over the light-emitting layer 913. As the electron-transport layer 914, 2mDBTBPDBq-II was evaporated to a thickness of 15 nm and then NBPhen was evaporated to a thickness of 10 nm.

The electron-injection layer 915 was formed over the electron-transport layer 914. The electron-injection layer 915 was formed to a thickness of 1 nm by evaporation of lithium fluoride (LiF).

After that, the second electrode 902 was formed over the electron-injection layer 915. The second electrode 902 was formed to a thickness of 150 nm by evaporation of aluminum. In this example, the second electrode 902 functions as a cathode.

Through the above process, the light-emitting device 4 was fabricated. Next, a method for fabricating the light-emitting devices 5 to 7 is described.

### <<Fabrication of Light-Emitting Device 5>>

The light-emitting device 5 is different from the light-emitting device 4 in that PCzN2 used for the hole-transport layer 912 was replaced with oFrBiF. The layers other than the hole-transport layer 912 were formed in a manner similar to that of the light-emitting device 4.

### <<Fabrication of Light-Emitting Device 6>>

The light-emitting device 6 is different from the light-emitting device 4 in that oFrBiF used for the hole-injection layer 911 and the hole-transport layer 912 was replaced with N-(2-biphenyl)-N-(9,9-dimethyl-9H-fluoren-2-yl)dibenzothipphen-4-amine (abbreviation: oThBiF) (Structural Formula (136)). The layers other than the hole-injection layer 911 and the hole-transport layer 912 were formed in a manner similar to that of the light-emitting device 4.

### <<Fabrication of Light-Emitting Device 7>>

The light-emitting device 7 is different from the light-emitting device 6 in that PCzN2 used for the hole-transport layer 912 was replaced with oThBiF. The layers other than the hole-transport layer 912 were formed in a manner similar to that of the light-emitting device 6.

The light-emitting devices 4 to 7 were sealed using a glass substrate in a glove box containing a nitrogen atmosphere so as not to be exposed to the air (a sealing material was applied to surround the device and UV treatment and heat treatment at 80° C. for 1 hour were performed at the time of sealing). Then, the initial characteristics of the light-emitting devices were measured.

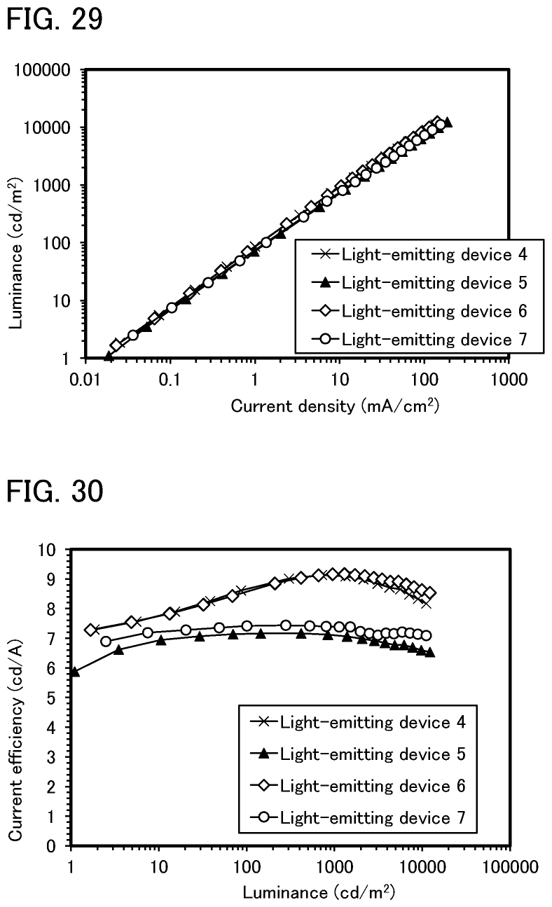

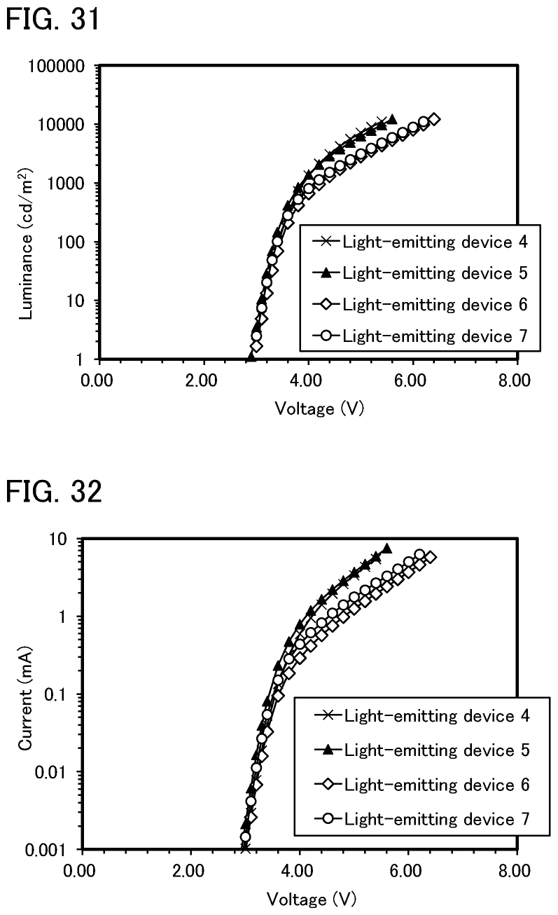

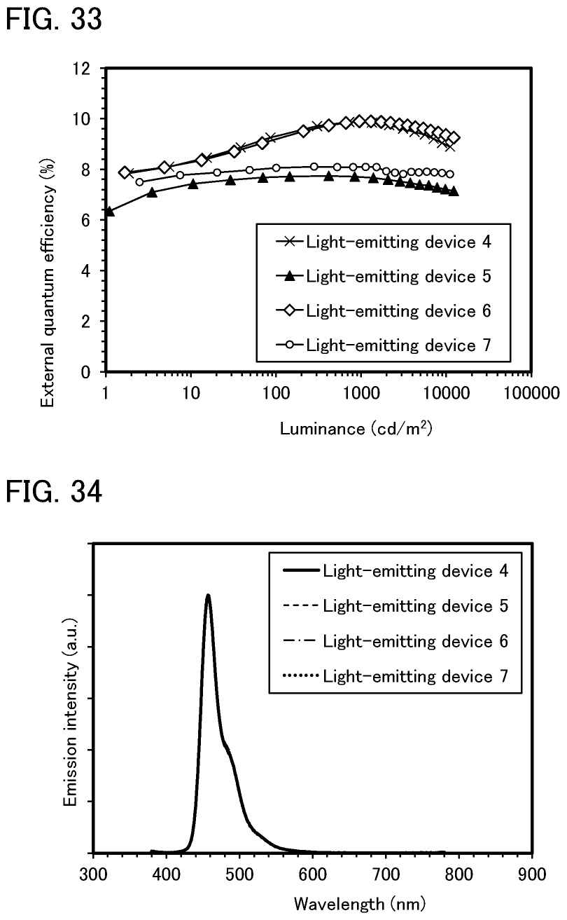

FIG. 29 shows the luminance-current density characteristics of the light-emitting devices 4 to 7. FIG. 30 shows the current efficiency-luminance characteristics thereof. FIG. 31 shows the luminance-voltage characteristics thereof. FIG. 32 shows the current-voltage characteristics thereof. FIG. 33 shows the external quantum efficiency-luminance characteristics thereof. FIG. 34 shows the emission spectrum thereof. Table 4 shows the main characteristics of the light-emitting devices at a luminance of approximately 1000 cd/m². Note that luminance, CIE chromaticity, and emission spectra were measured with a spectroradiometer (SR-UL1R manufactured by TOPCON TECHNOHOUSE CORPORATION). The external quantum efficiency was calculated from the luminance and the emission spectra measured with the spectroradiometer, on the assumption that the light-emitting devices had Lambertian light-distribution characteristics.

TABLE 4

| | | | | | | | External |
|---|---|---|---|---|---|---|---|
| | | | Current | | | Current | quantum |
| | Voltage | Current | density | Chromaticity | Chromaticity | efficiency | efficiency |
| | (V) | (mA) | (mA/cm²) | x | y | (cd/A) | (%) |
| Light-emitting | 3.8 | 0.32 | 7.9 | 0.139 | 0.104 | 9.14 | 9.87 |
| device 4 | | | | | | | |
| Light-emitting | 3.8 | 0.47 | 11.8 | 0.139 | 0.104 | 7.12 | 7.70 |
| device 5 | | | | | | | |
| Light-emitting | 4.2 | 0.42 | 10.4 | 0.138 | 0.105 | 9.17 | 9.89 |
| device 6 | | | | | | | |
| Light-emitting | 4.2 | 0.61 | 15.3 | 0.139 | 0.103 | 7.39 | 8.09 |
| device 7 | | | | | | | |

FIG. 29 to FIG. 34 shows that the light-emitting devices 4 to 7 containing oFrBiF or oThBiF in their EL layers have a low driving voltage of around 4.0 V at a luminance of 1000 cd/m², and can emit deep blue light with a CIE chromaticity y of 0.12 or less with a high external quantum efficiency of 10%. In other words, the light-emitting devices 4 to 7 are found to be light-emitting devices having favorable characteristics.

### Example 5

### Synthesis Example 3

This example will describe a method for synthesizing N-(9,9-diphenyl-9H-fluoren-2-yl)-N-(9,9′-spirobi[9H-fluoren]-4-yl)dibenzofuran-2-amine (abbreviation: Fr(2)FASF(4)-02), which is the organic compound represented by Structural Formula (272) in Embodiment 1. The structure of Fr(2)FASF(4)-02 is shown below.

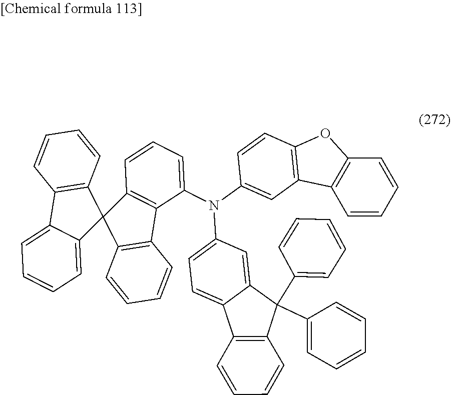 [SMILES: c1ccc(C2(c3ccccc3)c3ccccc3-c3ccc(N(c4ccc5oc6ccccc6c5c4)c4cccc5c4-c4ccccc4C54c5ccccc5-c5ccccc54)cc32)cc1]

### Step 1: Synthesis of Fr(2)FASF(4)-02

To a 200 mL three-neck flask were put 1.5 g (3.7 mmol) of 2-bromo-9,9-diphenyl-9H-fluorene, 2.2 g (4.4 mmol) of N-(9,9′-spirobi[9H-fluoren]-4-yl)dibenzofuran-2-amine, and 1.1 g (11 mmol) of sodium-tert-butoxide (abbreviation: tBuONa). To this mixture, 20 mL of toluene and 0.2 mL of a 10% hexane solution of tri(tert-butyl)phosphine (abbreviation: P(tBu)₃) were added, and this mixture was degassed by being stirred while the pressure was reduced. To this mixture, 21 mg (37 μmol) of bis(dibenzylideneacetone)palladium(0) (abbreviation Pd(dba)₂) was added, and heating and stirring was performed under a nitrogen stream at 120° C. for 1.5 hours.

After the stirring, toluene was added to the mixture, and the mixture was suction-filtered through Florisil, Celite, and alumina to give a filtrate. The obtained filtrate was concentrated to give an oily substance. This oily substance was purified by silica gel column chromatography (hexane and toluene at 1:2 in the developing solvent) to give a solid. The obtained solid was recrystallized with ethyl acetate/ethanol to give 1.9 g of a white solid in 63% yield.

By a train sublimation method, 1.8 g of the obtained solid was purified. The purification by sublimation was performed by heating at 320° C. under a pressure of 2.6 Pa with an argon flow rate of 15 mL/min. After the purification by sublimation, 1.6 g of a target white solid was obtained at a collection rate of 88%. The synthesis scheme is shown in (c-1) below.

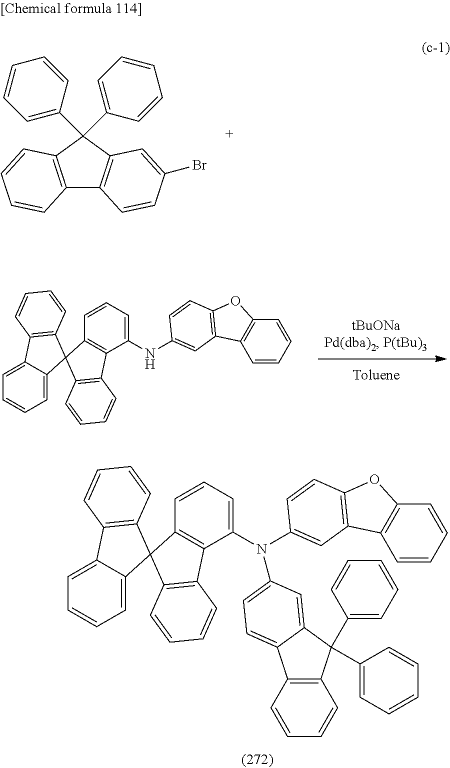 [SMILES: Brc1ccc2c(c1)C(c1ccccc1)(c1ccccc1)c1ccccc1-2 ; c1ccc2c(c1)-c1ccccc1C21c2ccccc2-c2c(Nc3ccc4oc5ccccc5c4c3)cccc21 ; c1ccc(C2(c3ccccc3)c3ccccc3-c3ccc(N(c4ccc5oc6ccccc6c5c4)c4cccc5c4-c4ccccc4C54c5ccccc5-c5ccccc54)cc32)cc1]

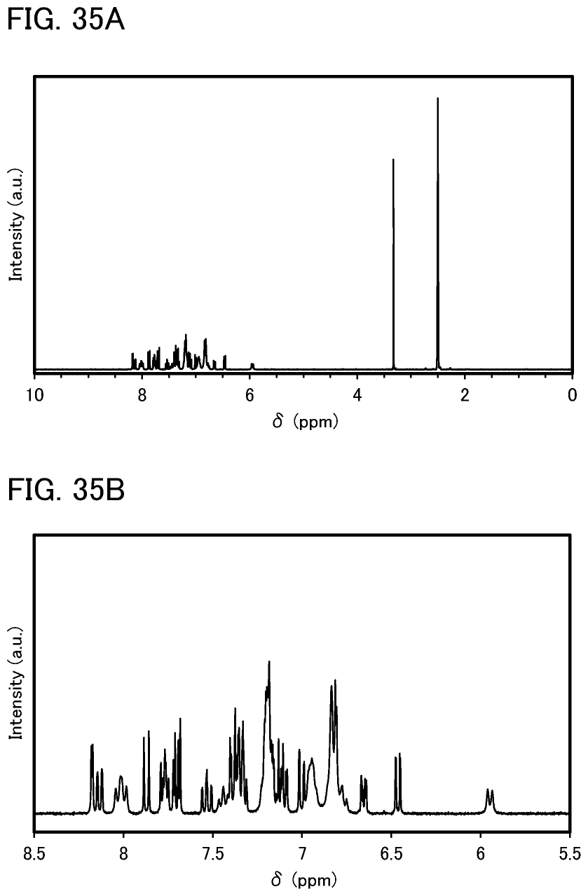

Analysis results by nuclear magnetic resonance (¹H-NMR) spectroscopy of the yellow solid obtained in the above Step 1 are shown below. FIGS. 35A and 35B show the ¹H-NMR chart. The result revealed that Fr(2)FASF(4)-02(Structural formula (272)) was obtained in this synthesis example.

¹H-NMR (DMSO-d₆, 300 MHz): δ=5.95 (d, J=7.8 Hz, 1H), 6.46 (dd, J₁=1.2 Hz, J₂=7.8 Hz, 1H), 6.64-6.67 (m, 1H), 6.75-7.02 ((m, 11H), 7.08-7.23 (m, 9H), 7.31-7.47 (m, 6H), 7.53 (dt, J₁=1.5 Hz, J₂=8.4 Hz, 1H), 7.70 (dd, J₁=3.3 Hz, J₂=8.7 Hz, 2H), 7.75-7.79 (m, 2H), 7.87 (d, J=8.4 Hz, 1H), 7.99-8.04 (m, 2H), 8.13 (d, J=7.5 Hz, 1H), 8.18 (d, J=2.4 Hz, 1H).

### <Tg Measurement>

The glass transition temperature (Tg) of Fr(2)FASF(4)-02 was measured. Note that Tg was measured with a differential scanning calorimeter (DSC8500 produced by PerkinElmer Japan Co., Ltd.) in a state where a powder was put on an aluminum cell. As a result, Tg of Fr(2)FASF(4)-02 was 180° C. This reveals that the compound of one embodiment of the present invention has extremely high heat resistance properties. With use of such a compound in a light-emitting device, a light-emitting device having high heat resistance can be provided.

Next, for comparison, Tg of N-(9,9-dimethyl-9H-fluoren-2-yl)-N-(9,9′-spirobi[9H-fluoren]-4-yl)dibenzofuran-2-amine (abbreviation: Fr(2)FASF(4)) (Structural Formula (250)) was also measured. The Tg measurement of Fr(2)FASF(4) was conducted with the same apparatus and the same measurement method as those used for Fr(2)FASF(4)-02. As a result, Tg of Fr(2)FASF(4) was 158° C. In comparison of Tg between Fr(2)FASF(4) and Fr(2)FASF(4)-02, Tg of Fr(2)FASF(4)-02 is higher by 22° C. than that of Fr(2)FASF(4) and this is considered to result from that Fr(2)FASF(4)-02 has a structure in which a dimethylfluorene skeleton of Fr(2)FASF(4) was replaced with a spirobi[9H-fluorene] skeleton. Accordingly, having a spirobi[9H-fluorene] skeleton can exhibit an excellent effect for provision of a heat-resistance device.

As described above, by using a structure containing a spirobi[9H-fluorene]skeleton for an organic compound of one embodiment of the present invention, the organic compound can have both a high heat-resistance and a high hole-transport property. The use of the organic compound having a low HOMO level and a high hole-transport property, which is one embodiment of the present invention, can provide a light-emitting device, a light-emitting apparatus, an electronic appliance, or a lighting device, each of which has a high heat-resistance, a high emission efficiency, and a low power consumption.

### Example 6

In this example, light-emitting devices 8 and 9, which are fluorescent light-emitting devices of embodiments of the present invention, were fabricated, and comparison results of the device characteristics are shown. Structural formulae of organic compounds used for the light-receiving devices 8 and 9 are shown below. Furthermore, device structures of the light-emitting devices 8 and 9 are shown.

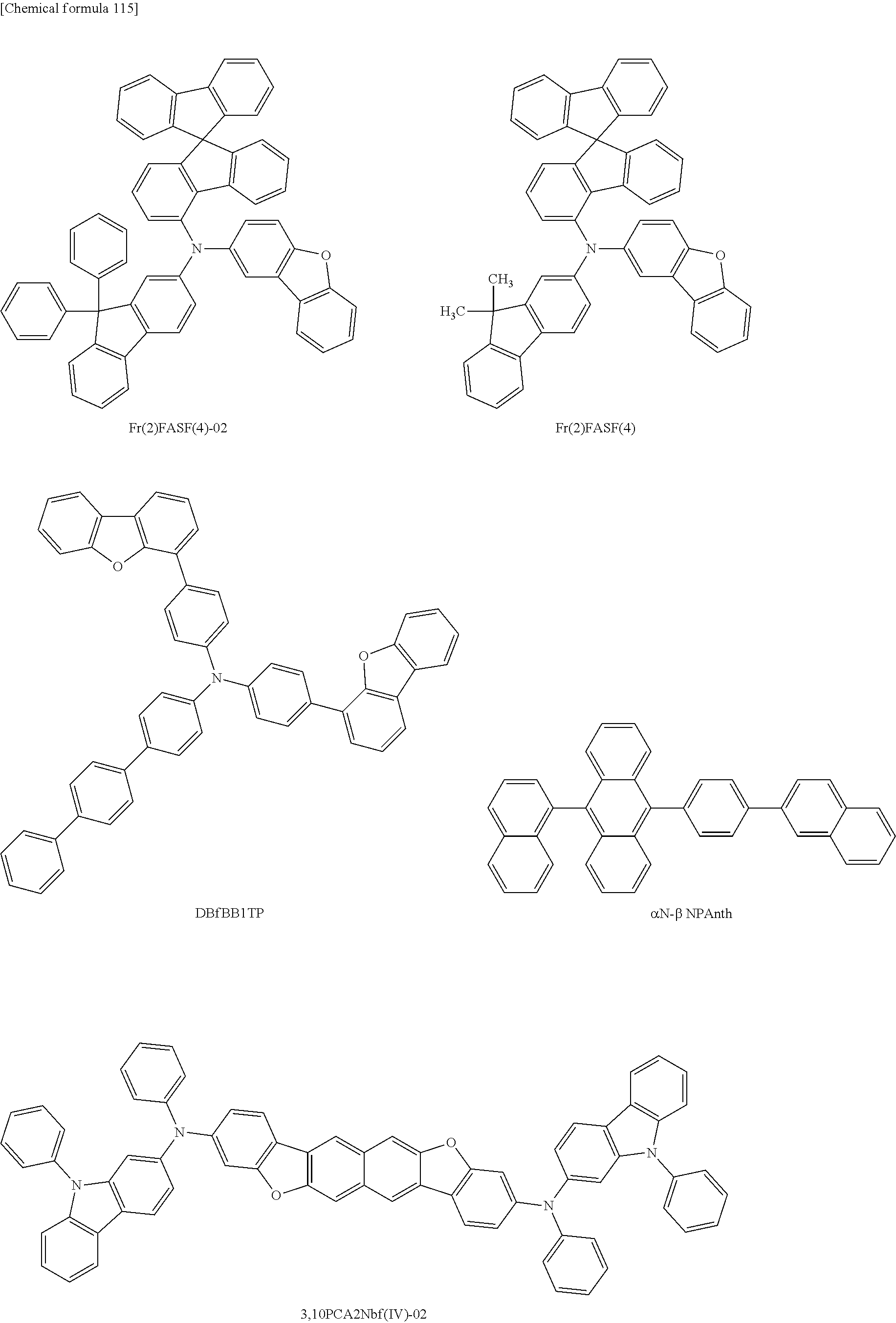

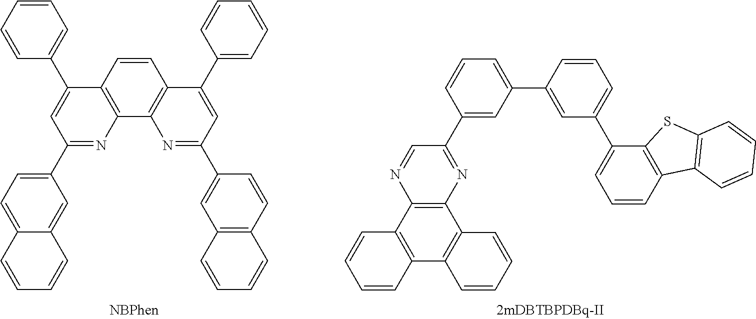 [SMILES: c1ccc(-c2cc(-c3ccc4ccccc4c3)nc3c2ccc2c(-c4ccccc4)cc(-c4ccc5ccccc5c4)nc23)cc1 ; c1cc(-c2cccc(-c3cccc4c3sc3ccccc34)c2)cc(-c2cnc3c4ccccc4c4ccccc4c3n2)c1]

TABLE 5

| | | Thick- | Light-emitting | Light-emitting |
|---|---|---|---|---|
| | | ness | device 8 | device 9 |
| Second | | 150 nm | Al | |
| electrode | | | | |
| Electron- | | 1 nm | LiF | |
| injection layer | | | | |
| Electron- | 2 | 15 nm | NBPhen | |
| transport | 1 | 10 nm | 2mDBTBPDBq-II | |
| layer | | | | |
| Light-emitting | | 25 nm | αN-β NPAnth:3,10PCA2Nbf(IV)- | |
| layer | | | 02 (1:0.015) | |
| Hole- | 2 | 10 nm | DBfBB1TP | |
| transport | 1 | 20 nm | Fr(2)FASF(4)-02 | Fr(2)FASF(4) |
| layer | | | | |

| | | | |
|---|---|---|---|
| Hole- | 10 nm | Fr(2)FASF(4)-02: | Fr(2)FASF(4): |
| injection | | OCHD-003 | OCHD-003 |
| layer | | (1:0.05) | (1:0.05) |

| | | |
|---|---|---|
| First electrode | 70 nm | ITSO |

### <<Fabrication of Light-Emitting Device 8>>

The light-emitting device 8 has a structure in which the first electrode 901, the hole-injection layer 911, the hole-transport layer 912, and the electron-transport layer 914 are changed from those of the light-emitting device 4 in Example 4. Specifically, the light-emitting device 8 differs from the light-emitting device 4 in that the first electrode had a thickness of 70 nm; oFrBiF used for the hole-injection layer 911 in the light-emitting device 4 was replaced with Fr(2)FASF(4)-02; oFrBiF used for the hole-transport layer 912 in the light-emitting device 4 was replaced with Fr(2)FASF(4)-02 with a thickness of 20 nm; PCzN2 used for the hole-transport layer 912 in the light-emitting device 4 was replaced with N,N-bis[4-(dibenzofuran-4-yl)phenyl]-4-amino-p-terphenyl (abbreviation: DBfBB1TP); and 10-nm-thick 2mDBTBPDBq-II and 15-nm-thick NBPhen were used as the electron-transport layer 914. The others of the light-emitting device 8 were fabricated as in the light-emitting device 4.

### <<Fabrication of Light-Emitting Device 9>>

The light-emitting device 9 is different from the light-emitting device 8 in that Fr(2)FASF(4)-02 used in the light-emitting device 8 was replaced with Fr(2)FASF(4). The others of the light-emitting device 9 were fabricated as in the light-emitting device 8.

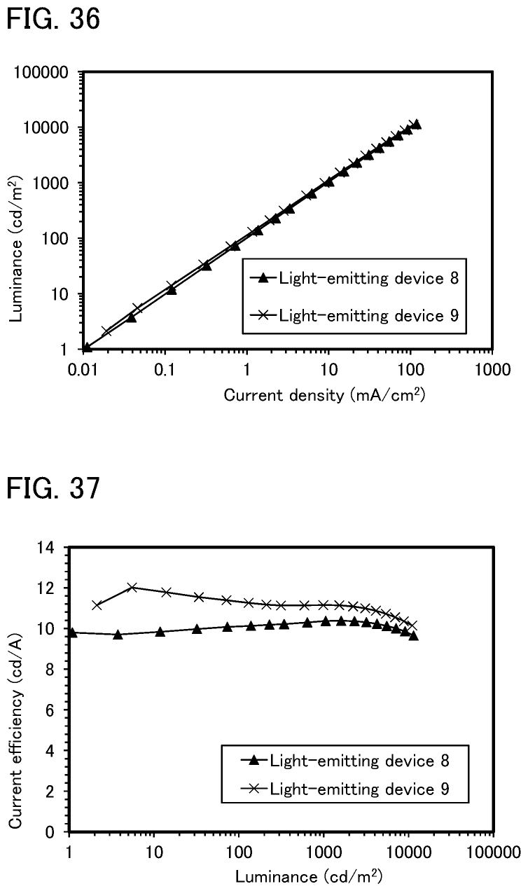

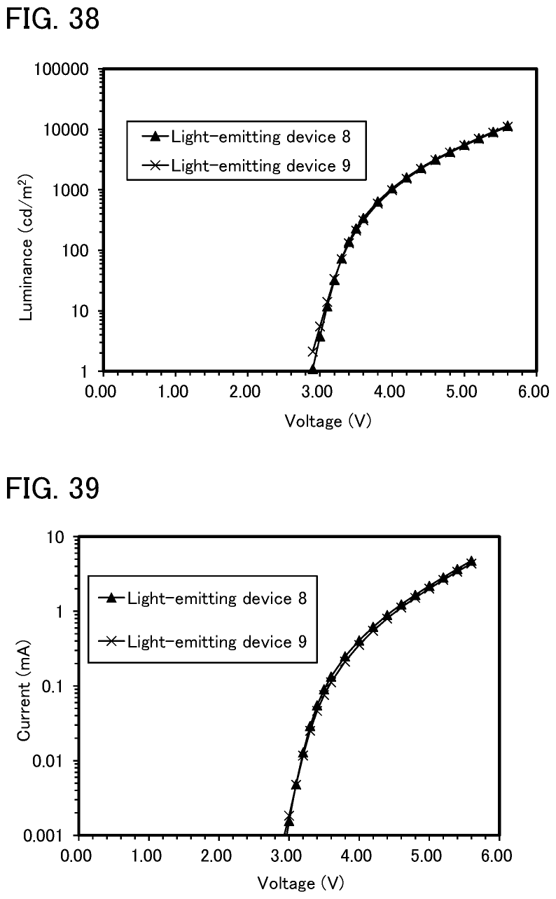

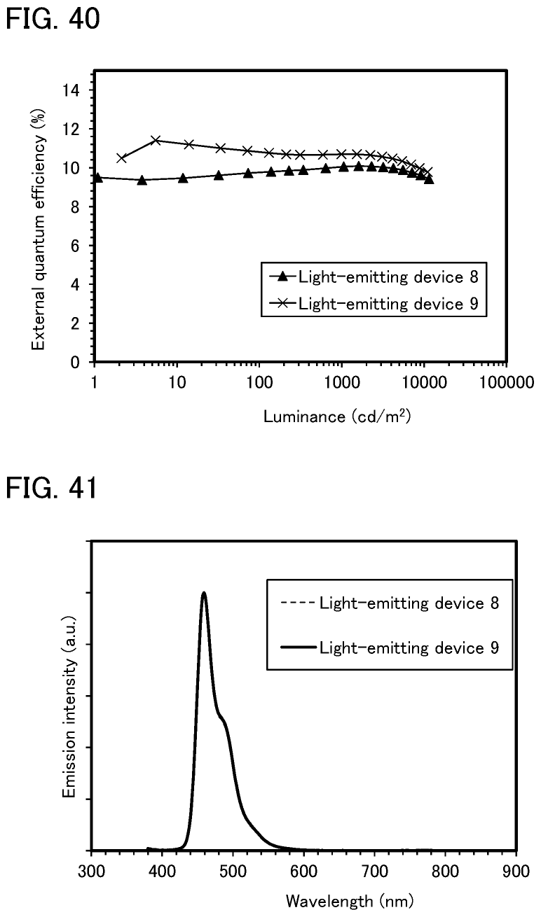

FIG. 36 shows the luminance-current density characteristics of the light-emitting device 8 and the light-emitting device 9. FIG. 37 shows the current efficiency-luminance characteristics thereof. FIG. 38 shows the luminance-voltage characteristics thereof. FIG. 39 shows the current-voltage characteristics thereof. FIG. 40 shows the external quantum efficiency-luminance characteristics thereof. FIG. 41 shows the emission spectra at a luminance of approximately 1000 cd/m². Table 6 shows the main characteristics of the light-emitting devices at a luminance of approximately 1000 cd/m². Note that luminance, CIE chromaticity, and emission spectra were measured with a spectroradiometer (SR-UL1R manufactured by TOPCON TECHNOHOUSE CORPORATION). The external quantum efficiency was calculated from the luminance and the emission spectra measured with the spectroradiometer, on the assumption that the light-emitting devices had Lambertian light-distribution characteristics.

TABLE 6

| | | | | | | | External |
|---|---|---|---|---|---|---|---|
| | | | Current | | | Current | quantum |
| | Voltage | Current | density | Chromaticity | Chromaticity | efficiency | efficiency |
| | (V) | (mA) | (mA/cm²) | x | y | (cd/A) | (%) |
| Light-emitting | 4.0 | 0.41 | 10.2 | 0.134 | 0.124 | 10.4 | 10.1 |
| device 8 | | | | | | | |
| Light-emitting | 4.0 | 0.35 | 8.9 | 0.133 | 0.126 | 11.1 | 10.7 |
| device 9 | | | | | | | |

FIG. 36 to FIG. 41 show that the light-emitting devices 8 and 9 containing Fr(2)FASF(4)-02 and Fr(2)FASF(4), respectively, in the EL layer have a low driving voltage of 4.0 V at a luminance of approximately 1000 cd/m², and can emit excellent blue light with a CIE chromaticity y of 0.13 or less with an extremely high external quantum efficiency of 10% or higher. Thus, the light-emitting devices 8 and 9 are found to be fluorescent light-emitting devices having an extremely high efficiency. Accordingly, the light-emitting devices 8 and 9 can be light-emitting devices having favorable characteristics.

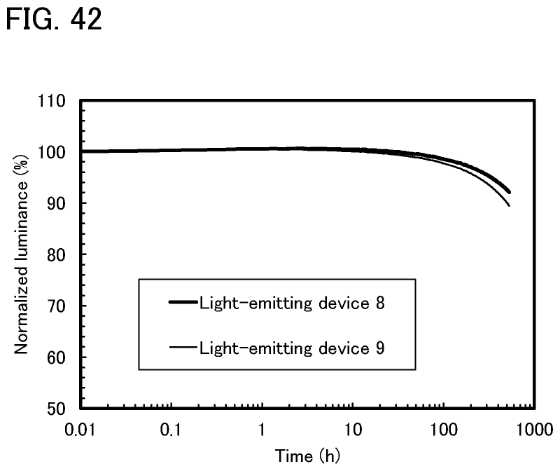

FIG. 42 shows luminance changes over driving time when the light-emitting devices 8 and 9 are driven at a constant current of 2 mA (50 mA/cm²⁾. As shown in FIG. 42, the light-emitting devices 8 and 9 are light-emitting devices with long lifetime. This application is based on Japanese Patent Application Serial No. 2021-134705 filed with Japan Patent Office on Aug. 20, 2021 and Japanese Patent Application Serial No. 2022-011987 filed with Japan Patent Office on Jan. 28, 2022, the entire contents of which are hereby incorporated by reference.

What is claimed is:

1. A light-emitting device comprising:
an anode;
a cathode; and
a light-emitting layer between the anode and the cathode,
wherein the light-emitting layer comprises a light-emitting substance,
wherein the light-emitting substance is a substance emitting fluorescent light,
wherein a first organic compound represented by General Formula (G3) is included between the anode and the cathode, 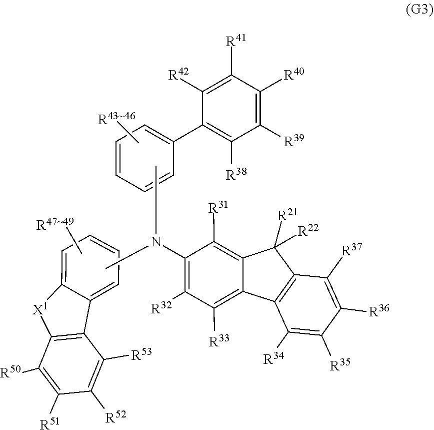
wherein X¹ represents oxygen or sulfur,
wherein R²¹, R²², and R³¹ to R³⁷ each independently represent hydrogen, an alkyl group having 1 to 4 carbon atoms, or an aryl group having 6 to 13 carbon atoms, R³⁸ to R⁴⁶ each independently represent hydrogen, an alkyl group having 1 to 4 carbon atoms, or an aryl group having 6 to 13 carbon atoms, and R⁴⁷ to R⁵³ each independently represent hydrogen, an alkyl group having 1 to 4 carbon atoms, or an aryl group having 6 to 13 carbon atoms, and
wherein the aryl group exclude a heteroaryl group.

2. The light-emitting device according to claim 1,
wherein at least two of substituents represented by R²¹, R²², and R³¹ to R³⁷ are bonded to each other to form a ring.

3. The light-emitting device according to claim 1,
wherein at least two of substituents represented by R³⁸ to R⁴⁶ are bonded to each other to form a ring.

4. The light-emitting device according to claim 1,
wherein at least two of substituents represented by R⁴⁷ to R⁵³ are bonded to each other to form a ring.

5. The light-emitting device according to claim 1,
wherein the light-emitting layer comprises the first organic compound.

6. The light-emitting device according to claim 1, further comprising a first layer between the light-emitting layer and the anode,
wherein the first layer comprises the first organic compound.

7. The light-emitting device according to claim 6,
wherein the first layer is in contact with the light-emitting layer.

8. The light-emitting device according to claim 6,
wherein the first layer is in contact with the anode.

9. The light-emitting device according to claim 1,
wherein the first organic compound is represented by any one of Structural Formulae (100), (101), (105), (136), (200), and (400),

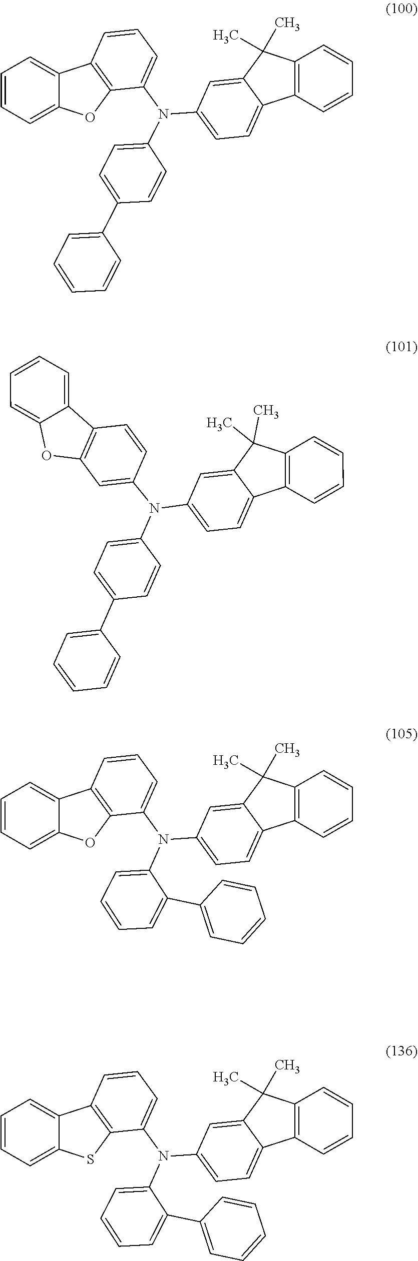 [SMILES: CC1(C)c2ccccc2-c2ccc(N(c3ccc(-c4ccccc4)cc3)c3cccc4c3oc3ccccc34)cc21 ; CC1(C)c2ccccc2-c2ccc(N(c3ccc(-c4ccccc4)cc3)c3ccc4c(c3)oc3ccccc34)cc21 ; CC1(C)c2ccccc2-c2ccc(N(c3ccccc3-c3ccccc3)c3cccc4c3oc3ccccc34)cc21 ; CC1(C)c2ccccc2-c2ccc(N(c3ccccc3-c3ccccc3)c3cccc4c3sc3ccccc34)cc21]

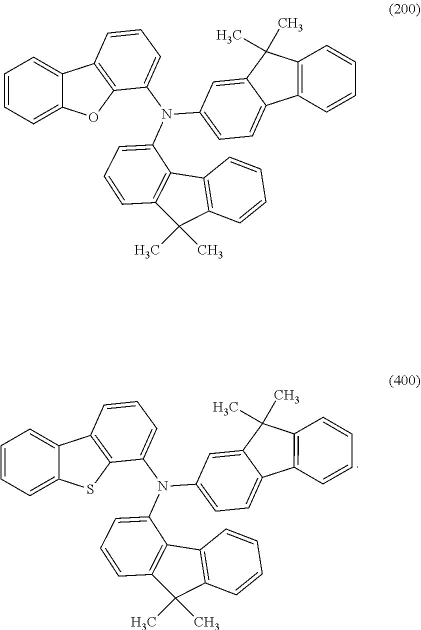 [SMILES: CC1(C)c2ccccc2-c2ccc(N(c3cccc4c3-c3ccccc3C4(C)C)c3cccc4c3oc3ccccc34)cc21 ; CC1(C)c2ccccc2-c2ccc(N(c3cccc4c3-c3ccccc3C4(C)C)c3cccc4c3sc3ccccc34)cc21]

10. The light-emitting device according to claim 1,
wherein a difference between a lowest singlet excited level and a lowest triplet excited level of the light-emitting substance is 0.3 eV or more.

11. The light-emitting device according to claim 1,
wherein the light-emitting substance is a substance emitting blue light.

12. A light-emitting apparatus comprising:
the light-emitting device according to claim 1; and
a light-receiving device,
wherein the light-receiving device comprises a light-receiving layer between a first electrode and a second electrode, and
wherein the light-receiving layer comprises the first organic compound.

13. A light-emitting apparatus comprising:
the light-emitting device according to claim 6; and
a light-receiving device,
wherein the light-receiving device comprises a light-receiving layer between a first electrode and a second electrode, and
wherein the light-receiving layer comprises an active layer, and the first layer between the active layer and the first electrode.

14. An organic compound represented by General Formula (G11), 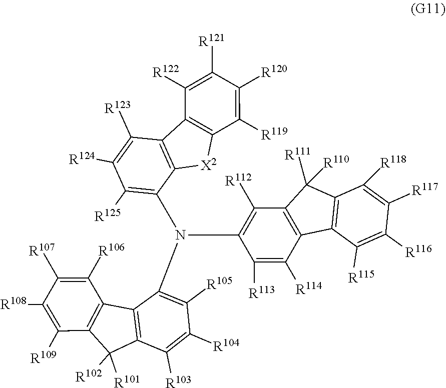
wherein X² represents oxygen or sulfur,
wherein R¹⁰³ to R¹⁰⁹, R¹¹² to R¹¹⁸, and R¹¹⁹ to R¹²⁵ each independently represent hydrogen, a substituted or unsubstituted alkyl group having 1 to 6 carbon atoms, or a substituted or unsubstituted aryl group having 6 to 13 carbon atoms,
wherein R¹⁰¹, R¹⁰², R¹¹⁰, and R¹¹¹ each independently represent hydrogen, a substituted or unsubstituted alkyl group having 1 to 6 carbon atoms, or a substituted or unsubstituted aryl group having 6 to 13 carbon atoms,
wherein R¹⁰⁶, R¹¹⁴, and R¹¹⁵ each represent a substituent other than an unsubstituted phenyl group,
wherein when R¹⁰¹ and R¹⁰² are each an unsubstituted phenyl group, R¹¹⁰ and R¹¹¹ each represent a substituent other than a methyl group, and
wherein when R¹¹⁰ and R¹¹¹ are each an unsubstituted phenyl group, R¹⁰¹ and R¹⁰² each represent a substituent other than a methyl group.

15. The organic compound according to claim 14,
wherein R¹⁰¹, R¹⁰², R¹¹⁰, and R¹¹¹ each independently represent hydrogen, a methyl group, or an unsubstituted phenyl group.

16. The organic compound according to claim 14,
wherein the organic compound is represented by General Formula (G12), 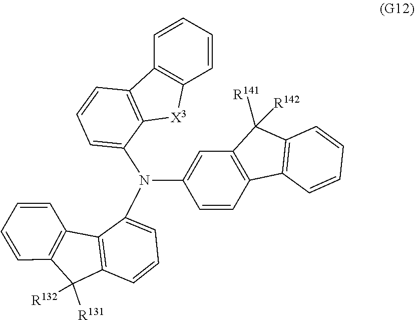
wherein X³ represents oxygen or sulfur, and
wherein three or all of R¹³¹, R¹³², R¹⁴¹, and R¹⁴² represent the same substituent, and the substituent represents hydrogen, a methyl group, or an unsubstituted phenyl group, and the other represents hydrogen, a methyl group, or an unsubstituted phenyl group.

17. The organic compound according to claim 14,
wherein the organic compound is represented by Structural Formula (200) or (400),

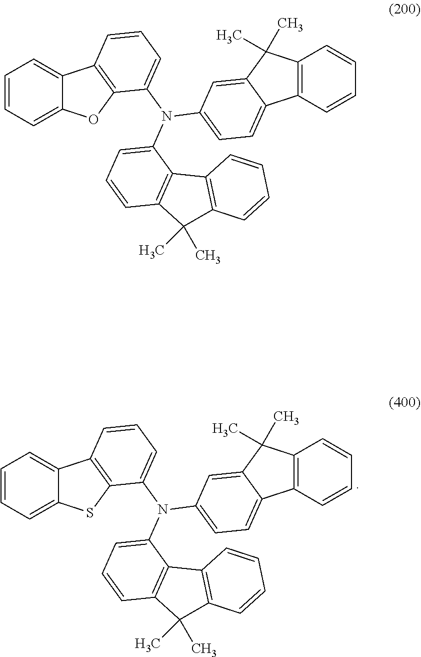 [SMILES: CC1(C)c2ccccc2-c2ccc(N(c3cccc4c3-c3ccccc3C4(C)C)c3cccc4c3oc3ccccc34)cc21 ; CC1(C)c2ccccc2-c2ccc(N(c3cccc4c3-c3ccccc3C4(C)C)c3cccc4c3sc3ccccc34)cc21]

18. An organic compound represented by General Formula (G21), 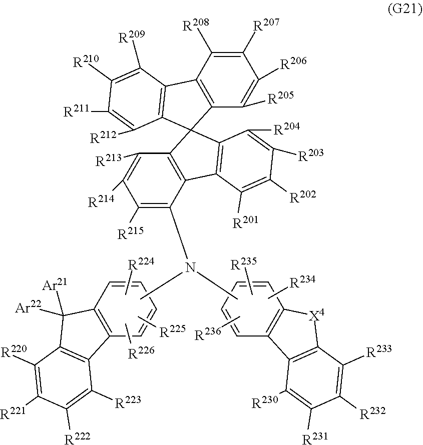
wherein Ar²¹ and Ar²² each independently represent a substituted or unsubstituted aryl group having 6 to 30 carbon atoms,
wherein R²⁰¹ to R²¹⁵, R²³⁰ to R²³⁶, and R²³⁰ to R²³⁶ each independently represent hydrogen, a substituted or unsubstituted alkyl group having 1 to 6 carbon atoms, a substituted or unsubstituted cycloalkyl group having 3 to 10 carbon atoms, a substituted or unsubstituted aryl group having 6 to 30 carbon atoms, or a substituted or unsubstituted heteroaryl group having 3 to 30 carbon atoms, and
wherein X⁴ represents sulfur or oxygen.

19. The organic compound according to claim 18,
wherein the organic compound is represented by General Formula (G22), 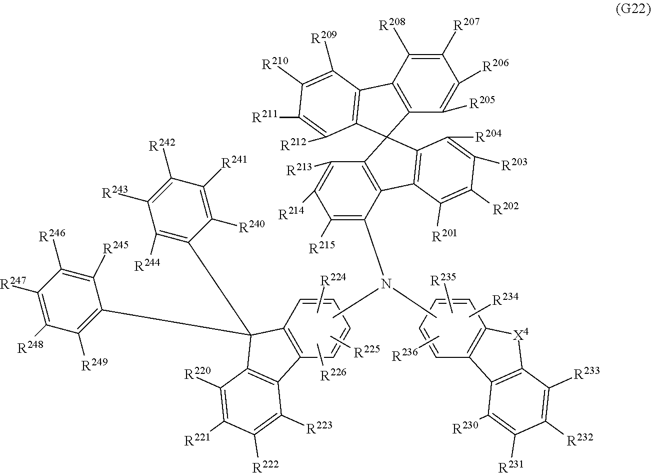
wherein R²⁰¹ to R²¹⁵, R²²⁰ to R²²⁶, R²³⁰ to R²³⁶, and R²⁴⁰ to R²⁴⁹ each independently represent hydrogen, a substituted or unsubstituted alkyl group having 1 to 6 carbon atoms, a substituted or unsubstituted cycloalkyl group having 3 to 10 carbon atoms, a substituted or unsubstituted aryl group having 6 to 30 carbon atoms, or a substituted or unsubstituted heteroaryl group having 3 to 30 carbon atoms, and
wherein X⁴ represents sulfur or oxygen.

20. The organic compound according to claim 18,
wherein the organic compound is represented by General Formula (G23),

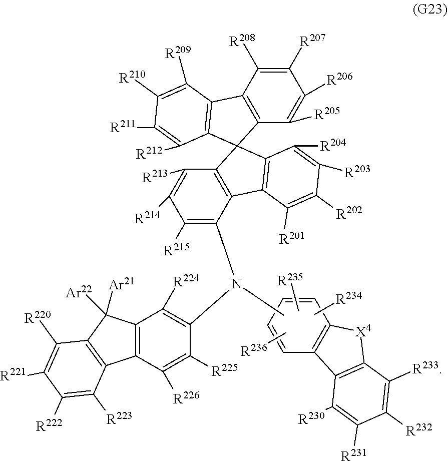

21. The organic compound according to claim 18,
wherein the organic compound is represented by General Formula (G24),

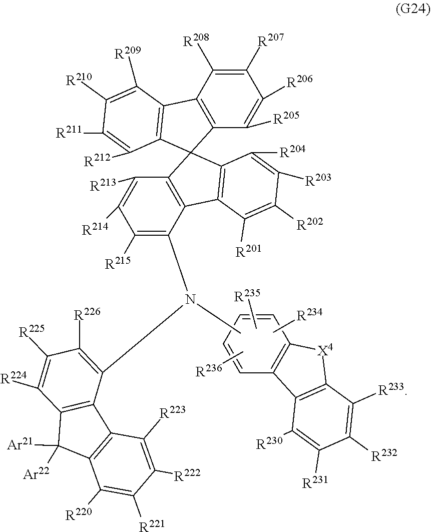

22. The organic compound according to claim 19,
wherein the organic compound is represented by General Formula (G25),

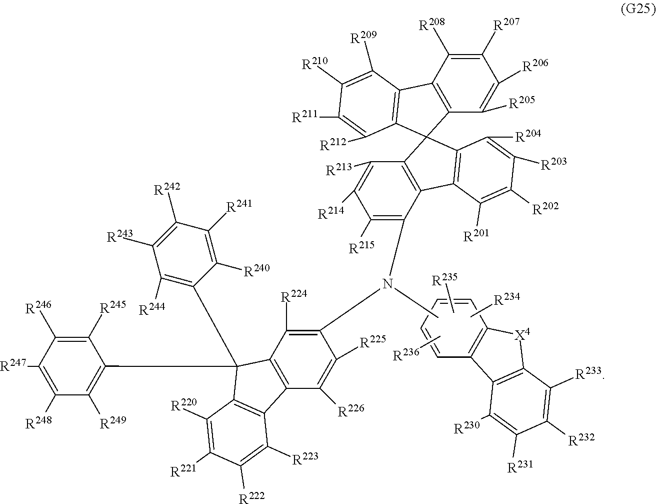

23. The organic compound according to claim 19,
wherein the organic compound is represented by General Formula (G26),

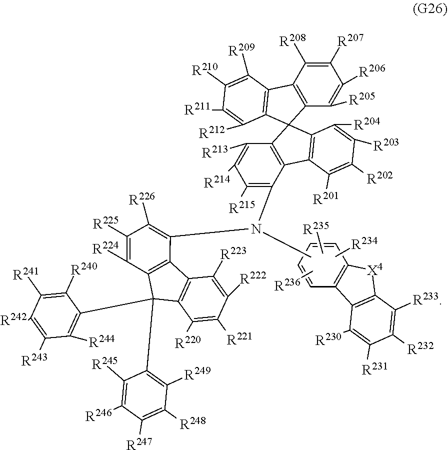

24. The organic compound according to claim 18,
wherein the organic compound is represented by General Formula (G27), 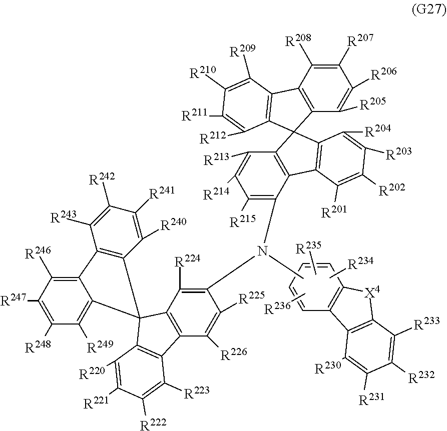
wherein R²⁰¹ to R²¹⁵, R²²⁰ to R²²⁶, R²³⁰ to R²³⁶, R²⁴⁰ to R²⁴³, and R²⁴⁶ to R²⁴⁹ each independently represent hydrogen, a substituted or unsubstituted alkyl group having 1 to 6 carbon atoms, a substituted or unsubstituted cycloalkyl group having 3 to 10 carbon atoms, a substituted or unsubstituted aryl group having 6 to 30 carbon atoms, or a substituted or unsubstituted heteroaryl group having 3 to 30 carbon atoms, and
wherein X⁴ represents sulfur or oxygen.

25. The organic compound according to claim 18,
wherein the organic compound is represented by General Formula (G28), 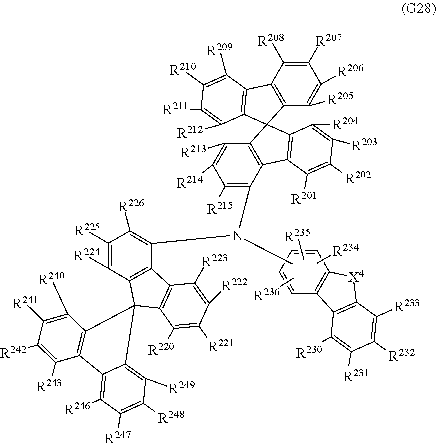
wherein R²⁰¹ to R²¹⁵, R²²⁰ to R²²⁶, R²³⁰ to R²³⁶, R²⁴⁰ to R²⁴³, and R²⁴⁶ to R²⁴⁹ each independently represent hydrogen, a substituted or unsubstituted alkyl group having 1 to 6 carbon atoms, a substituted or unsubstituted cycloalkyl group having 3 to 10 carbon atoms, a substituted or unsubstituted aryl group having 6 to 30 carbon atoms, or a substituted or unsubstituted heteroaryl group having 3 to 30 carbon atoms, and
wherein X⁴ represents sulfur or oxygen.

26. The organic compound according to claim 18,
wherein carbon at a 2-position of a dibenzofuranyl group or a dibenzothiophenyl group is bonded to nitrogen.

27. The organic compound according to claim 18,
wherein carbon at a 4-position of a dibenzofuranyl group or a dibenzothiophenyl group is bonded to nitrogen.

28. The organic compound according to claim 19,
wherein R²⁴⁰ and R²⁴⁵ are each a substituted or unsubstituted phenyl group.

29. The organic compound according to claim 19,
wherein R²⁴² and R²⁴⁷ are each a substituted or unsubstituted phenyl group.

30. A light-emitting device comprising the organic compound according to claim 18.
